(19) United States
(12) Patent Application Publication
(10) Pub. No.: US 2022/0144841 A1
(43) Pub. Date: May 12, 2022

(54) TRISUBSTITUTED PYRAZOLO [1,5-A] PYRIMIDINE COMPOUNDS AS CDK7 INHIBITORS

(71) Applicant: THE TRANSLATIONAL GENOMICS RESEARCH INSTITUTE, Phoenix, AZ (US)

(72) Inventors: Hariprasad VANKAYALAPATI, Draper, UT (US); Zhaoliang LI, Tempe, AZ (US); Trason THODE, Phoenix, AZ (US); Mohan KAADIGE, Scottsdale, AZ (US); Alexis WESTON, Phoenix, AZ (US); Sunil SHARMA, Phoenix, AZ (US)

(21) Appl. No.: 17/438,155

(22) PCT Filed: Mar. 13, 2020
(86) PCT No.: PCT/US2020/022708

§ 371 Date: Sep. 10, 2021

Related U.S. Application Data
(60) Provisional application No. 62/818,037, filed on Mar. 13, 2019.

(51) Int. Cl. C07D 487/04

(52) U.S. Cl. CPC C07D 487/04

(57) ABSTRACT

Compounds having activity as cancer agents are provided. The compounds have the following structure (I) or a pharmaceutically acceptable salts, stereoisomers, tautomers, thereof, wherein R¹, R², R³ and L are as defined herein. This disclosure provides methods associated with preparation and use of such compounds, pharmaceutical compositions comprising such compounds, and methods for treating a CDK7-dependent disease (e.g., cancer).

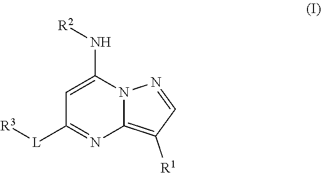

### BACKGROUND

### Technical Field

The present disclosure directed to novel pyrazolo[1,5-a]pyrimidine compounds of CDK7 and methods for their preparation and use as therapeutic or prophylactic agents, for example for treatment of cancer (e.g., solid tumors and hematological cancers).

### Description of the Related Art

Cancer is a group of diseases involving abnormal cell growth with a potential to spread to various parts of the body. Hundreds of types of cancers affect humans, and millions of people have been diagnosed and millions more are being diagnosed every year. The most common types of cancers include lung cancer, breast cancers, prostate cancers, colorectal cancers, among others. Treatment for cancers includes surgery, radiation therapy, chemotherapy, immunotherapy, hormone therapy, and stem cell replacement. Treatment options can be invasive and have a variety of undesirable side effects.

Accordingly, while the scientific community has made progress in this field, there remains a need in the art for improved compounds and methods for treatment of cancer. The present disclosure fulfills this need and provides further related advantages.

### BRIEF SUMMARY

In brief, embodiments of the present disclosure provide compounds, including pharmaceutically acceptable salts, stereoisomers, tautomers, isotopic forms or prodrugs thereof. Methods for use of such compounds for treatment of various diseases or conditions, such as cancers provided.

One embodiment provides a compound having the following structure (I):

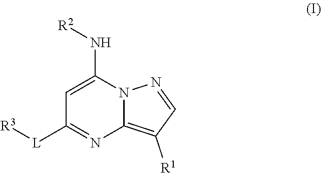

or a stereoisomer, tautomer, prodrug or pharmaceutically acceptable salt thereof, wherein R¹, R², R³ and L are as defined herein. Another embodiment provides pharmaceutical compositions comprising one or more compounds of structure (I) and a pharmaceutically acceptable carrier or excipient.

Other embodiments of the present disclosure provide a method for treatment of a disease (e.g., CDK7-dependent disease), the method comprising administering an effective amount of a compound of structure (I) or pharmaceutical composition comprising a compound of structure (I) to a subject in need thereof. These and other aspects of the disclosure will be apparent upon reference to the following detailed description.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5% or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

### Definitions

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Oxo” refers to the ═O substituent.

“Alkyl” refers to a saturated, straight or branched hydrocarbon chain radical consisting solely of carbon and hydrogen atoms, having from one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. Unless stated otherwise specifically in the specification, an alkyl group is optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double or an “alkenylene” and/or triple bonds or an “alkynylene”), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain is optionally substituted.

“Aromatic ring” refers to a cyclic planar portion of a molecule (i.e., a radical) with a ring of resonance bonds that exhibits increased stability relative to other connective arrangements with the same sets of atoms. Generally, aromatic rings contains a set of covalently bound co-planar atoms and comprises a number of π-electrons (for example, alternating double and single bonds) that is even but not a multiple of 4 (i.e., 4n+2 π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Aryl” refers to a carbocyclic ring system (i.e., a ring system wherein each ring atom is carbon) radical comprising 6 to 18 carbon ring atoms and at least one aromatic ring. For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Arylalkyl” refers to a radical of the formula —R_{b}R_{f} where R_{b} is an alkylene chain as defined above and R_{f} is an aryl radical as defined above. Unless stated otherwise specifically in the specification, an alkylaryl group is optionally substituted.

“Cycloalkyl” refers to a stable non-aromatic monocyclic or polycyclic carbocyclic radical, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Alkylsulfonyl” or “—S(O)₂alkyl” refers to a radical of the formula —S(O)₂Rₐ where Rₐ is an alkyl radical as defined above containing one to twelve carbon atoms. Unless stated specifically otherwise, an alkylsulfonyl is optionally substituted.

“Cycloalkylsulfonyl” or “—S(O)₂cycloalkyl” refers to a radical of the formula —S(O)₂Rₐ where Rₐ is an cycloalkyl radical as defined above containing one to twelve carbon atoms. Unless stated specifically otherwise, an cycloalkylsulfonyl is optionally substituted.

“Fused” refers to any ring structure described herein which is fused to an existing ring structure in the compounds of the disclosure. When the fused ring is a heterocyclyl ring or a heteroaryl ring, any carbon atom on the existing ring structure that becomes part of the fused heterocyclyl ring or the fused heteroaryl ring is replaced with a nitrogen atom.

“Halo” or “halogen” refers to bromo (Br), chloro (Cl), fluoro (F) or iodo (I).

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Heterocyclyl” or “heterocyclic ring” refers to a stable 3- to 18-membered non-aromatic ring radical having one to twelve ring carbon atoms (e.g., two to twelve) and from one to six ring heteroatoms selected from the group consisting of nitrogen, oxygen and sulfur. Unless stated otherwise specifically in the specification, the heterocyclyl radical is a monocyclic, bicyclic, tricyclic or tetracyclic ring system, which may include fused, spirocyclic (“spiro-heterocyclyl”) and/or bridged ring systems; and the nitrogen, carbon or sulfur atoms in the heterocyclyl radical is optionally oxidized; the nitrogen atom is optionally quaternized; and the heterocyclyl radical is partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,2,3,4-tetrahydroquinolinyl, and 1,1-dioxo-thiomorpholinyl. Unless stated otherwise specifically in the specification, a heterocyclyl group is optionally substituted.

“Hydroxyalkyl” refers to an alkyl group comprising at least one hydroxyl substituent. The —OH substituent may be on a primary, secondary or tertiary carbon. Unless stated otherwise specifically in the specification, a hydroxylalkyl group is optionally substituted.

“Trialkylsilyl” refers to a radical of formula —Si(Rₐ)(R_{b})(R_{c}) wherein Rₐ, R_{b}, and R_{c} are each independently an alkyl radical as defined above. For example, in some embodiments, each of Rₐ, R_{b}, and R_{c} is, independently C₁-C₆ alkyl. Examples of trialkylsilyl include but are not limited to trimethylsilyl, t-butyldimethylsilyl (TBS) or triisopropylsilyl (TIPS). Unless stated specifically otherwise, a trialkylsilyl is optionally substituted.

The term “substituted,” as it relates to any of the above groups, means that at least one hydrogen atom of the group (e.g., alkyl, alkylene, alkylsulfonyl, cycloalkylsulfonyl, aryl, alkylaryl, cycloalkyl, haloalkyl, heterocyclyl, hydroxyalkyl, and/or trialkylsilyl) is replaced by a bond to a non-hydrogen atom (or atoms). Examples of substituents include: halogens such as F, Cl, Br, and I; oxygen (e.g., hydroxyl groups, alkoxy groups, and ester groups); sulfur (e.g., thiol groups, thioalkyl groups, sulfone groups, sulfonyl groups, and sulfoxide groups); nitrogen (e.g., amines, amides, alkylamines, dialkylamines, arylamines, alkylarylamines, diarylamines, N-oxides, imides, and enamines); silicon (e.g., trialkylsilyl groups, dialkylarylsilyl groups, alkyldiarylsilyl groups, and triarylsilyl groups); and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with NR_{g}R_{b}, NR_{g}C(═O)Rₕ, NR_{g}C(═O)NR_{g}Rₕ, NR_{g}C(═O)ORₕ, NR_{g}SO₂Rₕ, OC(═O)NR_{g}Rₕ, OR_{g}, SR_{g}, SOR_{g}, SO₂R_{g}, OSO₂R_{g}, SO₂OR_{g}, ═NSO₂R_{g}, and SO₂NR_{g}Rₕ. “Substituted” also means any of the above groups in which one or more hydrogen atoms are replaced with C(═O)R_{g}, C(═O)OR_{g}, C(═O)NR_{g}Rₕ, CH₂SO₂R_{g}, CH₂SO₂NR_{g}Rₕ. In the foregoing, R_{g} and Rₕ are the same or different and independently hydrogen, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an aminyl, cyano, hydroxyl, imino, nitro, oxo, thioxo, halo, alkyl, alkoxy, alkylaminyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may be optionally substituted with one or more of the above substituents.

It is understood that each choice for R¹, R², R³, and L is optionally substituted as described above unless specifically stated otherwise, and provided that all valences are satisfied by the substitution. Specifically, each choice for R¹, R², R³, and L is optionally substituted unless specifically stated otherwise, and provided such substitution results in a stable molecule (e.g., groups such as H and halo are not optionally substituted).

The term “effective amount” or “therapeutically effective amount” refers to that amount of a compound described herein that is sufficient to effect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic benefit and/or a prophylactic benefit. “Therapeutic benefit” means eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The terms “antagonist” and “inhibitor” are used interchangeably, and they refer to a compound having the ability to inhibit a biological function of a target protein, whether by inhibiting the activity or expression of the protein, such as CDK7. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

As used herein, “agent” or “biologically active agent” refers to a biological, pharmaceutical, or chemical compound or other moiety. Non-limiting examples include a simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” refers to a biologically active agent refers to the agent's ability to preferentially reduce the target signaling activity as compared to off-target signaling activity, via direct or indirect interaction with the target.

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Radiation therapy” means exposing a subject, using routine methods and compositions known to the practitioner, to radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium-153-EDTMP, or high-energy radiation, including without limitation x-rays, gamma rays, and neutrons.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

Prodrugs of the disclosed compounds are included in various embodiments. “Prodrug” is meant to indicate a compound that may be converted under physiological conditions or by solvolysis to a biologically active compound described herein (e.g., compound of structure (I)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” includes any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of structure (I) being isotopically-labelled by having one or more atoms replaced by an atom having a different atomic mass or mass number (i.e., an “isotopic form” of a compound of structure (I)). Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically labeled compounds of structure (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence are preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Examples as set out below using an appropriate isotopically-labeled reagent in place of the non-labeled reagent previously employed.

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the embodiments include compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compound of the disclosure. As used herein, the term “solvate” refers to an aggregate that comprises one or more molecules of a compound of the disclosure with one or more molecules of solvent. In some embodiments, the solvent is water, in which case the solvate is a hydrate. Alternatively, in other embodiments, the solvent is an organic solvent. Thus, the compounds of structure (I) may exist as a hydrate, including a monohydrate, dihydrate, hemihydrate, sesquihydrate, trihydrate, tetrahydrate and the like, as well as the corresponding solvated forms. In some aspects, the compound of the disclosure is a true solvate, while in other cases, the compound of the disclosure merely retains adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The compounds of the disclosure (i.e., compounds of structure (I) and embodiments thereof), or their pharmaceutically acceptable salts may contain one or more centers of geometric asymmetry and may thus give rise to enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/solation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also included.

The present disclosure includes all manner of rotamers and conformationally restricted states of a compound of the disclosure.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Ultra Version 11.0.1 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

As detailed above, the present disclosure provides compounds showing significant activity as CDK7 inhibitors. Accordingly, one embodiment provides a compound having the following structure (I):

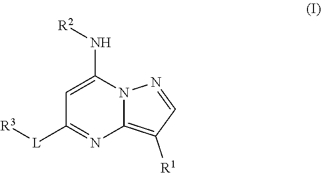

or stereoisomer, tautomer, prodrug, or pharmaceutically acceptable salt thereof, wherein:

R¹ is optionally substituted cycloalkyl, fluoro, chloro, or cyano;

R² is —C(═O)alkyl, optionally substituted aryl, or optionally substituted alkylaryl;

R³ is optionally substituted cycloalkyl or optionally substituted heterocyclyl;

L is —O(CH₂)ₙ— or —NH(CR^{a}R^{b})ₙ— wherein R^{a} and R^{b} are both hydrogen or R^{a} and R^{b} join together with the carbon to which they are attached to form oxo; and

n is 0 or 1,

wherein,

each cycloalkyl is independently unsubstituted or substituted with one or more substituents selected from hydroxyl, hydroxyalkyl, amino, and trialkylsilyl when R¹ is cycloalkyl and each cycloalkyl is independently unsubstituted or substituted with one or more substituents selected from hydroxyl, hydroxyalkyl, and trialkylsilyl when R¹ is chloro or cyano, and

each heterocyclyl, aryl, and arylalkyl is independently unsubstituted or substituted with one or more substituents selected from alkyl, alkenyl, alkynyl, halo, haloalkyl, alkoxy, haloalkoxy, cyano, hydroxyl, hydroxyalkyl, carboxy, heteroaryl, heterocyclyl, amino, —S(O₂)NH₂, —S(O₂)alkyl, —S(O₂)cycloalkyl, and trialkylsilyl.

In other embodiments of structure (I):

R¹ is optionally substituted cycloalkyl, chloro, or cyano;

R² is —C(═O)alkyl, optionally substituted aryl, or optionally substituted alkylaryl;

R³ is optionally substituted cycloalkyl or optionally substituted heterocyclyl;

L is —O(CH₂)ₙ— or —NH(CR^{a}R^{b})ₙ— wherein R^{a} and R^{b} are both hydrogen or R^{a} and R^{b} join together with the carbon to which they are attached to form oxo; and

n is 0 or 1,

wherein,

each cycloalkyl is independently unsubstituted or substituted with one or more substituents selected from hydroxyl, hydroxyalkyl, amino, and trialkylsilyl when R¹ is cycloalkyl and each cycloalkyl is independently unsubstituted or substituted with one or more substituents selected from hydroxyl, hydroxyalkyl, and trialkylsilyl when R¹ is chloro or cyano, and

each heterocyclyl, aryl, and arylalkyl is independently unsubstituted or substituted with one or more substituents selected from alkyl, alkenyl, alkynyl, halo, haloalkyl, alkoxy, haloalkoxy, cyano, hydroxyl, hydroxyalkyl, carboxy, heteroaryl, heterocyclyl, amino, —S(O₂)NH₂, —S(O₂)alkyl, —S(O₂)cycloalkyl, and trialkylsilyl.

In some embodiments, R¹ is optionally substituted cycloalkyl, for example unsubstituted cycloalkyl. In other embodiments, R¹ is chloro. In more embodiments R¹ is cyano. In other embodiments, R¹ is fluoro.

In some embodiments, R¹ is chloro, cyano, cyclopropyl, or cyclobutyl. In some embodiments, R¹ is substituted with one or more substituents. In some more specific embodiments, R¹ is substituted with halo. In some embodiments, R¹ is substituted with one or more fluoro substituents. In some more specific embodiments, the compound of structure (I) has one of the following structures (Ia), (Ib), (Ic), or (Id):

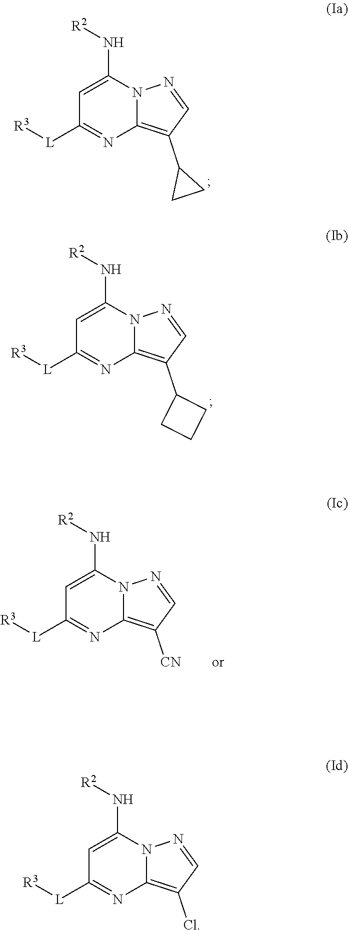

In some embodiments, the compound has structure (Ia). In some embodiments, the compound has structure (Ib). In some embodiments, the compound has structure (Ic). In some embodiments, the compound has structure (Id).

In some embodiments, R² is arylalkyl (e.g., benzyl). In some more specific embodiments, R² is substituted. In other specific embodiments, R² is substituted with one or more substituents selected from the group consisting of halo and alkyl. In some specific embodiments, R² is substituted with one or more substituents selected from the group consisting of fluoro, chloro, and methyl. In certain embodiments, R² has one of the following structures:

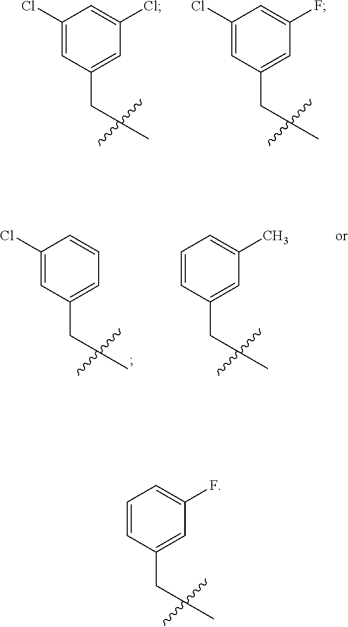

In some other embodiments, R² is aryl (e.g., phenyl). In some more specific embodiments, R² is substituted. In other specific embodiments, R² is substituted with one or more substituents selected from the group consisting of alkyl, halo, haloalkyl, cyano, —S(O₂)NH₂, and —S(O₂)alkyl. In more specific embodiments, R² is substituted with one or more substituents selected from the group consisting of fluoro, chloro, methyl, trifluoromethyl, trifluoroethyl, cyano,

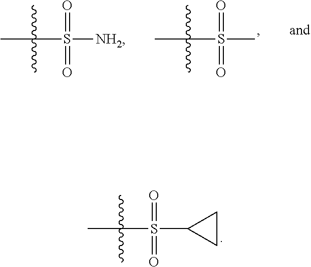

In certain specific embodiments, R² has one of the following structures:

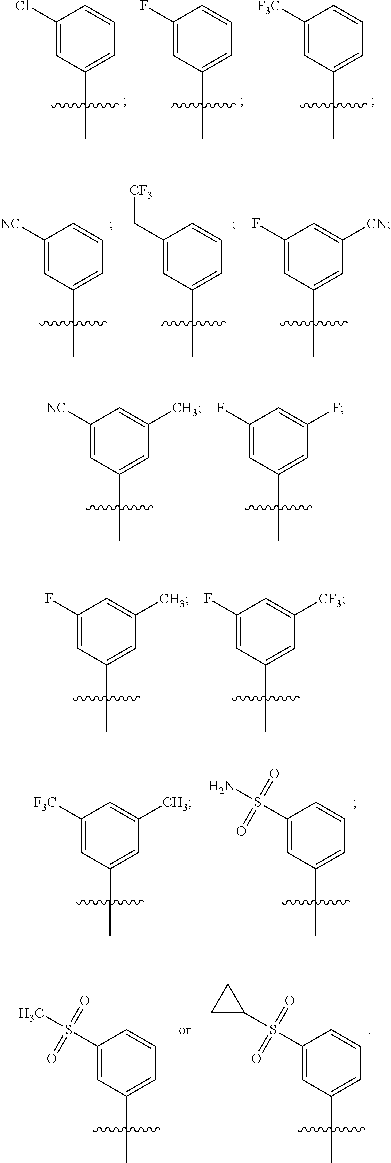

In some embodiments, R³ is heterocyclyl. (e.g., piperidinyl, azepinyl, or tetrahydropyranyl). In certain embodiments, R³ is unsubstituted. In more specific embodiments, R³ has one of the following structures:

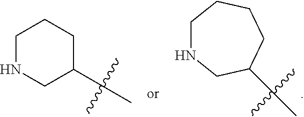 [SMILES: *C1CCCNC1 ; *C1CCCCNC1]

In some other embodiments, R³ is substituted. For example, in some embodiments, R³ is substituted piperidinyl, substituted azepinyl, or substituted tetrahydropyranyl. In more specific embodiments, R³ is substituted with one or more substituents selected from the group consisting of hydroxyl, and hydroxyalkyl. In still more specific embodiments, R³ has one of the following structures:

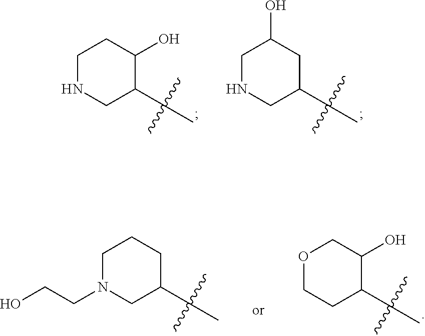 [SMILES: *C1CNCCC1O ; *C1CNCC(O)C1 ; *C1CCCN(CCO)C1 ; *C1CCOCC1O]

In some embodiments, R³ is cycloalkyl (e.g., cyclobutyl, cyclopentyl, or cyclohexyl). In more specific embodiments, R³ is substituted. In other embodiments, R³ is unsubstituted. In some more specific embodiments, R³ is substituted with one or more substituents selected from the group consisting of amino and trimethylsilyl. In still more specific embodiments, R³ has one of the following structures:

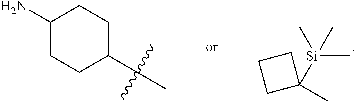

In some embodiments, L is —NH—, —N(H)CH₂— or —N(C═O)—. In other embodiments, L is —O— or —OCH₂—. In some specific embodiments, L is —NH—. In other embodiments, L is —N(H)CH₂—. In still other embodiments, L is —N(C═O)—. In some embodiments, L is —O—. In some embodiments, L is —OCH₂—.

In some embodiments, the compound of structure (I) has one of the following structures (Ia′) or (Ia″):

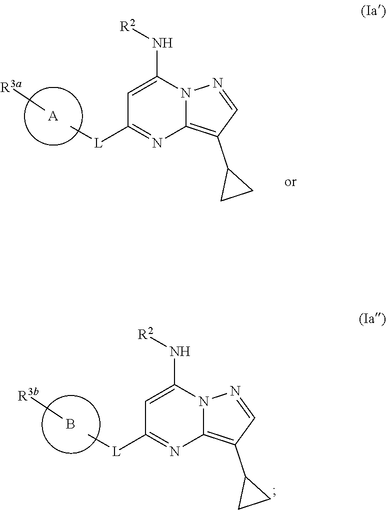

wherein

A is cycloalkyl;

B is heterocyclyl;

R^{3a} is hydrogen, hydroxyl, hydroxyalkyl, or trialkylsilyl; and

R^{3b} is hydrogen, hydroxyl, hydroxyalkyl, amine, and trialkylsilyl.

In more specific embodiments of structure (I), the compound has one of the following structures (Ia1), (Ia2), (Ia3), (Ia4), (Ia5), or (Ia6):

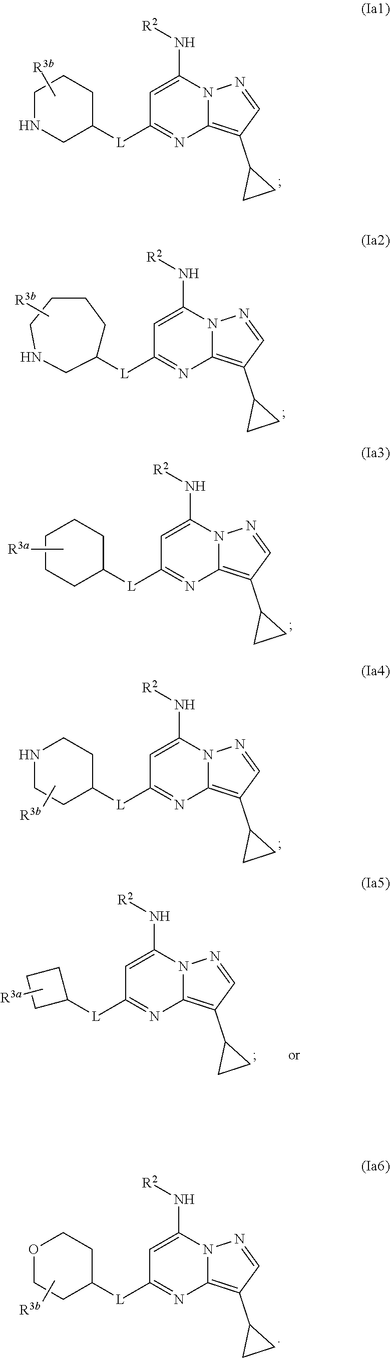

In other embodiments, the compound of structure (I) has one of the following structures (Ib1), (Ib2), (Ib3), or (Ib4):

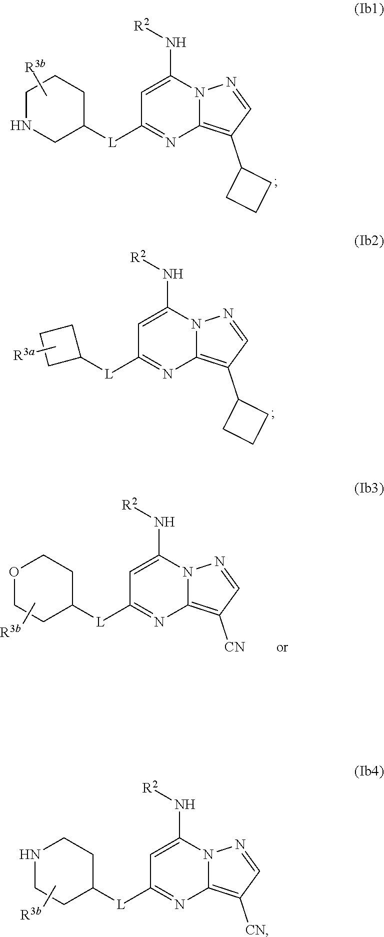

wherein

R^{3a} is hydrogen, hydroxyl, hydroxyalkyl, or trialkylsilyl; and

R^{3b} is hydrogen, hydroxyl, hydroxyalkyl, amine, and trialkylsilyl.

In still other embodiments of the compound of structure (I), the compound has the following structure (Ic1):

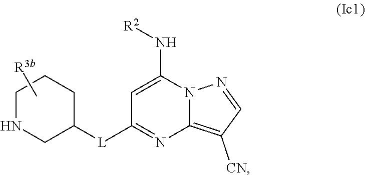

wherein

R^{3b} is hydrogen, hydroxyl, hydroxyalkyl, amine, and trialkylsilyl.

In some embodiments, the compound of structure (I) has one of the following structures (Id1) or (Id2):

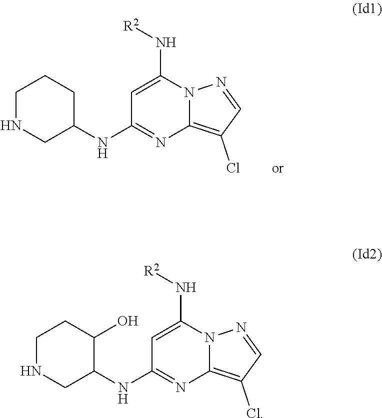

In some more specific embodiments of the compound of structure (I), the compound is selected from Table 1, below. In any one of the foregoing embodiments, a stereoisomer, tautomer, prodrug, or pharmaceutically acceptable salt thereof is also included.

TABLE 1
Representative compounds of Formula (I)

| No. | Structure | Name | Form |
|---|---|---|---|
| I-1 | 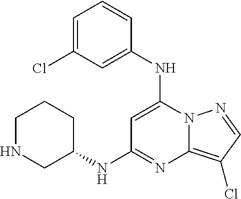 [SMILES: Clc1cccc(Nc2cc(N[C@H]3CCCNC3)nc3c(Cl)cnn23)c1] | (S)-3-chloro-N⁷-(3 - chlorophenyl)-N⁵-(piperidin- 3-yl)pyrazolo[1,5- a]pyrimidine-5,7-diamine | Free Base |
| I-2 | 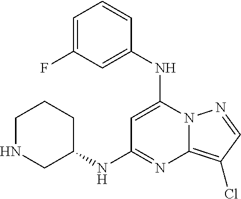 [SMILES: Fc1cccc(Nc2cc(N[C@H]3CCCNC3)nc3c(Cl)cnn23)c1] | (S)-3-chloro-N⁷-(3- fluorophenyl)-N⁵-(piperidin- 3-yl)pyrazolo[1,5- a]pyrimidine-5,7-diamine | Free Base |
| I-3 | 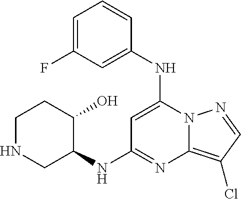 [SMILES: O[C@H]1CCNC[C@@H]1Nc1cc(Nc2cccc(F)c2)n2ncc(Cl)c2n1] | (3S,4S)-3-((3-chloro- 7-((3-fluorophenyl) amino)pyrazolo [1,5-a]pyrimidin-5- yl)amino)piperidin-4-ol | Free Base |
| I-4 | 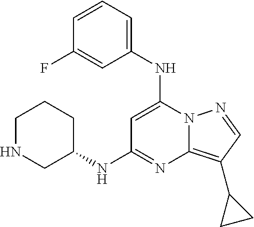 [SMILES: Fc1cccc(Nc2cc(N[C@H]3CCCNC3)nc3c(C4CC4)cnn23)c1] | (S)-3-cyclopropyl-N-(3- fluorophenyl)-5- ((piperidin-3- yl)oxy)pyrazolo[1,5- a]pyrimidin-7-amine | Free Base |
| I-5 | 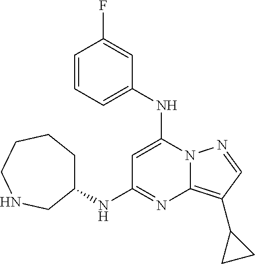 [SMILES: Fc1cccc(Nc2cc(N[C@H]3CCCCNC3)nc3c(C4CC4)cnn23)c1] | (S)-N⁵-(azepan-3-yl)-3- cyclopropyl-N⁷-(3- fluorophenyl)pyrazolo[1,5- a]pyrimidine-5,7-diamine | Free Base |
| I-6 | 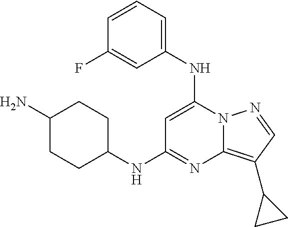 [SMILES: NC1CCC(Nc2cc(Nc3cccc(F)c3)n3ncc(C4CC4)c3n2)CC1] | Trans-N⁵-(4- aminocyclohexyl)-3- cyclopropyl-N⁷-(3- fluorophenyl)pyrazolo[1,5- a]pyrimidine-5,7-diamine | Free Base |
| I-7 | 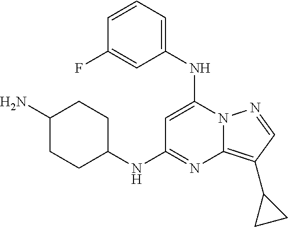 [SMILES: NC1CCC(Nc2cc(Nc3cccc(F)c3)n3ncc(C4CC4)c3n2)CC1] | Cis-N⁵-(4- aminocyclohexyl)- 3-cyclopropyl-N⁷-(3- fluorophenyl)pyrazolo[1,5- a]pyrimidine-5,7-diamine | Free Base |
| I-8 | 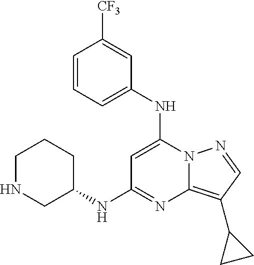 [SMILES: FC(F)(F)c1cccc(Nc2cc(N[C@H]3CCCNC3)nc3c(C4CC4)cnn23)c1] | (S)-3-cyclopropyl-N⁵- (piperidin-3-yl)-N⁷-((3- (trifluoromethyl)phenyl) pyrazolo[1,5-a]pyrimidine- 5,7-diamine | Free Base |
| I-9 | 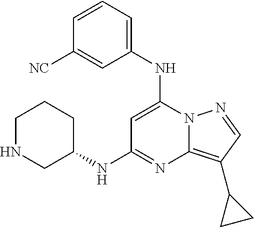 [SMILES: N#Cc1cccc(Nc2cc(N[C@H]3CCCNC3)nc3c(C4CC4)cnn23)c1] | (S)-3-((3-cyclopropyl-5- ((piperidin-3- yl)amino)pyrazolo[1,5- a]pyrimidin-7- yl)amino)benzonitrile | Free Base |
| I-10 | 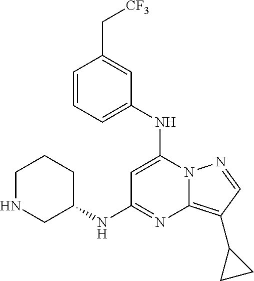 [SMILES: FC(F)(F)Cc1cccc(Nc2cc(N[C@H]3CCCNC3)nc3c(C4CC4)cnn23)c1] | (S)-3-cyclopropyl-N⁵- (piperidin-3-yl)-N⁷-(3-(2,2,2- trifluoroethyl)phenyl)pyrazolo [1,5-c]pyrimidine-5,7- diamine | Free Base |
| I-11 | 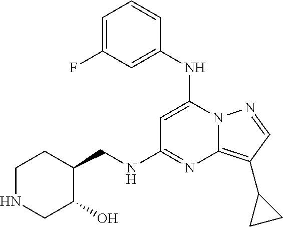 [SMILES: O[C@@H]1CNCC[C@H]1CNc1cc(Nc2cccc(F)c2)n2ncc(C3CC3)c2n1] | (3S,4S)-4-(((3-cyclopropyl-7- ((3- fluorophenyl)amino)pyrazolo [1,5-a]pyrimidin-5- yl)amino)methyl)piperidin- 3-ol | Free Base |
| I-12 | 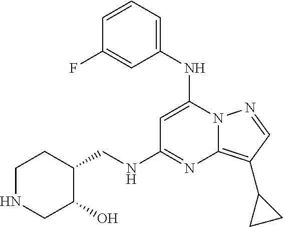 [SMILES: O[C@@H]1CNCC[C@@H]1CNc1cc(Nc2cccc(F)c2)n2ncc(C3CC3)c2n1] | (3S,4R)-4-((3-cyclopropyl-7- ((3-fluorophenyl) amino)pyrazolo [1,5-a]pyrimidin-5- yl)aminomethyl)piperidin- 3-ol | Free Base |
| I-13 | 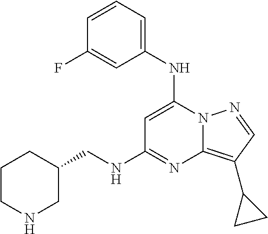 [SMILES: Fc1cccc(Nc2cc(NC[C@H]3CCCNC3)nc3c(C4CC4)cnn23)c1] | (S)-3-cyclopropyl-N⁷-(3- fluorophenyl)-N⁵-(piperidin- 3-ylmethyl)pyrazolo[1,5- c]pyrimidine-5,7-diamine | Free Base |
| I-14 | 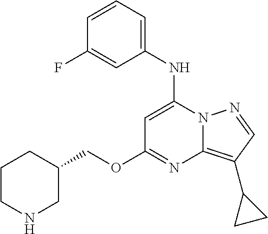 [SMILES: Fc1cccc(Nc2cc(OC[C@H]3CCCNC3)nc3c(C4CC4)cnn23)c1] | (S)-3-cyclopropyl-N-(3- fluorophenyl)-5-(piperidin-3- ylmethoxy)pyrazolo[1,5- c]pyrimidin-7-amine | Free Base |
| I-15 | 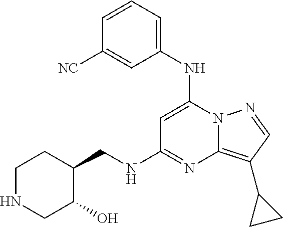 [SMILES: N#Cc1cccc(Nc2cc(NC[C@@H]3CCNC[C@H]3O)nc3c(C4CC4)cnn23)c1] | (3S,4S)-3-((3-cyclopropyl-5- (((3-hydroxy)piperidin-4- yl)methylamino)pyrazolo[1,5- a]pyrimidin-7- yl)amino)benzonitrile | Free Base |
| I-16 | 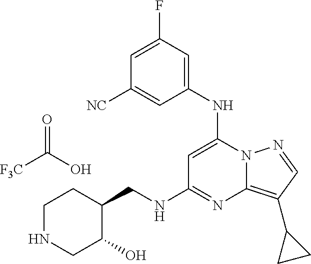 | (3S,4S)-3-((3-cyclopropyl-5- (((3-hydroxy)piperidin-4- yl)methylamino)pyrazolo[1,5- a]pyrimidin-7-yl)amino)-5- fluorobenzonitrile trifluoroacetate salt | TFA |
| I-17 | 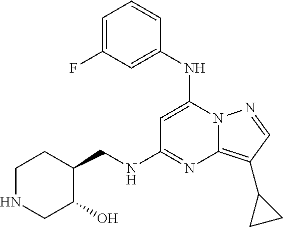 [SMILES: O[C@@H]1CNCC[C@H]1CNc1cc(Nc2cccc(F)c2)n2ncc(C3CC3)c2n1] | (3S,4S)-4-((3-cyclopropyl-7- ((3-fluorophenyl) amino)pyrazolo [1,5-alpyrimidin-5- yl)aminomethyl)piperidin- 3-ol | Free Base |
| I-18 | 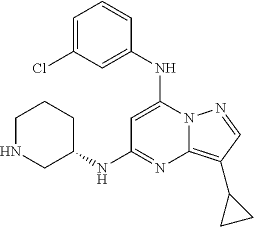 [SMILES: Clc1cccc(Nc2cc(N[C@H]3CCCNC3)nc3c(C4CC4)cnn23)c1] | (S)-N⁷-(3-chlorophenyl)-3- cyclopropyl-N⁵-(piperidin-3- yl)pyrazolo[1,5-c]pyrimidine- 5,7-diamine | Free Base |
| I-19 | 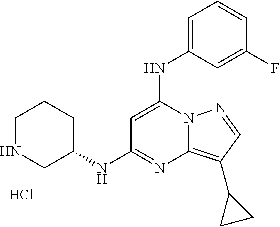 [SMILES: Cl ; Fc1cccc(Nc2cc(N[C@H]3CCCNC3)nc3c(C4CC4)cnn23)c1] | (S)-3-cyclopropyl-N⁷-(3- fluorophenyl)-N⁵-(piperidin- 3-yl)pyrazolo[1,5- c]pyrimidine-5,7-diamine hydrochloride | HCl |
| I-20 | 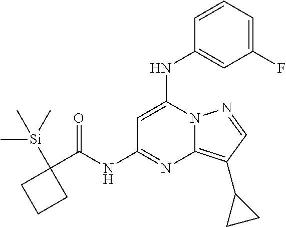 [SMILES: C[Si](C)(C)C1(C(=O)Nc2cc(Nc3cccc(F)c3)n3ncc(C4CC4)c3n2)CCC1] | N-(3-cyclopropyl-7-((3- fluorophenyl)amino)pyrazolo [1,5-a]pyrimidin-5-yl)-1- (trimethylsilyl)cyclobutane-1- carboxamide | Free Base |
| I-21 | 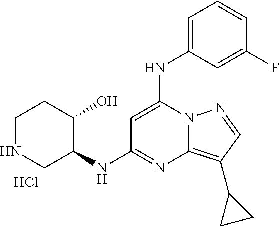 [SMILES: O[C@H]1CCNC[C@@H]1Nc1cc(Nc2cccc(F)c2)n2ncc(C3CC3)c2n1 ; Cl] | (3S,4S)-3-((3-cyclopropyl-7- ((3-fluorophenyl) amino)pyrazolo [1,5-a]pyrimidin-5- yl)amino)piperidin-4-ol hydrochloride | HCl |
| I-22 | 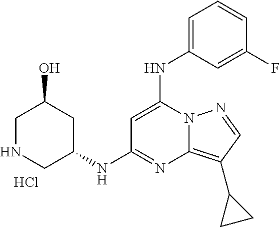 [SMILES: O[C@@H]1CNC[C@@H](Nc2cc(Nc3cccc(F)c3)n3ncc(C4CC4)c3n2)C1 ; Cl] | (3S,5S)-5-((3-cyclopropyl-7- ((3-fluorophenyl) amino)pyrazolo [1,5-c]pyrimidin-5- yl)amino)piperidin-3-ol hydrochloride | HCl |
| I-23 | 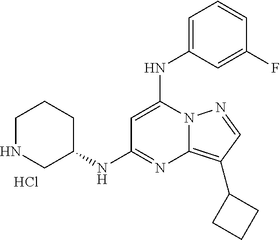 [SMILES: Fc1cccc(Nc2cc(N[C@H]3CCCNC3)nc3c(C4CCC4)cnn23)c1 ; Cl] | (S)-3-cyclobutyl-N⁷-(3- fluorophenyl)-N⁵-(piperidin- 3-yl)pyrazolo[1,5- a]pyrimidine-5,7-diamine hydrochloride | HCl |
| I-24 | 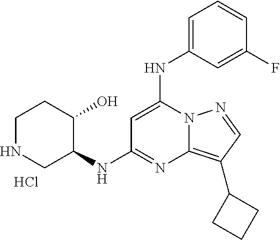 [SMILES: O[C@H]1CCNC[C@@H]1Nc1cc(Nc2cccc(F)c2)n2ncc(C3CCC3)c2n1 ; Cl] | (3S,4S)-3-((3-cyclobutyl-7- ((3-fluorophenyl) amino)pyrazolo [1,5-a]pyrimidin-5- yl)amino)piperidin-4-ol hydrochloride | HCl |
| I-25 | 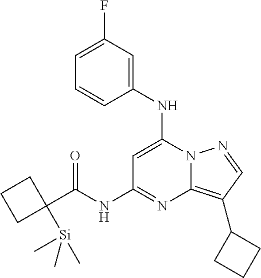 [SMILES: C[Si](C)(C)C1(C(=O)Nc2cc(Nc3cccc(F)c3)n3ncc(C4CCC4)c3n2)CCC1] | N-(3-cyclobutyl-7-((3- fluorophenyl)amino)pyrazolo [1,5-a]pyrimidin-5-yl)-1- (trimethylsilyl)cyclobutane- 1-carboxamide | Free Base |
| I-26 | 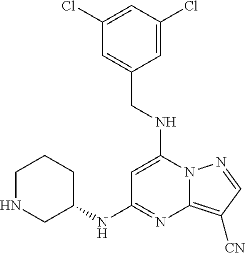 [SMILES: N#Cc1cnn2c(NCc3cc(Cl)cc(Cl)c3)cc(N[C@H]3CCCNC3)nc12] | (S)-7-((3,5- dichlorobenzyl)amino)-5- ((piperidin-3- yl)amino)pyrazolo[1,5- a]pyrimidine-3-carbonitrile | Free Base |
| I-27 | 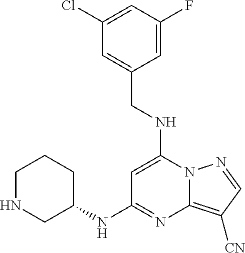 [SMILES: N#Cc1cnn2c(NCc3cc(F)cc(Cl)c3)cc(N[C@H]3CCCNC3)nc12] | (S)-7-((3-chloro-5- fluorobenzyl)amino)-5- ((piperidin-3- yl)amino)pyrazolo[1,5- a]pyrimidine-3-carbonitrile | Free Base |
| I-28 | 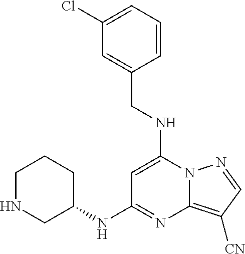 [SMILES: N#Cc1cnn2c(NCc3cccc(Cl)c3)cc(N[C@H]3CCCNC3)nc12] | (S)-7-((3- chlorobenzyl)amino)-5- ((piperidin-3- yl)amino)pyrazolo[1,5- a]pyrimidine-3-carbonitrile | Free Base |
| I-29 | 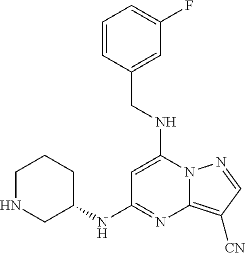 [SMILES: N#Cc1cnn2c(NCc3cccc(F)c3)cc(N[C@H]3CCCNC3)nc12] | (S)-7-((3- fluorobenzyl)amino)-5- ((piperidin-3- yl)amino)pyrazolo[1,5- a]pyrimidine-3-carbonitrile | Free Base |
| I-30 | 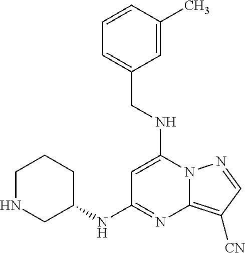 [SMILES: Cc1cccc(CNc2cc(N[C@H]3CCCNC3)nc3c(C#N)cnn23)c1] | (S)-7-((3- methylbenzyl)amino)-5- ((piperidin-3- yl)amino)pyrazolo[1,5- a]pyrimidine-3-carbonitrile | Free Base |
| I-31 | 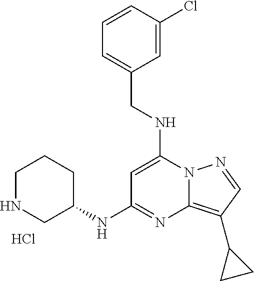 [SMILES: Clc1cccc(CNc2cc(N[C@H]3CCCNC3)nc3c(C4CC4)cnn23)c1 ; Cl] | (S)-N⁷-(3-chlorobenzyl)-3- cyclopropyl-N⁵-(piperidin-3- yl)pyrazolo[1,5-c]pyrimidine- 5,7-diamine hydrochloride | HCl |
| I-32 | 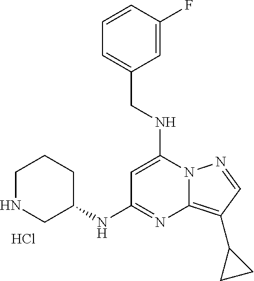 [SMILES: Fc1cccc(CNc2cc(N[C@H]3CCCNC3)nc3c(C4CC4)cnn23)c1 ; Cl] | (S)-3-cyclopropyl-N⁷-(3- fluorobenzyl)-N⁵-(piperidin- 3-yl)pyrazolo[1,5- a]pyrimidine-5,7-diamine hydrochloride | HCl |
| I-33 | 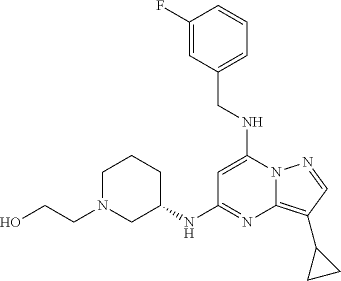 [SMILES: OCCN1CCC[C@H](Nc2cc(NCc3cccc(F)c3)n3ncc(C4CC4)c3n2)C1] | (S)-2-(3-((3-cyclopropyl-7- ((3-fluorobenzyl) amino)pyrazolo [1,5-a]pyrimidin-5- yl)amino)piperidin-1- yl)ethan-l-ol | Free Base |
| I-34 | 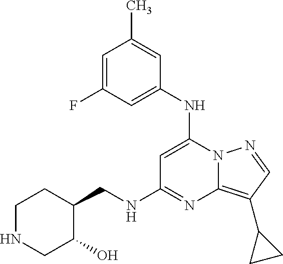 [SMILES: Cc1cc(F)cc(Nc2cc(NC[C@@H]3CCNC[C@H]3O)nc3c(C4CC4)cnn23)c1] | (3S,4S)-4-(((3-cyclopropyl-7- ((3-fluoro-5- methylphenyl)amino)pyrazolo [1,5-c]pyrimidin-5- yl)amino)methyl) piperidin-3-ol | Free Base |
| I-35 | 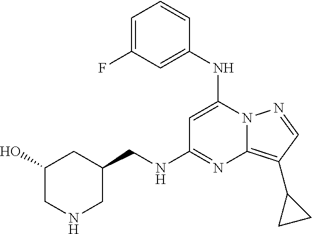 [SMILES: O[C@H]1CNC[C@H](CNc2cc(Nc3cccc(F)c3)n3ncc(C4CC4)c3n2)C1] | (3R,5R)-5-(((3-cyclopropyl-7- ((3-fluorophenyl) amino)pyrazolo [1,5-a]pyrimidin-5- yl)amino)methyl)piperidin- 3-ol | Free Base |
| I-36 | 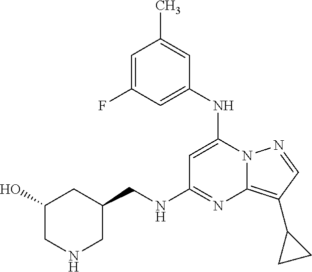 [SMILES: Cc1cc(F)cc(Nc2cc(NC[C@H]3CNC[C@H](O)C3)nc3c(C4CC4)cnn23)c1] | (3R,5R)-5-(((3-cyclopropyl-7- ((3-fluoro-5- methylphenyl)amino)pyrazolo [1,5-c]pyrimidin-5- yl)amino)methyl) piperidin-3-ol | Free Base |
| I-37 | 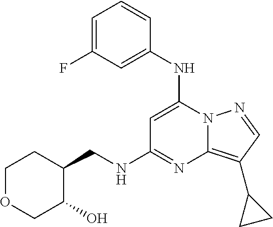 [SMILES: O[C@@H]1COCC[C@H]1CNc1cc(Nc2cccc(F)c2)n2ncc(C3CC3)c2n1] | (3S,4S)-4-(((3-cyclopropyl-7- ((3-fluorophenyl) amino)pyrazolo [1,5-c]pyrimidin-5- yl)amino)methyl) tetrahydro-2H-pyran-3-ol | Free Base |
| I-38 | 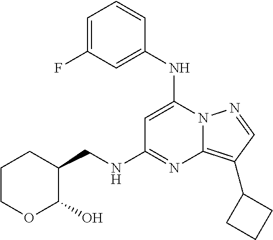 [SMILES: O[C@@H]1OCCC[C@H]1CNc1cc(Nc2cccc(F)c2)n2ncc(C3CCC3)c2n1] | (3S,4S)-4-(((3-cyclobutyl-7- ((3-fluorophenyl) amino)pyrazolo [1,5-a]pyrimidin-5- yl)amino)methyl)tetrahydro- 2H-pyran-3-ol | Free Base |
| I-39 | 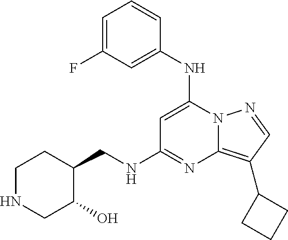 [SMILES: O[C@@H]1CNCC[C@H]1CNc1cc(Nc2cccc(F)c2)n2ncc(C3CCC3)c2n1] | (3S,4S)-4-(((3-cyclobutyl-7- ((3-fluorophenyl) amino)pyrazolo [1,5-a]pyrimidin-5- yl)amino)methyl)piperidin- 3-ol | Free Base |
| I-40 | 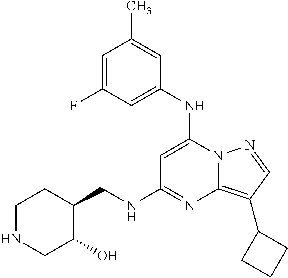 [SMILES: Cc1cc(F)cc(Nc2cc(NC[C@@H]3CCNC[C@H]3O)nc3c(C4CCC4)cnn23)c1] | (3S,4S)-4-(((3-cyclobutyl- 7-((3-fluoro-5- methylphenyl)amino)pyrazolo [1,5-a]pyrimidin-5- yl)amino)methyl)piperidin- 3-ol | Free Base |
| I-41 | 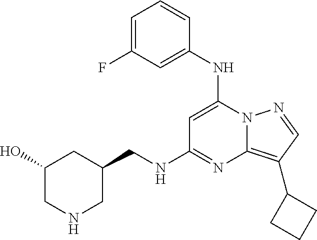 [SMILES: O[C@H]1CNC[C@H](CNc2cc(Nc3cccc(F)c3)n3ncc(C4CCC4)c3n2)C1] | (3R,5R)-5-(((3-cyclobutyl- 7-((3- fluorophenyl)amino)pyrazolo [1,5-a]pyrimidin-5- yl)amino)methyl)piperidin- 3-ol | Free Base |
| I-42 | 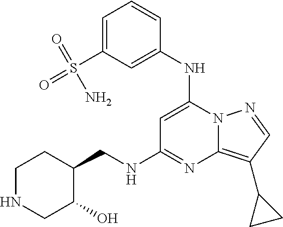 [SMILES: NS(=O)(=O)c1cccc(Nc2cc(NC[C@@H]3CCNC[C@H]3O)nc3c(C4CC4)cnn23)c1] | 3-((3-cyclopropyl-5- ((((3S,4S)-3- hydroxypiperidin-4- yl)methyl)amino)pyrazolo[1,5 -a]pyrimidin-7- yl)amino)benzenesulfonamide | Free Base |
| I-43 | 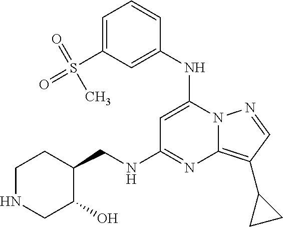 [SMILES: CS(=O)(=O)c1cccc(Nc2cc(NC[C@@H]3CCNC[C@H]3O)nc3c(C4CC4)cnn23)c1] | (3S,4S)-4-(((3-cyclopropyl- 7-((3- (methylsulfonyl)phenyl) amino)pyrazolo [1,5-a]pyrimidin-5- yl)amino)methyl)piperidin- 3-ol | Free Base |
| I-44 | 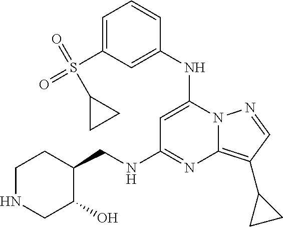 [SMILES: O=S(=O)(c1cccc(Nc2cc(NC[C@@H]3CCNC[C@H]3O)nc3c(C4CC4)cnn23)c1)C1CC1] | (3S,4S)-4-(((3-cyclopropyl- 7-((3- (cyclopropylsulfonyl) phenyl)amino) pyrazolo[1,5-a]pyrimidin-5- yl)amino)methyl)piperidin- 3-ol | Free Base |
| I-45 | 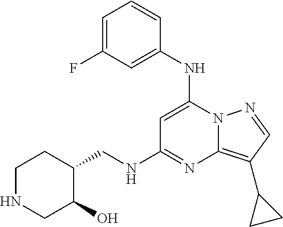 [SMILES: O[C@H]1CNCC[C@@H]1CNc1cc(Nc2cccc(F)c2)n2ncc(C3CC3)c2n1] | (3R,4R)-4-((3-cyclopropyl- 7-((3- fluorophenyl)amino)pyrazolo [1,5-a]pyrimidin-5- yl)aminomethyl)piperidin- 3-ol | Free Base |
| I-46 | 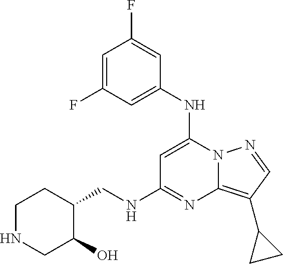 [SMILES: O[C@H]1CNCC[C@@H]1CNc1cc(Nc2cc(F)cc(F)c2)n2ncc(C3CC3)c2n1] | (3R,4R)-4-((3-cyclopropyl- 7-((3,5- difluorophenyl)amino) pyrazolo[1,5-a]pyrimidin- 5-yl)aminomethyl)piperidin- 3-ol | Free Base |
| I-47 | 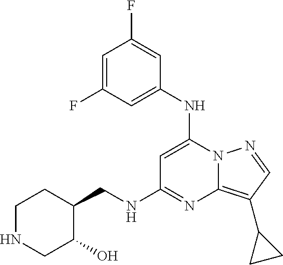 [SMILES: O[C@@H]1CNCC[C@H]1CNc1cc(Nc2cc(F)cc(F)c2)n2ncc(C3CC3)c2n1] | (3S,4S)-4-((3-cyclopropyl- 7-((3,5- difluorophenyl)amino) pyrazolo[1,5-a]pyrimidin-5- yl)aminomethyl)piperidin-3-ol | Free Base |
| I-48 | 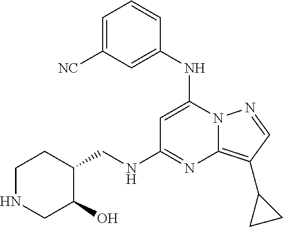 [SMILES: N#Cc1cccc(Nc2cc(NC[C@H]3CCNC[C@@H]3O)nc3c(C4CC4)cnn23)c1] | (3R,4R)-3-(((3-cyclopropyl- 5-(3-hydroxypiperidin-4- yl)methylamino)pyrazolo[1,5- a]pyrimidin-7- yl)amino)benzonitrile | Free Base |
| I-49 | 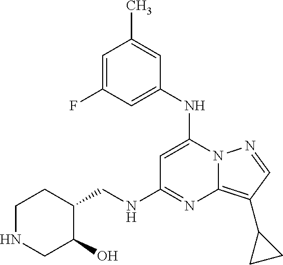 [SMILES: Cc1cc(F)cc(Nc2cc(NC[C@H]3CCNC[C@@H]3O)nc3c(C4CC4)cnn23)c1] | (3R,4R)-4-((3-cyclopropyl-7- ((3-fluoro-5- methylphenyl)amino)pyrazolo [1,5-a]pyrimidin-5- yl)aminomethyl)piperidin- 3-ol | Free Base |
| I-50 | 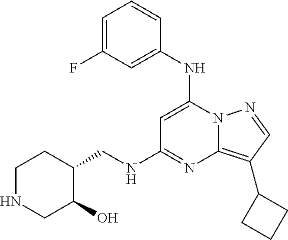 [SMILES: O[C@H]1CNCC[C@@H]1CNc1cc(Nc2cccc(F)c2)n2ncc(C3CCC3)c2n1] | (3R,4R)-4-((3-cyclobutyl- 7-((3- fluorophenyl)amino)pyrazolo [1,5-alpyrimidin-5- yl)aminomethyl)piperidin-3-ol | Free Base |
| I-51 | 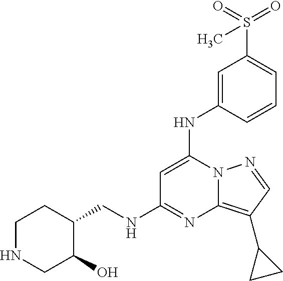 [SMILES: CS(=O)(=O)c1cccc(Nc2cc(NC[C@H]3CCNC[C@@H]3O)nc3c(C4CC4)cnn23)c1] | (3R,4R)-((3-cyclopropyl- 7-((3- methylsulfonylphenyl)amino) pyrazolo[1,5-a]pyrimidin-5- yl)aminomethyl)piperidin-3-ol | Free Base |
| I-52 | 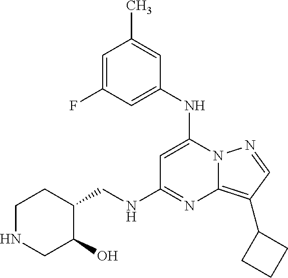 [SMILES: Cc1cc(F)cc(Nc2cc(NC[C@H]3CCNC[C@@H]3O)nc3c(C4CCC4)cnn23)c1] | (3R,4R)-((3-cyclobutyl-7- ((3-fluoro-5- methylphenyl)amino)pyrazolo [1,5-a]pyrimidin-5- yl)aminomethyl)piperidin-3-ol | Free Base |
| I-53 | 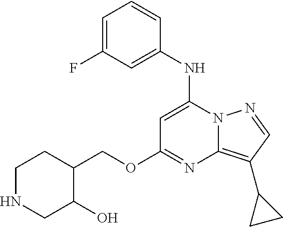 [SMILES: OC1CNCCC1COc1cc(Nc2cccc(F)c2)n2ncc(C3CC3)c2n1] | trans-4-((3-cyclopropyl-7-((3- fluorophenyl)amino)pyrazolo [1,5-a]pyrimidin-5- yl)oxymethyl)piperidin-3-ol | Free Base |
| I-54 | 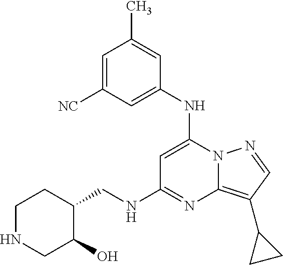 [SMILES: Cc1cc(C#N)cc(Nc2cc(NC[C@H]3CCNC[C@@H]3O)nc3c(C4CC4)cnn23)c1] | (3R,4R)-3-((3-cyclopropyl-5- ((3-hydroxypiperidin-4- yl)methylamino)pyrazolo[1,5- a]pyrimidin-7-yl)amino)-5- methylbenzonitrile | Free Base |
| I-55 | 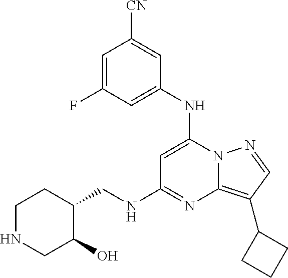 [SMILES: N#Cc1cc(F)cc(Nc2cc(NC[C@H]3CCNC[C@@H]3O)nc3c(C4CCC4)cnn23)c1] | (3R,4R)-3-((3-cyclobutyl-5- ((3-hydroxypiperidin-4- yl)methylamino)pyrazolo[1,5- a]pyrimidin-7-yl)amino)-5- fluorobenzonitrile | Free Base |
| I-56 | 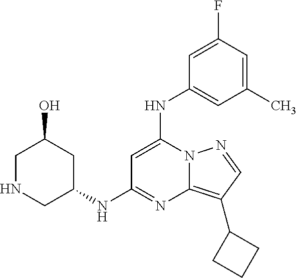 [SMILES: Cc1cc(F)cc(Nc2cc(N[C@@H]3CNC[C@@H](O)C3)nc3c(C4CCC4)cnn23)c1] | (3S,5S)-5-((3-cyclobutyl-7- ((3-fluoro-5- methylphenyl)amino)pyrazolo [1,5-a]pyrimidin-5- yl)amino)piperidin-3-ol | Free Base |
| I-57 | 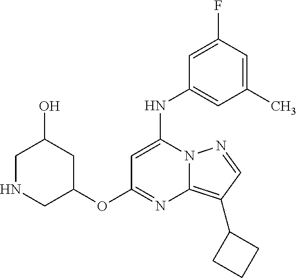 [SMILES: Cc1cc(F)cc(Nc2cc(OC3CNCC(O)C3)nc3c(C4CCC4)cnn23)c1] | trans-5-((3-cyclobutyl-7-((3- fluoro-5- methylphenyl)amino)pyrazolo [1,5-a]pyrimidin-5- yl)oxy)piperidin-3-ol | Free Base |
| I-58 | 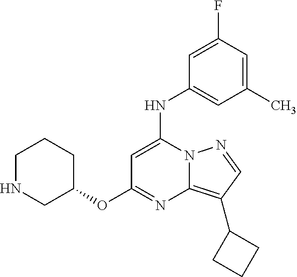 [SMILES: Cc1cc(F)cc(Nc2cc(O[C@H]3CCCNC3)nc3c(C4CCC4)cnn23)c1] | (S)-3-cyclobutyl-N-(3-fluoro- 5-methylphenyl)-5- ((piperidin-3- yl)oxy)pyrazolo[1,5- a]pyrimidin-7-amine | Free Base |
| I-59 | 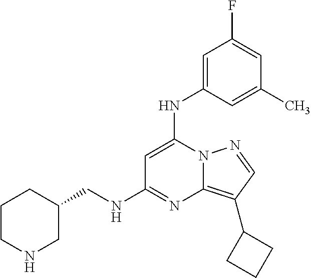 [SMILES: Cc1cc(F)cc(Nc2cc(NC[C@H]3CCCNC3)nc3c(C4CCC4)cnn23)c1] | (S)-3-cyclobutyl-N⁷-(3-fluoro- 5-methylphenyl)-N⁵- ((piperidin-3- yl)methyl)pyrazolo[1,5- a]pyrimidine-5,7-diamine | Free Base |
| I-60 | 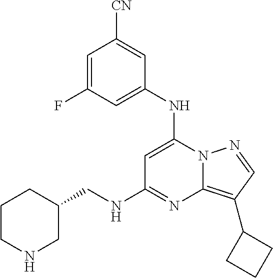 [SMILES: N#Cc1cc(F)cc(Nc2cc(NC[C@H]3CCCNC3)nc3c(C4CCC4)cnn23)c1] | (S)-3-((3-cyclobutyl-5- ((piperidin-3- yl)methoxy)pyrazolo[1,5- a]pyrimidin-7-yl)amino)-5- fluorobenzonitrile | Free Base |
| I-61 | 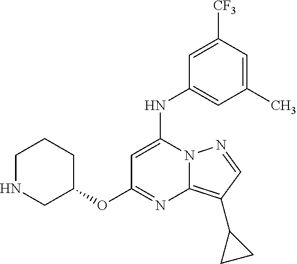 [SMILES: Cc1cc(Nc2cc(O[C@H]3CCCNC3)nc3c(C4CC4)cnn23)cc(C(F)(F)F)c1] | (S)-3-cyclopropyl-N-(5- methyl-3- trifluoromethylphenyl)-5- ((piperidin-3- yl)oxy)pyrazolo[1,5- a]pyrimidin-7-amine | Free Base |
| I-62 | 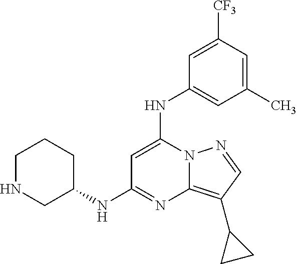 [SMILES: Cc1cc(Nc2cc(N[C@H]3CCCNC3)nc3c(C4CC4)cnn23)cc(C(F)(F)F)c1] | (S)-3-cyclopropyl-N⁷-(5- methyl-3- trifluoromethylphenyl)-N⁵- (piperidin-3-yl)pyrazolo[1,5- a]pyrimidine-5,7-diamine | Free Base |
| I-63 | 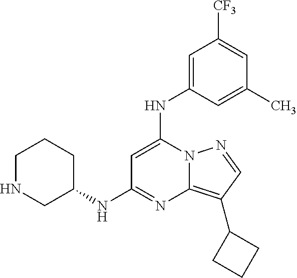 [SMILES: Cc1cc(Nc2cc(N[C@H]3CCCNC3)nc3c(C4CCC4)cnn23)cc(C(F)(F)F)c1] | (S)-3-cyclobutyl-N⁷-(5- methyl-3- trifluoromethylphenyl)-N⁵- (piperidin-3-yl)pyrazolo[1,5- a]pyrimidine-5,7-diamine | Free Base |
| I-64 | 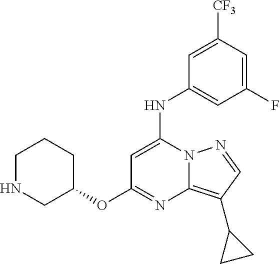 [SMILES: Fc1cc(Nc2cc(O[C@H]3CCCNC3)nc3c(C4CC4)cnn23)cc(C(F)(F)F)c1] | (S)-3-cyclopropyl-N-(5- fluoro-3- trifluoromethylphenyl)-5- ((piperidin-3- yl)oxy)pyrazolo[1,5- a]pyrimidin-7-amine | Free Base |
| I-65 | 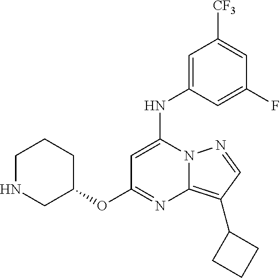 [SMILES: Fc1cc(Nc2cc(O[C@H]3CCCNC3)nc3c(C4CCC4)cnn23)cc(C(F)(F)F)c1] | (S)-3-cyclobutyl-N-(5-fluoro- 3-trifluoromethylphenyl)- 5-((piperidin-3- yl)oxy)pyrazolo[1,5- a]pyrimidin-7-amine | Free Base |
| I-66 | 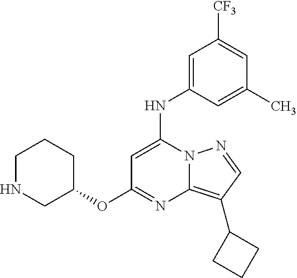 [SMILES: Cc1cc(Nc2cc(O[C@H]3CCCNC3)nc3c(C4CCC4)cnn23)cc(C(F)(F)F)c1] | (S)-3-cyclobutyl-N-(5-methyl- 3-trifluoromethylphenyl)-5- ((piperidin-3- yl)oxy)pyrazolo[1,5- a]pyrimidin-7-amine | Free Base |
| I-67 | 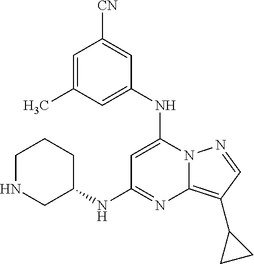 [SMILES: Cc1cc(C#N)cc(Nc2cc(N[C@H]3CCCNC3)nc3c(C4CC4)cnn23)c1] | (S)-3-((3-cyclopropyl-5- ((piperidin-3- yl)amino)pyrazolo[1,5- a]pyrimidin-7-yl)amino)-5- methylbenzonitrile | Free Base |
| I-68 | 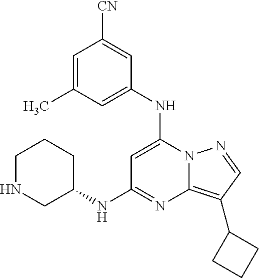 [SMILES: Cc1cc(C#N)cc(Nc2cc(N[C@H]3CCCNC3)nc3c(C4CCC4)cnn23)c1] | (S)-3-((3-cyclobutyl-5- ((piperidin-3- yl)amino)pyrazolo[1,5- a]pyrimidin-7-yl)amino)-5- methylbenzonitrile | Free Base |
| I-69 | 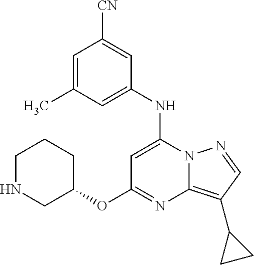 [SMILES: Cc1cc(C#N)cc(Nc2cc(O[C@H]3CCCNC3)nc3c(C4CC4)cnn23)c1] | (S)-3-((3-cyclopropyl-5- ((piperidin-3-yl)oxy)pyrazolo [1,5-a]pyrimidin-7-yl)amino)- 5-methylbenzonitrile | Free Base |
| I-70 | 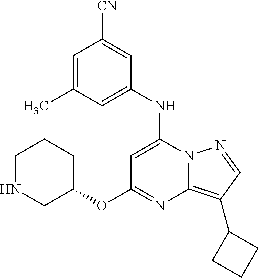 [SMILES: Cc1cc(C#N)cc(Nc2cc(O[C@H]3CCCNC3)nc3c(C4CCC4)cnn23)c1] | (S)-3-((3-cyclobutyl-5- ((piperidin-3- yl)oxy)pyrazolo[1,5- a]pyrimidin-7-yl)amino)-5- methylbenzonitrile | Free Base |

### Pharmaceutical Compositions

Other embodiments are directed to pharmaceutical compositions. The pharmaceutical composition comprises any one (or more) of the foregoing compounds of structure (I) (or pharmaceutically acceptable salt thereof) and a pharmaceutically acceptable carrier or excipient. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent. Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound of structure (I) is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound of structure (I) is provided in the form of a rapid release formulation, in the form of an extended release formulation, or in the form of an intermediate release formulation. In yet other embodiments, the compound of structure (I) is administered topically.

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that are used in some embodiments. An exemplary dosage is to 30 mg per day. The exact dosage will depend upon the route of administration, the form in which the compound is administered, the subject to be treated, the body weight of the subject to be treated, and the preference and experience of the attending physician.

In some embodiments, a compound of the disclosure is administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, a compound of the disclosure is administered in multiple doses. In some embodiments, dosing is about once, twice, three times, four times, five times, six times, or more than six times per day. In other embodiments, dosing is about once a month, once every two weeks, once a week, or once every other day. In another embodiment a compound of the disclosure and another agent are administered together about once per day to about 6 times per day. In another embodiment the administration of a compound of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the disclosure may continue as long as necessary. In some embodiments, a compound of the disclosure is administered for more than 1, 2, 3, 4, 5, 6, 7, 14, or 28 days. In some embodiments, a compound of the disclosure is administered for less than 28, 14, 7, 6, 5, 4, 3, 2, or 1 day. In some embodiments, a compound of the disclosure is administered chronically on an ongoing basis, e.g., for the treatment of chronic effects.

In some embodiments, the compounds of the disclosure are administered in dosages. It is known in the art that due to intersubject variability in compound pharmacokinetics, individualization of dosing regimen is necessary for optimal therapy. Dosing for a compound of the disclosure may be found by routine experimentation in light of the instant disclosure.

In some embodiments, the compounds of structure (I) are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound of structure (I) and a pharmaceutically acceptable diluent(s), excipient(s), or carrier(s). In certain embodiments, the compounds described are administered as pharmaceutical compositions in which compounds of structure (I) are mixed with other active ingredients, as in combination therapy. Encompassed herein are all combinations of actives set forth in the combination therapies section below and throughout this disclosure. In specific embodiments, the pharmaceutical compositions include one or more compounds of structure (I).

A pharmaceutical composition, as used herein, refers to a mixture of a compound of structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of structure (I) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds of structure (I) are used singly or in combination with one or more therapeutic agents as components of mixtures.

In one embodiment, one or more compounds of structure (I) is formulated in an aqueous solutions. In specific embodiments, the aqueous solution is selected from, by way of example only, a physiologically compatible buffer, such as Hank's solution, Ringer's solution, or physiological saline buffer. In other embodiments, one or more compound of structure (I) is/are formulated for transmucosal administration. In specific embodiments, transmucosal formulations include penetrants that are appropriate to the barrier to be permeated. In still other embodiments wherein the compounds of structure (I) are formulated for other parenteral injections, appropriate formulations include aqueous or non-aqueous solutions. In specific embodiments, such solutions include physiologically compatible buffers and/or excipients.

In another embodiment, compounds of structure (I) are formulated for oral administration. Compounds of structure (I) are formulated by combining the active compounds with, e.g., pharmaceutically acceptable carriers or excipients. In various embodiments, the compounds of structure (I) are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds of structure (I), optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds of structure (I) are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds of structure (I) are formulated for buccal or sublingual administration. Formulations suitable for buccal or sublingual administration include, by way of example only, tablets, lozenges, or gels. In still other embodiments, the compounds of structure (I) are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds (e.g., compounds of structure (I)) are prepared as appropriate oily injection suspensions. Suitable lipophilic solvents or vehicles for use in the pharmaceutical compositions of structure (I) include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the compounds of structure (I) are administered topically. The compounds of structure (I) are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds of structure (I) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of structure (I) is accomplished by means of iontophoretic patches and the like. In certain embodiments, transdermal patches provide controlled delivery of the compounds of structure (I). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the compounds of structure (I) are formulated for administration by inhalation. Various forms suitable for administration by inhalation include, but are not limited to, aerosols, mists or powders. Pharmaceutical compositions of any of compound of structure (I) are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

In still other embodiments, the compounds of structure (I) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of structure (I) are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of structure (I), as an active ingredient. The active ingredient is in free-acid or free-base form, or in a pharmaceutically acceptable salt form. In addition, the methods and pharmaceutical compositions of structure (I) include the use of N-oxides, crystalline forms (also known as polymorphs), as well as active metabolites of these compounds having the same type of activity. All tautomers of the compounds of structure (I) are included within the scope of the compounds presented herein. Additionally, the compounds of structure (I) encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Accordingly, one embodiment provides a pharmaceutically acceptable salt of any one of the compounds of structure (I) described herein. In more specific embodiments, the pharmaceutically acceptable salt is an acid addition salt (e.g., a trifluoroacetic acid salt or a hydrochloric acid salt).

Methods for the preparation of compositions comprising the compounds of structure (I) include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions of structure (I) include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound of structure (I) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds of structure (I) are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of the compound of structure (I) provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the compound of structure (I) provided in the pharmaceutical compositions is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the compound of structure (I) provided in the pharmaceutical compositions is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of the compound of structure (I) provided in the pharmaceutical compositions is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount the compound of structure (I) provided in the pharmaceutical compositions is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the compound of structure (I) provided in the pharmaceutical compositions is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of the compound of structure (I) provided in the pharmaceutical compositions is in the range of 0.0001-10 g, 0.0005-9 g, 0.001-8 g, 0.005-7 g, 0.01-6 g, 0.05-5 g, 0.1-4 g, 0.5-4 g, or 1-3 g.

### Kits/Articles of Manufacture

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds of structure (I), optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound of structure (I). Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods of Treatment and Administration

Embodiments of the present disclosure provide a method for treating CDK7-dependent diseases (e.g., cancer). One embodiment provides a method for treating a CDK7-dependent disease, the method comprising administering administering an effective amount of a compound of structure (I) (or pharmaceutically acceptable salt thereof) or a pharmaceutical composition comprising a compound of structure (I) as disclosed in any one of the embodiments herein to a subject in need thereof.

In some more specific embodiments, the CDK7-dependent disease is cancer. For example, in some embodiments, the cancer is a breast cancer. In some embodiments, the cancer is triple negative breast cancer (TNBC). In some embodiments, the cancer is pancreatic cancer. In some embodiments, the cancer is a sarcoma.

In some embodiments, the method of administering relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, [symbol] lobular carcinoma in situ (LCIS), [symbol] lung cancer, lymphoma, metastatic squamous neck cancer with occult primary, [symbol] midline tract carcinoma, [symbol] mouth cancer [symbol] multiple endocrine neoplasia syndromes, [symbol] multiple myeloma/plasma cell neoplasm, [symbol] mycosis fungoides, myelodysplastic syndromes, [symbol] myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, [symbol] nasopharyngeal cancer, neuroblastoma, [symbol] non-hodgkin lymphoma, [symbol] non-small cell lung cancer (NSCLC), oral cancer, lip and oral cavity cancer, oropharyngeal cancer, ovarian cancer, pancreatic cancer, papillomatosis, paraganglioma, [symbol] paranasal sinus and nasal cavity cancer, parathyroid cancer, [symbol] penile cancer, [symbol] pharyngeal cancer, [symbol] pleuropulmonary blastoma, primary central nervous system (CNS) lymphoma, [symbol] prostate cancer, rectal cancer, transitional cell cancer, [symbol] retinoblastoma, [symbol] rhabdomyosarcoma, salivary gland cancer, [symbol] skin cancer, stomach (gastric) cancer, small cell lung cancer, [symbol] small intestine cancer, [symbol] soft tissue sarcoma, T-Cell lymphoma, [symbol] testicular cancer, throat cancer, [symbol] thymoma and thymic carcinoma, thyroid cancer, transitional cell cancer of the renal pelvis and ureter, trophoblastic tumor, unusual cancers of childhood, [symbol] urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

In some more specific embodiments, the cancer is neuroblastoma, medulloblastoma, Ewing sarcoma, chordoma or combinations thereof. In some embodiments, the cancer is neuroblastoma. In some other embodiments, the cancer is medulloblastoma. In still other embodiments, the cancer is Ewing sarcoma. In certain embodiments, the cancer is chordoma.

In some embodiments, the cancer is a gastric. In some embodiments, the cancer is ovarian. In some embodiments, the cancer is glioblastoma. In some embodiments, the cancer is DIPG. In some embodiments, the cancer is pancreatic. In some embodiments, the cancer is colon cancer. In some embodiments, the cancer is small cell lung cancer (SCLC). In some embodiments, the cancer is AML. In some embodiments, the cancer comprises solid tumors. In some specific embodiments, the cancer is brain cancer. In some embodiments, the cancer comprises a cancer addicted to oncogenic super-enhancers that drive expression of onocogenes such as MYC.

Further therapeutic agents that can be combined with a compound of the disclosure are found in Goodman and Gilman's “The Pharmacological Basis of Therapeutics” Thirteenth Edition edited by Brunton, Hilal-Dandan and Knollmann or the Physician's Desk Reference, both of which are incorporated herein by reference in their entirety.

The compounds of structure (I) can be used in combination with the agents disclosed herein or other suitable agents, depending on the condition being treated. Hence, in some embodiments the one or more compounds of structure (I) will be co-administered with other agents as described above. When used in combination therapy, the compounds of structure (I) are administered with the second agent simultaneously or separately. This administration in combination can include simultaneous administration of the two agents in the same dosage form, simultaneous administration in separate dosage forms, and separate administration. That is, a compound of structure (I) and any of the agents described above can be formulated together in the same dosage form and administered simultaneously. Alternatively, a compound of structure (I) and any of the agents described above can be simultaneously administered, wherein both the agents are present in separate formulations. In another alternative, a compound of structure (I) can be administered just followed by and any of the agents described above, or vice versa. In some embodiments of the separate administration protocol, a compound of structure (I) and any of the agents described above are administered a few minutes apart, or a few hours apart, or a few days apart.

The compounds of structure (I) include a trisubstituted pyrazolo[1,5-a]-pyrimidine class of compounds as inhibitors of cyclin-dependent kinase (CDK7). CDK7 is a unique kinase that has the ability to regulate both cell-cycle progression and RNA polymerase-dependent transcription and is member of CDK family kinases including CDK1, 2, 4, 5, 6, 8, and 9 which regulates the cell cycle function and phosphorylation.

The present disclosure also provides methods for combination therapies in which an agent known to modulate other pathways, or other components of the same pathway, or even overlapping sets of target enzymes are used in combination with a compound of structure (I), or a stereoisomer, tautomer, prodrug, or pharmaceutically acceptable salt thereof. In one aspect, such therapy includes but is not limited to the combination of one or more compounds of structure (I) with chemotherapeutic agents, therapeutic antibodies, and radiation treatment, to provide a synergistic or additive therapeutic effect.

In various embodiments of the method, the additional therapeutic agent is an epidermal growth factor receptor (EGFR) inhibitor, phosphatidylinositol kinase (PI3K) inhibitor, insulin-like growth factor receptor (IGF1R) inhibitor, Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitors (such as irinotecan, etoposide, or asdoxorubicin), taxanes (such as anti-microtubule agents including paclitaxel and docetaxel), anti-metabolite agents (such as 5-FU or such as gemcitabine), alkylating agents (such as cisplatin or such as cyclophosphamide), or a taxane.

In some specific embodiments, the additional therapeutic agent is gemcitabine, cisplatin, 5-fluorouracil, nutlin, panobinostat, olaparib, or combinations thereof. In some more specific embodiments, the additional therapeutic agent is Gemcitabine. In some embodiments, the additional therapeutic agent is cisplatin. In some embodiments, the additional therapeutic agent is 5-fluorouracil. In some embodiments, the additional therapeutic agent is nutlin. In some embodiments, the additional therapeutic agent is panobinostat. In some embodiments, the additional therapeutic agent is olaparib.

In some embodiments, the additional therapeutic agent is an epidermal growth factor receptor (EGFR) inhibitor, such as Erlotinib or such as Afatinib. In some embodiments, the additional therapeutic agent is Iressa. In some embodiments, the additional therapeutic agent is a monoclonal antibody such as cetuximab (Erbitux) or panitumumab (Vectibix). In some embodiments the GFR inhibitor is a dual or pan-HER inhibitor. In other embodiments, the additional therapeutic agent is a phosphatidylinositol-3kinase (PI3K) inhibitor, such as GDC0941, MLN1117, BYL719 (Alpelisib) or BKM120 (Buparlisib). GDC0941 refers to 2-(1H-indazol-4-yl)-6-(4-methanesulfonyl-piperazin-1-ylmethyl)-4-morpholin-4-yl-thieno[3,2-d]pyrimidine or a salt thereof (e.g., bismesylate salt).

In still different embodiments, the additional therapeutic agent is an insulin-like growth factor receptor (IGF1R) inhibitor. For example, in some embodiments the insulin-like growth factor receptor (IGF1R) inhibitor is NVP-AEW541. In other embodiments, the additional therapeutic agent is IGOSI-906 (Linsitinib), BMS-754807, or in other embodiments the additional therapeutic agent is a neutralizing monoclonal antibodies specific to IGF1R such as AMG-479 (ganitumab), CP-751,871 (figitumumab), IMC-A12 (cixutumumab), MK-0646 (dalotuzumab), and R-1507 (robatumumab).

In some other embodiments, the additional therapeutic agent is a Janus kinase (JAK) inhibitor. In some embodiments, the additional therapeutic agent is CYT387, GLPG0634, Baricitinib, Lestaurtinib, momelotinib, Pacritinib, Ruxolitinib or TG101348

In some other embodiments, the additional therapeutic agent is an MET kinase inhibitor, such as Crizotinib, tivantinib, AMG337, cabozantinib, foretinib. In other embodiments, the additional therapeutic agent is a neutralizing monoclonal antibody to MET such as onartuzumab.

In more embodiments, the additional therapeutic agent is a SRC family non-receptor tyrosine kinase inhibitor. For example in some embodiments, the additional therapeutic agent is an inhibitor of the subfamily of SRC family non-receptor tyrosine kinases. Exemplary inhibitors in this respect include Dasatinib. Other examples in this regard include Ponatinib, saracatinib, and bosutinib

In yet different embodiments, the additional therapeutic agent is a mitogen-activated protein kinase (MEK) inhibitor. In some of these embodiments, the mitogen-activated protein kinase (MEK) inhibitor is trametinib, selumetinib, cobimetinib, PD0325901, or RO5126766. In other embodiments, the MEK inhibitor is GSK-1120212, also known as trametinib.

In yet different embodiments, the additional therapeutic agent is an extracellular-signal-regulated kinase (ERK) inhibitor. In some of these embodiments, the mitogen-activated protein kinase (MEK) inhibitor is SCH722984 or GDC-0994.

The exact method for administering the compound of structure (I) and the additional therapeutic agent will be apparent to one of ordinary skill in the art. In some exemplary embodiments, the compound of structure (I) and the additional therapeutic agent are co-administered. In other embodiments, the compound of structure (I) and the additional therapeutic agent are separately administered.

In some embodiments, the compound of structure (I) and the additional therapeutic agent are administered with the second agent simultaneously or separately. This administration in combination can include simultaneous administration of the two agents in the same dosage form, simultaneous administration in separate dosage forms, and separate administration. That is, the compound of structure (I) and any of the additional therapeutic agents described herein can be formulated together in the same dosage form and administered simultaneously. Alternatively, the compound of structure (I) and any of the additional therapeutic agents described herein can be simultaneously administered, wherein both the agents are present in separate formulations. In another alternative, the compound of structure (I) can be administered just followed by and any of the additional therapeutic agents described herein, or vice versa. In some embodiments of the separate administration protocol, the compound of structure (I) and any of the additional therapeutic agents described herein are administered a few minutes apart, or a few hours apart, or a few days apart.

In other embodiments, the additional therapeutic agent is a protein kinase inhibitor, such as Staurosporine or Midostaurin. In other embodiments, the protein kinase inhibitor is is Afatinib, Axitinib, Bevacizumab, Bostutinib, Cetuximab, Crizotinib, Dasatinib, Erlotinib, Fostamatinib, Gefitinib, Imatinib, Lapatinib, Lenvatinib, Ibrutinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib, or Vemurafenib.

In still more embodiments, the additional therapeutic agent is a topoisomerase inhibitor. In some of these embodiments, the topoisomerase inhibitor is Irinotecan. In some more embodiments, the additional therapeutic agent is a taxane. Exemplary taxanes include Taxol and Docetaxel.

In addition to the above additional therapeutic agent, other chemotherapeutics are presently known in the art and can be used in combination with the compounds of structure (I). In some embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomeRASe inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents.

Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomeRASe inhibitor RFS 2000; difluoromethylornithine (DMFO). Where desired, the compounds or pharmaceutical composition of of structure (I) can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

This disclosure further relates to a method for using the compounds of structure (I) or pharmaceutical compositions provided herein, in combination with radiation therapy for inhibiting abnormal cell growth or treating the hyperproliferative disorder in the mammal. Techniques for administering radiation therapy are known in the art, and these techniques can be used in the combination therapy described herein. The administration of the compound of structure (I) in this combination therapy can be determined as described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term includes exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. The radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

Without being limited by any theory, the compounds of structure (I) can render abnormal cells more sensitive to treatment with radiation for purposes of killing and/or inhibiting the growth of such cells. Accordingly, this disclosure further relates to a method for sensitizing abnormal cells in a mammal to treatment with radiation which comprises administering to the mammal an amount of a compound of structure (I) or a stereoisomer, tautomer, prodrug, or pharmaceutically acceptable salt thereof, which amount is effective is sensitizing abnormal cells to treatment with radiation. The amount of the compound of structure (I) in this method can be determined according to the means for ascertaining effective amounts of such compounds described herein.

In some embodiments, the compounds described herein are formulated or administered in conjunction with liquid or solid tissue barriers also known as lubricants. Examples of tissue barriers include, but are not limited to, polysaccharides, polyglycans, seprafilm, interceed and hyaluronic acid.

The examples and preparations provided below further illustrate and exemplify the compounds of of structure (I) and methods of preparing such compounds. It is understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers are obtained by methods known to those skilled in the art.

### Methods of Preparation

Compounds of structure (I) can be prepared according to methods known in the art and according to methods disclosed herein. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)).

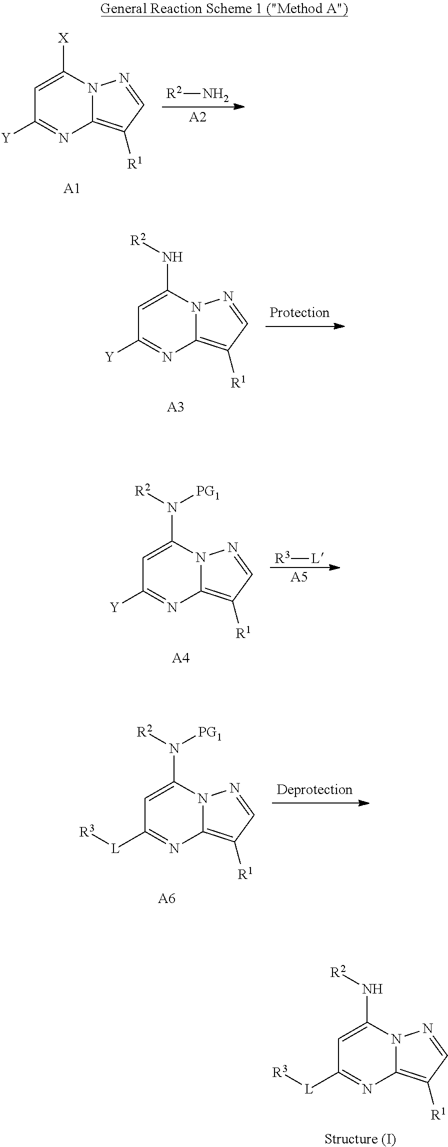

General Reaction Scheme 1 (“Method A”) provides an exemplary method for preparation of compounds of structure (I). R¹, R², R³, and L in General Reaction Scheme 1 are as defined herein. X and Y are reactive moieties selected to facilitate the desired reactions (e.g., halo). L′ is selected such that a desired L moiety results from the reaction between A5 and A4. Compounds of structure A1, A2 and A5 are purchased or prepared according to methods known in the art; or as described herein. Reaction of A1 with A2 under appropriate coupling conditions (e.g., use of base; or use of base in combination with heat and microwave irradiation) yields the product of the coupling reaction between A1 and A2, A3. A3 is then reacted under suitable conditions (e.g., (Boc)₂O, base) to introduce a protecting group (PG₁) on compound A3 to provide product A4. PG₁ can be selected (and, alternatively, modified if necessary) based on compatibility with other synthetic steps (e.g., the conditions required to couple A4 and A5 to form A6) in view of the entire reaction scheme. PG₁ may include, but is not limited to, Boc, benzyl, DMB, or Cbz. Reaction of A4 with A5 under appropriate coupling conditions (e.g., use of base; or use of base in combination with heat and microwave irradiation; or Buchwald-Hartwig coupling conditions, such as, Pd₂(dba)₃, (rac)-BINAP and base in toluene while heating) yields the product of the coupling reaction between A4 and A5, A6. A6 is then reacted under suitable protecting group removal conditions (e.g., DCM and TFA; or HCl and dioxane) to afford a compound of structure (I).

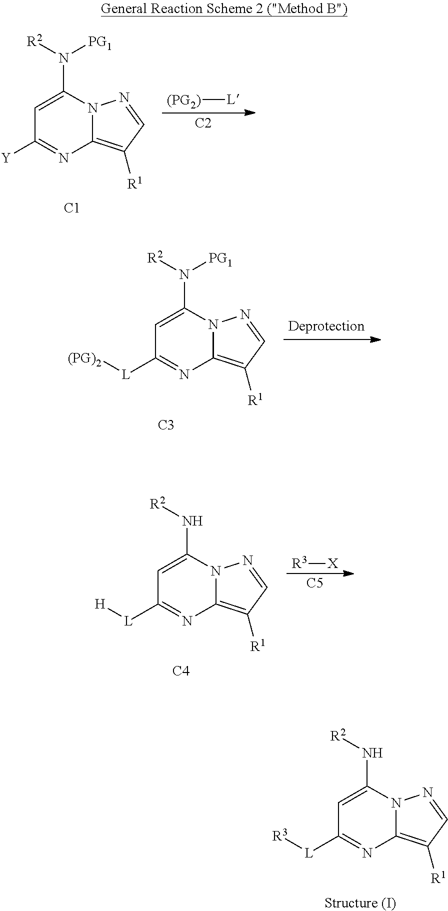

General Reaction Scheme 2 (“Method B”) provides an exemplary method for preparation of compounds of structure (I). R¹, R², R³, and L in General Reaction Scheme 2 are as defined herein. X and Y are reactive moieties selected to facilitate the desired reactions (e.g., halo). L′ is selected such that a desired L moiety results from the reaction between C2 and C1. Compounds of structure C1, C2 and C5 are purchased or prepared according to methods known in the art; or as described herein. PG₁ and PG₂ can be selected (and, alternatively, modified if necessary) based on compatibility with other synthetic steps (e.g., the conditions required to couple C1 and C2 to form C3) in view of the entire reaction scheme. PG₁ and PG₂ may include, but are not limited to, Boc, benzyl, DMB, or Cbz. Reaction of C1 with C2 under appropriate coupling conditions (e.g., solvent and heat; use of base; or use of base in combination with heat and microwave irradiation; or Buchwald-Hartwig coupling conditions, such as, Pd₂(dba)₃, (rac)-BINAP and base in toluene while heating) yields the product of the coupling reaction between C1 and C2, C3. C3 is then reacted under suitable protecting group removal conditions (e.g., DCM and TFA; or HCl and Dioxane) to provide product C4. This example shows both PG₁ and PG₂ being removed. However, those skilled in the art will recognize when it may be necessary to, for example, leave PG₁ in place to facilitate the next reaction step for certain compounds of structure (I). Orthogonal protecting group strategies can be employed by those skilled in the art to facilitate this need. Reaction of C4 with C5 under appropriate conditions (e.g., C5 is acyl halide with base or C5 is cycloalkyl halide with base) affords a compound of structure (I).

It should be noted that various alternative strategies for preparation of compounds of structure (I) are available to those of ordinary skill in the art. For example, other compounds of structure (I) can be prepared according to analogous methods using the appropriate starting material. It will also be appreciated by those skilled in the art that in the processes for preparing the compounds of structure (I), the functional groups of intermediate compounds may need to be protected by suitable protecting groups. Such functional groups include, but are not limited to, hydroxy, amino, mercapto and carboxylic acid.

Suitable protecting groups for hydroxy include, but are not limited to, trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl (“Boc”), benzyloxycarbonyl, and the like. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs”. Prodrugs of compounds of this disclosure are included within the scope of embodiments of the disclosure.

The examples and preparations provided below further illustrate and exemplify the compounds of structure (I) and methods of preparing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

### EXAMPLES

The following examples are provided for exemplary purposes. Methods for preparation of compounds of structure (I) are known in the art or can be derived by one of ordinary skill in the art.

### Example 1

### Synthesis of 5,7-dichloro-3-cyclopropylpyrazolo[1,5-a]pyrimidine

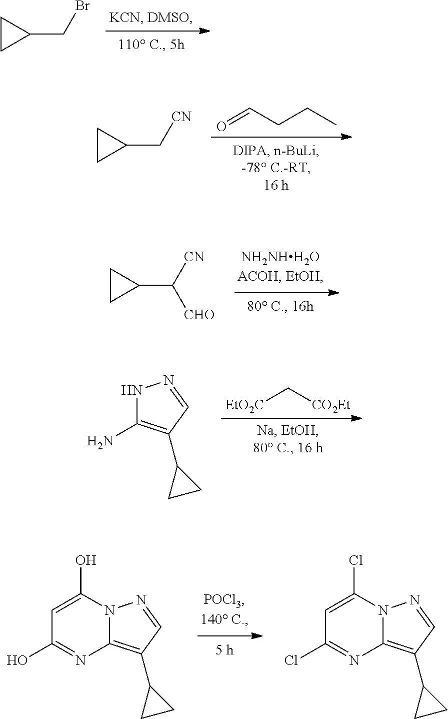

### 2-Cyclopropylacetonitrile

To a stirred solution of potassium cyanide (14.57 g, 223.88 mmol, 1.0 eq) in DMSO (1 mL), was added (bromomethyl)cyclopropane (30 g, 223.88 mmol, 1.0 eq) under nitrogen at room temperature. The reaction mixture was then heated at 110° C. and stirred for 5 h. Crude NMR indicated formation of the product. The reaction mixture was then quenched with ice cold water and extracted with ether (3×250 mL). The combined organic layer was washed with ice water (2×250 mL), brine (200 mL), dried over sodium sulfate, and concentrated to afford 2-cyclopropylacetonitrile as a colorless liquid (15.0 g, yield: 82%); TLC system: EtOAc:Hexane (10:90), (stained in KMNO₄) R_{f} value: ˜0.2; ¹HNMR (400 MHz, CDCl₃) δ2.37 (d, J=6.4 Hz, 2H), 1.15-0.95 (m, 1H), 0.65-0.55 (m, 2H), 0.40-0.25 (m, 2H).

### 2-Cyclopropyl-3-oxopropanenitrile

To a stirred solution of diisopropylamine (18.72 g, 185.18 mmol, 1.0 eq) in THF (200 mL) at −78° C. was added n-BuLi (2.5 M) (89 mL, 222.21 mmol, 1.2 eq) and the reaction was stirred for 30 min. To this reaction mixture, 2-cyclopropylacetonitrile (15.0 g, 185.18 mmol, 1.0 eq) was slowly added, followed by ethyl formate (16.44 g, 222.11 mmol, 1.0 eq) at −78° C. The reaction was warmed to room temperature and stirred for 16 h. After completion of reaction by TLC, the reaction mixture was quenched with 1N HCl to a tuned pH of 2-3 and extracted with ether (2×250 mL), washed with brine (100 mL), dried over sodium sulfate, and concentrated to afford 2-cyclopropyl-3-oxopropanenitrile as a brown liquid (18.0 g, crude). TLC system: EtoAc:Hexane (30:70), (stained in KMNO₄) R_{f} value: ˜0.3; ¹H NMR (400 MHz, CDCl₃) δ Aldehyde proton was observed at 9.60 (s, 1H) and the crude material was taken forward to the next step.

### 4-Cyclopropyl-1H-pyrazol-5-amine

To a stirred solution of 2-cyclopropyl-3-oxopropanenitrile (18.0 g, 165.13 mmol, 1.0 eq; crude) in EtOH (180 mL) at room temperature, hydrazine monohydrate (11.0 g, 214.6 mmol, 1.3 eq) was added, followed by acetic acid (15.8 g, 264.2 mmol, 1.6 eq). The reaction mixture was stirred at 80° C. for 16 h. After completion of reaction monitored by TLC, the reaction mixture was quenched with ice cold water, and extracted with EtOAc (2×200 mL). The combined organic layer was washed with brine (150 mL), dried over sodium sulfate, concentrated, and triturated with pentane to afford 4-cyclopropyl-1H-pyrazol-5-amine as an off white solid (17.0 g, yield: 75% over two steps). TLC system: Methanol/DCM (5:95), R_{f} value: ˜0.2; LCMS (m/z): 124.1 (M+H)⁺; ¹H NMR (400 MHz, DMSO-d₆) δ 11.12 (s, 1H), 6.96 (s, 1H), 4.35 (s, 2H), 1.47-1.41 (m, 1H), 0.71-0.66 (m, 2H), 0.36-0.32 (m, 2H).

### 3-Cyclopropylpyrazolo[1,5-a]pyrimidine-5,7-diol

To a stirred solution of Na metal (3.81 g, 165.85 mmol, 1.2 eq) in EtOH (170 mL) at room temperature 4-cyclopropyl-1H-pyrazol-5-amine (17.0 g, 138.21 mmol, 1.0 eq) was added, followed by addition of diethyl malonate (29.18 g, 151.8 mmol, 1.1 eq). The reaction mixture was stirred at 80° C. for 16 h. After completion of reaction by TLC, the reaction mixture was diluted with ice cold water and acidified with 5N HCl to pH of 2-3 and then stirred for 1 h. Then the reaction mass was filtered, washed with water and pentane, and dried to afford 4-cyclopropyl-1H-pyrazol-5-amine as an off white solid (15.0 g, yield: 57%). TLC system: Methanol/DCM (15:85), R_{f} value: ˜0.1; LCMS (m/z): 192.3 (M+H)⁺; ¹H NMR (400 MHz, DMSO-d₆) δ 12.17 (s, 1H), 11.55 (s, 1H), 7.49-7.46 (m, 2H), 1.78-1.77 (m, 1H), 0.84-0.81 (m, 2H), 0.59-0.57 (m, 2H). Aliphatic impurities were observed along with product.

### 5,7-Dichloro-3-cyclopropylpyrazolo[1,5-a]pyrimidine

3-cyclopropylpyrazolo[1,5-a]pyrimidine-5,7-diol (15.0 g, 0.266 mmol, 1.0 eq) was added to POCl₃ (240 mL) at room temperature and the reaction mixture was heated at 140° C. for 5 h. After completion of reaction by TLC, the reaction mixture was concentrated, quenched with ice cold water, and then extracted with DCM (2×200 mL). The combined organic layers were washed with brine (100 mL), dried over sodium sulfate, and concentrated. The crude compound was purified by column chromatography [gradient elution with 5-10%/Ethyl acetate/Hexane] to afford 5,7-dichloro-3-cyclopropylpyrazolo[1,5-a]pyrimidine as a yellow solid (4.5 g, yield: 25%). TLC system: EtOAc/hexane (10:90), R_{f} value: ˜0.6; LCMS (m/z): 228.3 (M+H)⁺; ¹H NMR (400 MHz, CDCl₃) δ 7.92 (s, 1H), 6.90 (s, 1H), 2.08-2.05 (m, 1H), 1.03-1.00 (m, 2H), 0.84-0.82 (m, 2H).

### Example 2

### Synthesis of 5,7-dichloro-3-cyclobutylpyrazolo[1,5-a]pyrimidine

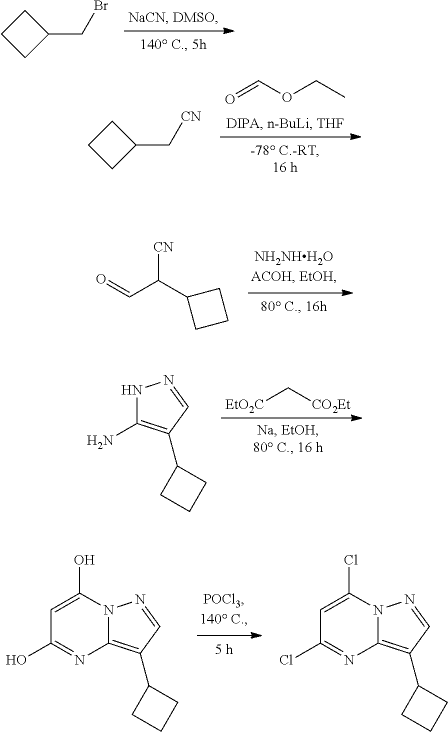

### 2-Cyclobutylacetonitrile

To a stirred solution of potassium cyanide (7.33 g, 149.65 mmol, 1.0 eq) in DMSO (150 mL), was added (bromomethyl)cyclobutane (22 g, 149.65 mmol, 1.0 eq) under nitrogen atmosphere at room temperature. The reaction mixture was then heated at 140° C. for 5 h. Crude NMR indicated formation of the product. The reaction mixture was then quenched with ice cold water and extracted with ether (3×200 mL). The combined organic layer was washed with ice water (2×200 mL), brine (150 mL), dried over sodium sulfate, and concentrated to afford 2-cyclobutylacetonitrile as a colorless liquid (12.6 g, yield: 70%); TLC system: EtoAc:Hexane (10:90), (stained in KMNO⁴); R_{f} value: ˜0.4; ¹HNMR (400 MHz, CDCl₃) δ 2.71-2.65 (m, 1H), 2.44 (d, J=7.6 Hz, 2H), 2.11-2.18 (m, 2H), 1.91-1.85 (m, 2H), 1.74-1.69 (m, 2H); diethyl ether solvent traces observed along with product.

### 2-Cyclobutyl-3-oxopropanenitrile

To a stirred solution of diisopropylamine (20.75 mL, 145.9 mmol, 1.1 eq) in THF (200 mL) at −78° C. was added n-BuLi (2.5 M) (63 mL, 159.1 mmol, 1.2 eq) and the reaction was stirred for 30 min. To this reaction mixture, 2-cyclobutylacetonitrile (12.6 g, 132.6 mmol, 1.0 eq) was slowly added, followed by ethyl formate (12.94 mL, 159.1 mmol, 1.2 eq) at −78° C. The reaction was then warmed to room temperature and stirred for 16 h. After completion of the reaction by TLC, the reaction mixture was quenched with 1N HCl to a pH of 2-3 and extracted with ether (2×250 mL). The organic layer was washed with brine (100 mL), dried over sodium sulfate, and concentrated to afford 2-cyclobutyl-3-oxopropanenitrile as a brown liquid (14.4 g, crude). TLC system: EoAc:Hexane (30:70), R_{f} value: ˜0.3; ¹H NMR (400 MHz, CDCl₃) δ Aldehyde proton was observed at 9.60 (s, 1H) and the crude material was taken forward to the next step.

### 4-Cyclobutyl-1H-pyrazol-5-amine

To a stirred solution of 2-cyclobutyl-3-oxopropanenitrile (14.4 g, 117.07 mmol, 1.0 eq; crude) in EtOH (180 mL) at room temperature was added hydrazine monohydrate (7.60 g, 152.19 mmol, 1.3 eq) and acetic acid (11.23 g, 187.3 mmol, 1.6 eq). The reaction mixture was stirred at 80° C. for 16 h. After completion of reaction by TLC, the reaction mixture was quenched with ice cold water, and extracted with EtOAc (2×200 mL). The combined organic layer was washed with brine (150 mL), dried over sodium sulfate, concentrated, and triturated with pentane to afford 4-cyclobutyl-1H-pyrazol-5-amine as a light brown solid (12.28 g, yield: 76% over two steps). TLC system: Methanol/DCM (5:95), R_{f} value: ˜0.2; LCMS (m/z): 138.1 (M+H)⁺; ¹H NMR (400 MHz, DMSO-d₆) δ 11.22 (brs, 1H), 7.20 (s, 1H), 4.25 (s, 2H), 3.22-3.20 (m, 1H), 2.53-2.08 (m, 2H), 1.93-1.75 (m, 4H).

### 3-Cyclobutylpyrazolo[1,5-a]pyrimidine-5,7-diol

To a stirred solution of Na metal (2.47 g, 102.9 mmol, 1.2 eq) in EtOH (150 mL) at room temperature 4-cyclobutyl-1H-pyrazol-5-amine (12.28 g, 89.63 mmol, 1.0 eq) was added, followed by addition of diethyl malonate (17.75 mL, 116.5 mmol, 1.3 eq). The reaction mixture was stirred at 80° C. for 16 h. After completion of reaction by TLC, the reaction mixture was diluted with ice cold water and acidified with 5N HCl to a pH of 2-3 and then stirred for 1 h. The reaction mass was filtered, washed with water, diethyl ether and dried to afford 3-cyclobutylpyrazolo[1,5-a]pyrimidine-5,7-diol as a light brown solid (10.25 g, yield: 56%). TLC system: Methanol/DCM (10:90), R_{f} value: ˜0.05; LCMS (m/z): 206.1 (M+H)⁺.

### 5,7-Dichloro-3-cyclobutylpyrazolo[1,5-a]pyrimidine

3-cyclobutylpyrazolo[1,5-a]pyrimidine-5,7-diol (2.25 g, 10.97 mmol, 1.0 eq) was added to POCl₃ (25 mL) at room temperature and the reaction mixture was heated at 140° C. for 5 h. After completion of reaction by TLC, the reaction mixture was concentrated, quenched with ice cold water and then extracted with DCM (2×100 mL). The combined organic layers were washed with brine (50 mL), dried over sodium sulfate and concentrated. The crude compound was purified by column chromatography [gradient elution with 5-10% Ethyl acetate/Hexane] to afford 5,7-dichloro-3-cyclobutylpyrazolo[1,5-a]pyrimidine as a yellow solid (0.92 g, yield: 35%). TLC system: EtOAc/hexane (10:90), R_{f} value: ˜0.6; LCMS (m/z): 242.0 (M+H)⁺; ¹H NMR (400 MHz, CDCl₃) δ 8.18 (s, 1H), 6.91 (s, 1H), 3.83-3.79 (m, 1H), 2.47-2.39 (m, 2H), 2.33-2.23 (m, 2H), 2.09-1.94 (m, 2H).

### Example 3

### Synthesis of (s)-3-chloro-N⁷-(3-chlorophenyl)-N⁵-(piperidin-3-yl)pyrazolo[1,5-a]pyrimidine-5,7-diamine (Compound I-1)

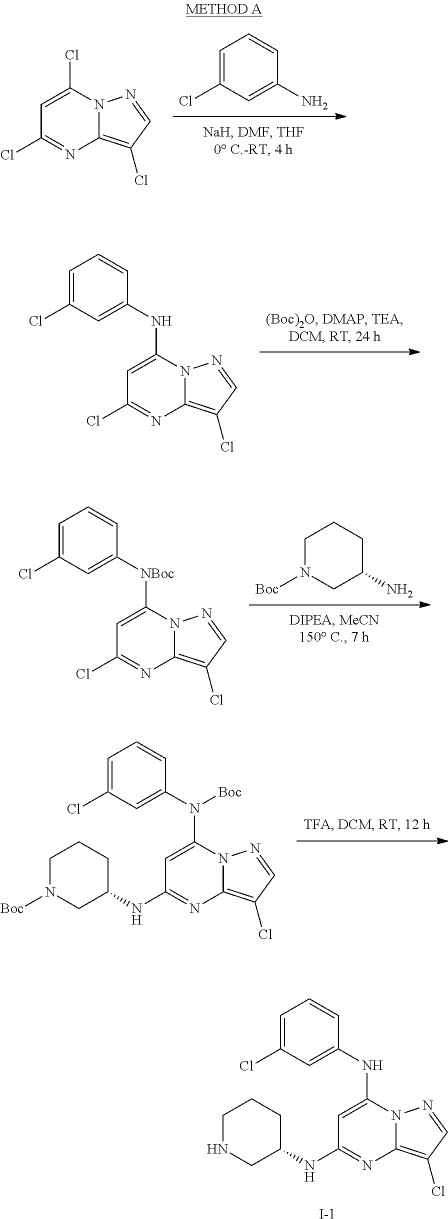

### N-(3-chlorophenyl)-3,5-Dichloropyrazolo[1,5-a]pyrimidin-7-amine

To a stirred solution of 3-chloroaniline (127 mg, 1.0 mmol, 1 eq) in DMF (3 mL) and THF (3 mL) at 0-5° C., was added NaH (60%, 80 mg, 2.0 mmol, 2.0 eq) portion-wise under nitrogen flush and the reaction was stirred for 30 min. To this mixture at 0° C. was added 3,5,7-trichloropyrazolo[1,5-a]pyrimidine (333 mg, 1.50 mmol, 1.5 eq) and the reaction was stirred at room temperature for 4 h. After completion of reaction by TLC, the reaction mixture was quenched with ice cold water and extracted with (3×50 mL) ethyl acetate. The combined organic layer was washed with water (30 mL), brine (30 mL), dried over sodium sulfate, and concentrated to afford crude product which was purified by using CombiFlash chromatography (4 g column) to afford N-(3-chlorophenyl)-3,5-dichloropyrazolo[1,5-a]pyrimidin-7-amine as yellow solid (255 mg; 81%). TLC system: EtOAc:Hexane (1:4), R_{f} value: ˜0.6; ¹HNMR (400 MHz, CDCl₃) δ 8.12 (s, 1H), 8.02 (s, 1H), 7.44 (t, J=8.0 Hz, 1H), 7.38 (t, J=2.0 Hz, 1H), 7.35-7.32 (m, 1H), 7.29-7.26 (m, 1H), 6.33 (s, 1H).

### Tert-Butyl (3,5-dichloropyrazolo[1,5-a]pyrimidin-7-yl)(3-chlorophenyl)carbamate

To a stirred solution of N-(3-chlorophenyl)-3,5-dichloropyrazolo[1,5-a]pyrimidin-7-amine (100 mg, 0.32 mmol, 1.0 eq) in DCM (5 mL) at room temperature was added triethylamine (65 mg, 0.64 mmol, 2.0 eq) and DMAP (2 mg 0.016 mmol), followed by addition of (Boc)₂O (91 mg, 0.42 mmol, 1.3 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (3,5-dichloropyrazolo[1,5-a]pyrimidin-7-yl)(3-chlorophenyl)carbamate as a yellow oil (130 mg, 95%). TLC system: EtOAc/hexane (1:4), R_{f} value: ˜0.6.

### (S)-tert-Butyl 3-(7-(tert-butoxycarbonyl)(3-chlorophenyl)amino-3-chloropyrazolo[1,5-a]pyrimidin-5-yl)aminopiperidine-1-carboxylate

To a stirred solution of (3,5-dichloropyrazolo[1,5-a]pyrimidin-7-yl)(3-chlorophenyl)carbamate (65 mg, 0.16 mmol, 1 eq) in acetonitrile (3 mL) in a microwave reaction vial, (S)-tert-butyl 3-aminopiperidine-1-carboxylate (39 mg, 0.19 mmol, 1.2 eq) and DIPEA (42 mg, 0.32 mmol, 2.0 eq) were added at room temperature. The reaction mixture was under nitrogen flush for 5 minutes and then the microwave vial was sealed and heated at 150° C. for 7 h. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford (S)-tert-butyl 3-(7-(tert-butoxycarbonyl)(3-chlorophenyl)amino-3-chloropyrazolo[1,5-a]pyrimidin-5-yl)aminopiperidine-1-carboxylate as a yellow oil (13 mg, 14%). TLC system: EtOAc/hexane (1:2), R_{f} value: ˜0.60; ¹HNMR (400 MHz, CDCl₃) δ 7.79 (s, 1H), 7.40 (d, J=8.0 Hz, 1H), 7.83 (s, 1H), 7.26-7.23 (m, 2H), 5.62 (s, 1H), 4.71-4.57 (m, 1H), 3.86-3.83 (m, 1H), 3.61-3.55 (m, 1H), 3.22-3.11 (m, 2H), 1.87-1.79 (m, 2H), 1.73-1.66 (m, 2H), 1.45 (s, 9H), 1.40 (s, 9H).

### (S)-3-chloro-N⁷-(3-chlorophenyl)-N⁵-(piperidin-3-yl)pyrazolo[1,5-a]pyrimidine-5,7-diamine (I-1)

To a solution of (S)-tert-butyl 3-(7-(tert-butoxycarbonyl)(3-chlorophenyl)amino-3-chloropyrazolo[1,5-a]pyrimidin-5-yl)aminopiperidine-1-carboxylate (13 mg, 0.02 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction, sodium bicarbonate in water was added and the mixture was stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-chloro-N⁷-(3-chlorophenyl)-N⁵-(piperidin-3-yl)pyrazolo[1,5-a]pyrimidine-5,7-diamine (I-1) as a yellow solid (5 mg, yield: 70%). TLC system: DCM/Methanol (1:1), R_{f} value: ˜0.2; ¹HNMR (400 Hz, MeOD-d4) δ 7.78 (s, 1H), 7.43-7.40 (m, 2H), 7.34-7.31 (m, 1H), 7.24-7.22 (m, 1H), 5.75 (s, 1H), 4.19-4.13 (m, 1H), 3.43-3.41 (m, 1H), 3.07-3.04 (m, 1H), 2.74-2.68 (m, 1H), 2.63-2.58 (m, 1H), 2.08-2.04 (m, 1H), 1.89-1.86 (m, 1H), 1.72-1.66 (m, 1H), 1.57-1.51 (m, 1H). HRMS (ESI) m/z [M+H]⁺ calcd for C₁₇H₁₉Cl₂N₆ 377.1043, found 377.1053. HPLC: 95%.

### Example 4

### Synthesis of (S)-3-chloro-N⁷-(3-fluorophenyl)-N⁵-(piperidin-3-yl)pyrazolo[1,5-a]pyrimidine-5,7-diamine (Compound I-2)

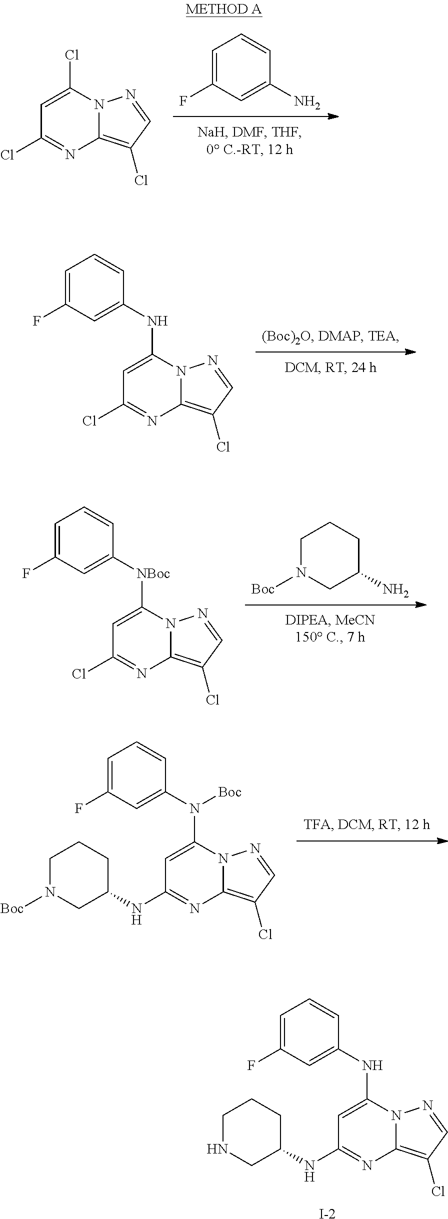

### 3,5-Dichloro-N-(3-fluorophenyl)pyrazolo[1,5-a]pyrimidin-7-amine

To a stirred solution of 3-fluoroaniline (144 mg, 1.30 mmol, 1.2 eq) in DMF (3 mL) and THF (3 mL) at 0-5° C., was added NaH (60%, 88 mg, 2.16 mmol, 2.0 eq) portion-wise under nitrogen flush and the mixture was stirred for 30 min. To this mixture at 0° C. was added 3,5,7-trichloropyrazolo[1,5-a]pyrimidine (240 mg, 1.08 mmol, 1.0 eq) and the reaction was stirred at room temperature for 12 h. After completion of reaction by TLC, the reaction mixture was quenched with ice cold water and extracted with (3×50 mL) ethyl acetate. The combined organic layer was washed with water (30 mL), brine (30 mL), dried over sodium sulfate, and concentrated to afford crude product which was purified by using CombiFlash chromatography (4 g column) to afford 3,5-dichloro-N-(3-fluorophenyl)pyrazolo[1,5-a]pyrimidin-7-amine as yellow solid (206 mg; 64%). TLC system: EtoAc:Hexane (1:4), R_{f} value: ˜0.4;

### Tert-Butyl (3,5-dichloropyrazolo[1,5-a]pyrimidin-7-yl)(3-fluorophenyl)carbamate

To a stirred solution of 3,5-dichloro-N-(3-fluorophenyl)pyrazolo[1,5-a]pyrimidin-7-amine (80 mg, 0.27 mmol, 1.0 eq) in DCM (5 mL) at room temperature was added triethylamine (55 mg, 0.54 mmol, 2.0 eq) and DMAP (2 mg 0.016 mmol), followed by addition of (Boc)₂O (76 mg, 0.35 mmol, 1.3 eq). The reaction was continued at room temperature for 16 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (3,5-dichloropyrazolo[1,5-a]pyrimidin-7-yl)(3-fluorophenyl)carbamate as a yellow oil (75 mg, 70%). TLC system: EtOAc/hexane (1:4), R_{f} value: ˜0.6; ¹HNMR (400 MHz, CDCl₃) δ 8.12 (s, 1H), 7.36-7.28 (m, 1H), 7.10-7.03 (m, 2H), 6.90-6.85 (m, 1H), 6.71 (s, 1H).

### Tert-Butyl (S)-3-(7-(tert-butoxycarbonyl)(3-fluorophenyl)amino-3-chloropyrazolo[1,5-a]pyrimidin-5-yl)aminopiperidine-1-carboxylate

To a stirred solution of tert-butyl (3,5-dichloropyrazolo[1,5-a]pyrimidin-7-yl)(3-fluorophenyl)carbamate (90 mg, 0.23 mmol, 1 eq) in acetonitrile (3 mL) in a microwave reaction vial, tert-butyl (S)-3-aminopiperidine-1-carboxylate (60 mg, 0.30 mmol, 1.2 eq) and DIPEA (65 mg, 0.50 mmol, 2.0 eq) were added at room temperature. The reaction mixture was under nitrogen flush for 5 minutes and then the microwave vial was sealed and heated at 150° C. for 7 h. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-(7-(tert-butoxycarbonyl)(3-fluorophenyl)amino-3-chloropyrazolo[1,5-a]pyrimidin-5-yl)aminopiperidine-1-carboxylate as a yellow oil (40 mg, 31%). TLC system: EtOAc/hexane (1:2), R_{f} value: ˜0.30; ¹HNMR (400 MHz, CDCl₃) δ 7.84 (s, 1H), 7.30-7.24 (m, 1H), 7.11-7.06 (m, 2H), 6.97-6.92 (m, 1H), 5.96 (s, 1H), 5.08-4.92 (m, 1H), 4.15-4.06 (m, 1H), 3.71-3.64 (m, 1H), 3.45-3.32 (m, 2H), 1.97-1.87 (m, 1H), 1.72-1.55 (m, 3H), 1.41 (s, 9H), 1.38 (s, 9H).

### (S)-3-chloro-N⁷-(3-fluorophenyl)-N⁵-(piperidin-3-yl)pyrazolo[1,5-a]pyrimidine-5,7-diamine (I-2)

To a solution of (S)-tert-butyl 3-(7-(tert-butoxycarbonyl)(3-fluorophenyl)amino-3-chloropyrazolo[1,5-a]pyrimidin-5-yl)aminopiperidine-1-carboxylate (40 mg, 0.07 mmol, 1.0 eq) in DCM (2 mL), TFA (1 mL) was added. The reaction mixture was stirred at room temperature for 12 h. After completion of reaction by TLC, the reaction mixture was concentrated. To the concentrated reaction, sodium bicarbonate in water was added and the mixture was stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-chloro-N⁷-(3-fluorophenyl)-N⁵-(piperidin-3-yl)pyrazolo[1,5-a]pyrimidine-5,7-diamine (I-2) as a yellow solid (20 mg, yield: 80%). TLC system: DCM/Methanol (1:1), R_{f} value: ˜0.2; ¹HNMR (400 Hz, MeOD-d4) δ 7.77 (s, 1H), 7.46-7.40 (m, 1H), 7.22-7.15 (m, 2H), 6.98-6.93 (m, 1H), 5.79 (s, 1H), 4.13-4.07 (m, 1H), 3.36-3.32 (m, 1H), 3.01-2.95 (m, 1H), 2.65-2.59 (m, 1H), 2.52-2.47 (m, 1H), 2.07-2.03 (m, 1H), 1.85-1.79 (m, 1H), 1.69-1.63 (m, 1H), 1.52-1.44 (m, 1H). HRMS (ESI) m/z [M+H]⁺ calcd for C₁₇H₁₉ClFN₆ 361.1338, found 361.1340. HPLC: 95%.

### Example 5

### Synthesis of (3S,4S)-3-((3-chloro-7-((3-fluorophenyl)amino)pyrazolo[1,5-a]pyrimidin-5-yl)amino)piperidin-4-ol (Compound I-3)

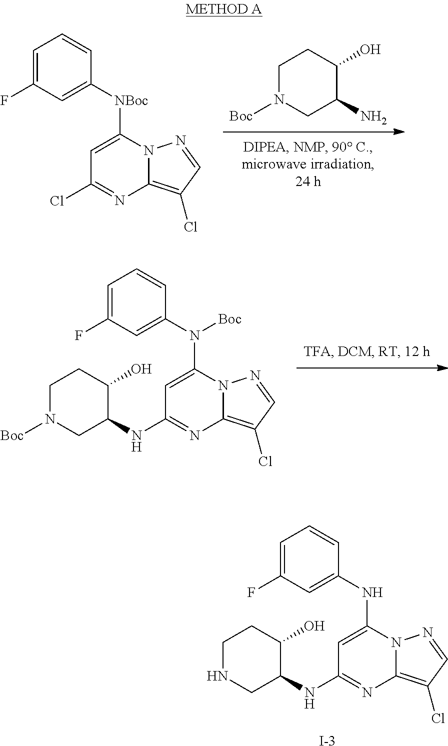

### Tert-Butyl (3S,4S)-3-(3-chloro-7-(tert-butoxycarbonyl)3-fluorophenyl)aminopyrazolo[1,5-a]pyrimidin-5-yl)amino-4-hydroxypiperidine-1-carboxylate

To a stirred solution of tert-butyl (3,5-dichloropyrazolo[1,5-a]pyrimidin-7-yl)(3-fluorophenyl)carbamate, prepared according to Example 4 above (77 mg, 0.19 mmol, 1 eq), in acetonitrile (3 mL) in a microwave reaction vial, tert-butyl (3S,4S)-3-amino-4-hydroxypiperidine-1-carboxylate (50 mg, 0.23 mmol, 1.2 eq) and DIPEA (49 mg, 0.38 mmol, 2.0 eq) were added at room temperature. The reaction mixture was under nitrogen flush for 5 minutes and stirring continued at 90° C. for 24 h under microwave irradiation. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (3S,4S)-3-(3-chloro-7-(tert-butoxycarbonyl)(3-fluorophenyl)aminopyrazolo[1,5-a]pyrimidin-5-yl)amino-4-hydroxypiperidine-1-carboxylate as a yellow oil (22 mg, 20%). TLC system: EtOAc/hexane (1:1), R_{f} value: ˜0.30; ¹HNMR (400 MHz, CDCl₃) δ 7.85 (s, 1H), 7.31-7.25 (m, 1H), 7.11-7.05 (m, 2H), 6.98-6.93 (m, 1H), 6.02 (s, 1H), 5.18-5.01 (m, 1H), 4.20-4.09 (m, 1H), 3.92-3.77 (m, 2H), 3.02-2.92 (m, 2H), 2.03-1.99 (m, 1H), 1.68-1.59 (m, 1H), 1.46 (s, 9H), 1.39 (s, 9H).

### (3S,4S)-3-((3-chloro-7-((3-fluorophenyl)amino)pyrazolo[1,5-a]pyrimidin-5-yl)amino)piperidin-4-ol (I-3)

To a solution of tert-butyl (3S,4S)-3-(3-chloro-7-(tert-butoxycarbonyl)(3-fluorophenyl)aminopyrazolo[1,5-a]pyrimidin-5-yl)amino-4-hydroxypiperidine-1-carboxylate (15 mg, 0.03 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and the mixture was stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (3S,4S)-3-((3-chloro-7-((3-fluorophenyl)amino)pyrazolo[1,5-a]pyrimidin-5-yl)amino)piperidin-4-ol (I-3) as a yellow solid (8 mg, yield: 73%). TLC system: DCM/Methanol (1:1), R_{f} value: ˜0.3; ¹HNMR (400 Hz, MeOD-d4) δ 7.78 (s, 1H), 7.47-7.41 (m, 1H), 7.23-7.17 (m, 2H), 6.99-6.94 (m, 1H), 5.85 (s, 1H), 3.98-3.93 (m, 1H), 3.59-3.53 (m, 1H), 3.38-3.34 (m, 1H), 3.10-3.05 (m, 1H), 2.67-2.60 (m, 1H), 2.49-2.43 (m, 1H), 2.07-2.01 (m, 1H), 1.65-1.55 (m, 1H). HRMS (ESI) m/z [M+H]⁺ calcd for C₁₇H₁₉ClFN₆O 377.1287, found 361.1296. HPLC: 95%.

### Example 6

### Synthesis of (S)-3-cyclopropyl-N-(3-fluorophenyl)-5-((piperidin-3-YL)oxy)pyrazolo[1,5-a]pyrimidin-7-amine (Compound I-4)

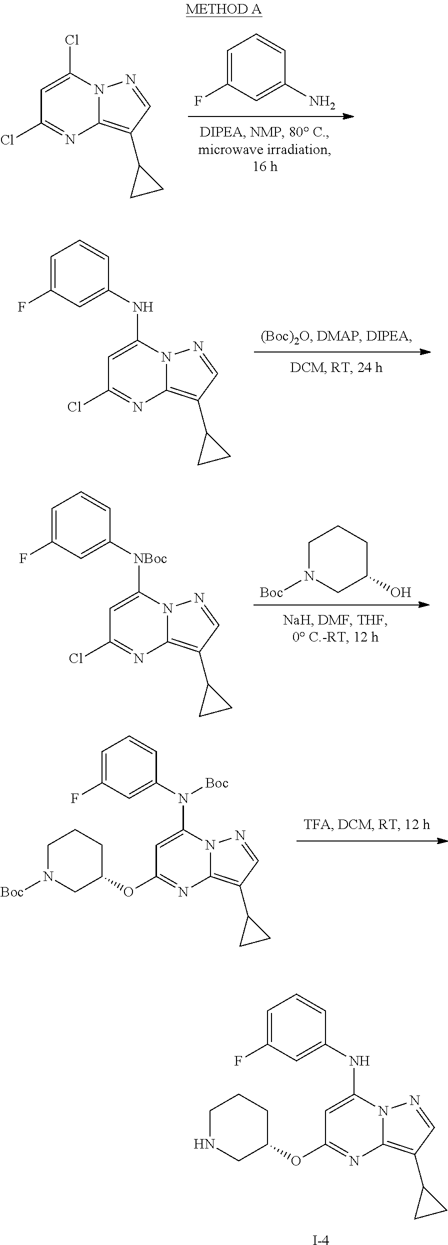

### 5-Chloro-3-cyclopropyl-N-(3-fluorophenyl)pyrazolo[1,5-a]pyrimidin-7-amine

To a stirred solution of 3-fluroaniline (248 mg, 2.23 mmol, 1.2 eq) in NMP (4 mL) was added DIPEA (312 mg, 2.42 mmol, 1.3 eq) and the solution was stirred for 5 min. To this mixture 5,7-dichloro-3-cyclopropylpyrazolo[1,5-a]pyrimidine, prepared according to Example 1 above (424 mg, 1.86 mmol, 1.0 eq), was added at room temperature and stirring continued at 80° C. for 16 h under microwave irradiation. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound without purification went to the next step, 5-chloro-3-cyclopropyl-N-(3-fluorophenyl)pyrazolo[1,5-a]pyrimidin-7-amine as a brown liquid. TLC system: EtoAc:Hexane (1:8), R_{f} value: ˜0.3; ¹HNMR (400 MHz, CDCl₃) δ 8.12 (s, 1H), 7.75 (s, 1H), 7.47-7.41 (m, 1H), 7.15-6.13 (m, 1H), 7.10-7.07 (m, 1H), 7.04-6.99 (m, 1H), 6.31 (s, 1H), 2.07-2.00 (m, 1H), 1.00-0.95 (m, 2H), 0.80-0.86 (m, 2H).

### Tert-Butyl (5-chloro-3-cyclopropylpyrazolo[1,5-a]pyrimidin-7-yl)(3-fluorophenyl)carbamate

To a stirred solution of 5-chloro-3-cyclopropyl-N-(3-fluorophenyl)pyrazolo[1,5-a]pyrimidin-7-amine (563 mg, 1.86 mmol, 1.0 eq) in DCM (5 mL) at room temperature was added DIPEA (481 mg, 3.72 mmol, 2 eq) and DMAP (2 mg 0.016 mmol), followed by addition of (Boc)₂O (812 mg, 3.72 mmol, 2 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyclopropylpyrazolo[1,5-a]pyrimidin-7-yl)(3-fluorophenyl)carbamate as a yellow oil (450 mg, 60% for two steps). TLC system: EtOAc/hexane (1:4), R_{f} value: ˜0.5; ¹HNMR (400 MHz, CDCl₃) δ 7.85 (s, 1H), 7.35-7.25 (m, 1H), 7.08-6.93 (m, 3H), 6.61 (s, 1H), 2.09-2.03 (m, 1H), 1.37 (s, 9H), 1.03-0.98 (m, 2H), 0.85-0.81 (m, 2H).

### Tert-Butyl (S)-3-((7-((tert-butoxycarbonyl)(3-fluorophenyl)amino)-3-cyclopropylpyrazolo[1,5-a]pyrimidin-5-yl)hydroxy)piperidine-1-carboxylate

To a stirred mixture of NaH (60%, 16 mg, 0.40 mmol, 2.0 eq) in DMF (3 mL), the mixture of tert-butyl (5-chloro-3-cyclopropylpyrazolo[1,5-a]pyrimidin-7-yl)(3-fluorophenyl)carbamate (81 mg, 0.20 mmol, 1.0 eq) and (S)-1-Boc-3-hydroxypiperidine (45 mg, 0.22 mmol, 1.1 eq) in THF (3 mL) was added at 0-5° C. and the reaction was stirred at room temperature for 12 h. After completion of reaction by TLC, the reaction mixture was quenched with ice cold water and extracted with (3×50 mL) ethyl acetate. The combined organic layer was washed with water (30 mL), brine (30 mL), dried over sodium sulfate, and concentrated to afford crude product which was purified by using CombiFlash chromatography (4 g column) to afford tert-butyl (S)-3-((7-((tert-butoxycarbonyl)(3-fluorophenyl)amino)-3-cyclopropylpyrazolo[1,5-a]pyrimidin-5-yl)hydroxy)piperidine-1-carboxylate as white oil (40 mg; 35%). TLC system: EtoAc:Hexane (1:4), R_{f} value: ˜0.4; ¹HNMR (400 MHz, CDCl₃) δ 7.70 (s, 1H), 7.40-7.34 (m, 1H), 7.10-7.04 (m, 2H), 6.95-6.90 (m, 1H), 5.83 (s, 1H), 5.18-5.15 (m, 1H), 3.68-3.62 (m, 2H), 3.45-3.35 (m, 2H), 2.02-1.96 (m, 1H), 1.94-1.79 (m, 3H), 1.59-1.52 (m, 1H), 1.35 (s, 9H), 1.28 (s, 9H), 0.90-0.84 (m, 4H).

### (S)-3-cyclopropyl-N-(3-fluorophenyl)-5-((piperidin-3-yl)oxy)pyrazolo[1,5-a]pyrimidin-7-amine (I-4)

To a solution of tert-butyl (S)-3-((7-((tert-butoxycarbonyl)(3-fluorophenyl)amino)-3-cyclopropylpyrazolo[1,5-a]pyrimidin-5-yl)hydroxy)piperidine-1-carboxylate (40 mg, 0.07 mmol, 1.0 eq) in DCM (2 mL), TFA (1 mL) was added. The reaction was stirred at room temperature for 12 h. After completion of reaction by TLC, the mixture was concentrated. To the concentrated material, sodium bicarbonate in water was added for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified using CombiFlash chromatography (4 g column) to afford (S)-3-cyclopropyl-N-(3-fluorophenyl)-5-((piperidin-3-yl)oxy)pyrazolo[1,5-a]pyrimidin-7-amine (I-4) as an off white solid (21 mg, yield: 81%). TLC system: DCM/Methanol (1:1), R_{f} value: ˜0.2; ¹HNMR (400 Hz, MeOD-d4) δ 7.71 (s, 1H), 7.46-7.40 (m, 1H), 7.23-7.14 (m, 2H), 6.99-6.94 (m, 1H), 5.83 (s, 1H), 5.27-5.22 (m, 1H), 3.24-3.20 (m, 1H), 3.05-3.00 (m, 1H), 2.90-2.87 (m, 2H), 2.84-1.99 (m, 1H), 1.94-1.84 (m, 3H), 1.68-1.60 (m, 1H), 0.87-0.81 (m, 4H). HRMS (ESI) m/z [M+H]⁺ calcd for C₂₀H₂₃FN₅O 368.1881, found 368.1884. HPLC: 95%.

### Example 7

### Synthesis of (S)—N⁵-(azepan-3-yl)-3-cyclopropyl-N⁷-(3-fluorophenyl)pyrazolo[1,5-a]pyrimidine-5,7-diamine (Compound I-5)

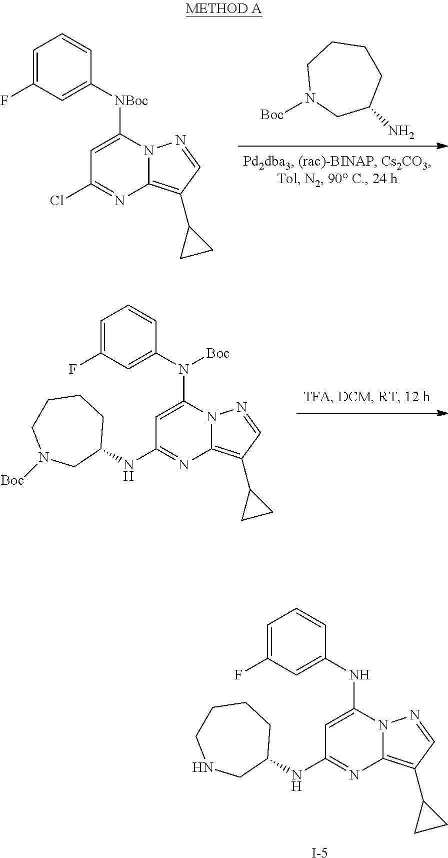

### Tert-Butyl (S)-3-((7-((tert-butoxycarbonyl)(3-cyanophenyl)amino)-3-cyclopropylpyrazolo[1,5-a]pyrimidin-5-yl)amino)azepane-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (50 mg, 0.12 mmol, 1 eq), and tert-Butyl (S)-3-aminoazepane-1-carboxylate (30 mg, 0.14 mmol, 1.2 eq) and Cs₂CO₃ (11 mg, 0.18 mmol, 1.5 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-((7-((tert-butoxycarbonyl)(3-cyanophenyl)amino)-3-cyclopropylpyrazolo[1,5-a]pyrimidin-5-yl)amino)azepane-1-carboxylate as a brown oil (20 mg, 29%). TLC system: EOAc/hexane (1:4), R_{f} value: 0.20; ¹HNMR (400 MHz, CDCl₃) δ 7.60 (s, 1H), 7.24-7.20 (m, 1H), 7.13-7.07 (m, 2H), 6.94-6.88 (m, 1H), 5.97 (s, 1H), 4.31-4.25 (m, 1H), 3.88-3.85 (m, 1H), 3.61-3.58 (m, 1H), 3.33-3.26 (m, 1H), 2.95-2.94 (m, 1H), 2.20-2.15 (m, 1H), 1.94-1.86 (m, 1H), 1.77-1.67 (m, 2H), 1.63-1.53 (m, 2H), 1.48 (s, 9H), 1.38 (s, 9H), 0.88-0.84 (m, 4H).

### (S)—N⁵-(azepan-3-yl)-3-cyclopropyl-N⁷-(3-fluorophenyl)pyrazolo[1,5-a]pyrimidine-5,7-diamine (I-5)

To a solution of tert-butyl (S)-3-((7-((tert-butoxycarbonyl)(3-cyanophenyl)amino)-3-cyclopropylpyrazolo[1,5-a]pyrimidin-5-yl)amino)azepane-1-carboxylate as a brown oil (20 mg, 0.03 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture was stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)—N⁵-(azepan-3-yl)-3-cyclopropyl-N⁷-(3-fluorophenyl)pyrazolo[1,5-a]pyrimidine-5,7-diamine (I-5) as an off white gummy solid (8 mg, yield: 73%). TLC system: DCM/Methanol (1:1), R_{f} value: 0.2; ¹HNMR (400 Hz, MeOD-d4) δ 7.56 (s, 1H), 7.43-7.38 (m, 1H), 7.19-7.12 (m, 2H), 6.94-6.89 (m, 1H), 4.86 (s, 1H), 4.18-4.12 (m, 1H), 3.24-3.19 (m, 1H), 2.99-2.87 (m, 3H), 2.07-2.00 (m, 1H), 1.87-1.60 (m, 6H), 0.85-0.81 (m, 2H), 0.77-0.74 (m, 2H). HRMS (ESI) m/z [M+H]⁺ calcd for C₂₁H₂₆FN₆ 381.2197, found 381.2200. HPLC: 95%.

### Example 8

### Synthesis of Trans-N⁵-(4-aminocyclohexyl)-3-cyclopropyl-N⁷-(3-fluorophenyl)pyrazolo[1,5-a]pyrimidine-5,7-diamine (Compound I-6)

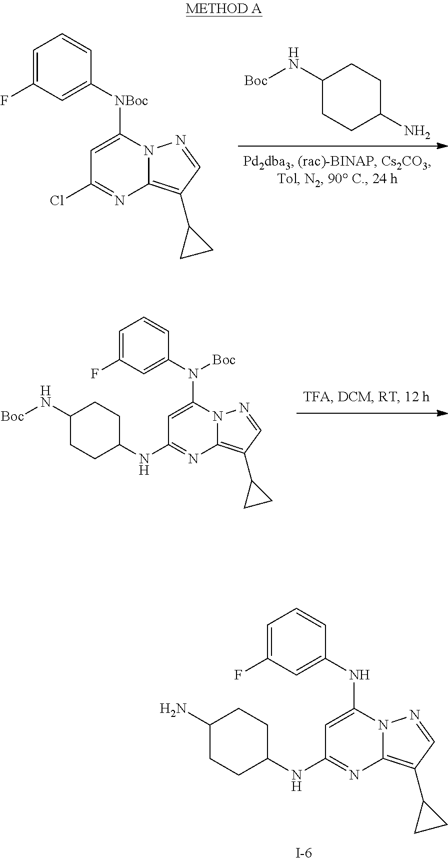

### Tert-Butyl bans-5-(4-((tert-butoxycarbonyl)amino)cyclohexyl)amino-3-cyclopropyl pyrazolo[1,5-a]pyrimidin-7-yl)(3-fluorophenyl)carbamate

To a stirred solution of tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (70 mg, 0.17 mmol, 1 eq), and 1-N-Boc-trans-1,4-cyclohexyldiamine (45 mg, 0.21 mmol, 1.2 eq) and Cs₂CO₃ (85 mg, 0.26 mmol, 1.5 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-Butyl trans-5-(4-((tert-butoxycarbonyl)amino)cyclohexyl)amino-3-cyclopropyl pyrazolo[1,5-a]pyrimidin-7-yl)(3-fluorophenyl)carbamate as a brown oil (13 mg, 13%). TLC system: EOAc/hexane (1:4), R_{f} value: ˜0.30.

### Trans-N⁵-(4-aminocyclohexyl)-3-cyclopropyl-N⁷-(3-fluorophenyl)pyrazolo[1,5-a]pyrimidine-5,7-diamine (I-6)

To a solution of tert-Butyl trans-5-(4-((tert-butoxycarbonyl)amino)cyclohexyl)amino-3-cyclopropyl pyrazolo[1,5-a]pyrimidin-7-yl)(3-fluorophenyl)carbamate (20 mg, 0.03 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford trans-N⁵-(4-aminocyclohexyl)-3-cyclopropyl-N⁷-(3-fluorophenyl)pyrazolo[1,5-a]pyrimidine-5,7-diamine (I-6) as an off white gummy solid (5 mg, yield: 63%). TLC system: DCM/Methanol (1:1), R_{f} value: ˜0.2; ¹HNMR (400 Hz, MeOD-d4) δ 7.56 (s, 1H), 7.44-7.38 (m, 1H), 7.19-7.12 (m, 2H), 6.94-6.89 (m, 1H), 5.74 (s, 1H), 3.90-3.83 (m, 1H), 3.02-2.94 (m, 1H), 2.20-2.17 (m, 2H), 2.05-2.02 (m, 2H), 1.87-1.82 (m, 1H), 1.54-1.44 (m, 2H), 1.37-1.30 (m, 2H), 0.85-0.81 (m, 2H), 0.79-0.75 (m, 2H). HRMS (ESI) m/z [M+H]⁺ calcd for C₂₁H₂₆FN₆ 381.2197, found 381.2200. HPLC: 95%.

### Example 9

### Synthesis of Cis-N⁵-(4-aminocyclohexyl)-3-cyclopropyl-N⁷-(3-fluorophenyl)pyrazolo[1,5-a]pyrimidine-5,7-diamine (Compound I-7)

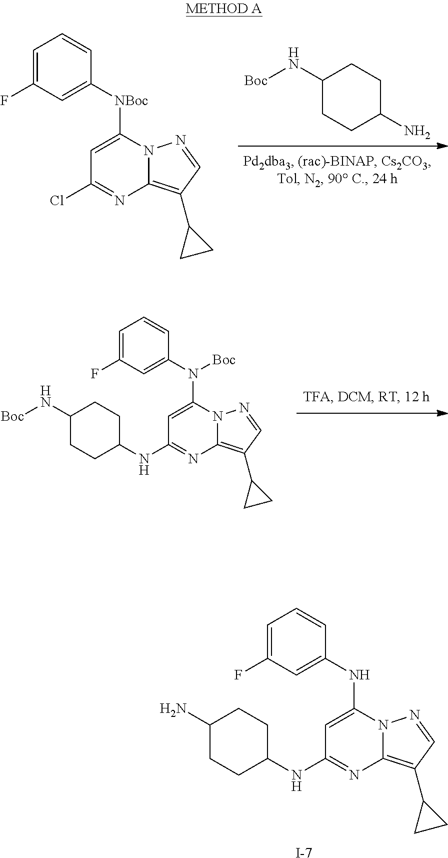

### Tert-Butyl (cis-4-(7-((tert-butoxycarbonyl)(3-fluorophenyl)amino)-3-cyclopropylpyrazolo[1,5-a]pyrimidin-5-yl)aminocyclohex-1-yl)amino-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (70 mg, 0.17 mmol, 1 eq), and 1-N-Boc-cis-1,4-cyclohexyldiamine (45 mg, 0.21 mmol, 1.2 eq) and Cs₂CO₃ (85 mg, 0.26 mmol, 1.5 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (cis-4-(7-((tert-butoxycarbonyl)(3-fluorophenyl)amino)-3-cyclopropylpyrazolo[1,5-a]pyrimidin-5-yl)aminocyclohex-1-yl)amino-1-carboxylate as a brown oil (20 mg, 20%). TLC system: EOAc/hexane (1:4), R_{f} value: ˜0.30.

### Cis-N⁵-(4-aminocyclohexyl)-3-cyclopropyl-N⁷-(3-fluorophenyl)pyrazolo[1,5-a]pyrimidine-5,7-diamine (I-7)

To a solution of tert-butyl (cis-4-(7-((tert-butoxycarbonyl)(3-fluorophenyl)amino)-3-cyclopropylpyrazolo[1,5-a]pyrimidin-5-yl)aminocyclohex-1-yl)amino-1-carboxylate (20 mg, 0.03 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford Cis-N⁵-(4-aminocyclohexyl)-3-cyclopropyl-N⁷-(3-fluorophenyl)pyrazolo[1,5-a]pyrimidine-5,7-diamine (I-7) as an off white gummy solid (8 mg, yield: 73%). TLC system: DCM/Methanol (1:1), R_{f} value: ˜0.21HNMR (400 Hz, MeOD-d4) δ 7.57 (s, 1H), 7.45-7.38 (m, 1H), 7.20-7.14 (m, 2H), 6.95-6.90 (m, 1H), 5.84 (s, 1H), 4.12-4.09 (m, 1H), 3.25-2.19 (m, 1H), 2.07-2.00 (m, 2H), 1.89-1.72 (m, 7H), 0.85-0.75 (m, 4H). HRMS (ESI) m/z [M+H]⁺ calcd for C₂₁H₂₆FN₆ 381.2197, found 381.2200. HPLC: 95%.

### Example 10

### Synthesis of (S)-3-cyclopropyl-N⁵-(piperidin-3-yl)-N⁷-((3-(trifluoromethyl)phenyl)pyrazolo[1,5-a]pyrimidine-5,7-diamine (Compound I-8)

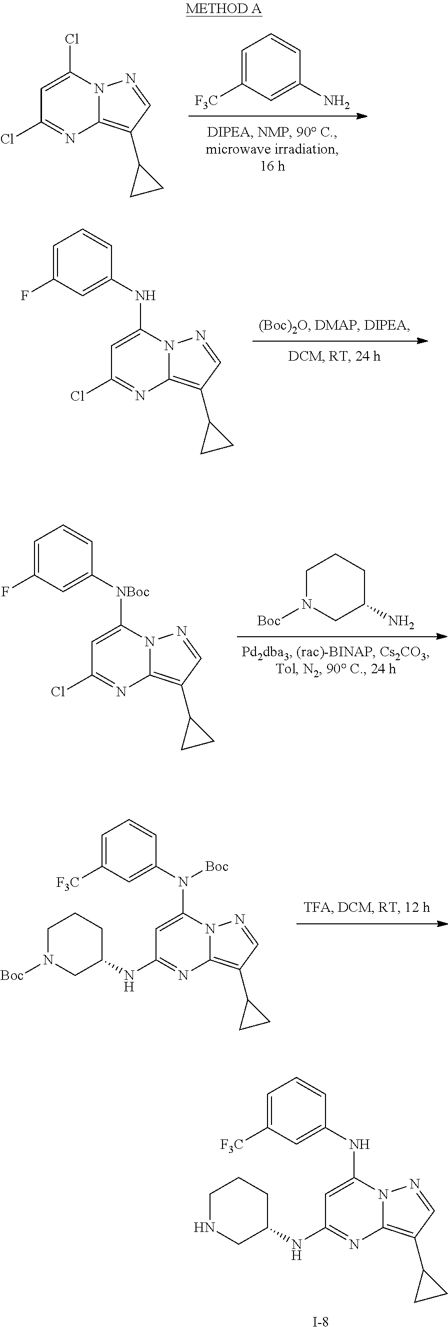

### 5-Chloro-3-cyclopropyl-N-(3-trifluoromethylphenyl)pyrazolo[1,5-a]pyrimidin-7-amine

To a stirred solution of 3-(trifluoromethyl)aniline (68 mg, 0.42 mmol, 1.2 eq) in NMP (4 mL) was added DIPEA (55 mg, 0.42 mmol, 1.2 eq) and the solution was stirred for 5 min. To this mixture 5,7-dichloro-3-cyclopropylpyrazolo[1,5-a]pyrimidine, prepared according to Example 1 above (80 mg, 0.35 mmol, 1.0 eq), was added at room temperature and stirring continued at 90° C. for 16 h under microwave irradiation. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound without purification went to the next step, 5-chloro-3-cyclopropyl-N-(3-trifluoromethylphenyl)pyrazolo[1,5-a]pyrimidin-7-amine as a brown liquid. TLC system: EtoAc:Hexane (1:4), R_{f} value: ˜0.5; ¹HNMR (400 MHz, CDCl₃) δ 8.17 (s, 1H), 7.77 (s, 1H), 7.63-7.56 (m, 4H), 6.26 (s, 1H), 2.06-2.02 (m, 1H), 1.00-0.96 (m, 2H), 0.81-0.77 (m, 2H).

### Tert-Butyl (5-chloro-3-cyclopropylpyrazolo[1,5-a]pyrimidin-7-yl)(3-trifluoromethylphenyl)carbamate

To a stirred solution of 5-chloro-3-cyclopropyl-N-(3-trifluoromethylphenyl)pyrazolo[1,5-a]pyrimidin-7-amine (123 mg, 0.35 mmol, 1.0 eq) in DCM (5 mL) at room temperature was added DIPEA (91 mg, 0.70 mmol, 2 eq) and DMAP (2 mg 0.016 mmol), followed by addition of (Boc)₂O (100 mg, 0.46 mmol 1.3 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyclopropylpyrazolo[1,5-a]pyrimidin-7-yl)(3-trifluoromethylphenyl)carbamate as a yellow oil (124 mg, 78% for two steps). TLC system: EtOAc/hexane (1:8), R_{f} value: ˜0.5.

### Tert-Butyl (S)-3-((7-((tert-butoxycarbonyl)(3-trifluoromethylphenyl)amino)-3-cyclopropylpyrazolo[1,5-a]pyrimidin-5-yl)amino)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-trifluoromethylphenyl)carbamate (110 mg, 0.24 mmol, 1 eq) and (S)-1-Boc-3-aminopiperidine (72 mg, 0.36 mmol, 1.5 eq) and Cs₂CO₃ (156 mg, 0.48 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-((7-((tert-butoxycarbonyl)(3-trifluoromethylphenyl)amino)-3-cyclopropylpyrazolo[1,5-a]pyrimidin-5-yl)amino)piperidine-1-carboxylate as a brown oil (75 mg, 51%). TLC system: EOAc/hexane (1:2), R_{f} value: ˜0.40; ¹HNMR (400 MHz, CDCl₃) δ 7.64 (s, 2H), 7.46-7.40 (m, 3H), 5.89 (s, 1H), 4.80-4.67 (m, 1H), 4.04-4.02 (m, 1H), 3.69-3.67 (m, 1H), 3.42-3.40 (m, 2H), 1.92-1.89 (m, 2H), 1.71-1.69 (m, 2H), 1.61-1.54 (m, 1H), 1.39 (s, 18H), 0.88-0.84 (m, 4H).

### (S)-3-cyclopropyl-N⁵-(piperidin-3-yl)-N⁷-((3-(trifluoromethyl)phenyl)pyrazolo[1,5-a]pyrimidine-5,7-diamine (I-8)

To a solution of tert-butyl (S)-3-((7-((tert-butoxycarbonyl)(3-trifluoromethylphenyl)amino)-3-cyclopropylpyrazolo[1,5-a]pyrimidin-5-yl)amino)piperidine-1-carboxylate (60 mg, 0.10 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture was stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-cyclopropyl-N⁵-(piperidin-3-yl)-N⁷-((3-(trifluoromethyl)phenyl)pyrazolo[1,5-a]pyrimidine-5,7-diamine (I-8) as an off white gummy solid (35 mg, yield: 83%). TLC system: DCM/Methanol (1:1), R_{f} value: ˜0.2; ¹HNMR (400 Hz, MeOD-d4) δ 7.63-7.59 (m, 3H), 7.57 (s, 1H), 7.47-7.45 (m, 1H), 4.87 (s, 1H), 4.16-4.06 (m, 1H), 3.46-3.42 (m, 1H), 3.10-3.05 (m, 1H), 2.82-2.71 (m, 2H), 2.09-2.04 (m, 1H), 1.93-1.82 (m, 2H), 1.77-1.67 (m, 1H), 1.61-1.52 (m, 1H), 0.85-0.74 (m, 4H). HRMS (ESI) m/z [M+H]⁺ calcd for C₂₁H₂₄F₃N₆ 417.2009, found 417.2009. HPLC: 95%.

### Example 11

### Synthesis of (S)-3-((3-cyclopropyl-5-((piperidin-3-yl)amino)pyrazolo[1,5-a]pyrimidin-7-yl)amino)benzonitrile (Compound I-9)

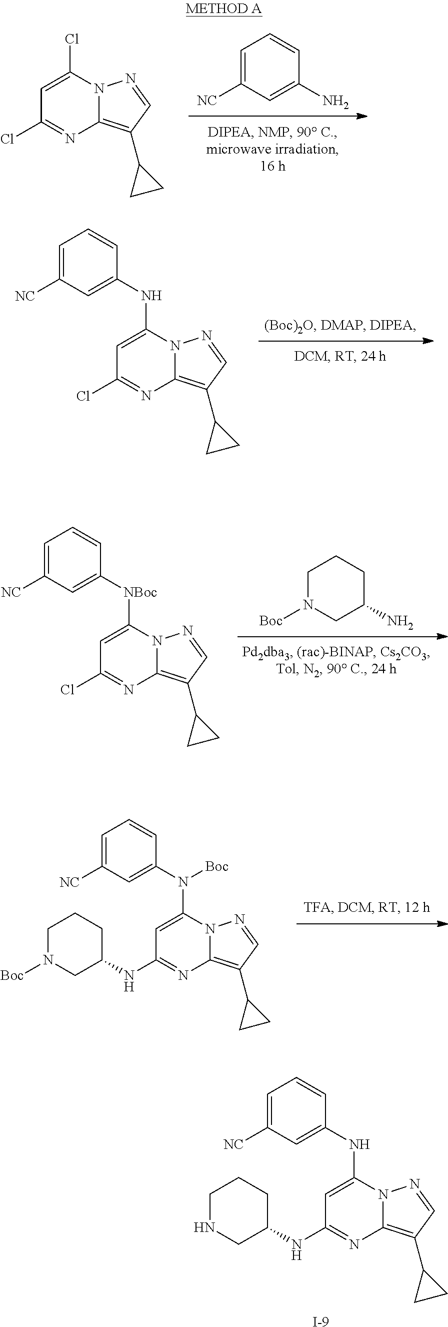

### 5-Chloro-3-cyclopropyl-N-(3-cyanophenyl)pyrazolo[1,5-a]pyrimidin-7-amine

To a stirred solution of 3-aminobenzonitrile (55 mg, 0.46 mmol, 1.3 eq) in NMP (4 mL) was added DIPEA (54 mg, 0.42 mmol, 1.2 eq) and the solution was stirred for 5 min. To this mixture, 5,7-dichloro-3-cyclopropylpyrazolo[1,5-a]pyrimidine, prepared according to Example 1 above (80 mg, 0.35 mmol, 1.0 eq), was added at room temperature and stirring continued at 90° C. for 16 h under microwave irradiation. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound without purification went to the next step, 5-chloro-3-cyclopropyl-N-(3-cyanophenyl)pyrazolo[1,5-a]pyrimidin-7-amine as a brown liquid. TLC system: EtoAc:Hexane (1:4), R_{f} value: ˜0.4; ¹HNMR (400 MHz, CDCl₃) δ 8.18 (s, 1H), 7.77 (s, 1H), 7.65 (s, 1H), 7.73-7.59 (m, 3H), 6.27 (s, 1H), 2.07-2.00 (m, 1H), 1.00-0.96 (s, 2H), 0.81-0.77 (m, 2H).

### Tert-Butyl (5-chloro-3-cyclopropylpyrazolo[1,5-a]pyrimidin-7-yl)(3-cyanophenyl)carbamate

To a stirred solution of 5-chloro-3-cyclopropyl-N-(3-cyanophenyl)pyrazolo[1,5-a]pyrimidin-7-amine (108 mg, 0.35 mmol, 1.0 eq) in DCM (5 mL) at room temperature was added DIPEA (90 mg, 0.7 mmol, 2 eq) and DMAP (2 mg 0.016 mmol), followed by addition of (Boc)₂O (100 mg, 0.46 mmol, 1.3 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyclopropylpyrazolo[1,5-a]pyrimidin-7-yl)(3-cyanophenyl)carbamate as a yellow oil (100 mg, 70% for two steps). TLC system: EtOAc/hexane (1:4), R_{f} value: ˜0.5; ¹HNMR (400 MHz, CDCl₃) δ 7.84 (s, 1H), 7.61-7.60 (m, 1H), 7.56-7.52 (m, 2H), 7.48-7.42 (s, 1H), 6.67 (s, 1H), 2.10-2.03 (m, 1H), 1.37 (s, 9H), 1.03-0.95 (m, 2H), 0.86-0.82 (m, 2H).

### Tert-Butyl (S)-3-((7-((tert-butoxycarbonyl)(3-cyanophenyl)amino)-3-cyclopropylpyrazolo[1,5-a]pyrimidin-5-yl)amino)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-cyanophenyl)carbamate (100 mg, 0.24 mmol, 1 eq) and (S)-1-Boc-3-aminopiperidine (73 mg, 0.37 mmol, 1.5 eq) and Cs₂CO₃ (156 mg, 0.48 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-((7-((tert-butoxycarbonyl)(3-cyanophenyl)amino)-3-cyclopropylpyrazolo[1,5-a]pyrimidin-5-yl)amino)piperidine-1-carboxylate as a brown oil (67 mg, 59%). TLC system: EOAc/hexane (1:2), R_{f} value: ˜0.20; ¹HNMR (400 MHz, CDCl₃) δ 7.63 (s, 1H), 7.62 (s, 1H), 7.59-7.56 (m, 1H), 7.50-7.47 (m, 1H), 7.40 (t, J=7.9 Hz, 1H), 5.91 (s, 1H), 4.85-4.72 (m, 1H), 4.07-4.03 (m, 1H), 3.70-3.66 (m, 1H), 3.42-3.39 (m, 2H), 1.94-1.87 (m, 2H), 1.75-1.70 (m, 1H), 1.62-1.57 (m, 2H), 1.41 (s, 9H), 1.38 (s, 9H), 0.89-0.82 (m, 4H).

### (S)-3-((3-cyclopropyl-5-((piperidin-3-yl)amino)pyrazolo[1,5-a]pyrimidin-7-yl)amino)benzonitrile (I-9)

To a solution of tert-butyl (S)-3-((7-((tert-butoxycarbonyl)(3-cyanophenyl)amino)-3-cyclopropylpyrazolo[1,5-a]pyrimidin-5-yl)amino)piperidine-1-carboxylate (50 mg, 0.09 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture was stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-((3-cyclopropyl-5-((piperidin-3-yl)amino)pyrazolo[1,5-a]pyrimidin-7-yl)amino)benzonitrile (I-9) (25 mg, yield: 74%). TLC system: DCM/Methanol (1:1), R_{f} value: ˜0.2; ¹HNMR (400 Hz, MeOD-d4) δ 7.75-7.45 (m, 5H), 4.87 (s, 1H), 4.17-4.10 (m, 1H), 3.48-3.44 (m, 1H), 3.12-3.07 (m, 1H), 2.84-2.73 (m, 2H), 2.09-2.05 (m, 1H), 1.94-1.83 (m, 2H), 1.78-1.69 (m, 1H), 1.62-1.53 (m, 1H), 0.85-0.74 (m, 4H). HRMS (ESI) m/z [M+H]⁺ calcd for C₂₁H₂₄N₇ 374.2088, found 374.2090. HPLC: 95%.

### Example 12

### Synthesis of (S)-3-cyclopropyl-7-(3-trifluoroethylphenyl)amino-5-(piperidin-3-yl)aminopyrazolo[1,5-a]pyrimidine (Compound I-10)

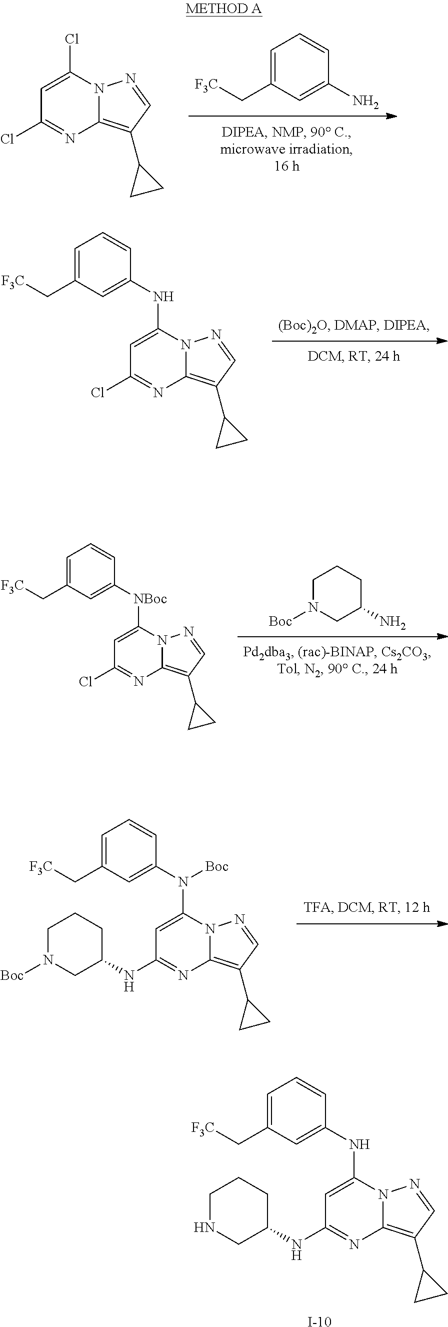

### 5-Chloro-3-cyclopropyl-N-(3-trifluoroethylphenyl)pyrazolo[1,5-a]pyrimidin-7-amine

To a stirred solution of 3-(2,2,2-trifluoroethyl)aniline (55 mg, 0.32 mmol, 1.2 eq) in NMP (4 mL) was added DIPEA (42 mg, 0.32 mmol, 1.2 eq) and the solution was stirred for 5 min. To this mixture 5,7-dichloro-3-cyclopropylpyrazolo[1,5-a]pyrimidine, prepared according to Example 1 above (60 mg, 0.26 mmol, 1.0 eq), was added at room temperature and stirring continued at 90° C. for 16 h under microwave irradiation. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound without purification went to the next step, 5-chloro-3-cyclopropyl-N-(3-trifluoroethylphenyl)pyrazolo[1,5-a]pyrimidin-7-amine as a brown liquid. TLC system: EoAc:Hexane (1:4), R_{f} value: ˜0.4. ¹HNMR (400 MHz, CDCl₃) δ 8.10 (s, 1H), 7.75 (s, 1H), 7.48 (t, J=7.8 Hz, 1H), 7.36-7.34 (m, 1H), 7.29-7.25 (m, 2H), 6.26 (s, 1H), 3.43 (q, J=10.6 Hz, 2H), 2.07-2.00 (m, 1H), 1.00-0.95 (s, 2H), 0.80-0.76 (m, 2H).

### Tert-Butyl (5-chloro-3-cyclopropylpyrazolo[1,5-a]pyrimidin-7-yl)(3-trifluoroethylphenyl)carbamate

To a stirred solution of 5-chloro-3-cyclopropyl-N-(3-trifluoroethylphenyl)pyrazolo[1,5-a]pyrimidin-7-amine (110 mg, 0.30 mmol, 1.0 eq) in DCM (5 mL) at room temperature was added DIPEA (77 mg, 0.6 mmol, 2 eq) and DMAP (2 mg 0.016 mmol), followed by addition of (Boc)₂O (85 mg, 0.39 mmol, 1.3 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyclopropylpyrazolo[1,5-a]pyrimidin-7-yl)(3-trifluoroethylphenyl)carbamate as a yellow oil (97 mg, 69% for two steps). TLC system: EOAc/hexane (1:4), R_{f} value: ˜0.6.

### Tert-Butyl (S)-3-((7-((tert-butoxycarbonyl)(3-trifluoroethylphenyl)amino)-3-cyclopropylpyrazolo[1,5-a]pyrimidin-5-yl)amino)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-trifluoroethylphenyl)carbamate (90 mg, 0.20 mmol, 1 eq) and (S)-1-Boc-3-aminopiperidine (60 mg, 0.30 mmol, 1.5 eq) and Cs₂CO₃ (130 mg, 0.40 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-((7-((tert-butoxycarbonyl)(3-trifluoroethylphenyl)amino)-3-cyclopropylpyrazolo[1,5-a]pyrimidin-5-yl)amino)piperidine-1-carboxylate as a brown oil (46 mg, 44%). TLC system: EOAc/hexane (1:1), R_{f} value: ˜0.30; ¹HNMR (400 MHz, CDCl₃) δ 7.64 (s, 1H), 7.32 (s, 1H), 7.29-7.23 (m, 1H), 7.17-7.15 (m, 2H), 5.87 (s, 1H), 4.75-4.70 (m, 1H), 4.04-4.01 (m, 1H), 3.71-3.68 (m, 1H), 3.44-3.30 (m, 4H), 1.94-1.87 (m, 2H), 1.73-1.64 (m, 2H), 1.58-1.54 (m, 1H), 1.39 (s, 9H), 1.37 (s, 9H), 0.89-0.82 (m, 4H).

### (S)-3-Cyclopropyl-7-(3-trifluoroethylphenyl)amino-5-(piperidin-3-yl)aminopyrazolo[1,5-a]pyrimidine (I-10)

To a solution of tert-butyl (S)-3-((7-((tert-butoxycarbonyl)(3-trifluoroethylphenyl)amino)-3-cyclopropylpyrazolo[1,5-a]pyrimidin-5-yl)amino)piperidine-1-carboxylate (40 mg, 0.07 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-cyclopropyl-7-(3-trifluoroethylphenyl)amino-5-(piperidin-3-yl)aminopyrazolo[1,5-a]pyrimidin (I-10) as an off white gummy solid (20 mg, yield: 71%). TLC system: DCM/Methanol (1:1), R_{f} value: ˜0.2; ¹HNMR (400 Hz, MeOD-d4) δ 7.55 (s, 1H), 7.42-7.32 (m, 2H), 7.29 (s, 1H), 7.17 (d, J=7.4 Hz, 1H), 4.88 (s, 1H), 4.07-4.00 (m, 1H), 3.50 (d, J=11.1 Hz, 2H), 3.36-3.30 (m, 1H), 3.00-2.96 (m, 1H), 2.69-2.56 (m, 2H), 2.07-2.00 (m, 1H), 1.88-1.79 (m, 2H), 1.69-1.59 (m, 1H), 1.53-1.45 (m, 1H), 0.84-0.80 (m, 2H), 0.76-0.73 (m, 2H). HRMS (ESI) m/z [M+H]⁺ calcd for C₂₁H₂₆F₃N₆ 431.2166, found 431.2171. HPLC: 95%.

### Example 13

### Synthesis of (3S,4S)-3-cyclopropyl-7-(3-fluorophenyl)amino-5-(3-hydroxypiperidin-4-yl)methylaminopyrazolo[1,5-a]pyrimidine (Compound I-11)

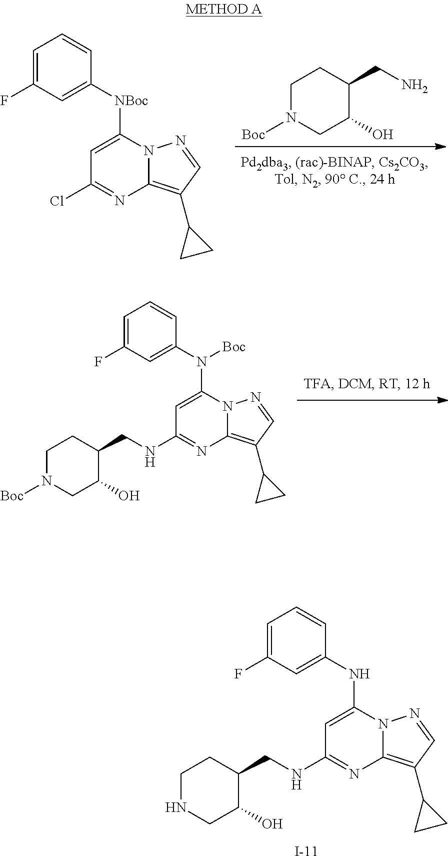

### Tert-Butyl (3S,4S)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (100 mg, 0.25 mmol, 1 eq), and tert-butyl (3S,4S)-4-(aminomethyl)-3-hydroxy-1-piperidinecarboxylate (69 mg, 0.30 mmol, 1.2 eq) and Cs₂CO₃ (163 mg, 0.50 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (3S,4S)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate as a brown oil (45 mg, 30%). TLC system: EtOAc/hexane (1:1), R_{f} value: ˜0.40; ¹HNMR (400 MHz, CDCl₃) δ 7.60 (s, 1H), 7.28-7.22 (m, 1H), 7.11-7.07 (m, 2H), 6.95-6.90 (m, 1H), 5.97 (s, 1H), 4.31-4.24 (m, 2H), 4.14-4.09 (m, 1H), 3.24-3.21 (m, 1H), 3.03-2.93 (m, 1H), 2.62-2.53 (m, 2H), 1.87-1.81 (m, 1H), 1.57-1.50 (m, 3H), 1.45 (s, 9H), 1.39 (s, 9H), 0.93-0.88 (m, 2H), 0.66-0.62 (m, 2H).

### (3S,4S)-3-Cyclopropyl-7-(3-fluorophenyl)amino-5-(3-hydroxypiperidin-4-yl)methylaminopyrazolo[1,5-a]pyrimidine (I-11)

To a solution of tert-butyl (3S,4S)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate (40 mg, 0.07 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (3S,4S)-3-cyclopropyl-7-(3-fluorophenyl)amino-5-(3-hydroxy-4-piperidin-4-yl)methylaminopyrazolo[1,5-a]pyrimidine (I-11) as an off white gummy solid (25 mg, yield: 89%). TLC system: DCM/Methanol (1:1), R_{f} value: ˜0.2; ¹HNMR (400 Hz, MeOD-d4) δ 7.55 (s, 1H), 7.45-7.39 (m, 1H), 7.20-7.13 (m, 2H), 6.96-6.91 (m, 1H), 5.78 (s, 1H), 4.05-4.00 (m, 1H), 3.44-3.39 (m, 1H), 3.26-3.22 (m, 1H), 3.18-3.12 (m, 2H), 2.76-2.69 (m, 1H), 2.58 (dd, J=11.8 10.7 Hz, 1H), 1.80-1.73 (m, 2H), 1.59-1.53 (m, 2H), 0.87-0.82 (m, 2H), 0.65-0.58 (m, 2H). HRMS (ESI) m/z [M+H]⁺ calcd for C₂₁H₂₆FN₆O 397.2147, found 397.2150. HPLC: 95%.

### Example 14

### Synthesis of (3S,4R)-4-((3-cyclopropyl-7-((3-fluorophenyl)amino)pyrazolo[1,5-a]pyrimidin-5-yl)aminomethyl)piperidin-3-ol (Compound I-12)

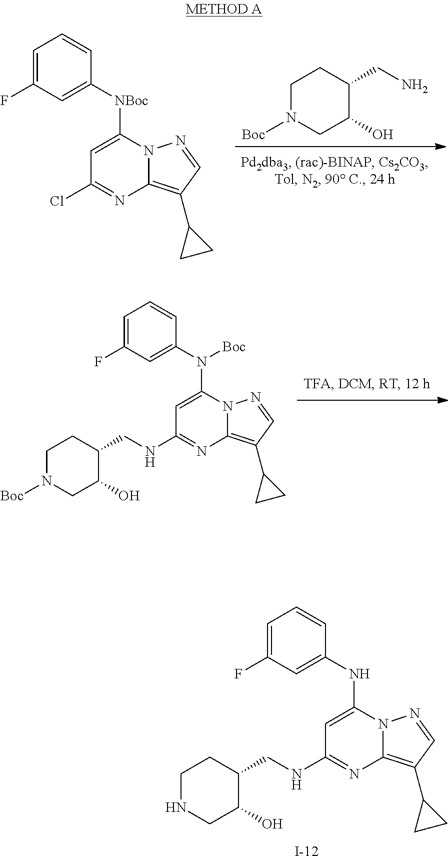

### Tert-Butyl (3S,4R)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (100 mg, 0.25 mmol, 1 eq), and cis-1-Boc-4-aminomethyl-3-hydroxypiperidine (69 mg, 0.30 mmol, 1.2 eq) and Cs₂CO₃ (163 mg, 0.50 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (3S,4R)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate as a brown oil (90 mg, 60%). TLC system: EtOAc/hexane (1:1), R_{f} value: ˜0.40; ¹HNMR (400 MHz, CDCl₃) δ 7.52 (s, 1H), 7.22-7.17 (m, 1H), 7.17-7.01 (m, 2H), 6.90-6.84 (m, 1H), 5.93 (s, 1H), 4.20-4.06 (m, 2H), 3.75-3.71 (m, 1H), 3.30-3.20 (m, 1H), 3.02-2.92 (m, 1H), 2.76-2.64 (m, 2H), 1.89-1.80 (m, 1H), 1.70-1.55 (m, 3H), 1.41 (s, 9H), 1.34 (s, 9H), 0.87-0.82 (m, 2H), 0.65-0.62 (m, 2H).

### (3S,4R)-4-((3-cyclopropyl-7-((3-fluorophenyl)amino)pyrazolo[1,5-a]pyrimidin-5-yl)aminomethyl)piperidin-3-ol (I-12)

To a solution of tert-butyl (3S,4R)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate (80 mg, 0.13 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (3S,4R)-4-((3-cyclopropyl-7-((3-fluorophenyl)amino)pyrazolo[1,5-a]pyrimidin-5-yl)aminomethyl)piperidin-3-ol (I-12) as an off white gummy solid (23 mg, yield: 44%). TLC system: DCM/Methanol (1:1), R_{f} value: ˜0.2; ¹HNMR (400 Hz, MeOD-d4) δ 7.54 (s, 1H), 7.43-7.38 (m, 1H), 7.19-7.12 (m, 2H), 6.95-6.90 (m, 1H), 5.76 (s, 1H), 3.87-3.86 (m, 1H), 3.71-3.65 (m, 1H), 3.28-3.24 (m, 2H), 3.10-3.06 (m, 1H), 2.97-2.93 (m, 1H), 2.89-2.82 (m, 1H), 1.83-1.76 (m, 3H), 1.59-1.54 (m, 1H), 0.86-0.82 (m, 2H), 0.66-0.62 (m, 2H). HRMS (ESI) m/z [M+H]⁺ calcd for C21H26FN6O 397.2147, found 397.2150. HPLC: 95%.

### Example 15

### Synthesis of (S)-3-Cyclopropyl-7-(3-fluorophenyl)amino-5-(piperidin-3-yl)methylaminopyrazolo[1,5-a]pyrimidine (Compound I-13)

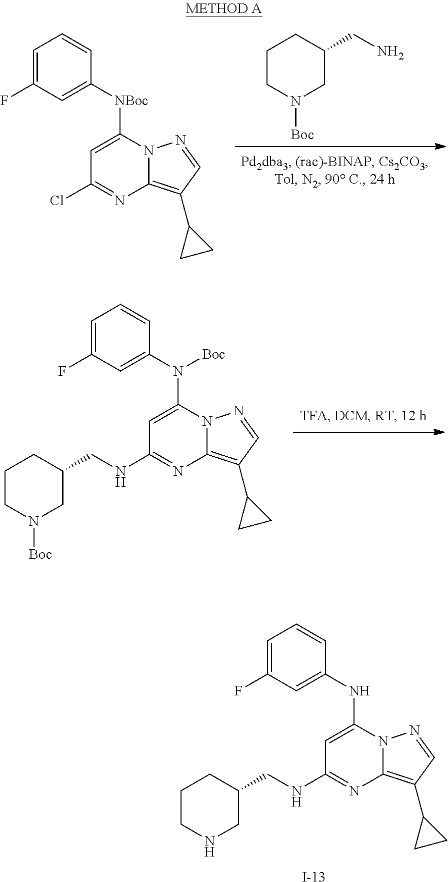

### Tert-Butyl (R)-3-((7-((tert-butoxycarbonyl)(3-fluorophenyl)amino)-3-cyclopropylpyrazolo[1,5-a]pyrimidin-5-yl)aminomethyl)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (100 mg, 0.25 mmol, 1 eq), and (R)-1-Boc-3-(aminomethyl)piperidine (65 mg, 0.30 mmol, 1.2 eq) and Cs₂CO₃ (163 mg, 0.50 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (R)-3-((7-((tert-butoxycarbonyl)(3-fluorophenyl)amino)-3-cyclopropylpyrazolo[1,5-a]pyrimidin-5-yl)aminomethyl)piperidine-1-carboxylate as a brown oil (85 mg, 59%). TLC system: EtOAc/hexane (1:2), R_{f} value: ˜0.50; ¹HNMR (400 MHz, CDCl₃) δ 7.60 (s, 1H), 7.24-7.20 (m, 1H), 7.09-7.03 (m, 2H), 6.90-6.85 (m, 1H), 5.88 (s, 1H), 3.78-4.70 (m, 3H), 2.95-2.86 (m, 2H), 2.73-2.67 (m, 1H), 1.90-1.80 (m, 3H), 1.77-1.73 (m, 1H), 1.66-1.59 (m, 2H), 1.41 (s, 9H), 1.35 (s, 9H), 0.86-0.82 (m, 4H).

### (S)-3-Cyclopropyl-7-(3-fluorophenyl)amino-5-(piperidin-3-yl)methylamino pyrazolo[1,5-a]pyrimidine (I-13

To a solution of tert-butyl (R)-3-((7-((tert-butoxycarbonyl)(3-fluorophenyl)amino)-3-cyclopropylpyrazolo[1,5-a]pyrimidin-5-yl)aminomethyl)piperidine-1-carboxylate (80 mg, 0.14 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-cyclopropyl-7-(3-fluorophenyl)amino-5-(piperidin-3-yl)methylaminopyrazolo[1,5-a]pyrimidine (I-13) (40 mg, yield: 75%). TLC system: DCM/Methanol (1:1), R_{f} value: ˜0.2; ¹HNMR (400 Hz, MeOD-d4) δ 7.56 (s, 1H), 7.43-7.37 (m, 1H), 7.18-7.11 (m, 2H), 6.94-6.89 (m, 1H), 5.78 (s, 1H), 3.40-3.38 (m, 2H), 3.32-3.30 (m, 1H), 3.23-3.19 (m, 1H), 2.91-2.85 (m, 1H), 2.73-2.68 (m, 1H), 2.12-2.06 (m, 1H), 1.95-1.91 (m, 2H), 1.87-1.81 (m, 1H), 1.75-1.65 (m, 1H), 1.39-1.29 (m, 1H), 0.85-0.82 (m, 2H), 0.78-0.73 (m, 2H). HRMS (ESI) m/z [M+H]⁺ calcd for C₂₁H₂₆FN₆ 381.2197, found 381.2201. HPLC: 95%.

### Example 16

### Synthesis of (S)-3-cyclopropyl-7-(3-fluorophenyl)amino-5-(piperidin-3-yl)methoxylpyrazolo[1,5-a]pyrimidine (Compound I-14)

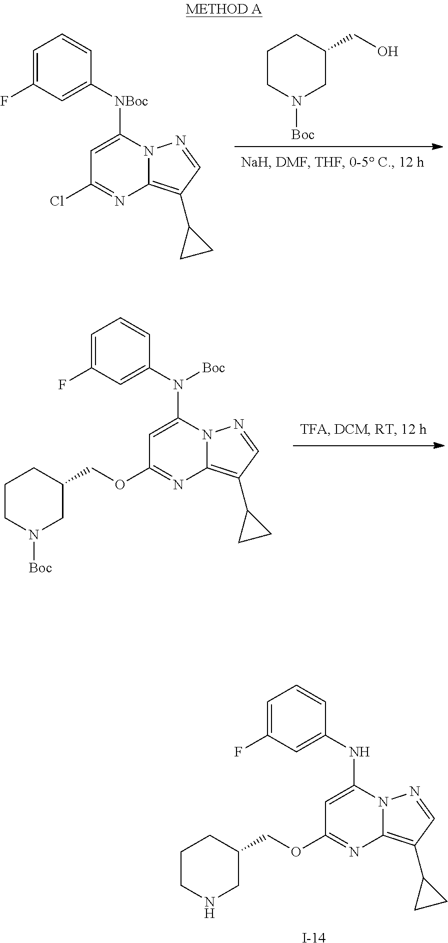

### Tert-Butyl (S)-3-((7-((tert-butoxycarbonyl)(3-fluorophenyl)amino)-3-cyclopropylpyrazolo[1,5-a]pyrimidin-5-yl)hydroxymethyl)piperidine-1-carboxylate

To a stirred mixture of NaH (60%, 20 mg, 0.48 mmol, 2.3 eq) in DMF (3 mL), the mixture of tert-butyl (5-chloro-3-cyclopropylpyrazolo[1,5-a]pyrimidin-7-yl)(3-fluorophenyl)carbamate, prepared according to Example 6 above (85 mg, 0.21 mmol, 1.0 eq), and (S)-1-Boc-3-(hydroxymethyl)piperidine (55 mg, 0.25 mmol, 1.2 eq) in THF (3 mL) was added at 0-5° C. and the reaction was stirred at room temperature for 12 h. After completion of reaction by TLC, the reaction mixture was quenched with ice cold water and extracted with (3×50 mL) ethyl acetate. The combined organic layer was washed with water (30 mL), brine (30 mL), dried over sodium sulfate, and concentrated to afford crude product which was purified by using CombiFlash chromatography (4 g column) to afford tert-butyl (S)-3-((7-((tert-butoxycarbonyl)(3-fluorophenyl)amino)-3-cyclopropylpyrazolo[1,5-a]pyrimidin-5-yl)hydroxymethyl)piperidine-1-carboxylate as yellow oil (55 mg; 45%). TLC system: EtoAc:Hexane (1:4), R_{f} value: ˜0.3; ¹HNMR (400 MHz, CDCl₃) δ 7.73 (s, 1H), 7.29-7.23 (m, 1H), 7.10-7.17 (m, 2H), 6.96-6.90 (m, 1H), 6.17 (s, 1H), 4.32-4.20 (m, 2H), 3.90-3.86 (m, 1H), 2.89-2.75 (m, 2H), 2.02-1.86 (m, 4H), 1.72-1.67 (m, 1H), 1.54-1.48 (m, 1H), 1.44 (s, 9H), 1.38 (s, 9H), 0.94-0.84 (m, 4H).

### (S)-3-Cyclopropyl-7-(3-fluorophenyl)amino-5-(piperidin-3-yl)methoxylpyrazolo[1,5-a]pyrimidine (I-14)

To a solution of afford tert-butyl (S)-3-((7-((tert-butoxycarbonyl)(3-fluorophenyl)amino)-3-cyclopropylpyrazolo[1,5-a]pyrimidin-5-yl)hydroxymethyl)piperidine-1-carboxylate (55 mg, 0.09 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-cyclopropyl-7-(3-fluorophenyl)amino-5-(piperidin-3-yl)methoxylpyrazolo[1,5-a]pyrimidine (I-14) as an off white solid (25 mg, yield: 74%). TLC system: DCM/Methanol (1:1), R_{f} value: ˜0.2; ¹HNMR (400 Hz, MeOD-d4) δ 7.69 (s, 1H), 7.44-7.38 (m, 1H), 7.19-7.11 (m, 2H), 6.97-6.93 (m, 1H), 5.76 (s, 1H), 4.29-4.25 (m, 1H), 4.19-4.14 (m, 1H), 3.31-3.28 (m, 1H), 3.17-3.13 (m, 1H), 2.75-2.68 (m, 1H), 2.64-2.58 (m, 1H), 2.17-2.08 (m, 1H), 1.92-1.80 (m, 3H), 1.70-1.58 (m, 1H), 1.39-1.29 (m, 1H), 0.89-0.84 (m, 2H), 0.83-0.79 (m, 2H). HRMS (ESI) m/z [M+H]⁺ calcd for C₂₁H₂₅FN₅O 382.2038, found 382.2042, HPLC: 95%.

### Example 17

### Synthesis of (3S,4S)-3-((3-cyclopropyl-5-(((3-hydroxy)piperidin-4-yl)methylamino)pyrazolo[1,5-a]pyrimidin-7-yl)amino)benzonitrile (Compound I-15)

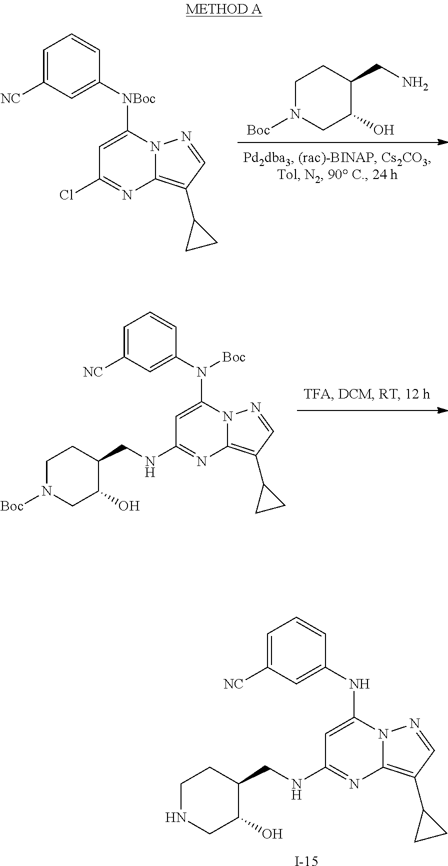

### Tert-Butyl (3S,4S)-4-((7-((tert-butoxycarbonyl)(3-cyanophenyl)amino)-3-cyclopropylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-cyanophenyl)carbamate, prepared according to Example 11 above (80 mg, 0.20 mmol, 1 eq), and tert-butyl (3S,4S)-4-(aminomethyl)-3-hydroxy-1-piperidinecarboxylate (55 mg, 0.24 mmol, 1.2 eq) and Cs₂CO₃ (130 mg, 0.40 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (3S,4S)-4-((7-((tert-butoxycarbonyl)(3-cyanophenyl)amino)-3-cyclopropylpyrazolo[1,5-a]pyrimidin-5-yl)aminomethyl)-3-hydroxypiperidine-1-carboxylate as a brown oil (40 mg, 33%). TLC system: EtOAc/hexane (1:2), R_{f} value: ˜0.20; ¹HNMR (400 MHz, CDCl₃) δ 7.65 (s, 1H), 7.59-7.55 (m, 2H), 7.53-7.49 (m, 1H), 7.42 (t, J=7.9 Hz, 1H), 5.93 (s, 1H), 5.09-5.06 (m, 1H), 4.41-4.37 (m, 2H), 3.28-3.25 (m, 1H), 3.11-3.06 (m, 1H), 2.68-2.54 (m, 2H), 1.87-1.79 (m, 1H), 1.62-1.55 (m, 3H), 1.41 (s, 9H), 1.39 (s, 9H), 0.93-0.90 (m, 2H), 0.66-0.62 (m, 2H).

### (3S,4S)-3-((3-cyclopropyl-5-(((3-hydroxy)piperidin-4-yl)methylamino)pyrazolo[1,5-a]pyrimidin-7-yl)amino)benzonitrile (I-15)

To a solution of tert-butyl (3S,4S)-4-((7-((tert-butoxycarbonyl)(3-cyanophenyl)amino)-3-cyclopropylpyrazolo[1,5-a]pyrimidin-5-yl)aminomethyl)-3-hydroxypiperidine-1-carboxylate (30 mg, 0.05 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (3S,4S)-3-((3-cyclopropyl-5-(((3-hydroxy)piperidin-4-yl)methylamino)pyrazolo[1,5-a]pyrimidin-7-yl)amino)benzonitrile (I-15) as an off white gummy solid (15 mg, yield: 75%). TLC system: DCM/Methanol (1:1), R_{f} value: ˜0.2; ¹HNMR (400 Hz, MeOD-d4) δ 7.87 (s, 1H), 7.79-7.66 (m, 4H), 5.70 (s, 1H), 3.64-3.58 (m, 2H), 3.45-3.37 (m, 3H), 3.01-2.93 (m, 1H), 2.84-2.78 (m, 1H), 2.12-2.07 (m, 1H), 1.89-1.82 (m, 2H), 1.66-1.58 (m, 1H), 0.99-0.94 (m, 2H), 0.67-0.64 (m, 2H). HRMS (ESI) m/z [M+H]⁺ calcd for C₂₂H₂₆C₁₃N₇O 404.2193, found 404.2195. HPLC: 95%.

### Example 18

### Synthesis of (3S,4S)-3-((3-cyclopropyl-5-(((3-hydroxy)piperidin-4-yl)methylamino)pyrazolo[1,5-a]pyrimidin-7-yl)amino)-5-fluorobenzonitrile trifluoroacetate salt (Compound I-16)

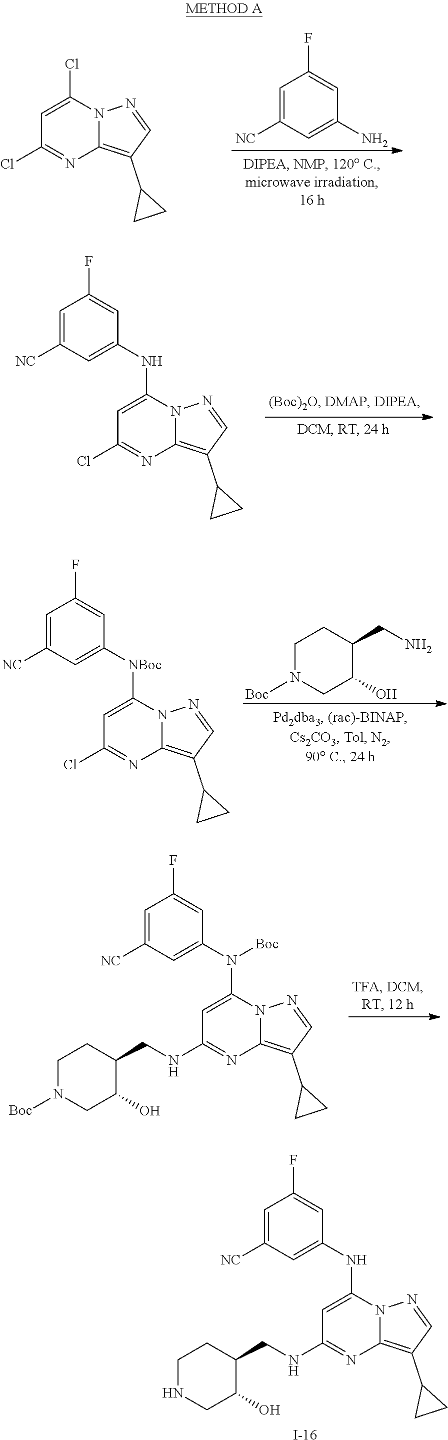

### 5-Chloro-3-cyclopropyl-N-(3-cyano-5-fluorophenyl)pyrazolo[1,5-a]pyrimidin-7-amine

To a stirred solution of 5-amino-3-fluorobenzonitrile (57 mg, 0.42 mmol, 1.2 eq) in NMP (4 mL) was added DIPEA (59 mg, 0.46 mmol, 1.3 eq) and the solution was stirred for 5 min. To this mixture 5,7-dichloro-3-cyclopropylpyrazolo[1,5-a]pyrimidine, prepared according to Example 1 above (80 mg, 0.35 mmol, 1.0 eq), was added at room temperature and stirring continued at 120° C. for 16 h under microwave irradiation. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound without purification went to the next step, 5-chloro-3-cyclopropyl-N-(3-cyano-5-fluorophenyl)pyrazolo[1,5-a]pyrimidin-7-amine as a brown liquid. TLC system: EtoAc:Hexane (1:4), R_{f} value: ˜0.3.

### Tert-Butyl (5-chloro-3-cyclopropylpyrazolo[1,5-a]pyrimidin-7-yl)(3-cyano-5-fluorophenyl)carbamate

To a stirred solution of 5-chloro-3-cyclopropyl-N-(3-cyano-5-fluorophenyl)pyrazolo[1,5-a]pyrimidin-7-amine (115 mg, 0.35 mmol, 1.0 eq) in DCM (5 mL) at room temperature was added DIPEA (90 mg, 0.7 mmol, 2 eq) and DMAP (2 mg 0.016 mmol), followed by addition of (Boc)₂O (100 mg, 0.46 mmol, 1.3 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyclopropylpyrazolo[1,5-a]pyrimidin-7-yl)(3-cyano-5-fluorophenyl)carbamate as a yellow oil (120 mg, 80% for two steps). TLC system: EtOAc/hexane (1:4), R_{f} value: ˜0.5; ¹HNMR (400 MHz, CDCl₃) δ 7.84 (s, 1H), 7.36-7.34 (m, 2H), 7.25-7.22 (m, 1H), 6.69 (s, 1H), 2.10-2.05 (m, 1H), 1.45 (s, 9H), 1.37 (s, 9H), 1.04-0.99 (m, 2H), 0.87-0.83 (m, 2H).

### Tert-Butyl (3S,4S)-4-((7-((tert-butoxycarbonyl)(3-cyano-5-fluorophenyl)amino)-3-cyclopropylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-cyano-5-fluorophenyl)carbamate (80 mg, 0.20 mmol, 1 eq) and tert-butyl (3S,4S)-4-(aminomethyl)-3-hydroxy-1-piperidinecarboxylate (55 mg, 0.24 mmol, 1.2 eq) and Cs₂CO₃ (130 mg, 0.40 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (3S,4S)-4-((7-((tert-butoxycarbonyl)(3-cyano-5-fluorophenyl)amino)-3-cyclopropylpyrazolo[1,5-a]pyrimidin-5-yl)aminomethyl)-3-hydroxypiperidine-1-carboxylate as a brown oil (64 mg, 52%). TLC system: EtOAc/hexane (1:1), R_{f} value: ˜0.30.

### (3S,4S)-3-((3-cyclopropyl-5-(((3-hydroxy)piperidin-4-yl)methylamino)pyrazolo[1,5-a]pyrimidin-7-yl)amino)-5-fluorobenzonitrile trifluoroacetate Salt (I-16)

To a solution of tert-butyl (3S,4S)-4-((7-((tert-butoxycarbonyl)(3-cyano-5-fluorophenyl)amino)-3-cyclopropylpyrazolo[1,5-a]pyrimidin-5-yl)aminomethyl)-3-hydroxypiperidine-1-carboxylate (50 mg, 0.08 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (3S,4S)-3-((3-cyclopropyl-5-(((3-hydroxy)piperidin-4-yl)methylamino)pyrazolo[1,5-a]pyrimidin-7-yl)amino)-5-fluorobenzonitrile trifluoroacetate salt (I-16) as an off white gummy solid (30 mg, yield: 88%). TLC system: DCM/Methanol (1:1), R_{f} value: ˜0.2; ¹HNMR (400 Hz, DMSO-d₆) δ 7.57 (s, 1H), 7.55 (s, 1H), 7.50-7.46 (m, 1H), 7.34-7.32 (m, 1H), 5.89 (s, 1H), 4.03-3.98 (m, 1H), 3.44-3.40 (m, 1H), 3.28-3.15 (m, 3H), 2.78-2.72 (m, 1H), 2.64-2.58 (m, 1H), 1.85-1.79 (m, 1H), 1.78-1.74 (m, 1H), 1.63-1.56 (m, 2H), 0.87-0.84 (m, 2H), 0.65-0.61 (m, 2H). HRMS (ESI) m/z [M+H]⁺ calcd for C₂₂H₂₅FN₇O 422.2099, found 422.2101. HPLC: 95%.

### Example 19

### Synthesis of (3S,4S)-4-((3-cyclopropyl-7-((3-fluorophenyl)amino)pyrazolo[1,5-a]pyrimidin-5-yl)aminomethyl)piperidin-3-ol (Compound I-17)

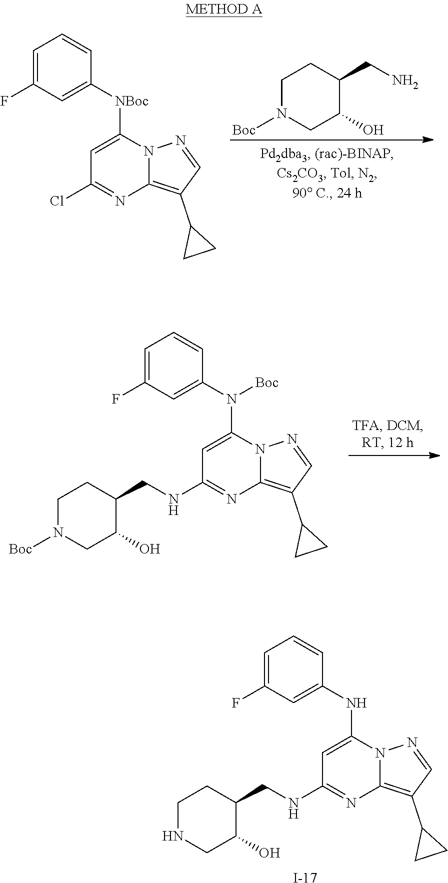

### Tert-Butyl (3S,4S)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (100 mg, 0.25 mmol, 1 eq), and tert-butyl (3S,4S)-4-(aminomethyl)-3-hydroxy-1-piperidinecarboxylate (69 mg, 0.30 mmol, 1.2 eq) and Cs₂CO₃ (163 mg, 0.50 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (3S,4S)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate as a brown oil (60 mg, 40%). TLC system: EtOAc/hexane (1:1), R_{f} value: ˜0.40; ¹HNMR (400 MHz, CDCl₃) δ 7.61 (s, 1H), 7.29-7.23 (m, 1H), 7.12-7.06 (m, 2H), 6.96-6.90 (m, 1H), 5.89 (s, 1H), 4.32-4.24 (m, 2H), 4.16-4.09 (m, 1H), 3.24-3.20 (m, 1H), 3.03-2.94 (m, 1H), 2.62-2.51 (m, 2H), 1.89-1.81 (m, 1H), 1.56-1.50 (m, 3H), 1.45 (s, 9H), 1.39 (s, 9H), 0.93-0.89 (m, 2H), 0.66-0.62 (m, 2H).

### (3S,4S)-4-((3-cyclopropyl-7-((3-fluorophenyl)amino)pyrazolo[1,5-a]pyrimidin-5-yl)aminomethyl)piperidin-3-ol (I-17)

To a solution of tert-butyl (3S,4S)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate (60 mg, 0.10 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (3S,4S)-4-((3-cyclopropyl-7-((3-fluorophenyl)amino)pyrazolo[1,5-a]pyrimidin-5-yl)aminomethyl)piperidin-3-ol (I-17) as an off white gummy solid (30 mg, yield: 74%). TLC system: DCM/Methanol (1:1), R_{f} value: ˜0.2; ¹HNMR (400 Hz, MeOD-d4) δ 7.55 (s, 1H), 7.45-7.39 (m, 1H), 7.20-7.13 (m, 2H), 6.96-6.91 (m, 1H), 5.78 (s, 1H), 4.05-4.00 (m, 1H), 3.44-3.39 (m, 1H), 3.26-3.22 (m, 1H), 3.18-3.12 (m, 2H), 2.76-2.69 (m, 1H), 2.58 (dd, J=11.8 10.7 Hz, 1H), 1.80-1.73 (m, 2H), 1.59-1.53 (m, 2H), 0.87-0.82 (m, 2H), 0.65-0.58 (m, 2H). HRMS (ESI) m/z [M+H]⁺ calcd for C₂₁H₂₆FN₆O 397.2147, found 397.2150. HPLC: 95%.

### Example 20

### Synthesis of (S)—N⁷-(3-Chlorophenyl)-3-Cyclopropyl-N⁵-(piperidin-3-yl)pyrazolo[1,5-a]pyrimidine-5,7-diamine (Compound I-18)

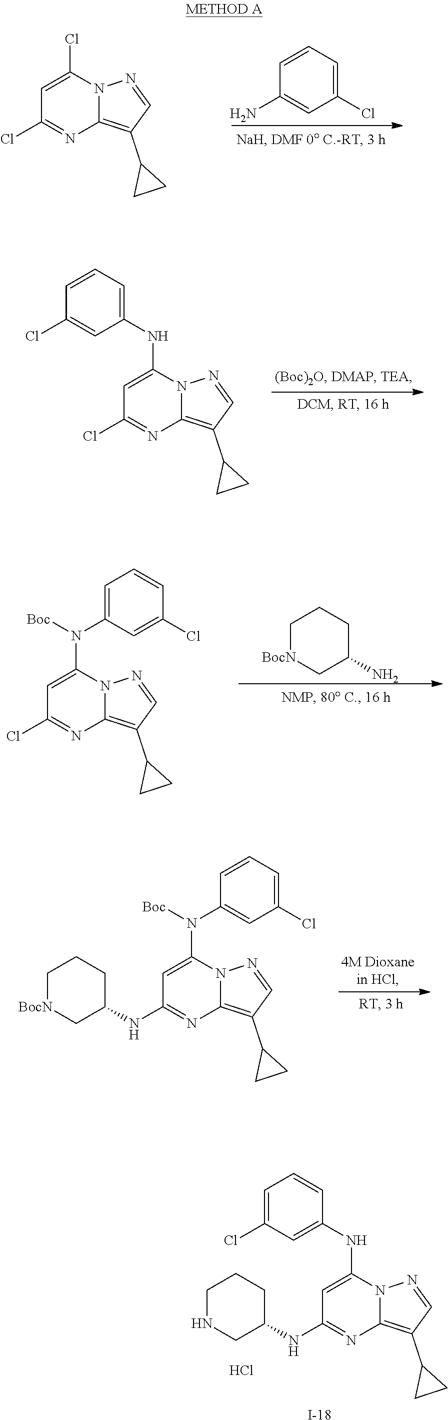

### 5-Chloro-N-(3-chlorophenyl)-3-cyclopropylpyrazolo[1,5-a]pyrimidin-7-amine

To a stirred solution of 3-chloroaniline (0.742 g, 2.202 mmol, 1.0 eq) in DMF (5 mL) cooled to 0-5° C., NaH (55%, 105 mg, 4.404 mmol, 2.0 eq) was added portion-wise under nitrogen flush and the mixture was stirred for 10 min. To this mixture at 0° C. was added 5,7-dichloro-3-cyclopropylpyrazolo[1,5-a]pyrimidine, prepared according to Example 1 above (500 mg, 2.202 mmol, 1.0 eq), and the reaction was stirred at room temperature for 3 h. After completion of reaction by TLC, the reaction mixture was quenched with ice cold water and extracted with (2×50 mL) ethyl acetate. The combined organic layer was washed with water (30 mL), brine (20 mL), dried over sodium sulfate, and concentrated. The crude compound was purified by grace column chromatography [gradient elution with 10-20% Ethyl acetate/Hexane] to afford 5-chloro-N-(3-chlorophenyl)-3-cyclopropyl pyrazolo[1,5-a]pyrimidin-7-amine as yellow solid (200 mg, yield: 28%). TLC system: EtoAc:Hexane (10:90), R_{f} value: ˜0.5; LCMS (m/z): 319.0 (M+H)⁺; ¹HNMR (400 MHz, DMSO-d₆) δ 10.35 (s, 1H), 8.03 (s, 1H), 7.55-7.50 (m, 1H), 7.48-7.46 (m, 2H), 7.37-7.35 (m, 1H), 6.14 (s, 1H), 1.99-1.92 (m, 1H), 0.93-0.89 (m, 2H), 0.81-0.77 (m, 2H).

### Tert-Butyl (5-chloro-3-cyclopropylpyrazolo[1,5-a]pyrimidin-7-yl)(3-chlorophenyl)carbamate

To a stirred solution of 5-chloro-N-(3-chlorophenyl)-3-cyclopropylpyrazolo[1,5-a]pyrimidin-7-amine (200 mg, 0.628 mmol, 1.0 eq) in DCM (10 mL) at room temperature, triethylamine (0.17 mL, 1.257 mmol, 2.0 eq) and DMAP (3.8 mg 0.031 mmol, 0.05 eq) were added, followed by drop-wise addition of (Boc)₂O (150 mg, 0.690 mmol, 1.1 eq), under nitrogen flush at room temperature for 16 h. After completion of reaction by TLC, the reaction mixture was quenched with ice cold water and extracted with (2×50 mL) DCM. The combined organic layer was washed with water (20 mL), brine (20 mL), dried over sodium sulfate, and concentrated. The crude compound was purified by grace column chromatography [gradient elution with 10-20% Ethyl acetate/Hexane] to afford tert-butyl (5-chloro-3-cyclopropylpyrazolo[1,5-a]pyrimidin-7-yl) (3-chlorophenyl) carbamate as a yellow solid (100 mg, 38%). TLC system: EtOAc/hexane (10:90), R_{f} value: ˜0.6; LCMS (m/z): 419.0 (M+H)⁺.

### Tert-Butyl (S)-3-((7-((tert-butoxycarbonyl)(3-chlorophenyl)amino)-3-cyclopropylpyrazolo[1,5-a]pyrimidin-5-yl)amino)piperidine-1-carboxylate

To a stirred solution of tert-butyl (5-chloro-3-cyclopropylpyrazolo[1,5-a]pyrimidin-7-yl)(3-chlorophenyl) carbamate (100 mg, 0.239 mmol, 1.0 eq) in NMP (0.2 mL) tert-butyl (S)-3-aminopiperidine-1-carboxylate (71.7 mg, 0.358 mmol, 1.5 eq) was added at room temperature. The reaction mixture was then heated to 80° C. and stirring continued for 16 h. After completion of reaction by TLC, the reaction mixture was quenched with ice cold water and extracted with (2×50 mL) DCM. The combined organic layer was washed with water (20 mL), brine (20 mL), dried over sodium sulfate, and concentrated to provide crude product which was purified by grace chromatography [gradient elution with 40-50% Ethyl acetate/Hexane] to afford tert-butyl (S)-3-((7-((tert-butoxycarbonyl) (3-chlorophenyl)amino)-3-cyclopropyl pyrazolo[1,5-a]pyrimidin-5-yl)amino) piperidine-1-carboxylate) as an off white gummy solid (50 mg, 35%). TLC system: EtOAc/hexane (50:50), R_{f} value: ˜0.2; LCMS (m/z): 583.3 (M+H)⁺.

### (S)—N⁷-(3-Chlorophenyl)-3-cyclopropyl-N⁵-(piperidin-3-yl)pyrazolo[1,5-a]pyrimidine-5,7-diamine (I-18)

A solution of tert-butyl (S)-3-((7-((tert-butoxycarbonyl) (3-chlorophenyl)amino)-3-cyclopropyl pyrazolo[1,5-a]pyrimidin-5-yl)amino) piperidine-1-carboxylate (50 mg, 0.085 mmol, 1.0 eq) in 4M HCl in Dioxane (2 mL) was stirred at room temperature for 3 h. After completion of reaction by TLC, the reaction mixture was concentrated. The crude compound was purified by trituration with diethyl ether followed by lyophilization afforded (S)—N⁷-(3-chlorophenyl)-3-cyclopropyl-N⁵-(piperidin-3-yl) pyrazolo[1,5-a]pyrimidine-5,7-diamine (I-18) as an off white solid (15 mg, yield: 41%). TLC system: Methanol/DCM (10:90), R_{f value}: ˜0.1; LCMS (m/z): 383.1 (M+H−HCl)⁺; ¹HNMR (400 MHz, DMSO-d₆) δ 10.25-9.99 (br, 1H), 9.25 (brs, 1H), 9.02 (brs, 1H), 7.77 (s, 1H), 7.50-7.41 (m, 3H), 7.32 (d, J=8 Hz, 1H), 5.70 (s, 1H), 4.24 (brs, 1H), 3.39-3.36 (m, 1H), 3.15-3.13 (m, 1H), 2.91-2.78 (m, 2H), 2.05-1.90 (m, 3H), 1.75-1.73 (m, 1H), 1.57-1.54 (m, 1H), 0.88-0.86 (m, 2H), 0.79-0.75 (m, 2H).

### Example 21

### Synthesis of (S)-3-cyclopropyl-N⁷-(3-fluorophenyl)-N⁵-(piperidin-3-yl)pyrazolo[1,5-a]pyrimidine-5,7-diamine hydrochloride (Compound I-19)

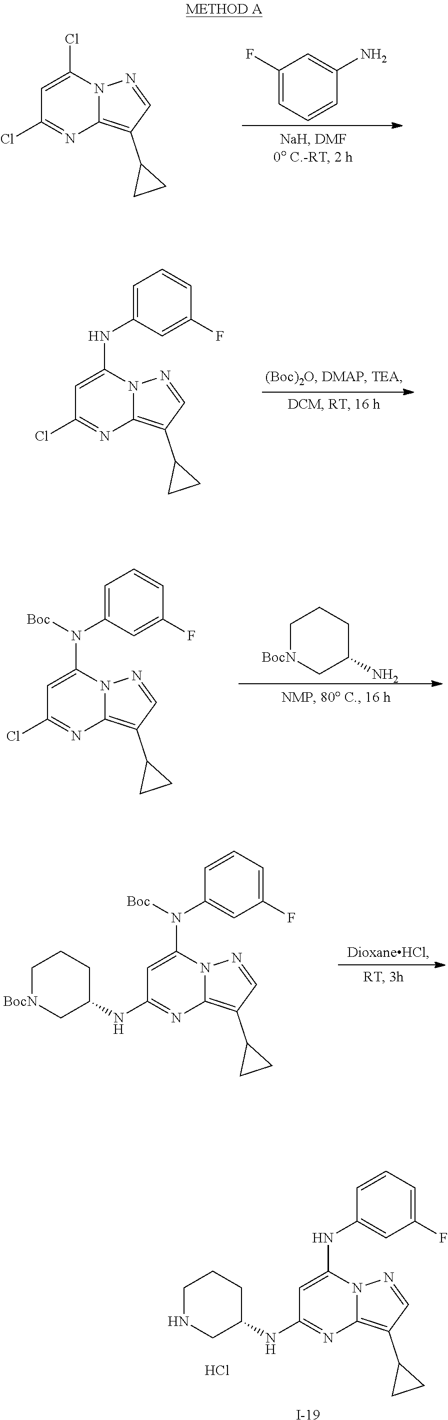

### 5-Chloro-3-cyclopropyl-N-(3-fluorophenyl)pyrazolo[1,5-a]pyrimidin-7-amine

To a stirred solution of 3-fluoroaniline (0.110 g, 0.99 mmol, 0.9 eq) in DMF (5 mL) at 0-5° C., was added NaH (55%, 52.8 mg, 2.20 mmol, 2.0 eq) portion-wise under nitrogen flush and the mixture was stirred for 10 min. To this mixture at 0° C. was added 5,7-dichloro-3-cyclopropylpyrazolo[1,5-a]pyrimidine, prepared according to Example 1 above (250 mg, 1.10 mmol, 1.0 eq), and the reaction was stirred at room temperature for 2 h. After completion of reaction by TLC, the reaction mixture was quenched with ice cold water and extracted with (3×50 mL) ethyl acetate. The combined organic layer was washed with water (30 mL), brine (30 mL), dried over sodium sulfate, and concentrated to afford crude product which was purified by column chromatography [gradient elution with 5-10% Ethyl acetate/Hexane] to afford 5-chloro-3-cyclopropyl-N-(3-fluorophenyl)pyrazolo[1,5-a]pyrimidin-7-amine as yellow solid (200 mg; 60%). TLC system: EtoAc:Hexane (10:90), R_{f} value: ˜0.5; LCMS (m/z): 303.3 (M+H)⁺; 1HNMR (400 MHz, DMSO-d₆) δ 10.36 (s, 1H), 8.03 (s, 1H), 7.60-7.45 (m, 1H), 7.40-7.30 (m, 2H), 7.20-7.10 (m, 1H), 6.19 (s, 1H), 2.07-1.90 (m, 1H), 0.97-0.86 (m, 2H), 0.82-0.75 (m, 2H).

### Tert-Butyl (5-chloro-3-cyclopropylpyrazolo[1,5-a]pyrimidin-7-yl)(3-fluorophenyl)

To a stirred solution of 5-chloro-3-cyclopropyl-N-(3-fluorophenyl)pyrazolo[1,5-a]pyrimidin-7-amine (200 mg, 0.66 mmol, 1.0 eq) in DCM (10 mL) at room temperature was added triethylamine (0.184 mL, 1.32 mmol, 2.0 eq) and DMAP (4 mg 0.03 mmol, 0.05 eq), followed by drop-wise addition of (Boc)₂O (0.166 mL, 0.72 mmol, 1.1 eq), under nitrogen flush. The reaction was stirred at room temperature for 16 h. After completion of reaction by TLC, the reaction mixture was diluted with ice cold water and extracted with (2×50 mL) DCM. The combined organic layer was washed with water (20 mL), brine (20 mL), dried over sodium sulfate, and concentrated. The crude compound was purified by grace column chromatography [gradient elution with 5-10%/Ethyl acetate/Hexane] to afford tert-butyl (5-chloro-3-cyclopropylpyrazolo[1,5-a]pyrimidin-7-yl)(3-fluorophenyl)carbamate as a yellow gummy solid (170 mg, 64%). TLC system: EtOAc/hexane (10:90), R_{f} value: ˜0.7; LCMS (m/z): 403.2 (M+H)⁺; ¹HNMR (400 MHz, DMSO-d₆) δ 8.12 (s, 1H), 7.43-7.39 (m, 1H), 7.33-7.29 (m, 2H), 7.18-7.14 (m, 2H), 2.02-1.97 (m, 1H), 1.29 (s, 9H), 0.98-0.93 (m, 2H), 0.84-0.81 (m, 2H).

### (S)-tert-Butyl 3-((7-((tert-butoxycarbonyl)(3-fluorophenyl)amino)-3-cyclopropylpyrazolo[1,5-a]pyrimidin-5-yl)amino)piperidine-1-carboxylate

To a stirred solution of tert-butyl (5-chloro-3-cyclopropylpyrazolo[1,5-a]pyrimidin-7-yl)(3-fluorophenyl)carbamate (150 mg, 0.37 mmol, 1.0 eq) in NMP (1 mL), was added tert-butyl (S)-3-aminopiperidine-1-carboxylate (82 mg, 0.41 mmol, 1.1 eq) at room temperature. The reaction mixture was heated at 80° C. for 16 h. After completion of reaction by TLC, the reaction mixture was diluted with ice cold water and extracted with (2×100 mL) DCM. The combined organic layer was washed with water (50 mL), brine (50 mL), dried over sodium sulfate, and concentrated to provide crude product which was purified by PREP-HPLC to afford (S)-tert-butyl 3-((7-((tert-butoxycarbonyl)(3-fluorophenyl)amino)-3-cyclopropylpyrazolo[1,5-a]pyrimidin-5-yl)amino)piperidine-1-carboxylate as an off white solid (35 mg, 16%). TLC system: EtOAc/hexane (50:50), R_{f} value: ˜0.3; LCMS (m/z): 567.4 (M+H)⁺. ¹HNMR (400 MHz, DMSO-d₆) δ 7.66 (s, 1H), 7.39 (d, J=8.0 Hz, 14.8 Hz, 1H), 7.22-7.19 (m, 2H), 7.14-7.08 (m, 2H), 6.26 (s, 1H), 5.75 (s, 1H), 3.82 (brs, 1H), 1.98-1.81 (m, 2H), 1.75-1.73 (m, 1H), 1.59-1.50 (m, 1H), 1.46-1.38 (m, 3H), 1.30 (s, 9H), 1.19-1.16 (m, 6H), 0.84-0.75 (m, 4H). 4 protons merged with solvent peaks of DMSO-d₆.

### (S)-3-Cyclopropyl-N⁷-(3-fluorophenyl)-N⁵-(piperidin-3-yl)pyrazolo[1,5-a]pyrimidine-5,7-diamine hydrochloride (I-19)

A solution of (S)-tert-butyl 3-((7-((tert-butoxycarbonyl)(3-fluorophenyl)amino)-3-cyclopropylpyrazolo[1,5-a]pyrimidin-5-yl)amino)piperidine-1-carboxylate (35 mg, 0.06 mmol, 1.0 eq) in 4M HCl in Dioxane (2 mL) was stirred at room temperature for 3 h. After completion of reaction by TLC, the reaction mixture was concentrated. The crude compound was purified by trituration with diethyl ether followed by lyophilization afforded (S)-3-cyclopropyl-N⁷-(3-fluorophenyl)-N⁵-(piperidin-3-yl)pyrazolo[1,5-a]pyrimidine-5,7-diamine hydrochloride (I-19) as an off white solid (15 mg, yield: 68%). LCMS (m/z): 367.3 (M+H−HCl)⁺; ¹HNMR (400 MHz, DMSO-d₆) δ 9.42 (brs, 1H), 8.68-8.62 (m, 2H), 7.66 (s, 1H), 7.45 (dd, J=14.6 Hz, 7.6 Hz, 1H), 7.27-7.25 (m, 2H), 7.03-6.96 (m, 2H), 5.78 (s, 1H), 4.12 (brs, 1H), 3.39-3.31 (m, 1H), 3.15-3.13 (m, 1H), 2.92-2.80 (m, 2H), 1.95-1.84 (m, 3H), 1.74-1.70 (m, 1H), 1.54-1.49 (m, 1H), 0.83-0.80 (m, 4H).

### Example 22

### Synthesis of N-(3-cyclopropyl-7-((3-fluorophenyl)amino)pyrazolo[1,5-a]pyrimidin-5-yl)-1-(trimethylsilyl)cyclobutanecarboxamide (Compound I-20)

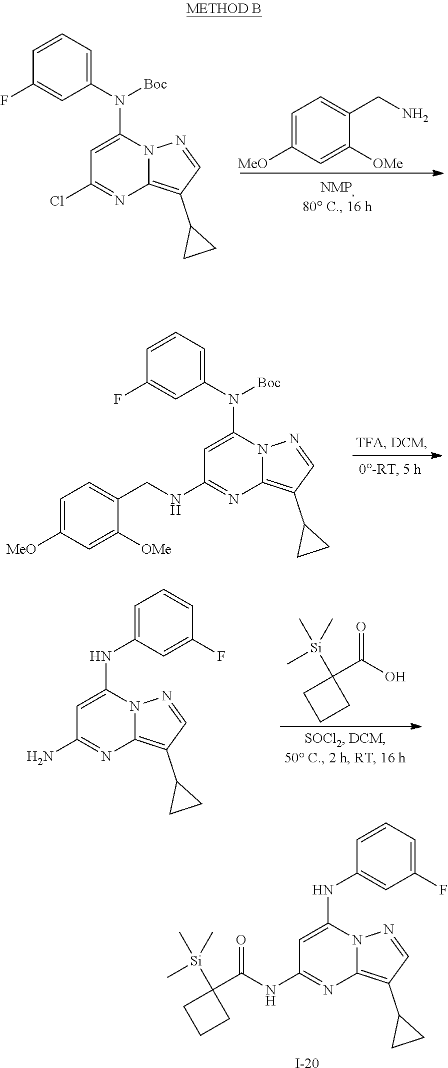

### Tert-Butyl (3-cyclopropyl-5-((2,4-dimethoxybenzyl)amino)pyrazolo[1,5-a]pyrimidin-7-yl)(3-fluorophenyl)carbamate

To a stirred solution of tert-butyl (5-chloro-3-cyclopropylpyrazolo[1,5-a]pyrimidin-7-yl)(3-fluorophenyl)carbamate, prepared according to Example 6 above (1.4 g, 3.48 mmol, 1.0 eq), in NMP (5 mL) was added (2,4-dimethoxyphenyl)methanamine (1.16 g, 6.96 mmol, 2.0 eq) at room temperature. The reaction mixture was heated at 80° C. for 16 h. After completion of reaction by TLC, the reaction mixture was diluted with ice cold water and extracted with (2×50 mL) EtOAc. The combined organic layer was washed with water (20 mL), brine (20 mL), dried over sodium sulfate, and concentrated. The crude compound was purified by Grace silica column chromatography [gradient elution with 15-20% Ethyl acetate/Hexane] to afford tert-butyl (3-cyclopropyl-5-((2,4-dimethoxybenzyl)amino)pyrazolo[1,5-a]pyrimidin-7-yl)(3-fluorophenyl)carbamate as brown gummy solid (1.1 g, 60%). TLC system: EtOAc/hexane (20:80), R_{f} value: ˜0.2; LCMS (m/z): 534.4 (M+H)⁺.

### 3-Cyclopropyl-N⁷-(3-fluorophenyl)pyrazolo[1,5-a]pyrimidine-5,7-diamine

To a stirred solution of tert-butyl (3-cyclopropyl-5-((2,4-dimethoxybenzyl)amino)pyrazolo[1,5-a]pyrimidin-7-yl)(3-fluorophenyl)carbamate (1.1 g, 1.0 mmol, 1.0 eq) in DCM (5 mL) at 0° C. as added TFA (1.1 mL) under nitrogen flush. The reaction mixture was then warmed to room temperature and stirred for 16 h. After completion of reaction by TLC, the reaction mixture was concentrated, diluted with ice water and extracted with (2×50 mL) DCM. The combined organic layer was washed with sodium bicarbonate solution (20 mL), brine (20 mL), dried over sodium sulfate, and concentrated. The crude product was purified by trituration with n-pentane to afford 3-cyclopropyl-N⁷-(3-fluorophenyl)pyrazolo[1,5-a]pyrimidine-5,7-diamine as brown solid (500 mg, yield: 85%). LCMS (m/z): 284.1 (M+1)⁺; ¹HNMR (400 MHz, DMSO-d₆) δ 9.32 (s, 1H), 7.59 (s, 1H), 7.47-7.41 (m, 1H), 7.27-7.24 (m, 2H), 7.01-6.97 (m, 1H), 6.30 (s, 2H), 5.78 (s, 1H), 1.78-1.74 (m, 1H), 0.80-0.75 (m, 2H), 0.67-0.62 (m, 2H).

### N-(3-Cyclopropyl-7-((3-fluorophenyl)amino)pyrazolo[1,5-a]pyrimidin-5-yl)-1-(trimethylsilyl)cyclobutanecarboxamide (I-20)

To a solution of 1-(trimethylsilyl)cyclobutanecarboxylic acid (2×400 mg, 2.32 mmol, 1.0 eq) in DCM (10 mL) was added thionyl chloride (305 mg, 2.55 mmol, 1.1 eq) drop-wise. The reaction was heated at 50° C. for 2 h. After completion of reaction by TLC, the reaction mixture was concentrated under nitrogen atmosphere. The obtained residue was dissolved in DCM (5 mL), and was added to a solution of 3-cyclopropyl-N⁷-(3-fluorophenyl)pyrazolo[1,5-a]pyrimidine-5,7-diamine (132 mg, 0.46 mmol, 0.2 eq) in DCM (5 mL) at 0° C. The reaction was stirred for 16 h at room temperature. After completion of reaction by TLC, all the volatiles were evaporated to provide crude compound. The crude compound was purified by grace silica column chromatography [gradient elution with 5-10% Ethyl acetate/Hexane] to afford N-(3-cyclopropyl-7-((3-fluorophenyl)amino)pyrazolo[1,5-a]pyrimidin-5-yl)-1-(trimethylsilyl)cyclobutanecarboxamide (I-20) as an off-white solid (20 mg, yield: 2%). TLC system: EtoAc:Hexane (10:90), R_{f value}: ˜0.3; LCMS (m/z): 438.3 (M+1)⁺; ¹HNMR (400 MHz, DMSO-d₆) δ 9.89 (s, 1H), 9.44 (s, 1H), 7.84 (s, 1H), 7.52-7.46 (m, 2H), 7.33-7.29 (m, 2H), 7.08-7.03 (m, 1H), 2.49-2.47 (m, 2H), 2.27-2.20 (m, 2H), 1.94-1.90 (m, 1H), 1.83-1.76 (m, 2H), 0.88-0.84 (m, 2H), 0.74-0.70 (m, 2H), 0.08 (s, 9H

### Example 23

### Synthesis of (3S,4S)-3-((3-cyclopropyl-7-((3-fluorophenyl)amino)pyrazolo[1,5-a]pyrimidin-5-yl)amino)piperidin-4-ol hydrochloride (Compound I-21)

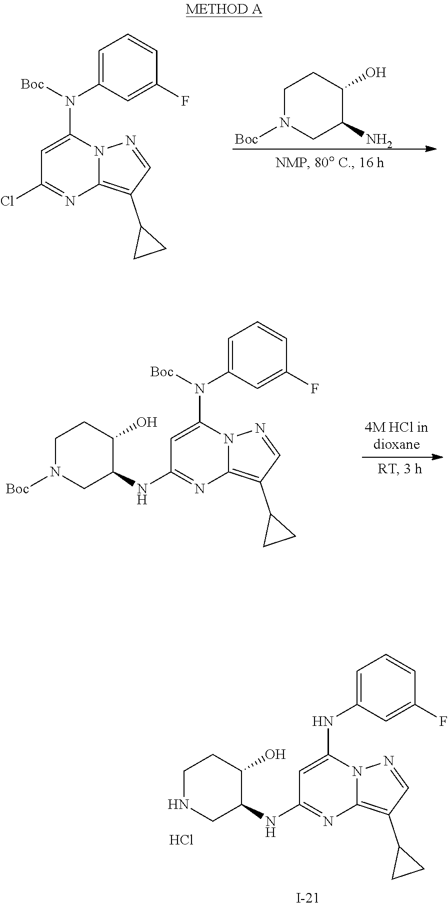

### (3S,4S)-tert-Butyl 3-((7-((tert-butoxycarbonyl)(3-fluorophenyl)amino)-3-cyclopropylpyrazolo[1,5-a]pyrimidin-5-yl)amino)-4-hydroxypiperidine-1-carboxylate

To a stirred solution of tert-butyl (5-chloro-3-cyclopropylpyrazolo[1,5-a]pyrimidin-7-yl)(3-fluorophenyl)carbamate, prepared according to Example 6 above (200 mg, 0.49 mmol, 1.0 eq), in NMP (2 mL) was added (3S,4S)-tert-butyl 3-amino-4-hydroxypiperidine-1-carboxylate (211 mg, 0.98 mmol, 2.0 eq) at room temperature. The reaction mixture was heated at 80° C. for 16 h. After completion of reaction by TLC, the reaction mixture was diluted with ice cold water and extracted with (2×30 mL) DCM. The combined organic layer was washed with water (10 mL), brine (10 mL), dried over sodium sulfate and concentrated to provide crude which was purified by Prep-HPLC to afford (3S,4S)-tert-butyl 3-((7-((tert-butoxycarbonyl)(3-fluorophenyl)amino)-3-cyclopropylpyrazolo[1,5-a]pyrimidin-5-yl)amino)-4-hydroxypiperidine-1-carboxylate as an off white solid (35 mg, 12%). TLC system: EtOAc/hexane (50:50), R_{f} value: ˜0.2, LCMS (m/z): 583.4 (M+H)⁺.

### (3S,4S)-3-((3-Cyclopropyl-7-((3-fluorophenyl)amino)pyrazolo[1,5-a]pyrimidin-5-yl)amino)piperidin-4-ol hydrochloride (I-21)

A solution of (3S,4S)-tert-butyl 3-((7-((tert-butoxycarbonyl)(3-fluorophenyl)amino)-3-cyclopropylpyrazolo[1,5-a]pyrimidin-5-yl)amino)-4-hydroxypiperidine-1-carboxylate (35 mg, 0.060 mmol, 1.0 eq) in 4M HCl in Dioxane (0.5 mL) was stirred at room temperature for 3 h. After completion of reaction by TLC, the reaction mixture was concentrated and purified by trituration with diethyl ether followed by lyophilization afforded (3S,4S)-3-((3-cyclopropyl-7-((3-fluorophenyl)amino)pyrazolo[1,5-a]pyrimidin-5-yl)amino)piperidin-4-ol hydrochloride (I-21) as an off white solid (20 mg, yield: 80%). LCMS (m/z): 383.3 (M+H−HCl)⁺; ¹HNMR (400 MHz, DMSO-d₆) δ 9.70-9.50 (br, 1H), 8.84 (brs, 2H), 8.43 (s, 1H), 7.70 (s, 1H), 7.55-7.44 (m, 1H), 7.28-7.26 (m, 2H), 7.05 (t, J=8.0 Hz, 1H), 5.84 (s, 1H), 4.02-4.01 (brs, 1H), 3.69-3.63 (m, 2H), 3.23-3.16 (m, 2H), 2.96-2.86 (m, 2H), 2.09-2.05 (m, 1H), 1.95-1.88 (m, 1H), 1.70-1.67 (m, 1H), 0.85-0.80 (m, 2H), 0.77-0.72 (m, 2H).

### Example 24

### Synthesis of (3S,5S)-5-((3-cyclopropyl-7-((3-fluorophenyl)amino)pyrazolo[1,5-a]pyrimidin-5-yl)amino)piperidin-3-ol hydrochloride (Compound I-22)

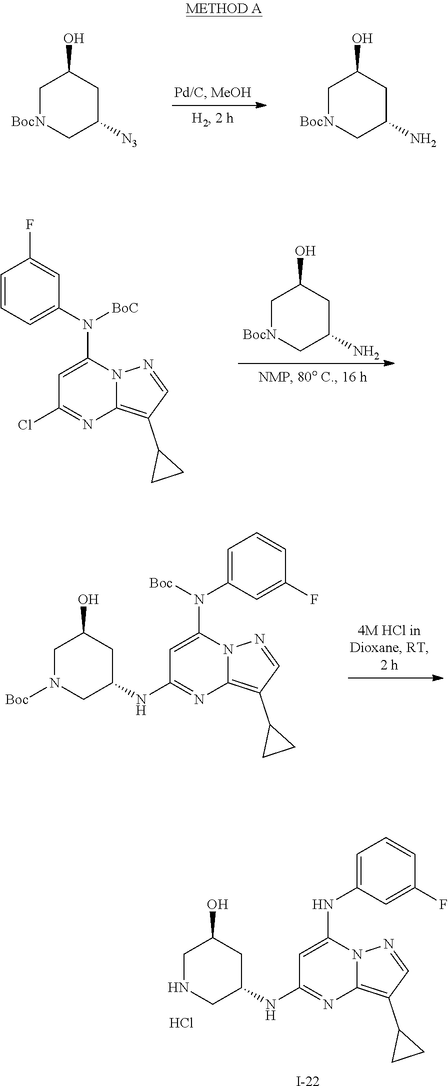

### Tert-Butyl (3S,5S)-3-amino-5-hydroxypiperidine-1-carboxylate

To a stirred solution of tert-butyl (3S,5S)-3-azido-5-hydroxypiperidine-1-carboxylate (250 mg, 1.03 mmol, 1.0 eq) in methanol (2.5 mL), was added Pd/C (10%) (120 mg) and the mixture was stirred for 2 h at RT under H₂ balloon. After completion of reaction monitored by TLC, the reaction mixture was filtered through Celite bed, washed with methanol and concentrated to provide crude. The crude compound was triturated with n-pentane to afford tert-butyl (3S,5S)-3-amino-5-hydroxypiperidine-1-carboxylate as a colorless semi solid (200 mg, yield: 89%). TLC system: Methanol/DCM (10:90), Stain: PMA active, R_{f} value: ˜0.1; ¹H NMR (400 MHz, CDCl₃) δ 4.05 (m, 1H), 3.91-3.87 (m, 1H), 3.77-3.74 (m, 1H), 3.24-3.11 (m, 2H), 2.72-2.67 (m, 1H), 1.99-1.97 (m, 1H), 1.46 (s, 11H).

### Tert-Butyl (3S,5S)-3-((7-((tert-butoxycarbonyl)(3-fluorophenyl)amino)-3-cyclopropylpyrazolo[1,5-a]pyrimidin-5-yl)amino)-5-hydroxypiperidine-1-carboxylate

A solution of tert-butyl (5-chloro-3-cyclopropylpyrazolo[1,5-a]pyrimidin-7-yl)(3-fluorophenyl)carbamate, prepared according to Example 6 above (160 mg, 0.39 mmol, 1 eq), and tert-butyl (3S,5S)-3-amino-5-hydroxypiperidine-1-carboxylate (163 mg, 0.75 mmol, 1.9 eq) in NMP (0.8 mL) was stirred at 80° C. for 16 h. After completion of reaction monitored by TLC, the reaction mixture was diluted with water and extracted with ethyl acetate (2×15 mL). The combined organic layer was washed with water (10 mL), brine (5 mL), dried over sodium sulfate, and concentrated. The crude compound was purified by grace column followed by prep-HPLC to afford tert-butyl (3S,5S)-3-((7-((tert-butoxycarbonyl)(3-fluorophenyl)amino)-3-cyclopropylpyrazolo[1,5-a]pyrimidin-5-yl)amino)-5-hydroxypiperidine-1-carboxylate as a gummy solid (15 mg, yield: 6%). TLC system: Ethyl acetatel/Hexane (50:50), R_{f} value: ˜0.2; LCMS (m/z): 583.4 (M+H)⁺.

### (3S,5S)-5-((3-Cyclopropyl-7-((3-fluorophenyl)amino)pyrazolo[1,5-a]pyrimidin-5-yl)amino)piperidin-3-ol hydrochloride (I-22)

A solution of tert-butyl (3S,5S)-3-((7-((tert-butoxycarbonyl)(3-fluorophenyl)amino)-3-cyclopropylpyrazolo[1,5-a]pyrimidin-5-yl)amino)-5-hydroxypiperidine-1-carboxylate (15 mg, 0.02 mmol, 1.0 eq) in 4M HCl in Dioxane (0.8 mL) was stirred at room temperature for 2 h. After completion of reaction monitored by TLC, the reaction mixture was concentrated. The crude compound was purified by trituration with diethyl ether followed by lyophilization afforded (3S,5S)-5-((3-cyclopropyl-7-((3-fluorophenyl)amino)pyrazolo[1,5-a]pyrimidin-5-yl)amino)piperidin-3-ol hydrochloride (I-22) as an off white solid (4.8 mg, yield: 48%). TLC system: Methanol/DCM (10:90), R_{f} value: ˜0.1; LCMS (m/z): 383.3 (M+H−HCl)⁺; ¹HNMR (400 MHz, DMSO-d₆) δ 9.41 (s, 1H), 8.85 (s, 2H), 7.65 (s, 1H), 7.48-7.42 (m, 1H), 7.27-7.24 (m, 2H), 7.03-6.99 (m, 1H), 6.94 (brs, 1H), 5.77 (s, 1H), 4.45-4.42 (m, 1H), 4.15 (brs, 1H), 3.07-2.95 (m, 2H), 2.73-2.71 (m, 1H), 1.98-1.95 (m, 1H), 1.90-1.85 (m, 1H), 1.71-1.66 (m, 1H), 0.84-0.74 (m, 4H).

### Example 25

### Synthesis of (S)-3-cyclobutyl-N⁷-(3-fluorophenyl)-N⁵-(piperidin-3-yl)pyrazolo[1,5-a]pyrimidine-5,7-diamine hydrochloride (Compound I-23)

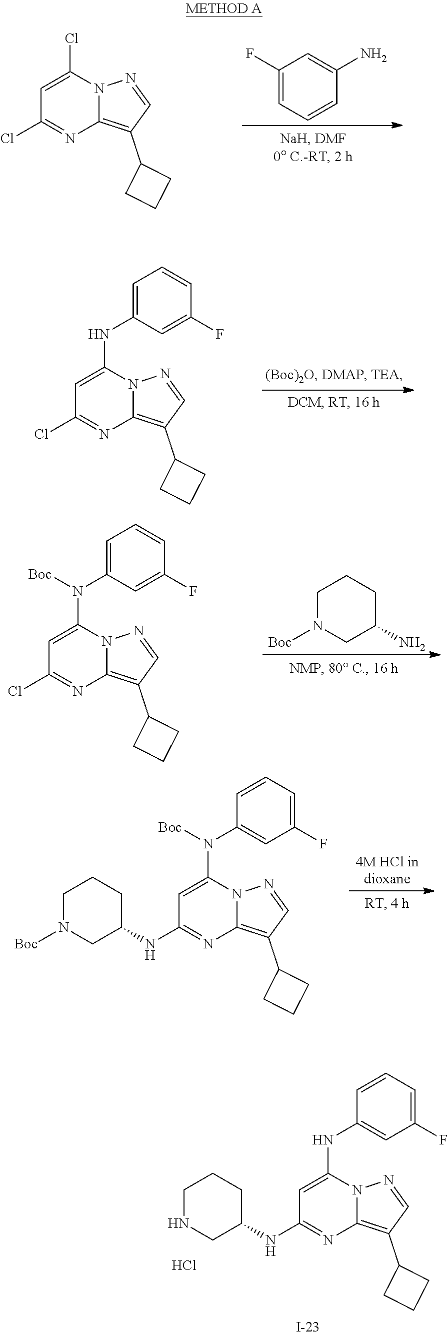

### 5-Chloro-3-cyclobutyl-N-(3-fluorophenyl)pyrazolo[1,5-a]pyrimidin-7-amine

To a stirred solution of 3-fluoroaniline (0.227 g, 2.05 mmol, 1.1 eq) in DMF (10 mL) cooled to 0-5° C., NaH (0.111 g, 4.65 mmol, 2.5 eq) was added portion-wise under nitrogen flush and the mixture was stirred for 10 min. To this mixture at 0° C. was added 5,7-dichloro-3-cyclobutylpyrazolo[1,5-a]pyrimidine, prepared according to Example 2 above (450 mg, 1.86 mmol, 1.0 eq), and the reaction stirred at room temperature for 2 h. After completion of reaction by TLC, the reaction mixture was quenched with ice cold water and extracted with (2×50 mL) ethyl acetate. The combined organic layer was washed with water (30 mL), brine (20 mL), dried over sodium sulfate, and concentrated. The crude compound was purified by grace column chromatography [gradient elution with 10-20% Ethyl acetate/Hexane] to afford 5-chloro-3-cyclobutyl-N-(3-fluorophenyl)pyrazolo[1,5-a]pyrimidin-7-amine as yellow solid (350 mg, yield: 59%). TLC system: EtoAc:Hexane (10:90), R_{f} value: ˜0.5; LCMS (m/z): 317.2 (M+H)⁺; ¹HNMR (400 MHz, DMSO-d₆) δ 10.38 (s, 1H), 8.26 (s, 1H), 7.54-7.48 (m, 1H), 7.37-7.33 (m, 2H), 7.16-7.12 (m, 1H), 6.21 (s, 1H), 3.67 (p, J=8.4 Hz, 1H), 2.32-2.28 (m, 4H), 2.00-1.89 (m, 2H).

### Tert-Butyl (5-chloro-3-cyclobutylpyrazolo[1,5-a]pyrimidin-7-yl)(3-fluorophenyl)carbamate

To a stirred solution of 5-chloro-3-cyclobutyl-N-(3-fluorophenyl)pyrazolo[1,5-a]pyrimidin-7-amine (350 mg, 1.11 mmol, 1.0 eq) in DCM (10 mL) at room temperature was added triethylamine (0.39 mL, 2.77 mmol, 2.5 eq), DMAP (13.54 mg 0.111 mmol, 0.1 eq) and (Boc)₂O (0.382 mL, 1.66 mmol, 1.5 eq), under nitrogen flush and the reaction was stirred at room temperature for 16 h. After completion of reaction by TLC, the reaction mixture was quenched with ice cold water and extracted with (2×50 mL) DCM. The combined organic layer was washed with water (20 mL), brine (20 mL), dried over sodium sulfate, and concentrated. The crude compound was purified by grace column chromatography [gradient elution with 10-15% Ethyl acetate/Hexane] to afford tert-butyl(5-chloro-3-cyclobutylpyrazolo[1,5-a]pyrimidin-7-yl)(3-fluorophenyl) carbamate as a yellow solid (380 mg, 82%). TLC system: EtOAc/hexane (10:90), R_{f} value: ˜0.6; LCMS (m/z): 417.2 (M+H)⁺; ¹HNMR (400 MHz, CDCl₃) δ 8.11 (s, 1H), 7.33-7.27 (m, 1H), 7.10-7.04 (m, 2H), 6.99-6.98 (m, 1H), 6.61 (s, 1H), 3.83 (p, J=8.8 Hz, 1H), 2.45-2.40 (m, 2H), 2.31-2.26 (m, 2H), 2.07-2.04 (m, 2H), 1.38 (s, 9H).

### (S)-tert-Butyl 3-((7-((tert-butoxycarbonyl)(3-fluorophenyl)amino)-3-cyclobutylpyrazolo[1,5-a]pyrimidin-5-yl)amino)piperidine-1-carboxylate

To a stirred solution of tert-butyl (5-chloro-3-cyclobutylpyrazolo[1,5-a]pyrimidin-7-yl)(3-fluorophenyl)carbamate (200 mg, 0.48 mmol, 1.0 eq) in NMP (2 mL) was added tert-butyl (S)-3-aminopiperidine-1-carboxylate (192 mg, 0.96 mmol, 2.0-eq) at room temperature. The reaction mixture was then heated at 80° C. and stirring continued for 16 h. After completion of reaction by TLC, the reaction mixture was quenched with ice cold water and extracted with (2×50 mL) DCM. The combined organic layer was washed with water (20 mL), brine (20 mL), dried over sodium sulfate, and concentrated to provide crude product which was purified by grace chromatography [gradient elution with 40-50% Ethyl acetate/Hexane] to afford (S)-tert-butyl 3-((7-((tert-butoxycarbonyl) (3-fluorophenyl)amino)-3-cyclobutyl pyrazolo[1,5-a]pyrimidin-5-yl)amino) piperidine-1-carboxylate as an off white solid (30 mg, 10%). TLC system: EtOAc/hexane (50:50), R_{f} value: ˜0.2; LCMS (m/z): 581.7 (M+H)⁺.

### (S)-3-Cyclobutyl-N⁷-(3-fluorophenyl)-N⁵-(piperidin-3-yl)pyrazolo[1,5-a]pyrimidine-5,7-diamine hydrochloride (I-23)

A solution of (S)-tert-butyl 3-((7-((tert-butoxycarbonyl)(3-fluorophenyl)amino)-3-cyclobutylpyrazolo[1,5-a]pyrimidin-5-yl)amino)piperidine-1-carboxylate (30 mg, 0.05 mmol, 1.0 eq) in 4M HCl in Dioxane (1 mL) was stirred at room temperature for 4 h. After completion of reaction by TLC, the reaction mixture was concentrated. The crude compound was purified by trituration with diethyl ether followed by lyophilization afforded (S)-3-cyclobutyl-N⁷-(3-fluorophenyl)-N⁵-(piperidin-3-yl)pyrazolo[1,5-a]pyrimidine-5,7-diamine hydrochloride (I-23) as an off white solid (19 mg, yield: 19%). LCMS (m/z): 381.3 (M+H−HCl)⁺; ¹HNMR (400 MHz, DMSO-d₆) δ 9.56 (br, 1H), 8.82 (brs, 2H), 7.89 (s, 1H), 7.46 (ABq, J=8.0 Hz, 15.6 Hz, 1H), 7.28-7.26 (m, 2H), 7.03 (t, J=8.0 Hz, 1H), 5.79 (s, 1H), 4.14 (brs, 1H), 3.62-3.61 (m, 1H), 3.42-3.41 (m, 1H), 3.17-3.16 (m, 1H), 2.89-2.81 (m, 2H), 2.33-2.25 (m, 4H), 1.99-1.88 (m, 4H), 1.74-1.70 (m, 1H), 1.54-1.51 (m, 1H).

### Example 26

### Synthesis of (3S,4S)-3-((3-Cyclobutyl-7-((3-fluorophenyl)amino)pyrazolo[1,5-a]pyrimidin-5-yl)amino)piperidin-4-ol hydrochloride (Compound I-24)

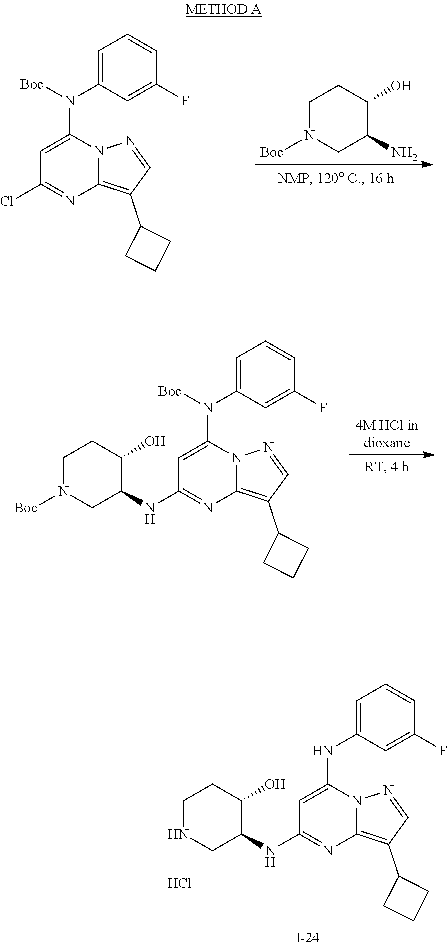

### (3S,4S)-tert-Butyl 3-((7-((tert-butoxycarbonyl)(3-fluorophenyl)amino)-3-cyclobutylpyrazolo[1,5-a]pyrimidin-5-yl)amino)-4-hydroxypiperidine-1-carboxylate

To a stirred solution of tert-butyl (5-chloro-3-cyclobutylpyrazolo[1,5-a]pyrimidin-7-yl)(3-fluorophenyl)carbamate, prepared according to Example 25 above (100 mg, 0.24 mmol, 1.0 eq), in NMP (1 mL) was added (3S,4S)-tert-butyl 3-amino-4-hydroxypiperidine-1-carboxylate (104 mg, 0.48 mmol, 2.0 eq) at room temperature. The reaction mixture was heated at 120° C. for 16 h. After completion of reaction by TLC, the reaction mixture was diluted with ice cold water and extracted with (2×20 mL) DCM. The combined organic layer was washed with water (10 mL), brine (10 mL), dried over sodium sulfate, and concentrated to provide crude which was purified by Prep-HPLC to afford (3S,4S)-tert-butyl 3-((7-((tert-butoxycarbonyl)(3-fluorophenyl)amino)-3-cyclobutylpyrazolo[1,5-a]pyrimidin-5-yl)amino)-4-hydroxypiperidine-1-carboxylate as an off white solid (15 mg, 10%). TLC system: EtOAc/hexane (50:50), R_{f} value: ˜0.2; LCMS (m/z): 597.5 (M+H)⁺.

### (3S,4S)-3-((3-Cyclobutyl-7-((3-fluorophenyl)amino)pyrazolo[1,5-a]pyrimidin-5-yl)amino)piperidin-4-ol hydrochloride (I-24)

A solution of (3S,4S)-tert-butyl 3-((7-((tert-butoxycarbonyl)(3-fluorophenyl)amino)-3-cyclobutylpyrazolo[1,5-a]pyrimidin-5-yl)amino)-4-hydroxypiperidine-1-carboxylate (15 mg, 0.025 mmol, 1.0 eq) in 4M HCl in Dioxane (0.2 mL) was stirred at room temperature for 4 h. After completion of reaction by TLC, the reaction mixture was concentrated and purified by trituration with diethyl ether followed by lyophilization afforded (3S,4S)-3-((3-cyclobutyl-7-((3-fluorophenyl)amino)pyrazolo[1,5-a]pyrimidin-5-yl)amino)piperidin-4-ol hydrochloride (I-24) as an off white solid (10 mg, yield: 91%). LCMS (m/z): 397.3 (M+H−HCl)⁺; ¹HNMR (400 MHz, DMSO-d₆) δ 9.61 (br, 1H), 8.85 (s, 2H), 8.07 (s, 1H), 7.71-7.69 (m, 1H), 7.29-7.26 (m, 2H), 7.05 (t, J=8.0 Hz, 1H), 5.85 (s, 1H), 3.99 (brs, 1H), 3.70-3.65 (m, 3H), 3.24-3.18 (m, 1H), 3.02-2.89 (m, 2H), 2.33-2.21 (m, 4H), 2.09-2.06 (m, 1H), 1.99-1.85 (m, 2H), 1.70-1.67 (m, 1H).

### Example 27

### Synthesis of N-(3-cyclobutyl-7-((3-fluorophenyl)amino)pyrazolo[1,5-a]pyrimidin-5-yl)-1-(trimethylsilyl)cyclobutanecarboxamide (Compound I-25)

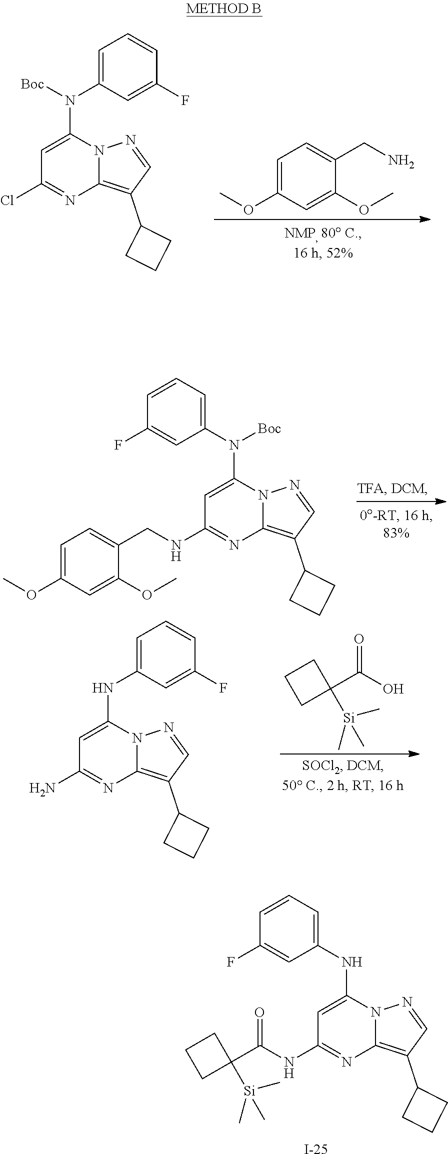

### Tert-Butyl (3-cyclobutyl-5-((2,4-dimethoxybenzyl)amino)pyrazolo[1,5-a]pyrimidin-7-yl)(3-fluorophenyl)carbamate

To a stirred solution of tert-butyl (5-chloro-3-cyclobutylpyrazolo[1,5-a]pyrimidin-7-yl)(3-fluorophenyl)carbamate, prepared according to Example 25 above (800 mg, 1.92 mmol, 1.0 eq), in NMP (10 mL) was added (2,4-dimethoxyphenyl)methanamine (642 mg, 3.84 mmol, 2.0 eq) at room temperature. The reaction mixture was heated at 80° C. for 16 h. After completion of reaction by TLC, the reaction mixture was diluted with ice cold water and extracted with (2×50 mL) DCM. The combined organic layer was washed with water (20 mL), brine (20 mL), dried over sodium sulfate, and concentrated. The crude compound was purified by Grace silica column chromatography [gradient elution with 15-20% Ethyl acetate/Hexane] to afford tert-butyl (3-cyclobutyl-5-((2,4-dimethoxybenzyl)amino)pyrazolo[1,5-a]pyrimidin-7-yl)(3-fluorophenyl)carbamate as brown gummy solid (550 mg, 52%). TLC system: EtOAc/hexane (20:80), R_{f} value: ˜0.2; LCMS (m/z): 548.4 (M+H)⁺.

### N-(3-Cyclobutyl-7-((3-fluorophenyl)amino)pyrazolo[1,5-a]pyrimidin-5-yl)-1-(trimethylsilyl)cyclobutanecarboxamide (I-25)

To a stirred solution of tert-butyl (3-cyclobutyl-5-((2,4-dimethoxybenzyl)amino)pyrazolo[1,5-a]pyrimidin-7-yl)(3-fluorophenyl)carbamate (550 mg, 1 mmol, 1.0 eq) in DCM (5 mL) at 0° C. was added TFA (0.5 mL) under nitrogen flush. The reaction mixture was warmed to room temperature and stirred for 16 h. After completion of reaction by TLC, the reaction mixture was concentrated. diluted with ice water and extracted with (2×50 mL) DCM. The combined organic layer was washed with sodium bicarbonate solution (20 mL), brine (20 mL), dried over sodium sulfate, and concentrated. The crude product was purified by trituration with n-pentane to afford N-(3-cyclobutyl-7-((3-fluorophenyl)amino)pyrazolo[1,5-a]pyrimidin-5-yl)-1-(trimethylsilyl)cyclobutanecarboxamide (I-25) as an off white solid (250 mg, yield: 83%). LCMS (m/z): 298.4 (M+1)⁺; ¹HNMR (400 MHz, CDCl₃) δ 7.91 (s, 1H), 7.85 (s, 1H), 7.39-7.35 (m, 1H), 7.10-7.05 (m, 2H), 6.96-6.93 (m, 1H), 5.68 (s, 1H), 4.63 (s, 2H), 3.69-3.65 (m, 1H), 2.45-2.35 (m, 2H), 2.24-2.19 (m, 2H), 2.01-1.89 (m, 2H).

### Example 28

### Synthesis of (S)-7-((3,5-dichlorobenzyl)amino)-5-((piperidin-3-yl)amino)pyrazolo[1,5-a]pyrimidine-3-carbonitrile (Compound I-26)

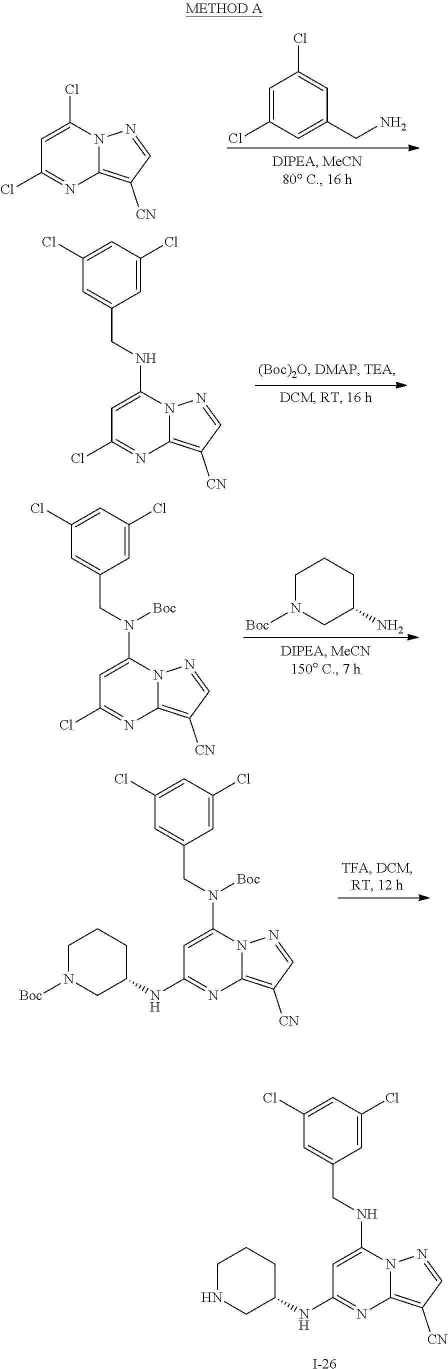

### 5-Chloro-3-cyano-N-(3,5-dichlorobenzyl)pyrazolo[1,5-a]pyrimidin-7-amine

To a stirred solution of 3,5-dichlorobenzylamine (58 mg, 0.33 mmol, 1.1 eq) in acetonitrile (5 mL) was added DIPEA (77 mg, 0.60 mmol, 2.0 eq) and the solution was stirred for 5 min. To this mixture 5,7-dichloropyrazolo[1,5-a]pyrimidine-3-carbonitrile (64 mg, 0.30 mmol, 1.0 eq) was added at room temperature and stirring continued at 80° C. for 16 h. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound was purified by using CombiFlash chromatography (4 g column) to afford 5-chloro-3-cyano-N-(3,5-dichlorobenzyl)pyrazolo[1,5-a]pyrimidin-7-amine as a yellow solid (100 mg, yield: 94%). TLC system: EtoAc:Hexane (1:2), R_{f} value: ˜0.5.

### Tert-Butyl (5-chloro-3-cyanopyrazolo[1,5-a]pyrimidin-7-yl)(3,5-dichlorobenzyl) Carbamate

To a stirred solution of 5-chloro-3-cyano-N-(3,5-dichlorobenzyl)pyrazolo[1,5-a]pyrimidin-7-amine (88 mg, 0.25 mmol, 1.0 eq) in DCM (5 mL) at room temperature was added triethylamine (50 mg, 0.50 mmol, 2.0 eq) and DMAP (2 mg 0.016 mmol), followed by addition of (Boc)₂O (71 mg, 0.33 mmol, 1.3 eq). The reaction was continued at room temperature for 16 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyanopyrazolo[1,5-a]pyrimidin-7-yl)(3,5-dichlorobenzyl)carbamate as a white oil (77 mg, 68%). TLC system: EOAc/hexane (1:4), R_{f} value: ˜0.3; ¹HNMR (400 MHz, CDCl₃) δ 8.48 (s, 1H), 7.35 (t, J=1.9 Hz, 1H), 7.27 (s, 2H), 6.98 (s, 1H), 5.07 (s, 2H), 1.47 (s, 9H).

### Tert-Butyl (S)-3-((3-cyano-7-(tert-butoxycarbonyl)(3,5-dichlorobenzyl)aminopyrazolo[1,5-a]pyrimidin-5-yl)amino)piperidine-1-carboxylate

To a stirred solution of tert-butyl (5-chloro-3-cyanopyrazolo[1,5-a]pyrimidin-7-yl)(3,5-dichlorobenzyl)carbamate (77 mg, 0.17 mmol, 1 eq) in acetonitrile (3 mL) in a microwave reaction vial, (S)-tert-butyl 3-aminopiperidine-1-carboxylate (41 mg, 0.20 mmol, 1.2 eq) and DIPEA (44 mg, 0.34 mmol, 2.0 eq) were added at room temperature. The reaction mixture was under nitrogen flush for 5 minutes and then the microwave vial was sealed and heated at 150° C. for 7 h. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-((3-cyano-7-(tert-butoxycarbonyl)(3,5-dichlorobenzyl)aminopyrazolo[1,5-a]pyrimidin-5-yl)amino)piperidine-1-carboxylate as a yellow oil (83 mg, 79%). TLC system: EtOAc/hexane (1:2), R_{f} value: ˜0.30; ¹HNMR (400 MHz, CDCl₃) δ 7.96 (s, 1H), 7.29 (t, J=1.9 Hz, 1H), 7.24 (t, J=1.9 Hz, 2H), 7.18 (s, 1H), 4.51 (s, 2H), 3.58-3.22 (m, 5H), 1.92-1.87 (m, 1H), 1.70-1.66 (m, 2H), 1.58-1.54 (m, 1H), 1.42 (s, 9H), 1.38 (s, 9H).

### (S)-3-Cyano-7-((3,5-dichlorophen-1-yl)methyl)amino-5-((piperidin-3-yl)amino)pyrazolo[1,5-a]pyrimidine (I-26)

To a solution of tert-butyl (S)-3-((3-cyano-7-(tert-butoxycarbonyl)(3,5-dichlorobenzyl)aminopyrazolo[1,5-a]pyrimidin-5-yl)amino)piperidine-1-carboxylate (20 mg, 0.03 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-3-cyano-7-((3,5-dichlorophen-1-yl)methyl)amino-5-((piperidin-3-yl)amino)pyrazolo[1,5-a]pyrimidine (I-26) as an off white solid (15 mg, yield: 88%). TLC system: DCM/Methanol (1:1), R_{f} value: ˜0.2; ¹H NMR (400 Hz, MeOD-d4) δ 8.06 (s, 1H), 7.38-7.34 (m, 3H), 5.22 (s, 1H), 4.53 (s, 2H), 4.09-3.99 (m, 1H), 3.27-3.21 (m, 1H), 2.94-2.89 (m, 1H), 2.57-2.51 (m, 1H), 2.43-2.37 (m, 1H), 2.04-1.98 (m, 1H), 1.78-1.72 (m, 1H), 1.64-1.57 (m, 1H), 1.47-1.38 (m, 1H). HRMS (ESI) m/z [M+H]⁺ calcd for C₁₉H₂₀Cl₂N₇ 416.1152, found 416.1166. HPLC: 95%.

### Example 29

### Synthesis of (S)-7-((3-chloro-5-fluorobenzyl)amino)-5-((piperidin-3-yl)amino)pyrazolo[1,5-a]pyrimidine-3-carbonitrile (Compound I-27)

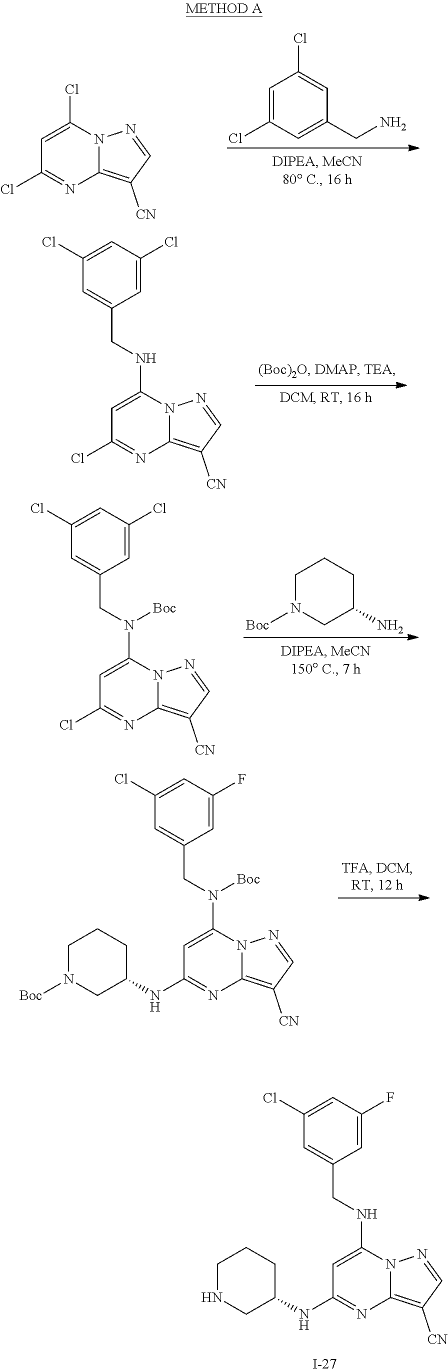

### 5-Chloro-3-cyano-N-(3-chloro-5-fluorobenzyl)pyrazolo[1,5-a]pyrimidin-7-amine

To a stirred solution of 3-chloro-5-fluorobenzylamine (53 mg, 0.33 mmol, 1.1 eq) in acetonitrile (5 mL) was added DIPEA (77 mg, 0.60 mmol, 2.0 eq) and the solution was stirred for 5 min. To this mixture 5,7-dichloropyrazolo[1,5-a]pyrimidine-3-carbonitrile (64 mg, 0.30 mmol, 1.0 eq) was added at room temperature and stirring continued at 80° C. for 16 h. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound was purified by using CombiFlash chromatography (4 g column) to afford 5-chloro-3-cyano-N-(3-chloro-5-fluorobenzyl)pyrazolo[1,5-a]pyrimidin-7-amine as a white solid (86 mg, yield: 86%). TLC system: EtoAc:Hexane (1:2), R_{f} value: ˜0.4.

### Tert-Butyl (5-chloro-3-cyanopyrazolo[1,5-a]pyrimidin-7-yl)(3-chloro-5-fluorobenzyl)carbamate

To a stirred solution of 5-chloro-3-cyano-N-(3-chloro-5-fluorobenzyl)pyrazolo[1,5-a]pyrimidin-7-amine (80 mg, 0.24 mmol, 1.0 eq) in DCM (5 mL) at room temperature was added triethylamine (49 mg, 0.48 mmol, 2.0 eq) and DMAP (2 mg 0.016 mmol), followed by addition of (Boc)₂O (68 mg, 0.31 mmol, 1.3 eq). The reaction was continued at room temperature for 16 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyanopyrazolo[1,5-a]pyrimidin-7-yl)(3-chloro-5-fluorobenzyl)carbamate as a white oil (86 mg, 82%). TLC system: EtOAc/hexane (1:2), R_{f} value: ˜0.7.

### Tert-Butyl (S)-3-(7-(tert-butoxycarbonyl)(3-chloro-5-fluorobenzyl)amino-3-cyanopyrazolo[1,5-a]pyrimidin-5-yl)aminopiperidine-1-carboxylate

To a stirred solution of tert-butyl (5-chloro-3-cyanopyrazolo[1,5-a]pyrimidin-7-yl)(3-chloro-5-fluorobenzyl)carbamate (80 mg, 0.19 mmol, 1 eq) in acetonitrile (3 mL) in a microwave reaction vial, (S)-tert-butyl 3-aminopiperidine-1-carboxylate (46 mg, 0.23 mmol, 1.2 eq) and DIPEA (49 mg, 0.38 mmol, 2.0 eq) were added at room temperature. The reaction mixture was under nitrogen flush for 5 minutes and then the microwave vial was sealed and heated at 150° C. for 7 h. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-(7-(tert-butoxycarbonyl)(3-chloro-5-fluorobenzyl)amino-3-cyanopyrazolo[1,5-a]pyrimidin-5-yl)aminopiperidine-1-carboxylate as a yellow oil (92 mg, 80%). TLC system: EtOAc/hexane (1:2), R_{f} value: ˜0.30.

### (S)-7-((3-Chloro-5-fluorobenzyl)amino)-5-((piperidin-3-yl)amino)pyrazolo[1,5-a]pyrimidine-3-carbonitrile (I-27)

To a solution of tert-butyl (S)-3-(7-(tert-butoxycarbonyl)(3-chloro-5-fluorobenzyl)amino-3-cyanopyrazolo[1,5-a]pyrimidin-5-yl)aminopiperidine-1-carboxylate (60 mg, 0.10 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-7-((3-chloro-5-fluorobenzyl)amino)-5-((piperidin-3-yl)amino)pyrazolo[1,5-a]pyrimidine-3-carbonitrile (I-27) as an off white solid (13 mg, yield: 33%). TLC system: DCM/Methanol (1:1), R_{f} value: ˜0.2; ¹H NMR (400 Hz, MeOD-d4) δ 8.06 (s, 1H), 7.24 (s, 1H), 7.11-7.08 (m, 2H), 5.26 (s, 1H), 4.54 (s, 2H), 4.19-4.09 (m, 1H), 3.37-3.32 (m, 1H), 3.09-3.02 (m, 1H), 2.74-2.67 (m, 1H), 2.61-2.55 (m, 1H), 2.04-1.99 (m, 1H), 1.88-1.82 (m, 1H), 1.73-1.63 (m, 1H), 1.54-1.44 (m, 1H). HRMS (ESI) m/z [M+H]⁺ calcd for C₁₉H₂₀ClFN₇ 400.1447, found 400.1477. HPLC: 95%.

### Example 30

### Synthesis of (S)-7-((3-chlorobenzyl)amino)-5-((piperidin-3-yl)amino)pyrazolo[1,5-a]pyrimidine-3-carbonitrile (Compound I-28)

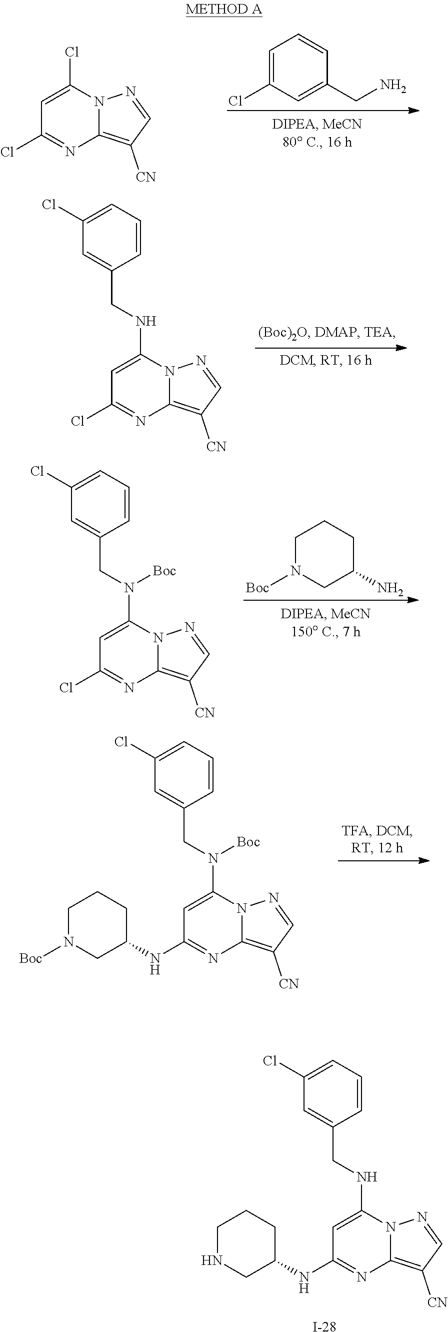

### 5-Chloro-3-cyano-N-(3-chlorobenzyl)pyrazolo[1,5-a]pyrimidin-7-amine

To a stirred solution of 3-chlorobenzylamine (47 mg, 0.33 mmol, 1.1 eq) in acetonitrile (5 mL) was added DIPEA (77 mg, 0.60 mmol, 2.0 eq) and the solution was stirred for 5 min. To this mixture 5,7-dichloropyrazolo[1,5-a]pyrimidine-3-carbonitrile (64 mg, 0.30 mmol, 1.0 eq) was added at room temperature and stirring continued at 80° C. for 16 h. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound was purified by using CombiFlash chromatography (4 g column) to afford 5-chloro-3-cyano-N-(3-chlorobenzyl)pyrazolo[1,5-a]pyrimidin-7-amine as a white solid (90 mg, yield: 95%). TLC system: EtoAc:Hexane (1:2), R_{f} value: ˜0.4.

### Tert-Butyl (5-chloro-3-cyanopyrazolo[1,5-a]pyrimidin-7-yl)(3-chlorobenzyl)carbamate

To a stirred solution of 5-chloro-3-cyano-N-(3-chlorobenzyl)pyrazolo[1,5-a]pyrimidin-7-amine (100 mg, 0.32 mmol, 1.0 eq) in DCM (5 mL) at room temperature was added triethylamine (65 mg, 0.64 mmol, 2.0 eq) and DMAP (2 mg 0.016 mmol), followed by addition of (Boc)₂O (91 mg, 0.42 mmol, 1.3 eq). The reaction was continued at room temperature for 16 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyanopyrazolo[1,5-a]pyrimidin-7-yl)(3-chlorobenzyl)carbamate as a white oil (94 mg, 70%). TLC system: EOAc/hexane (1:2), R_{f} value: ˜0.5.

### Tert-Butyl (S)-3-(7-(tert-butoxycarbonyl)(3-chlorobenzyl)amino-3-cyanopyrazolo[1,5-a]pyrimidin-5-yl)aminopiperidine-1-carboxylate

To a stirred solution of tert-butyl (5-chloro-3-cyanopyrazolo[1,5-a]pyrimidin-7-yl)(3-chlorobenzyl)carbamate (80 mg, 0.19 mmol, 1 eq) in acetonitrile (3 mL) in a microwave reaction vial, (S)-tert-butyl 3-aminopiperidine-1-carboxylate (46 mg, 0.23 mmol, 1.2 eq) and DIPEA (49 mg, 0.38 mmol, 2.0 eq) were added at room temperature. The reaction mixture was under nitrogen flush for 5 minutes and then the microwave vial was sealed and heated at 150° C. for 7 h. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-(7-(tert-butoxycarbonyl)(3-chlorobenzyl)amino-3-cyanopyrazolo[1,5-a]pyrimidin-5-yl)aminopiperidine-1-carboxylate as a yellow oil (98 mg, 89%). TLC system: EtOAc/hexane (1:2), R_{f} value: ˜0.30.

### (S)-7-((3-Chlorobenzyl)amino)-5-((piperidin-3-yl)amino)pyrazolo[1,5-a]pyrimidine-3-carbonitrile (I-28)

To a solution of tert-butyl (S)-3-(7-(tert-butoxycarbonyl)(3-chlorobenzyl)amino-3-cyanopyrazolo[1,5-a]pyrimidin-5-yl)aminopiperidine-1-carboxylate (60 mg, 0.11 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-7-((3-chlorobenzyl)amino)-5-((piperidin-3-yl)amino)pyrazolo[1,5-a]pyrimidine-3-carbonitrile (I-28) as an off white solid (30 mg, yield: 77%). TLC system: DCM/Methanol (1:1), R_{f} value: ˜0.2; ¹H NMR (400 Hz, MeOD-d4) δ 8.05 (s, 1H), 7.39 (s, 1H), 7.34-7.28 (m, 3H), 5.24 (s, 1H), 4.54 (s, 2H), 4.08-3.96 (m, 1H), 3.25-3.22 (m, 1H), 2.95-2.89 (m, 1H), 2.58-2.51 (m, 1H), 2.42-2.36 (m, 1H), 2.02-1.97 (m, 1H), 1.78-1.72 (m, 1H), 1.66-1.56 (m, 1H), 1.46-1.36 (m, 1H). HRMS (ESI) m/z [M+H]⁺ calcd for C₁₉H₂₁ClN₇ 382.1541, found 382.1554. HPLC: 95%.

### Example 31

### Synthesis of (S)-7-((3-fluorobenzyl)amino)-5-((piperidin-3-yl)amino)pyrazolo[1,5-a]pyrimidine-3-carbonitrile (Compound I-29)

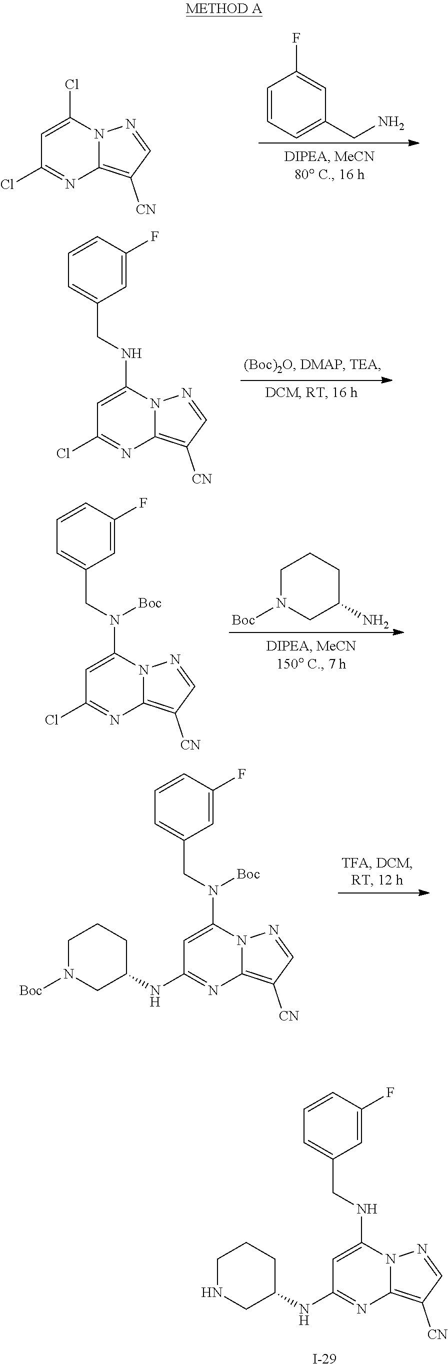

### 5-Chloro-3-cyano-N-(3-fluorobenzyl)pyrazolo[1,5-a]pyrimidin-7-amine

To a stirred solution of 3-fluorobenzylamine (42 mg, 0.33 mmol, 1.1 eq) in acetonitrile (5 mL) was added DIPEA (77 mg, 0.60 mmol, 2.0 eq) and the solution was stirred for 5 min. To this mixture 5,7-dichloropyrazolo[1,5-a]pyrimidine-3-carbonitrile (64 mg, 0.30 mmol, 1.0 eq) was added at room temperature and stirring continued at 80° C. for 16 h. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound was purified by using CombiFlash chromatography (4 g column) to afford 5-chloro-3-cyano-N-(3-fluorobenzyl)pyrazolo[1,5-a]pyrimidin-7-amine as a white solid (87 mg, yield: 96%). TLC system: EtoAc:Hexane (1:2), R_{f} value: ˜0.4; ¹H NMR (400 MHz, CDCl₃) δ 8.23 (s, 1H), 7.43-7.38 (m, 1H), 7.16-7.13 (m, 1H), 7.10-7.05 (m, 2H), 7.00-6.95 (m, 1H), 4.64 (d, J=6.0 Hz, 2H).

### Tert-Butyl (5-chloro-3-cyanopyrazolo[1,5-a]pyrimidin-7-yl)(3-fluorobenzyl)carbamate

To a stirred solution of 5-chloro-3-cyano-N-(3-fluorobenzyl)pyrazolo[1,5-a]pyrimidin-7-amine (80 mg, 0.27 mmol, 1.0 eq) in DCM (5 mL) at room temperature was added triethylamine (55 mg, 0.54 mmol, 2.0 eq) and DMAP (2 mg 0.016 mmol), followed by addition of (Boc)₂O (77 mg, 0.35 mmol, 1.3 eq). The reaction was continued at room temperature for 16 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyanopyrazolo[1,5-a]pyrimidin-7-yl)(3-fluorobenzyl)carbamate as a yellow oil (70 mg, 65%). TLC system: EOAc/hexane (1:2), R_{f} value: ˜0.5; ¹H NMR (400 MHz, CDCl₃) δ 8.38 (s, 1H), 7.30-7.24 (m, 1H), 6.99-6.95 (m, 3H), 6.81 (s, 1H), 5.04 (s, 2H), 1.39 (s, 9H).

### Tert-Butyl (S)-3-(7-(tert-butoxycarbonyl)(3-fluorobenzyl)amino-3-cyanopyrazolo[1,5-a]pyrimidin-5-yl)aminopiperidine-1-carboxylate

To a stirred solution of tert-butyl (5-chloro-3-cyanopyrazolo[1,5-a]pyrimidin-7-yl)(3-fluorobenzyl)carbamate (60 mg, 0.15 mmol, 1 eq) in acetonitrile (3 mL) in a microwave reaction vial, (S)-tert-butyl 3-aminopiperidine-1-carboxylate (45 mg, 0.23 mmol, 1.5 eq) and DIPEA (39 mg, 0.30 mmol, 2.0 eq) were added at room temperature. The reaction mixture was under nitrogen flush for 5 minutes and then the microwave vial was sealed and heated at 150° C. for 7 h. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-(7-(tert-butoxycarbonyl)(3-fluorobenzyl)amino-3-cyanopyrazolo[1,5-a]pyrimidin-5-yl)aminopiperidine-1-carboxylate as a yellow oil (50 mg, 59%). TLC system: EOAc/hexane (1:1), R_{f} value: ˜0.30; ¹HNMR (400 MHz, CDCl₃) δ 7.97 (s, 1H), 7.38-7.32 (m, 1H), 7.16-7.14 (m, 1H), 7.09-7.04 (m, 1H), 7.02-6.95 (m, 1H), 6.61 (s, 1H), 4.55 (s, 2H), 3.76-3.21 (m, 5H), 2.19-2.16 (m, 1H), 1.92-1.88 (m, 1H), 1.70-1.66 (m, 1H), 1.58-1.54 (m, 1H), 1.43 (s, 9H), 1.39 (s, 9H).

### (S)-7-((3-Fluorobenzyl)amino)-5-((piperidin-3-yl)amino)pyrazolo[1,5-a]pyrimidine-3-carbonitrile (I-29)

To a solution of tert-butyl (S)-3-(7-(tert-butoxycarbonyl)(3-fluorobenzyl)amino-3-cyanopyrazolo[1,5-a]pyrimidin-5-yl)aminopiperidine-1-carboxylate (50 mg, 0.09 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-7-((3-fluorobenzyl)amino)-5-((piperidin-3-yl)amino)pyrazolo[1,5-a]pyrimidine-3-carbonitrile (I-29) as an off white solid (20 mg, yield: 50%). TLC system: DCM/Methanol (1:1), R_{f} value: ˜0.2; ¹H NMR (400 Hz, MeOD-d4) δ 8.06 (s, 1H), 7.39-7.33 (m, 1H), 7.19 (d, J=7.6 Hz, 1H), 7.11 (d, J=9.8 Hz, 1H), 7.02-6.97 (m, 1H), 5.27 (s, 1H), 4.55 (s, 2H), 4.16-4.06 (m, 1H), 3.40-3.35 (m, 1H), 3.07-3.04 (m, 1H), 2.74-2.67 (m, 1H), 2.60-2.55 (m, 1H), 2.03-2.00 (m, 1H), 1.89-1.84 (m, 1H), 1.74-1.67 (m, 1H), 1.57-1.48 (m, 1H). HRMS (ESI) m/z [M+H]⁺ calcd for C₁₉H₂₁FN₇ 366.1837, found 366.1858. HPLC: 95%.

### Example 32

### Synthesis of (S)-7-((3-methylbenzyl)amino)-5-((piperidin-3-yl)amino)pyrazolo[1,5-a]pyrimidine-3-carbonitrile (Compound I-30)

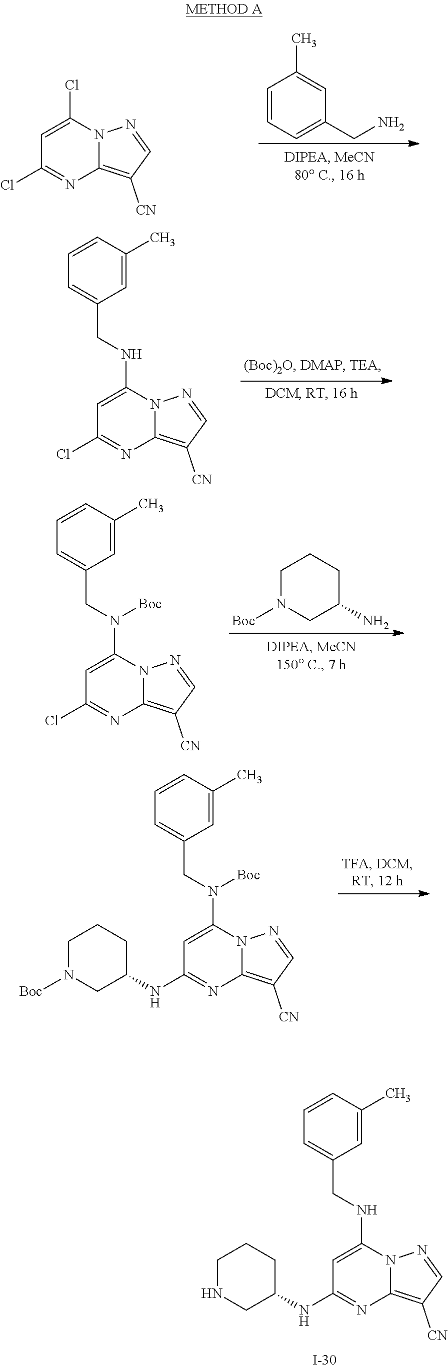

### 5-Chloro-3-cyano-N-(3-methylbenzyl)pyrazolo[1,5-a]pyrimidin-7-amine

To a stirred solution of 3-methybenzylamine (40 mg, 0.33 mmol, 1.1 eq) in acetonitrile (5 mL) was added DIPEA (77 mg, 0.60 mmol, 2.0 eq) and the solution was stirred for 5 min. To this mixture 5,7-dichloropyrazolo[1,5-a]pyrimidine-3-carbonitrile (64 mg, 0.30 mmol, 1.0 eq) was added at room temperature and stirring continued at 80° C. for 16 h. After completion of reaction by TLC, the reaction mixture was diluted with ice cold water and extracted with (3×30 mL) ethyl acetate. The combined organic layer was washed with water (50 mL), brine (20 mL), dried over sodium sulfate, and concentrated. The crude compound was purified by using CombiFlash chromatography (4 g column) to afford 5-chloro-3-cyano-N-(3-methylbenzyl)pyrazolo[1,5-a]pyrimidin-7-amine as a white solid (83 mg, yield: 92%). TLC system: EtoAc:Hexane (1:4), R_{f} value: ˜0.3.

### Tert-Butyl (5-chloro-3-cyanopyrazolo[1,5-a]pyrimidin-7-yl)(3-methylbenzyl)carbamate

To a stirred solution of 5-chloro-3-cyano-N-(3-methylbenzyl)pyrazolo[1,5-a]pyrimidin-7-amine (80 mg, 0.27 mmol, 1.0 eq) in DCM (5 mL) at room temperature was added triethylamine (55 mg, 0.54 mmol, 2.0 eq) and DMAP (2 mg 0.016 mmol), followed by addition of (Boc)₂O (77 mg, 0.35 mmol, 1.3 eq). The reaction was continued at room temperature for 16 h. After completion of reaction monitored by TLC, the reaction mixture was quenched with sodium bicarbonate in water and extracted with (2×30 mL) DCM. The combined organic layer was washed with water (20 mL), brine (20 mL), dried over sodium sulfate, and concentrated. The crude compound was purified by using CombiFlash chromatography (4 g column) to afford tert-butyl (5-chloro-3-cyanopyrazolo[1,5-a]pyrimidin-7-yl)(3-methylbenzyl)carbamate as a yellow oil (80 mg, 74%). TLC system: EtOAc/hexane (1:2), R_{f} value: ˜0.6.

### Tert-Butyl (S)-3-(7-(tert-butoxycarbonyl)(3-methylbenzyl)amino-3-cyanopyrazolo[1,5-a]pyrimidin-5-yl)aminopiperidine-1-carboxylate

To a stirred solution of tert-butyl (5-chloro-3-cyanopyrazolo[1,5-a]pyrimidin-7-yl)(3-methylbenzyl)carbamate (70 mg, 0.18 mmol, 1 eq) in acetonitrile (3 mL) in a microwave reaction vial, (S)-tert-butyl 3-aminopiperidine-1-carboxylate (54 mg, 0.27 mmol, 1.5 eq) and DIPEA (46 mg, 0.36 mmol, 2.0 eq) were added at room temperature. The reaction mixture was under nitrogen flush for 5 minutes and then the microwave vial was sealed and heated at 150° C. for 7 h. After completion of reaction by TLC, the reaction mixture was diluted with ethyl acetate and water. The mixture was extracted with (2×30 mL) ethyl acetate. The combined organic layer was washed with brine (30 mL), dried over sodium sulfate, and concentrated to provide crude product which was purified by using CombiFlash chromatography (4 g column) to afford tert-butyl (S)-3-(7-(tert-butoxycarbonyl)(3-methylbenzyl)amino-3-cyanopyrazolo[1,5-a]pyrimidin-5-yl)aminopiperidine-1-carboxylate as a yellow oil (60 mg, 60%). TLC system: EtOAc/hexane (1:2), R_{f} value: ˜0.50; ¹H NMR (400 MHz, CDCl₃) δ 7.95 (s, 1H), 7.28-7.24 (m, 1H), 7.18-7.13 (m, 2H), 7.08-7.01 (m, 1H), 5.83 (s, 1H), 4.46 (s, 2H), 3.69-3.25 (m, 5H), 2.35 (s, 3H), 1.91-1.86 (m, 2H), 1.73-1.68 (m, 1H), 1.59-1.53 (m, 1H), 1.43 (s, 9H), 1.39 (s, 9H).

### (S)-7-((3-Methylbenzyl)amino)-5-((piperidin-3-yl)amino)pyrazolo[1,5-a]pyrimidine-3-carbonitrile (I-30)

To a solution of tert-butyl (S)-3-(7-(tert-butoxycarbonyl)(3-methylbenzyl)amino-3-cyanopyrazolo[1,5-a]pyrimidin-5-yl)aminopiperidine-1-carboxylate (80 mg, 0.09 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction, sodium bicarbonate in water was added and the mixture stirred for 30 minutes. Then the reaction mixture was extracted with (2×30 mL) ethyl acetate. The combined organic layer was washed with water (30 mL), brine (30 mL), dried over sodium sulfate, and concentrated to provide crude product which was purified by using CombiFlash chromatography (4 g column) to afford (S)-7-((3-methylbenzyl)amino)-5-((piperidin-3-yl)amino)pyrazolo[1,5-a]pyrimidine-3-carbonitrile (I-30) as an off white solid (25 mg, yield: 78%). TLC system: DCM/Methanol (1:1), R_{f} value: ˜0.2; ¹H NMR (400 Hz, MeOD-d4) δ 8.08 (s, 1H), 7.29-7.13 (m, 3H), 7.10-7.06 (m, 1H), 5.31 (s, 1H), 4.50 (s, 2H), 4.35-4.27 (m, 1H), 3.64-3.60 (m, 1H), 3.30-3.26 (m, 1H), 2.99-2.93 (m, 1H), 2.88-2.83 (m, 1H), 2.32 (s, 3H), 2.09-2.00 (m, 2H), 1.88-1.82 (m, 1H), 1.67-1.58 (m, 1H). HRMS (ESI) m/z [M+H]⁺ calcd for C₂₀H₂₄N₇ 362.2088, found 362.2090. HPLC: 95%.

### Example 33

### Synthesis of (S)—N⁷-(3-chlorobenzyl)-3-cyclopropyl-N⁵-(piperidin-3-yl)pyrazolo[1,5-a]pyrimidine-5,7-diamine hydrochloride (Compound I-31)

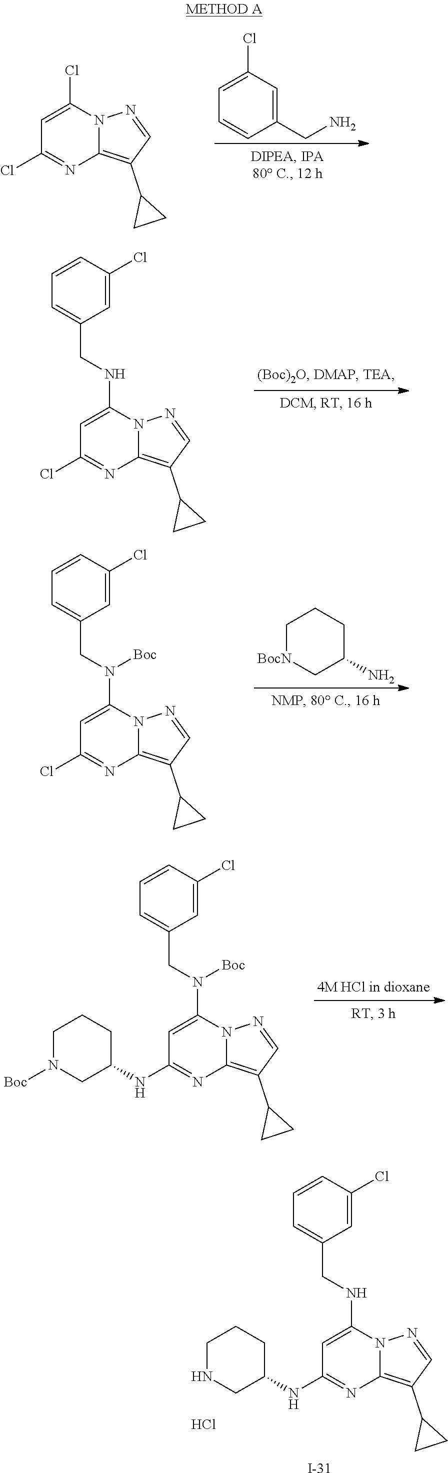

### 5-Chloro-N-(3-chlorobenzyl)-3-cyclopropylpyrazolo[1,5-a]pyrimidin-7-amine

To a stirred solution of (3-chlorophenyl)methanamine (500 mg, 3.546 mmol, 1.0 eq) in IPA (5 mL) was added DIPEA (1.23 mL, 7.09 mmol, 2.0 eq) under nitrogen flush and the solution was stirred for 10 min. To this mixture 5,7-dichloro-3-cyclopropylpyrazolo[1,5-a]pyrimidine, prepared according to Example 1 above (885 mg, 3.90 mmol, 1.1 eq), was added at room temperature and stirring continued at 80° C. for 12 h. After completion of reaction by TLC, the reaction mixture was diluted with ice cold water and extracted with (3×50 mL) ethyl acetate. The combined organic layer was washed with water (50 mL), brine (20 mL), dried over sodium sulfate, and concentrated. The crude compound was purified by grace column chromatography [gradient elution with 10-20% Ethyl acetate/Hexane] to afford (5-chloro-N-(3-chlorobenzyl)-3-cyclopropylpyrazolo[1,5-a]pyrimidin-7-amine as a yellow gummy solid (400 mg, yield: 54%). TLC system: EtoAc:Hexane (10:90), R_{f} value: ˜0.5; LCMS (m/z): 333.0 (M+H)⁺.

### Tert-Butyl (5-chloro-3-cyclopropylpyrazolo[1,5-a]pyrimidin-7-yl)(3-chlorobenzyl)carbamate

To a stirred solution of 5-chloro-N-(3-chlorobenzyl)-3-cyclopropylpyrazolo[1,5-a]pyrimidin-7-amine (320 mg, 0.963 mmol, 1.0 eq) in DCM (20 mL) at room temperature was added triethylamine (0.26 mL, 1.927 mmol, 2.0 eq) and DMAP (11.74 mg 0.096 mmol, 0.10 eq), followed by drop-wise addition of (Boc)₂O (0.256 mL, 1.307 mmol, 1.1 eq), under nitrogen flush. The reaction was continued at room temperature for 16 h. After completion of reaction monitored by TLC, the reaction mixture was quenched with ice cold water and extracted with (2×50 mL) DCM. The combined organic layer was washed with water (20 mL), brine (20 mL), dried over sodium sulfate, and concentrated. The crude compound was purified by grace column chromatography [gradient elution with 10-20% Ethyl acetate/Hexane] to afford tert-butyl (5-chloro-3-cyclopropylpyrazolo[1,5-a]pyrimidin-7-yl)(3-chlorobenzyl)carbamate as a yellow gummy solid (300 mg, 72%). TLC system: EtOAc/hexane (10:90), R_{f} value: ˜0.7; LCMS (m/z): 433.1 (M+H)⁺. ¹H NMR (400 MHz, DMSO-d₆) δ 8.10 (s, 1H), 7.41 (s, 1H), 7.31-7.28 (m, 3H), 7.20 (s, 1H), 4.97 (s, 2H), 1.99-1.97 (m, 1H), 1.28-1.23 (s, 9H), 0.95-0.91 (m, 2H), 0.80-0.78 (m, 2H).

### Tert-Butyl (S)-3-((7-((tert-butoxycarbonyl)(3-chlorobenzyl)amino)-3-cyclopropylpyrazolo[1,5-a]pyrimidin-5-yl)amino)piperidine-1-carboxylate

To a stirred solution of tert-butyl (5-chloro-3-cyclopropylpyrazolo[1,5-a]pyrimidin-7-yl)(3-chlorobenzyl)carbamate (300 mg, 0.692 mmol, 1.0 eq) in NMP (0.1 mL) tert-butyl (S)-3-aminopiperidine-1-carboxylate (152 mg, 0.762 mmol, 1.1 eq) was added at room temperature. The reaction mixture was then heated at 80° C. for 16 h. After completion of reaction by TLC, the reaction mixture was diluted with ice cold water and extracted with (2×100 mL) DCM. The combined organic layer was washed with water (50 mL), brine (50 mL), dried over sodium sulfate, and concentrated to provide crude product which was purified by grace chromatography [gradient elution with 40-50% Ethyl acetate/Hexane] to afford tert-butyl (S)-3-((7-((tert-butoxycarbonyl)(3-chlorobenzyl)amino)-3-cyclopropylpyrazolo[1,5-a]pyrimidin-5-yl)amino)piperidine-1-carboxylate as an off white gummy solid (50 mg, 12%). TLC system: EOAc/hexane (50:50), R_{f} value: ˜0.3; LCMS (m/z): 597.2 (M+H)⁺. ¹HNMR (400 MHz, DMSO-d₆) δ 7.64 (s, 1H), 7.39-7.32 (m, 2H), 7.26 (d, J=7.2 Hz, 1H), 7.16 (d, J=7.2 Hz, 1H), 6.10 (s, 1H), 4.82-4.79 (m, 2H), 4.04-3.79 (brs, 1H), 1.82-1.73 (m, 4H), 1.38-1.16 (m, 19H), 0.87-0.77 (m, 4H). (5 protons are less which might merged with NMR solvent peaks)

### (S)—N⁷-(3-Chlorobenzyl)-3-cyclopropyl-N⁵-(piperidin-3-yl)pyrazolo[1,5-a]pyrimidine-5,7-diamine Hydrochloride (I-31)

A solution of tert-butyl (S)-3-((7-((tert-butoxycarbonyl)(3-chlorobenzyl)amino)-3-cyclopropyl pyrazolo[1,5-a]pyrimidin-5-yl)amino)piperidine-1-carboxylate (50 mg, 0.083 mmol, 1.0 eq) in 4M HCl in Dioxane (2 mL) was stirred at room temperature for 3 h. After completion of reaction by TLC, the reaction mixture was concentrated. The crude compound was purified by trituration with diethyl ether followed by lyophilization afforded (S)—N⁷-(3-chlorobenzyl)-3-cyclopropyl-N⁵-(piperidin-3-yl)pyrazolo[1,5-a]pyrimidine-5,7-diamine hydrochloride (I-31) as an off white solid (25 mg, yield: 75%). LCMS (m/z): 397.1 (M−HCl+H)⁺; ¹H NMR (400 MHz, DMSO-d₆) δ 9.42-9.01 (brs, 2H), 7.76 (s, 1H), 7.53-7.33 (m, 4H), 5.70-5.50 (brs, 1H), 4.64 (brs, 2H), 4.22 (brs, 1H), 3.41-3.32 (m, 2H), 2.91-2.79 (m, 2H), 1.96-1.78 (m, 4H), 1.59-1.58 (m, 1H), 0.86-0.85 (m, 2H), 0.68-0.66 (m, 2H). NH protons are clearly evident in the spectrum.

### Example 34

### Synthesis of (S)-3-cyclopropyl-N⁷-(3-fluorobenzyl)-N⁵-(piperidin-3-yl)pyrazolo[1,5-a]pyrimidine-5,7-diamine Hydrochloride (Compound I-32)

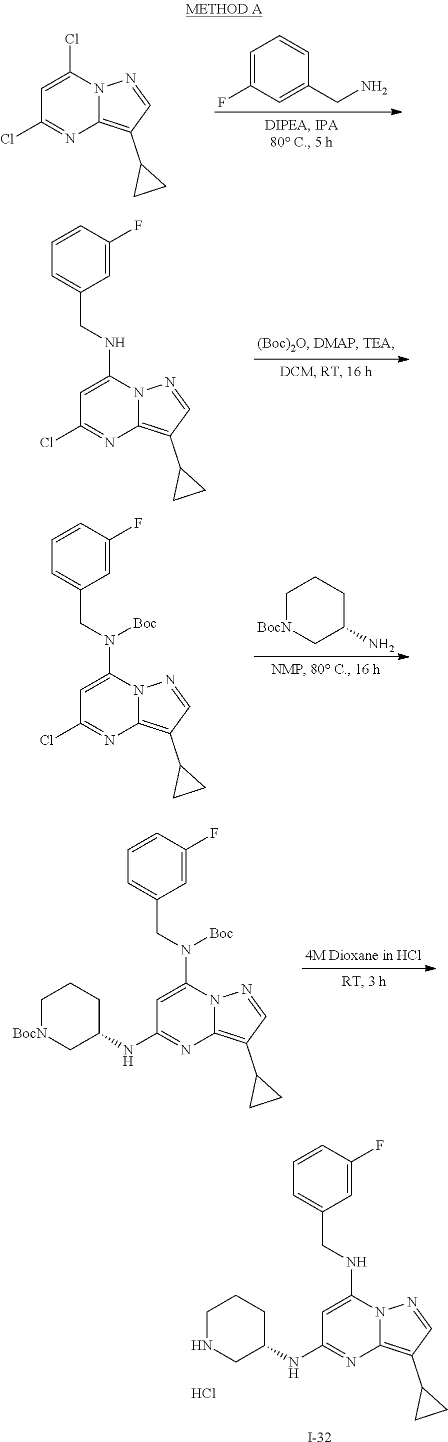

### 5-Chloro-3-cyclopropyl-N-(3-fluorobenzyl)pyrazolo[1,5-a]pyrimidin-7-amine

To a stirred solution of (3-fluorophenyl)methanamine (400 mg, 3.20 mmol, 1.0 eq) in IPA (10 mL) was added DIPEA (1.11 mL, 6.40 mmol, 2.0 eq) under nitrogen flush and the solution was stirred for 10 min. To this mixture 5,7-dichloro-3-cyclopropylpyrazolo[1,5-a]pyrimidine, prepared according to Example 1 above (799 mg, 3.52 mmol, 1.1 eq), was added at room temperature and stirring continued at 80° C. for 4 h. After completion of reaction by TLC, the reaction mixture was diluted with ice cold water and extracted with (3×50 mL) ethyl acetate. The combined organic layer was washed with water (50 mL), brine (20 mL), dried over sodium sulfate, and concentrated. The crude compound was purified by grace column chromatography [gradient elution with 10-20% Ethyl acetate/Hexane] to afford 5-chloro-3-cyclopropyl-N-(3-fluorobenzyl)pyrazolo[1,5-a]pyrimidin-7-amine as a yellow solid (370 mg, yield: 36%). TLC system: EtoAc:Hexane (10:90), R_{f} value: ˜0.5; LCMS (m/z): 317.1 (M+H)⁺. ¹HNMR (400 MHz, CDCl₃) δ 7.70 (s, 1H), 7.39-7.33 (m, 1H), 7.13 (d, J=7.2 Hz, 1H), 7.06-6.99 (m, 2H), 6.73 (brs, 1H), 5.89 (s, 1H), 4.58 (d, J=6.0 Hz, 2H), 2.05-1.99 (m, 1H), 0.98-0.93 (m, 2H), 0.77-0.75 (m, 2H).

### Tert-Butyl (5-chloro-3-cyclopropylpyrazolo[1,5-a]pyrimidin-7-yl)(3-fluorobenzyl)carbamate

To a stirred solution of 5-chloro-3-cyclopropyl-N-(3-fluorobenzyl)pyrazolo[1,5-a]pyrimidin-7-amine (370 mg, 1.17 mmol, 1.0 eq) in DCM (20 mL) at room temperature was added triethylamine (0.31 mL, 2.34 mmol, 2.0 eq) and DMAP (7.1 mg 0.05 mmol, 0.05 eq), followed by drop-wise addition of (Boc)₂O (0.29 mL, 1.28 mmol, 1.1 eq), under nitrogen flush. The reaction was continued at room temperature for 16 h. After completion of reaction by TLC, the reaction mixture was diluted with ice cold water and extracted with (2×50 mL) DCM. The combined organic layer was washed with water (20 mL), brine (20 mL), dried over sodium sulfate, and concentrated. The crude compound was purified by grace column chromatography [gradient elution with 05-10% Ethyl acetate/Hexane] to afford tert-butyl (5-chloro-3-cyclopropylpyrazolo[1,5-a]pyrimidin-7-yl)(3-fluorobenzyl)carbamate as a yellow solid (340 mg, 70%). TLC system: EtOAc/hexane (10:90), R_{f} value: ˜0.7; LCMS (m/z): 417.2 (M+H)⁺.

### Tert-Butyl (S)-3-((7-((tert-butoxycarbonyl)(3-fluorobenzyl)amino)-3-cyclopropylpyrazolo[1,5-a]pyrimidin-5-yl)amino)piperidine-1-carboxylate

To a stirred solution of tert-butyl (5-chloro-3-cyclopropylpyrazolo[1,5-a]pyrimidin-7-yl)(3-fluorobenzyl)carbamate (320 mg, 0.77 mmol, 1.0 eq) in NMP (2 mL) tert-butyl (S)-3-aminopiperidine-1-carboxylate (169 mg, 0.84 mmol, 1.1 eq) was added at room temperature. The reaction mixture was then heated at 80° C. for 16 h. After completion of reaction by TLC, the reaction mixture was diluted with ice cold water and extracted with (2×50 mL) DCM. The combined organic layer was washed with water (50 mL), brine (50 mL), dried over sodium sulfate, and concentrated to provide crude product which was purified PREP-HPLC to afford tert-butyl (S)-3-((7-((tert-butoxycarbonyl)(3-fluorobenzyl)amino)-3-cyclopropyl pyrazolo[1,5-a]pyrimidin-5-yl)amino)piperidine-1-carboxylate as an off white solid (110 mg, 24%). TLC system: EtOAc/hexane (50:50), R_{f} value: ˜0.3; LCMS (m/z): 581.4 (M+H)⁺. ¹HNMR (400 MHz, DMSO-d₆) δ 7.64 (s, 1H), 7.37 (ABq, J=7.6 Hz, 14 Hz, 1H), 7.16-7.07 (m, 4H), 6.11 (s, 1H), 4.86-4.81 (m, 2H), 3.80 (brs, 1H), 1.89-1.72 (m, 3H), 1.51-1.35 (m, 2H), 1.29-1.18 (m, 18H), 0.81-0.78 (m, 4H). Two sets of N—CH₂'s might merge with solvent DMSO-d₆ peaks.

### (S)-3-Cyclopropyl-N⁷-(3-fluorobenzyl)-N⁵-(piperidin-3-yl)pyrazolo[1,5-a]pyrimidine-5,7-diamine Hydrochloride (I-32)

A solution of tert-butyl (S)-3-((7-((tert-butoxycarbonyl)(3-fluorobenzyl)amino)-3-cyclopropyl pyrazolo[1,5-a]pyrimidin-5-yl)amino)piperidine-1-carboxylate (100 mg, 0.17 mmol, 1.0 eq) in 4M HCl in Dioxane (2 mL) was stirred at room temperature for 3 h. After completion of reaction by TLC, the reaction mixture was concentrated. The crude compound was purified by trituration with diethyl ether followed by lyophilization afforded (S)-3-cyclopropyl-N⁷-(3-fluorobenzyl)-N⁵-(piperidin-3-yl)pyrazolo[1,5-a]pyrimidine-5,7-diamine hydrochloride (I-32) as an off white solid (60 mg, yield: 93%). LCMS (m/z): 381.3 (M+H−HCl)⁺; ¹H NMR (400 MHz, DMSO-d₆) δ 9.80-9.60 (br, 2H), 9.11 (brs, 1H), 7.78 (brs, 1H), 7.42-7.33 (m, 3H), 7.10 (t, J=8.4 Hz, 1H), 5.62 (brs, 1H), 4.67 (brs, 2H), 4.24 (brs, 1H), 3.39-3.21 (m, 2H), 2.90-2.76 (m, 2H), 1.99-1.77 (m, 4H), 1.60-1.58 (m, 1H), 0.87-0.85 (m, 2H), 0.67-0.65 (m, 2H).

### Example 35

### Synthesis of (S)-2-(3-((3-cyclopropyl-7-((3-fluorobenzyl)amino)pyrazolo[1,5-a]pyrimidin-5-yl)amino)piperidin-1-yl)ethan-1-ol (Compound I-33)

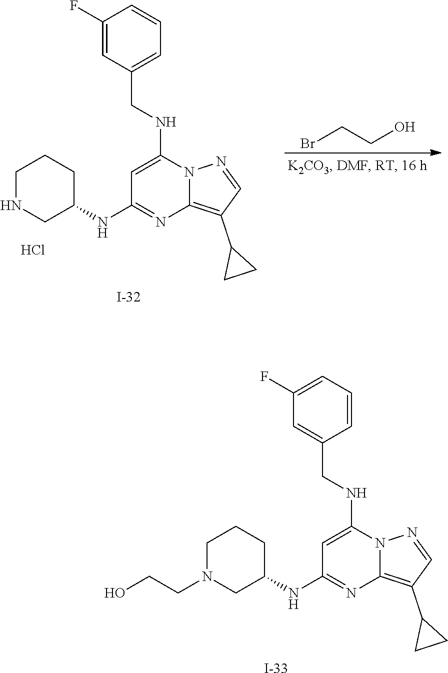

[[hydrochloride in Table 1]] A solution of (S)-3-cyclopropyl-N⁷-(3-fluorobenzyl)-N⁵-(piperidin-3-yl)pyrazolo[1,5-a]pyrimidine-5,7-diamine (I-32), prepared according to Example 35 above (35 mg, 0.09 mmol, 1.0 eq) in DMF (2 mL), was added K₂CO₃ (19 mg, 0.13, 1.5 eq) followed by bromo ethanol (0.07 mL, 0.06 mmol, 1.2 eq) and the reaction stirred at room temperature for 16 h. After completion of reaction by TLC, the reaction mixture was quenched with ice cold water and extracted with (2×50 mL) DCM. The combined organic layer was washed with water (20 mL), brine (20 mL), dried over sodium sulfate, and concentrated. The crude compound was purified by grace column chromatography [gradient elution with 10-20% Methanol/DCM] afforded (S)-2-(3-((3-cyclopropyl-7-((3-fluorobenzyl)amino)pyrazolo[1,5-a]pyrimidin-5-yl)amino)piperidin-1-yl)ethan-1-ol (I-33) as an off white solid (12 mg, yield: 30%). TLC system: Methanol/DCM] (10:90), R_{f} value: ˜0.3; LCMS (m/z): 425.3 (M+H)⁺; ¹HNMR (400 MHz, DMSO-d₆) δ 7.83-7.82 (m, 1H), 7.54 (s, 1H), 7.38 (ABq, J=8.0 Hz, 14.4 Hz, 1H), 7.19-7.13 (m, 2H), 7.10-7.05 (m, 1H), 6.46 (d, J=6.4 Hz, 1H), 5.11 (s, 1H), 4.44 (d, J=6.4 Hz, 2H), 3.93 (brs, 1H), 3.50-3.49 (brs, 2H), 2.95 (brs, 1H), 2.67-2.64 (m, 1H), 2.46-2.45 (m, 2H), 2.16-1.91 (m, 2H), 1.78-1.72 (m, 2H), 1.65-1.63 (m, 1H), 1.55-1.52 (m, 1H), 1.24-1.22 (m, 2H), 0.76-0.74 (m, 4H).

### Example 36

### Synthesis of (3R,4R)-4-((3-cyclopropyl-7-((3-fluorophenyl)amino)pyrazolo[1,5-a]pyrimidin-5-yl)aminomethyl)piperidin-3-ol (Compound I-45)

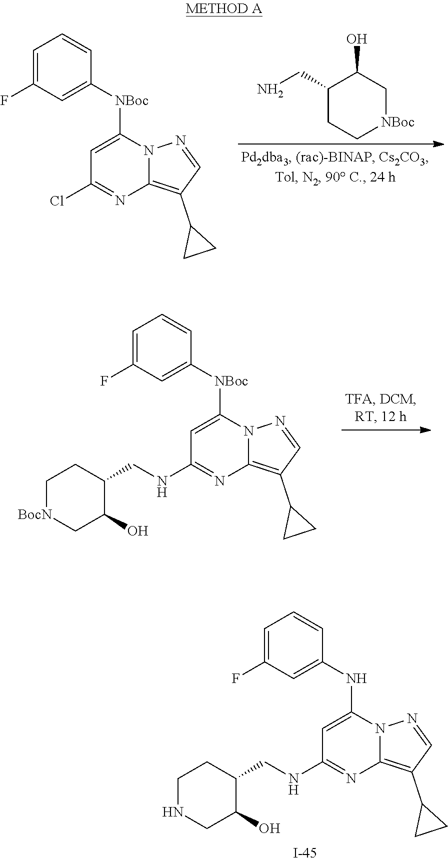

### Tert-Butyl (3R,4R)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3-fluorophenyl)carbamate, prepared according to Example 6 above (100 mg, 0.25 mmol, 1.0 eq), tert-butyl (3R,4R)-4-(aminomethyl)-3-hydroxypiperidine-1-carboxylate (69 mg, 0.30 mmol, 1.2 eq) and Cs₂CO₃ (163 mg, 0.5 mmol, 2 eq) were added at room temperature. The reaction mixture was under nitrogen flush for 5 minutes and then the microwave vial was sealed and heated at 90° C. for 24 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude (tert-butyl (3R,4R)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate as a brown oil (60 mg, 40%). TLC system: EOAc/hexane (1:1), R_{f} value: ˜0.30.

### (3R,4R)-4-((3-Cyclopropyl-7-((3-fluorophenyl)amino)pyrazolo[1,5-a]pyrimidin-5-yl)aminomethyl)piperidin-3-ol (I-45)

To a solution of crude tert-butyl (3R,4R)-4-((7-((tert-butoxycarbonyl)(3-fluorophenyl)amino)-3-cyclopropylpyrazolo[1,5-a]pyrimidin-5-yl)aminomethyl)-3-hydroxypiperidine-1-carboxylate (120 mg, 0.20 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-4-((3-cyclopropyl-7-((3-fluorophenyl)amino)pyrazolo[1,5-a]pyrimidin-5-yl)aminomethyl)piperidin-3-ol (I-45) as an off white solid (65 mg, yield: 81%). TLC system: DCM/Methanol (1:1), R_{f} value: ˜0.1; ¹HNMR (400 Hz, MeOD-d4) δ 7.54 (s, 1H), 7.44-7.38 (s, 1H), 7.18-7.12 (m, 2H), 6.93 (td, J=8.5, 2.3 Hz, 1H), 5.78 (s, 1H), 4.04-4.00 (m, 1H), 3.42-3.35 (m, 1H), 3.24-3.20 (m, 1H), 3.17-3.09 (m, 2H), 2.73-2.67 (m, 1H), 2.58-2.53 (m, 1H), 1.80-1.73 (m, 2H), 1.58-1.55 (m, 2H), 0.88-0.82 (m, 2H), 0.63-0.59 (m, 2H). HRMS (ESI) m/z [M+H]⁺ calcd for C₂₁H₂₆FN₆O 397.2147, found 397.2163. HPLC: 97%.

### Example 37

### Synthesis of (3R,4R)-4-((3-cyclopropyl-7-((3,5-difluorophenyl)amino)pyrazolo[1,5-a]pyrimidin-5-yl)aminomethyl)piperidin-3-ol (COMPOUND I-46)

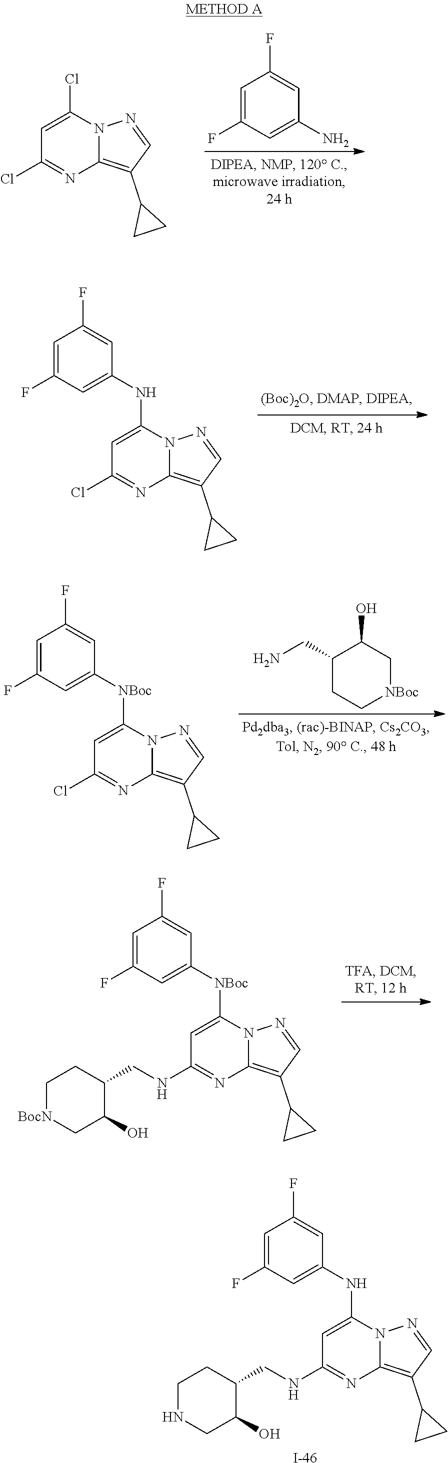

### 5-Chloro-3-cyclopropyl-N-(3,5-difluorophenyl)pyrazolo[1,5a]pyrimidin-7-amine

To a stirred solution of 3,5-difluroaniline (60 mg, 0.42 mmol, 1.0 eq) in NMP (4 mL) DIPEA (71 mg, 0.55 mmol, 1.3 eq) was added and stirred for 5 min. To this mixture 5,7-dichloro-3-cyclopropylpyrazolo[1,5-a]pyrimidine (115 mg, 0.50 mmol, 1.2 eq) was added at room temperature and continued stirring at 120° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclopropyl-N-(3,5-difluorophenyl)pyrazolo[1,5-a]pyrimidin-7-amine as a brown liquid. TLC system: EoAc:Hexane (1:6), R_{f} value: ˜0.4.

### Tert-Butyl (5-chloro-3-cyclopropylpyrazolo[1,5-a]pyrimidin-7-yl)(3,5-difluorophenyl)carbamate

To a stirred solution of 5-chloro-3-cyclopropyl-N-(3,5-difluorophenyl)pyrazolo[1,5-a]pyrimidin-7-amine (218 mg, 0.68 mmol, 1.0 eq) in DCM (5 mL) at room temperature, DIPEA (176 mg, 1.36 mmol, 2 eq) and DMAP (2 mg 0.016 mmol) were added, followed by addition of (Boc)₂O (192 mg, 0.88 mmol, 1.3 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by using combiflash chromatography (4 g column) to afford tert-butyl (5-chloro-3-cyclopropylpyrazolo[1,5-a]pyrimidin-7-yl)(3,5-difluorophenyl)carbamate as a yellow oil (250 mg, 87% for two steps). TLC system: EOAc/hexane (1:4), R_{f} value: ˜0.5; ¹HNMR (400 MHz, CDCl₃) δ 7.85 (s, 1H), 6.86-6.83 (m, 1H), 6.75-6.70 (m, 2H), 6.64 (s, 1H), 2.10-2.03 (m, 1H), 1.37 (s, 9H), 1.04-0.99 (m, 2H), 0.86-0.82 (m, 2H).

### Tert-Butyl (3R,4R)-4-((7-((tert-butoxycarbonyl)(3,5-difluorophenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3,5-difluorophenyl)carbamate (100 mg, 0.24 mmol, 1.0 eq), tert-butyl (3R,4R)-4-(aminomethyl)-3-hydroxypiperidine-1-carboxylate (67 mg, 0.29 mmol, 1.2 eq) and Cs₂CO₃ (156 mg, 0.48 mmol, 2 eq) were added at room temperature. The reaction mixture was under nitrogen flush for 5 minutes and then the microwave vial was sealed and heated at 90° C. for 48 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3R,4R)-4-((7-((tert-butoxycarbonyl)(3,5-difluorophenyl)amino)-3-cyclobutylpyrazolo[1,5-a]pyrimidin-5-yl)aminomethyl)-3-hydroxypiperidine-1-carboxylate as a brown oil (60 mg, 41%). TLC system: EOAc/hexane (1:1), R_{f} value: ˜0.30.

### (3R,4R)-4-((3-Cyclopropyl-7-((3,5-difluorophenyl)amino)pyrazolo[1,5-a]pyrimidin-5-yl)aminomethyl)piperidin-3-ol (I-46)

To a solution of crude tert-butyl (3R,4R)-4-((7-((tert-butoxycarbonyl)(3,5-difluorophenyl)amino)-3-cyclobutylpyrazolo[1,5-a]pyrimidin-5-yl)aminomethyl)-3-hydroxypiperidine-1-carboxylate (50 mg, 0.08 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide product (3R,4R)-4-((3-cyclopropyl-7-((3,5-difluorophenyl)amino)pyrazolo[1,5-a]pyrimidin-5-yl)aminomethyl)piperidin-3-ol (I-46) as yellow solid (20 mg, yield: 59%). TLC system: DCM/Methanol (1:1), R_{f} value: ˜0.1; ¹HNMR (400 Hz, MeOD-d4) δ 7.55 (s, 1H), 7.02-6.99 (m, 2H), 6.78-6.72 (m, 1H), 5.89 (s, 1H), 4.04-4.00 (m, 1H), 3.42-3.36 (m, 1H), 3.23-3.16 (m, 2H), 3.13-3.09 (m, 1H), 2.73-2.66 (m, 1H), 2.58-2.52 (m, 1H), 1.80-1.73 (m, 2H), 1.59-1.54 (m, 2H), 0.88-0.83 (m, 2H), 0.65-0.59 (m, 2H). HRMS (ESI) m/z [M+H]⁺ calcd for C₂₁H₂₅F₂N₆O 415.2052, found 415.2062. HPLC: 95%.

### Example 38

### Synthesis of (3S,4S)-4-((3-cyclopropyl-7-((3,5-difluorophenyl)amino)pyrazolo[1,5-a]pyrimidin-5-yl)aminomethyl)piperidin-3-ol (Compound I-47)

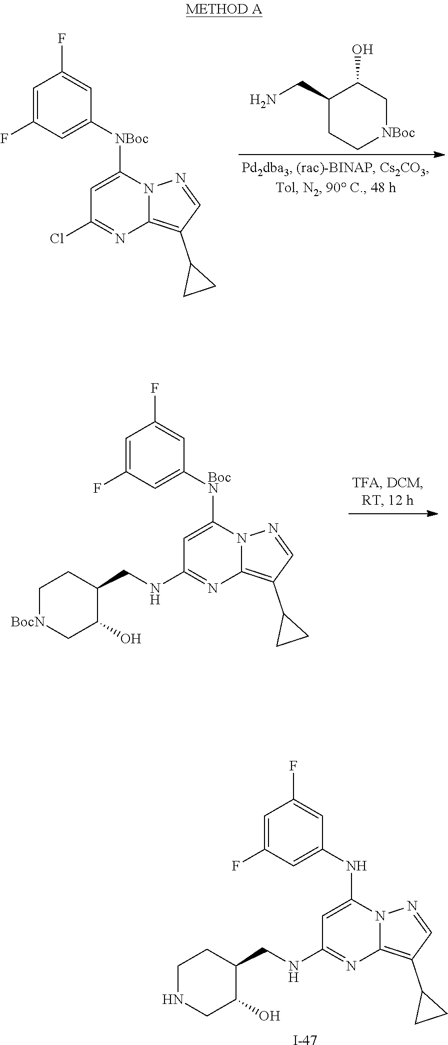

### Tert-Butyl (3S,4S)-4-((7-((tert-butoxycarbonyl)(3,5-difluorophenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial, tert-butyl (5-chloro-3-cyclopropylpyrazolo[1,5-a]pyrimidin-7-yl)(3,5-difluorophenyl)carbamate, prepared according to Example 6 above (102 mg, 0.24 mmol, 1.0 eq), tert-butyl (3S,4S)-4-(aminomethyl)-3-hydroxypiperidine-1-carboxylate (67 mg, 0.29 mmol, 1.2 eq) and Cs₂CO₃ (156 mg, 0.48 mmol, 2 eq) were added at room temperature. The reaction mixture was under nitrogen flush for 5 minutes and then the microwave vial was sealed and heated at 90° C. for 48 h. The reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3S,4S)-4-((7-((tert-butoxycarbonyl)(3,5-difluorophenyl)amino)-3-cyclobutylpyrazolo[1,5-a]pyrimidin-5-yl)aminomethyl)-3-hydroxypiperidine-1-carboxylate as a brown oil (60 mg, 41%). TLC system: EOAc/hexane (1:1), R_{f} value: ˜0.30.

### (3S,4S4-((3-Cyclopropyl-7-((3,5-difluorophenyl)amino)pyrazolo[1,5-a]pyrimidin-5-yl)aminomethyl)piperidin-3-ol (I-47)

To a solution of crude tert-butyl (3S,4S)-4-((7-((tert-butoxycarbonyl)(3,5-difluorophenyl)amino)-3-cyclobutylpyrazolo[1,5-a]pyrimidin-5-yl)aminomethyl)-3-hydroxypiperidine-1-carboxylate (50 mg, 0.08 mmol, 1.0 eq) in DCM (2 mL), TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide product (3S,4S)-4-((3-cyclopropyl-7-((3,5-difluorophenyl)amino)pyrazolo[1,5-a]pyrimidin-5-yl)aminomethyl)piperidin-3-ol (I-47) as yellow solid (20 mg, yield: 59%). TLC system: DCM/Methanol (1:1), R_{f} value: ˜0.1; ¹HNMR (400 Hz, MeOD-d4) δ 7.54 (s, 1H), 7.02-7.69 (m, 2H), 6.78-6.72 (m, 1H), 5.89 (s, 1H, the area is less than 0.5), 4.04-4.00 (m, 1H), 3.38-3.83 (m, 1H), 3.19-3.14 (m, 2H), 3.07-3.03 (m, 1H), 2.67-2.60 (m, 1H), 2.52-2.46 (m, 1H), 1.80-1.73 (m, 2H), 1.55-1.45 (m, 2H), 0.88-0.82 (m, 2H), 0.66-0.58 (m, 2H). HRMS (ESI) m/z [M+H]⁺ calcd for C₂₁H₂₄F₂N₆O 415.2052, found 415.2071. HPLC: 93%.

### Example 39

### Synthesis of (3R,4R)-3-(((3-cyclopropyl-5-(3-Hydroxypiperidin-4-yl)methylamino)pyrazolo[1,5-a]pyrimidin-7-yl)amino)benzonitrile (Compound I-48)

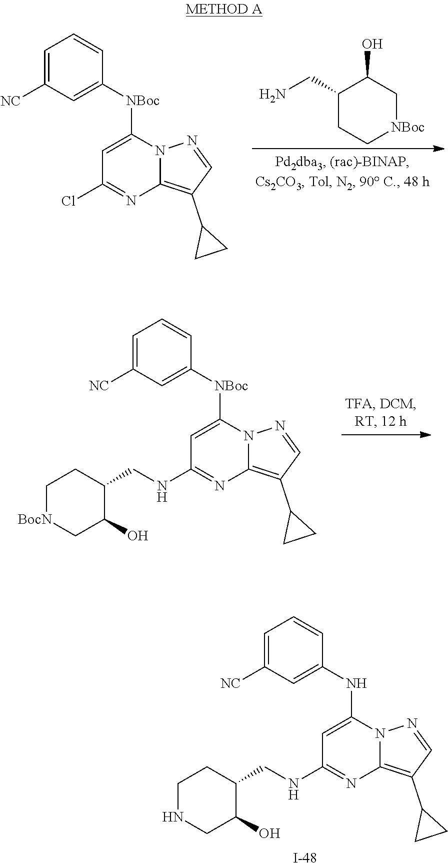

### Tert-Butyl (3R,4R)-4-((7-((tert-butoxycarbonyl)(3-cyanophenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclopropylpyrazolo[1,5-a]pyrimidin-7-yl)(3-cyanophenyl)carbamate, prepared according to Example 11 above (98 mg, 0.24 mmol, 1.0 eq), tert-butyl (3R,4R)-4-(aminomethyl)-3-hydroxypiperidine-1-carboxylate (67 mg, 0.29 mmol, 1.2 eq) and Cs₂CO₃ (156 mg, 0.48 mmol, 2 eq) were added at room temperature. The reaction mixture was under nitrogen flush for 5 minutes and then the microwave vial was sealed and heated at 90° C. for 24 h. Then, the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3R,4R)-4-((7-((tert-butoxycarbonyl)(3-cyanophenyl)amino)-3-cyclobutylpyrazolo[1,5-a]pyrimidin-5-yl)aminomethyl)-3-hydroxypiperidine-1-carboxylate as a brown oil (70 mg, 48%). TLC system: EOAc/hexane (1:1), R_{f} value: ˜0.25.

### (3R,4R)-3-(((3-Cyclopropyl-5-(3-hydroxypiperidin-4-yl)methylamino)pyrazolo[1,5-a]pyrimidin-7-yl)amino)benzonitrile (I-48)

To a solution of crude tert-butyl (3R,4R)-4-((7-((tert-butoxycarbonyl)(3-cyanophenyl)amino)-3-cyclobutylpyrazolo[1,5-a]pyrimidin-5-yl)aminomethyl)-3-hydroxypiperidine-1-carboxylate (50 mg, 0.08 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-3-(((3-cyclopropyl-5-(3-hydroxypiperidin-4-yl)methylamino)pyrazolo[1,5-a]pyrimidin-7-yl)amino)benzonitrile

(I-48) as pink solid (25 mg, yield: 76%). TLC system: DCM/Methanol (1:1), R_{f} value: ˜0.1; ¹HNMR (400 Hz, MeOD-d4) δ 7.70 (s, 1H), 7.68-7.63 (m, 1H), 7.59-7.51 (m, 3H), 5.77 (s, 1H, the area is less than 0.5), 4.02-3.98 (m, 1H), 3.46-3.40 (m, 1H), 3.27-3.23 (m, 1H), 3.21-3.14 (m, 2H), 2.78-2.71 (m, 1H), 2.63-2.57 (m, 1H), 1.83-1.73 (m, 2H), 1.63-1.53 (m, 2H), 0.87-0.81 (m, 2H), 0.66-0.58 (m, 2H). HRMS (ESI) m/z [M+H]⁺ calcd for C₂₂H₂₅N₇O 404.2193, found 404.2136. HPLC: 96%.

### Example 40

### Synthesis of (3R,4R)-4-((3-cyclopropyl-7-((3-fluoro-5-methylphenyl)amino)pyrazolo[1,5-a]pyrimidin-5-yl)aminomethyl)piperidin-3-ol (Compound I-49)

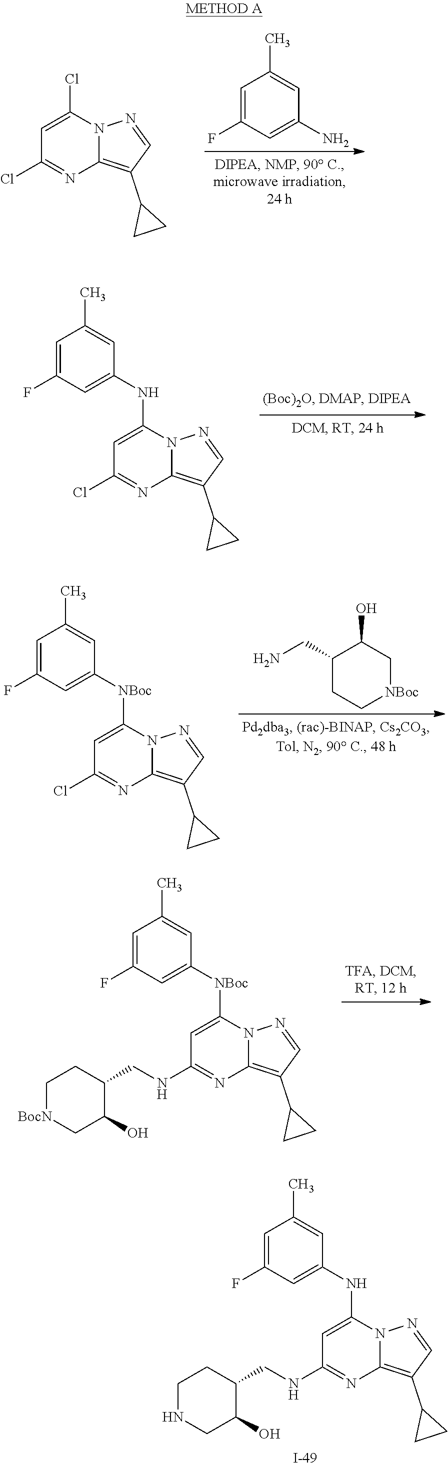

### 5-Chloro-3-cyclopropyl-N-(3-fluoro-5-methylphenyl)pyrazolo[1,5-a]pyrimidin-7-amine

To a stirred solution of 3-fluoro-5-methylaniline (80 mg, 0.64 mmol, 1.0 eq) in NMP (4 mL) DIPEA (152 mg, 1.95 mmol, 1.3 eq) was added and stirred for 5 min. To this mixture 5,7-dichloro-3-cyclopropylpyrazolo[1,5-a]pyrimidine (169 mg, 0.74 mmol, 1.0 eq) was added at room temperature and continued stirring at 90° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclopropyl-N-(3-fluoro-5-methylphenyl)pyrazolo[1,5-a]pyrimidin-7-amine as a brown liquid. TLC system: EtoAc:Hexane (1:4), R_{f} value: ˜0.4.

### (5-Chloro-3-cyclopropylpyrazolo[1,5-a]pyrimidin-7-yl)(3-fluoro-5-methylphenyl)carbamate

To a stirred solution of 5-chloro-3-cyclopropyl-N-(3-fluoro-5-methylphenyl)pyrazolo[1,5-a]pyrimidin-7-amine (203 mg, 0.64 mmol, 1.0 eq) in DCM (5 mL) at room temperature DIPEA (166 mg, 1.28 mmol, 2 eq) and DMAP (2 mg 0.016 mmol) were added, followed by addition of (Boc)₂O (181 mg, 0.83 mmol, 1.3 eq). The reaction was continued at room temperature for 24 h. Then, (Boc)₂O (135 mg, 0.62 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by using combiflash chromatography (4 g column) to afford (5-chloro-3-cyclopropylpyrazolo[1,5-a]pyrimidin-7-yl)(3-fluoro-5-methylphenyl)carbamate as a yellow oil (240 mg, 90% for two steps). TLC system: EtOAc/hexane (1:4), R_{f} value: ˜0.5; ¹HNMR (400 MHz, CDCl₃) δ 7.86 (s, 1H), 6.90-6.80 (s, 3H), 6.58 (s, 1H), 2.30 (s, 3H), 2.10-2.05 (m, 1H), 1.36 (s, 9H), 1.03-0.98 (m, 2H), 0.85-0.81 (m, 2H).

### Tert-Butyl (3R,4R)-4-((7-((tert-butoxycarbonyl)(3-fluoro-5-methylphenyl)amino)-3-cyclopropylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial (5-chloro-3-cyclopropylpyrazolo[1,5-a]pyrimidin-7-yl)(3-fluoro-5-methylphenyl)carbamate (100 mg, 0.24 mmol, 1.0 eq), tert-butyl (3R,4R)-4-(aminomethyl)-3-hydroxypiperidine-1-carboxylate (67 mg, 0.29 mmol, 1.2 eq) and Cs₂CO₃ (156 mg, 0.48 mmol, 2 eq) were added at room temperature. The reaction mixture was under nitrogen flush for 5 minutes and then the microwave vial was sealed and heated at 90° C. for 48 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3R,4R)-4-((7-((tert-butoxycarbonyl)(3-fluoro-5-methylphenyl)amino)-3-cyclopropylpyrazolo[1,5-a]pyrimidin-5-yl)aminomethyl)-3-hydroxypiperidine-1-carboxylate as a brown oil (60 mg, 41%). TLC system: EOAc/hexane (1:1), R_{f} value: ˜0.30.

### (3R,4R)-4-((3-Cyclopropyl-7-((3-fluoro-5-methylphenyl)amino) pyrazolo[1,5-a]pyrimidin-5-yl)aminomethyl)piperidin-3-ol (I-49)

To a solution of crude tert-butyl (3R,4R)-4-((7-((tert-butoxycarbonyl)(3-fluoro-5-methylphenyl)amino)-3-cyclopropylpyrazolo[1,5-a]pyrimidin-5-yl)aminomethyl)-3-hydroxypiperidine-1-carboxylate (50 mg, 0.08 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-4-((3-cyclopropyl-7-((3-fluoro-5-methylphenyl)amino)pyrazolo[1,5-a]pyrimidin-5-yl)aminomethyl)piperidin-3-ol (I-49) as a brown solid (20 mg, yield: 59%). TLC system: DCM/Methanol (2:1), R_{f} value: ˜0.1; ¹HNMR (400 Hz, MeOD-d4) δ 7.54 (s, 1H), 6.99 (s, 1H), 6.93 (d, J=10.1 Hz, 1H), 6.77 (d, J=9.4 Hz, 1H), 5.78 (s, 1H), 4.05-4.00 (m, 1H), 3.44-3.38 (m, 1H), 3.26-3.22 (m, 1H), 3.17-3.12 (m, 2H), 2.76-2.70 (m, 1H), 2.61-2.55 (m, 1H), 2.37 (s, 3H), 1.81-1.73 (m, 2H), 1.62-1.54 (m, 2H), 0.87-0.82 (m, 2H), 0.65-0.57 (m, 2H). HRMS (ESI) m/z [M+H]⁺ calcd for C₂₂H₂₇FN₆O 411.2303, found 411.2262. HPLC: 97%.

### Example 41

### Synthesis of (3R,4R)-4-((3-cyclobutyl-7-((3-fluorophenyl)amino)pyrazolo[1,5-a]pyrimidin-5-yl)aminomethyl)piperidin-3-ol (Compound I-50)

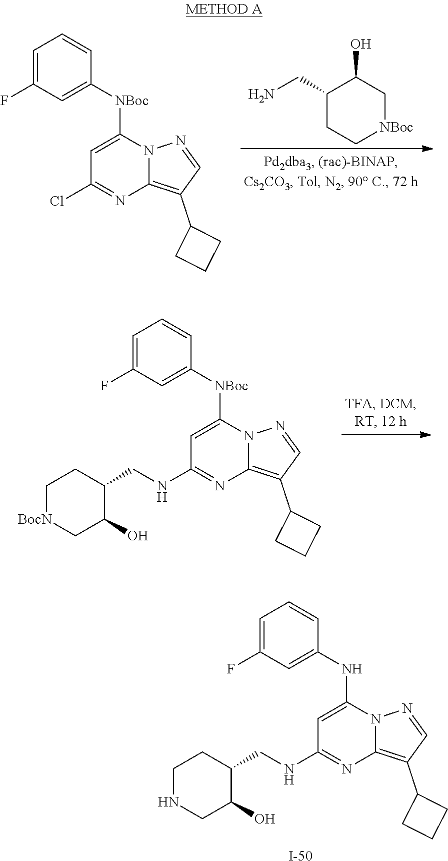 [SMILES: CC(C)(C)OC(=O)N(c1cccc(F)c1)c1cc(Cl)nc2c(C3CCC3)cnn12 ; CC(C)(C)OC(=O)N1CC[C@H](CN)[C@@H](O)C1 ; CC(C)(C)OC(=O)N1CC[C@H](CNc2cc(N(C(=O)OC(C)(C)C)c3cccc(F)c3)n3ncc(C4CCC4)c3n2)[C@@H](O)C1 ; O[C@H]1CNCC[C@@H]1CNc1cc(Nc2cccc(F)c2)n2ncc(C3CCC3)c2n1 ; "I-50"]

### Tert-Butyl (3R,4R)-4-((7-((tert-butoxycarbonyl)(3-fluorophenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclobutylpyrazolo[1,5-a]pyrimidin-7-yl)(3-fluorophenyl)carbamate, prepared according to Example 25 above (100 mg, 0.24 mmol, 1.0 eq), tert-butyl (3R,4R)-4-(aminomethyl)-3-hydroxypiperidine-1-carboxylate (67 mg, 0.29 mmol, 1.2 eq) and Cs₂CO₃ (156 mg, 0.48 mmol, 2 eq) were added at room temperature. The reaction mixture was under nitrogen flush for 5 minutes and then the microwave vial was sealed and heated at 90° C. for 72 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3R,4R)-4-((7-((tert-butoxycarbonyl)(3-fluorophenyl)amino)-3-cyclobutylpyrazolo[1,5-a]pyrimidin-5-yl)aminomethyl)-3-hydroxypiperidine-1-carboxylate as a brown oil (60 mg, 41%). TLC system: EOAc/hexane (1:1), R_{f} value: ˜0.30.

### (3R,4R)-4-((3-Cyclobutyl-7-((3-fluorophenyl)amino)pyrazolo[1,5-a]pyrimidin-5-yl)aminomethyl)piperidin-3-ol (I-50)

To a solution of crude tert-butyl (3R,4R)-4-((7-((tert-butoxycarbonyl)(3-fluorophenyl)amino)-3-cyclobutylpyrazolo[1,5-a]pyrimidin-5-yl)aminomethyl)-3-hydroxypiperidine-1-carboxylate (50 mg, 0.08 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-4-((3-cyclobutyl-7-((3-fluorophenyl)amino)pyrazolo[1,5-a]pyrimidin-5-yl)aminomethyl)piperidin-3-ol (I-50) as a brown solid (25 mg, yield: 74%). TLC system: DCM/Methanol (2:1), R_{f} value: ˜0.1; ¹HNMR (400 Hz, DMSO-d₆) δ 7.82 (s, 1H), 7.47-7.41 (m, 1H), 7.28-7.24 (m, 2H), 7.00-6.94 (m, 1H), 5.88 (s, 1H), 3.56-3.47 (m, 2H), 3.38-3.32 (m, 1H), 3.16-3.10 (m, 1H), 2.99-2.95 (m, 1H), 2.87-2.84 (m, 1H), 2.42-2.36 (m, 1H), 2.27-2.18 (m, 5H), 1.98-1.82 (m, 2H), 1.66-1.63 (m, 1H), 1.45-1.39 (m, 1H), 1.27-1.16 (m, 1H). HRMS (ESI) m/z [M+H]⁺ calcd for C₂₂H₂₇FN₆O 411.2303, found 411.2256. HPLC: 94%.

### Example 42

### Synthesis of (3R,4R)-4-((3-cyclopropyl-7-((3-methylsulfonylphenyl)amino)pyrazolo[1,5-a]pyrimidin-5-yl)aminomethyl)piperidin-3-ol (Compound I-51)

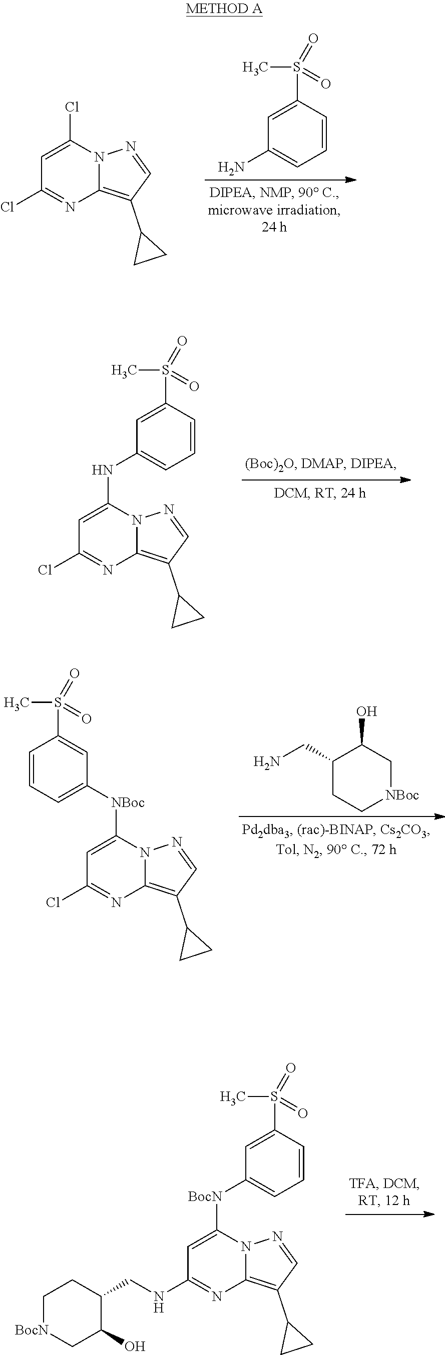 [SMILES: Clc1cc(Cl)n2ncc(C3CC3)c2n1 ; CS(=O)(=O)c1cccc(N)c1 ; CS(=O)(=O)c1cccc(Nc2cc(Cl)nc3c(C4CC4)cnn23)c1 ; CC(C)(C)OC(=O)N(c1cccc(S(C)(=O)=O)c1)c1cc(Cl)nc2c(C3CC3)cnn12 ; CC(C)(C)OC(=O)N1CC[C@H](CN)[C@@H](O)C1 ; CC(C)(C)OC(=O)N1CC[C@H](CNc2cc(N(C(=O)OC(C)(C)C)c3cccc(S(C)(=O)=O)c3)n3ncc(C4CC4)c3n2)[C@@H](O)C1]

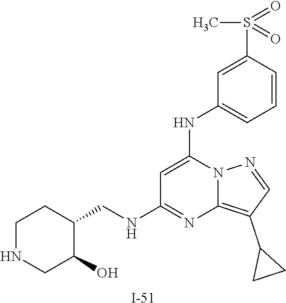

### 5-Chloro-3-cyclopropyl-N-(3-methylsulfonylphenyl)pyrazolo[1,5-a]pyrimidin-7-amine

To a stirred solution of 3-(methylsulfonyl)aniline (50 mg, 0.29 mmol, 1.0 eq) in NMP (4 mL) was added DIPEA (49 mg, 0.38 mmol, 1.3 eq) and stirred for 5 min. To this mixture 5,7-dichloro-3-cyclopropylpyrazolo[1,5-a]pyrimidine (75 mg, 0.33 mmol, 1.15 eq) was added at room temperature and continued stirring at 90° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclopropyl-N-(3-methylsulfonylphenyl)pyrazolo[1,5-a]pyrimidin-7-amine as a brown liquid. TLC system: EoAc:Hexane (1:4), R_{f} value: ˜0.3.

### Tert-Butyl (5-chloro-3-cyclopropylpyrazolo[1,5-a]pyrimidin-7-yl)(3-methylsulfonylphenyl)carbamate

To a stirred solution of 5-chloro-3-cyclopropyl-N-(3-methylsulfonylphenyl)pyrazolo[1,5-a]pyrimidin-7-amine (105 mg, 0.29 mmol, 1.0 eq) in DCM (5 mL) at room temperature was added DIPEA (75 mg, 0.58 mmol, 2 eq) and DMAP (2 mg 0.016 mmol), followed by addition of (Boc)₂O (83 mg, 0.38 mmol, 1.3 eq). The reaction was continued at room temperature for 24 h. Then, (Boc)₂O (83 mg, 0.38 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by using combiflash chromatography (4 g column) to afford tert-butyl (5-chloro-3-cyclopropylpyrazolo[1,5-a]pyrimidin-7-yl)(3-methylsulfonylphenyl)carbamate as a yellow oil (110 mg, 82% for two steps). TLC system: EOAc/hexane (1:4), R_{f} value: ˜0.5; ¹HNMR (400 MHz, CDCl₃) δ 7.93-7.91 (m, 1H), 7.84-7.81 (m, 2H), 7.56-7.49 (m, 2H), 6.65 (s, 1H), 3.05 (s, 3H), 2.10-2.00 (m, 1H), 1.37 (s, 9H), 1.03-0.98 (m, 2H), 0.85-0.81 (m, 2H).

### Tert-Butyl (3R,4R)-4-((7-((tert-butoxycarbonyl)(3-methylsulfonylphenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclopropylpyrazolo[1,5-a]pyrimidin-7-yl)(3-methylsulfonylphenyl)carbamate (108 mg, 0.23 mmol, 1.0 eq), tert-butyl (3R,4R)-4-(aminomethyl)-3-hydroxypiperidine-1-carboxylate (64 mg, 0.28 mmol, 1.2 eq) and Cs₂CO₃ (150 mg, 0.46 mmol, 2 eq) were added at room temperature. The reaction mixture was under nitrogen flush for 5 minutes and then the microwave vial was sealed and heated at 90° C. for 24 h. After completion of reaction by TLC, the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3R,4R)-4-((7-((tert-butoxycarbonyl)(3-methylsulfonylphenyl)amino)-3-cyclobutylpyrazolo[1,5-a]pyrimidin-5-yl)aminomethyl)-3-hydroxypiperidine-1-carboxylate as a brown oil (90 mg, 60%). TLC system: EOAc/hexane (1:1), R_{f} value: ˜0.10.

### (3R,4R)-4-((3-Cyclopropyl-7-((3-methylsulfonylphenyl)amino)pyrazolo[1,5-a]pyrimidin-5-yl)aminomethyl)piperidin-3-ol (I-51)

To a solution of crude tert-butyl (3R,4R)-4-((7-((tert-butoxycarbonyl)(3-methylsulfonylphenyl)amino)-3-cyclobutylpyrazolo[1,5-a]pyrimidin-5-yl)aminomethyl)-3-hydroxypiperidine-1-carboxylate (80 mg, 0.13 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-4-((3-cyclopropyl-7-((3-methylsulfonylphenyl)amino)pyrazolo[1,5-a]pyrimidin-5-yl)aminomethyl)piperidin-3-ol (I-51) as a brown solid (25 mg, yield: 48%). TLC system: DCM/Methanol (2:1), R_{f} value: ˜0.2; ¹HNMR (400 Hz, DMSO-d6) δ 7.92 (s, 1H), 7.77-7.731 (m, 1H), 7.70-7.68 (m, 2H), 7.61 (s, 1H), 5.81 (s, 1H), 3.51-3.47 (m, 1H), 3.39-3.35 (m, 1H), 3.26 (s, 3H), 3.23-3.16 (m, 1H), 3.04-3.00 (m, 1H), 2.94-2.90 (m, 1H), 2.46-2.42 (m, 1H), 2.34-2.28 (m, 1H), 1.81-1.74 (m, 1H), 1.71-1.68 (m, 1H), 1.47-1.44 (m, 1H), 1.31-1.23 (m, 1H), 0.81-0.78 (m, 2H), 0.72-0.69 (m, 2H). HRMS (ESI) ml/z [M+H]⁺ calcd for C₂₂H28N₆O₃S 457.2016, found 457.1963. HPLC: 98%.

### Example 43

### Synthesis of (3R,4R)-4-((3-cyclobutyl-7-((3-fluoro-5-methylphenyl)amino)pyrazolo[1,5-a]pyrimidin-5-yl)aminomethyl)piperidin-3-ol (Compound I-52)

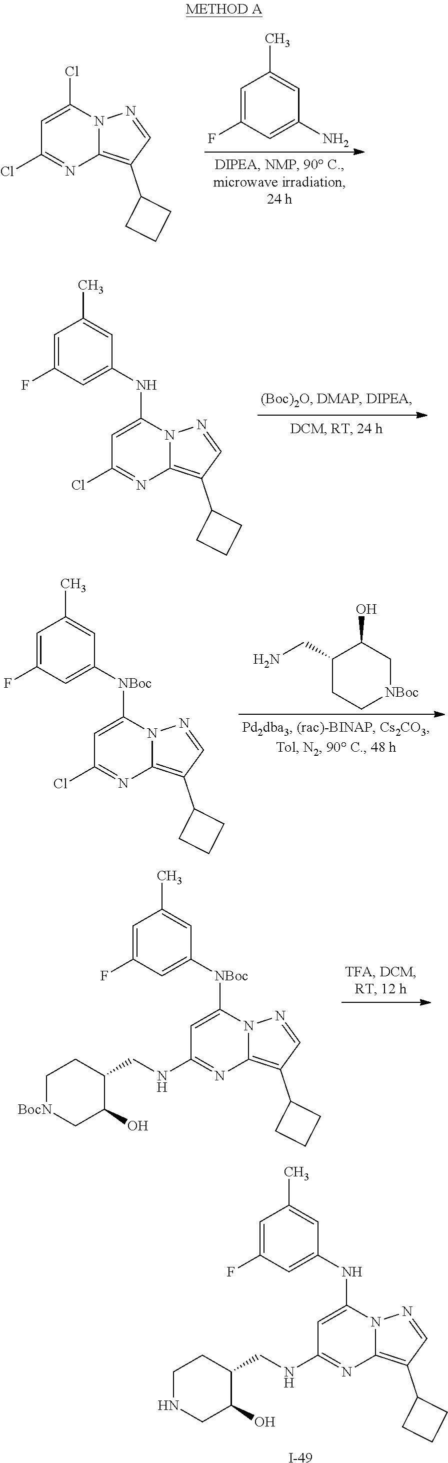

### 5-Chloro-3-cyclobutyl-N-(3-fluoro-5-methylphenyl)pyrazolo[1,5-a]pyrimidin-7-amine

To a stirred solution of 3-fluoro-5-methylaniline (60 mg, 0.48 mmol, 1.0 eq) in NMP (4 mL) was added DIPEA (80 mg, 0.62 mmol, 1.3 eq) and stirred for 5 min. To this mixture 5,7-dichloro-3-cyclobutylpyrazolo[1,5-a]pyrimidine (133 mg, 0.55 mmol, 1.15 eq) was added at room temperature and continued stirring at 90° C. for 16 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclobutyl-N-(3-fluoro-5-methylphenyl)pyrazolo[1,5-a]pyrimidin-7-amine as a brown liquid. TLC system: EA:Hexane (1:8), R_{f} value: ˜0.4.

### Tert-Butyl (5-chloro-3-cyclobutylpyrazolo[1,5-a]pyrimidin-7-yl)(3-fluoro-5-methylphenyl)carbamate

To a stirred solution of 5-chloro-3-cyclobutyl-N-(3-fluoro-5-methylphenyl)pyrazolo[1,5-a]pyrimidin-7-amine (158 mg, 0.48 mmol, 1.0 eq) in DCM (5 mL) at room temperature was added DIPEA (124 mg, 0.96 mmol, 2 eq) and DMAP (3 mg 0.025 mmol), followed by addition of (Boc)₂O (135 mg, 0.62 mmol, 1.3 eq). The reaction was continued at room temperature for 24 h. Then, (Boc)₂O (135 mg, 0.62 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclobutylpyrazolo[1,5-a]pyrimidin-7-yl)(3-fluoro-5-methylphenyl)carbamate as a yellow oil (180 mg, 87% for two steps). TLC system: EtOAc/hexane (1:8), R_{f} value: ˜0.4; ¹HNMR (400 MHz, CDCl₃) δ 8.12 (s, 1H), 6.90-6.86 (m, 2H), 6.82-6.79 (m, 1H), 6.58 (s, 1H), 3.88-3.79 (m, 1H), 2.45-2.40 (m, 2H), 2.32-2.26 (m, 2H), 2.31 (s, 3H), 2.09-2.02 (m, 1H), 1.99-1.94 (m, 1H), 1.37 (s, 9H).

### Tert-Butyl (3R,4R)-4-((7-((tert-butoxycarbonyl)(3-fluoro-5-methylphenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclobutylpyrazolo[1,5-a]pyrimidin-7-yl)(3-fluoro-5-methylphenyl)carbamate (224 mg, 0.52 mmol, 1.0 eq), tert-butyl (3R,4R)-4-(aminomethyl)-3-hydroxy-1-piperidinecarboxylate (143 mg, 0.62 mmol, 1.2 eq) and Cs₂CO₃ (339 mg, 1.04 mmol, 2 eq) were added at room temperature. The reaction mixture was under nitrogen flush for 5 minutes and then the microwave vial was sealed and heated at 90° C. for 48 h. After completion of reaction by TLC, the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3R,4R)-4-((7-((tert-butoxycarbonyl)(3-fluoro-5-methylphenyl)amino)-3-cyclobutylpyrazolo[1,5-a]pyrimidin-5-yl)aminomethyl)-3-hydroxypiperidine-1-carboxylate as a brown oil (90 mg, 28%). TLC system: EOAc/hexane (1:1), R_{f} value: ˜0.40; ¹HNMR (400 MHz, CDCl₃) δ 7.87 (s, 1H), 6.91-6.89 (m, 2H), 6.77-6.75 (m, 1H), 5.85 (s, 1H), 5.02-4.99 (m, 1H), 4.88-4.85 (m, 1H), 4.30-4.24 (m, 3H), 4.14-4.09 (m, 1H), 3.72-3.64 (m, 1H), 3.26-3.18 (m, 1H), 3.05-3.00 (m, 1H), 2.90-2.86 (m, 1H), 2.64-2.56 (m, 1H), 2.44-2.37 (m, 2H), 2.23-2.11 (m, 2H), 2.07-2.00 (m, 1H), 1.92-1.85 (m, 1H), 1.41 (s, 9H), 1.39 (s, 9H).

### (3R,4R)-4-((3-Cyclobutyl-7-((3-fluoro-5-methylphenyl)amino)pyrazolo[1,5-a]pyrimidin-5-yl)aminomethyl)piperidin-3-ol (I-52)

To a solution of crude tert-butyl (3R,4R)-4-((7-((tert-butoxycarbonyl)(3-fluoro-5-methylphenyl)amino)-3-cyclobutylpyrazolo[1,5-a]pyrimidin-5-yl)aminomethyl)-3-hydroxypiperidine-1-carboxylate (68 mg, 0.11 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-4-((3-cyclobutyl-7-((3-fluoro-5-methylphenyl)amino)pyrazolo[1,5-a]pyrimidin-5-yl)aminomethyl)piperidin-3-ol (I-52) as an off white solid (20 mg, yield: 45%). TLC system: DCM/Methanol (1:1), R_{f} value: ˜0.2; ¹HNMR (400 Hz, MeOD-d4) δ 7.80 (s, 1H), 7.00 (s, 1H), 6.94 (d, J=10.1 Hz, 1H), 6.78 (d, J=9.3 Hz, 1H), 5.77 (s, 1H), 4.00-3.96 (m, 1H), 3.64-3.56 (m, 1H), 3.46-3.40 (m, 1H), 3.29-3.25 (m, 1H), 3.22-3.17 (m, 2H), 2.84-2.77 (m, 1H), 2.68-2.63 (m, 1H), 2.37 (s, 3H), 2.36-2.32 (m, 2H), 2.25-2.17 (m, 2H), 2.07-1.98 (m, 1H), 1.94-1.83 (m, 2H), 1.67-1.56 (m, 2H). HRMS (ESI) m/z [M+H]⁺ calcd for C₂₃H₃₀FN₆O 425.2460, found 425.2404. HPLC: 98%.

### Example 44

### Synthesis of Trans-4-((3-cyclopropyl-7-((3-fluorophenyl)amino)pyrazolo[1,5-a]pyrimidin-5-yl)oxymethyl)piperidin-3-ol (Compound I-53)

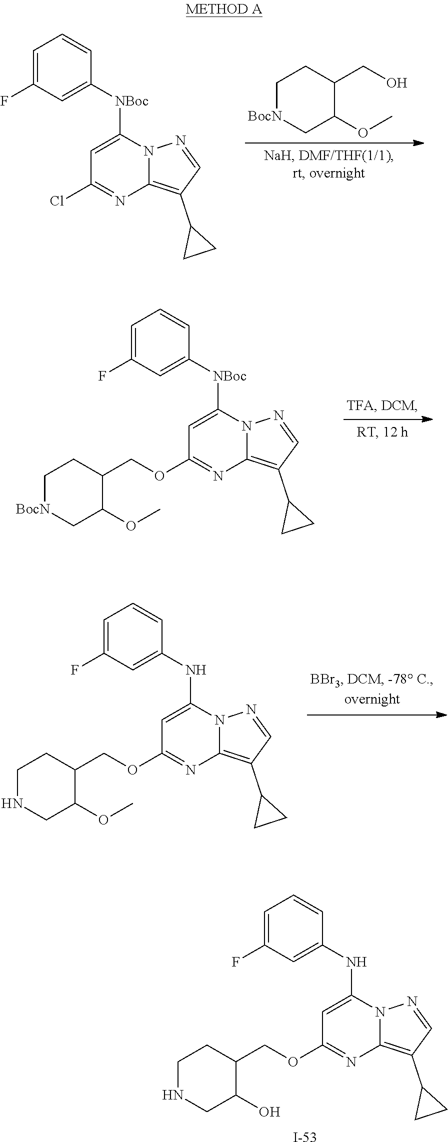

### Tert-Butyl Trans-4-(7-(tert-butoxycarbonyl)(3-fluorophenyl)amino-3-cyclopropylpyrazolo[1,5-a]pyrimidin-5-yl)oxymethyl-3-methoxylpiperidine-1-carboxylate

To a stirred solution of 60% of NaH (16 mg, 0.40 mmol, 2.0 eq) in DMF (2 mL), the mixture of tert-butyl (5-chloro-3-cyclopropylpyrazolo[1,5-a]pyrimidin-7-yl)(3-fluorophenyl)carbamate, prepared according to Example 6 above (81 mg, 0.20 mmol, 1.0 eq), and tert-butyl trans-4-(hydroxymethyl)-3-methoxypiperidine-1-carboxylate (54 mg, 0.22 mmol, 1.1 eq) in THF (2 mL) was added at 0-5° C. and stirred at room temperature for 12 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford crude product which was purified by using combiflash chromatography (4 g column) to afford crude tert-butyl trans-4-(7-(tert-butoxycarbonyl)(3-fluorophenyl)amino-3-cyclopropylpyrazolo[1,5-a]pyrimidin-5-yl)oxymethyl-3-methoxylpiperidine-1-carboxylate as white oil (94 mg; 77%). TLC system: EtoAc:Hexane (1:4), R_{f} value: ˜0.3; ¹HNMR (400 MHz, CDCl₃) δ 7.73 (s, 1H), 7.29-7.23 (m, 1H), 7.11-7.06 (m, 2H), 6.95-6.91 (m, 1H), 6.16 (s, 1H), 4.56-4.51 (m, 1H), 4.45-4.41 (m, 1H), 4.09-3.99 (m, 2H), 3.40 (s, 3H), 3.15-3.09 (m, 1H), 2.77-2.68 (m, 1H), 2.54-2.45 (m, 1H), 1.99-1.93 (m, 1H), 1.89-1.76 (m, 3H), 1.46 (s, 9H), 1.37 (s, 9H), 0.95-0.89 (m, 2H), 0.88-0.84 (m, 2H).

### Trans-3-Cyclopropyl-N-(3-fluorophenyl)-5-((3-methyoxylpiperidin-4-yl)methoxy)pyrazolo[1,5-a]pyrimidin-7-amine

To a solution of crude tert-butyl trans-4-(7-(tert-butoxycarbonyl)(3-fluorophenyl)amino-3-cyclopropylpyrazolo[1,5-a]pyrimidin-5-yl)oxymethyl-3-methoxylpiperidine-1-carboxylate (104 mg, 0.17 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford trans-3-cyclopropyl-N-(3-fluorophenyl)-5-((3-methyoxylpiperidin-4-yl)methoxy)pyrazolo[1,5-a]pyrimidin-7-amine as light yellow oil (70 mg, yield: 100%). TLC system: DCM:MeOD (1:2), R_{f} value: ˜0.3; ¹HNMR (400 MHz, CDCl₃) δ 8.05-7.83 (broad, 1H), 7.67 (s, 1H), 7.40-7.35 (m, 1H), 7.10-7.04 (m, 2H), 6.94-6.90 (m, 1H), 5.87 (s, 1H), 4.53-4.43 (m, 2H), 3.43-3.39 (m, 1H), 3.38 (s, 3H), 3.20-3.15 (m, 1H), 3.03-2.99 (m, 1H), 2.62-2.55 (m, 1H), 2.44-2.39 (m, 1H), 2.19-2.06 (broad, 1H), 1.98-1.93 (m, 1H), 1.91-1.81 (m, 2H), 1.56-1.46 (m, 1H), 0.93-0.83 (m, 4H).

### Trans-4-((3-Cyclopropyl-7-((3-fluorophenyl)amino)pyrazolo[1,5-a]pyrimidin-5-yl)oxymethyl)piperidin-3-ol (I-53)

To a stirred solution of trans-3-cyclopropyl-N-(3-fluorophenyl)-5-((3-methyoxylpiperidin-4-yl)methoxy)pyrazolo[1,5-a]pyrimidin-7-amine (28 mg, 0.068 mmol, 1.0 eq) in DCM (4 mL), BBr₃ (35 mg, 0.14 mmol, 2.0 eq) in DCM (1 mL) was added at −78° C. under N₂, stirring for 30 min at this temperature and then 12 h at ambient temperature. After completion of reaction by TLC, the reaction mixture was quenched with ice cold water and NaHCO₃ (5% aq.) and then extracted with (3×50 mL) DCM. The combined organic layer was washed with water (30 mL), brine solution (30 mL), dried over sodium sulfate, and concentrated to afford crude product which was purified by using Combi-Flash chromatography (4 g column) to afford trans-4-((3-cyclopropyl-7-((3-fluorophenyl)amino)pyrazolo[1,5-a]pyrimidin-5-yl)oxymethyl)piperidin-3-ol (I-53) as light green powder (10 mg; 36%). TLC system: DCM:MeOD (1:2), R_{f} value: ˜0.2; ¹HNMR (500 MHz, MeOD-d4) δ 7.72 (s, 1H), 7.48-7.44 (m, 1H), 7.24-7.22 (m, 1H), 7.19-7.16 (m, 1H), 7.02-6.98 (m, 1H), 5.80 (s, 1H), 4.56-4.50 (m, 2H), 3.76-3.72 (m, 1H), 3.36-3.33 (m, 1H), 3.28-3.24 (m, 1H), 2.92-2.86 (m, 1H), 2.74-2.70 (m, 1H), 2.11-2.06 (m, 1H), 1.97-1.87 (m, 2H), 1.74-1.66 (m, 1H), 0.90-0.87 (m, 2H), 0.82-0.79 (m, 2H). HRMS (ESI) m/z [M+H]⁺ calcd for C₂₁H₂₄FN₅O₂ 398.1987, found 398.1991; HPLC: 96%.

### Example 45

### Synthesis of (3R,4R)-3-((3-cyclopropyl-5-((3-hydroxypiperidin-4-yl)methylamino)pyrazolo[1,5-a]pyrimidin-7-yl)amino)-5-methylbenzonitrile (Compound I-54)

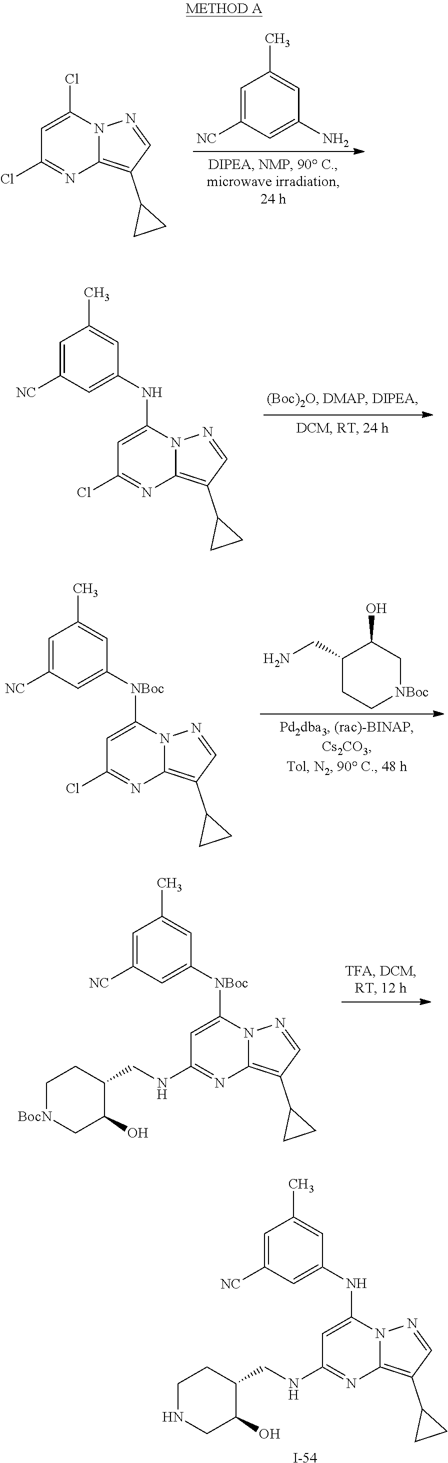

### 5-Chloro-N-(3-cyano-5-methylphenyl)-3-cyclopropylpyrazolo[1,5-a]pyrimidin-7-amine

To a stirred solution of 3-amino-5-methylbenzonitrile (145 mg, 1.10 mmol, 1.0 eq) in NMP (4 mL) was added DIPEA (252 mg, 1.95 mmol, 1.3 eq) and stirred for 5 min. To this mixture 5,7-dichloro-3-cyclopropylpyrazolo[1,5-a]pyrimidine (228 mg, 1.00 mmol, 1.0 eq) was added at room temperature and continued stirring at 90° C. for 16 h under microwave irradiation. After completion of reaction by TLC, the reaction mixture was diluted with ice cold water and extracted with (3×30 mL) ethyl acetate. The combined organic layer was washed with 10% HCl aqueous two times, water (50 mL), brine solution (20 mL), dried over sodium sulfate, and concentrated. The crude compound without purification went to the next step, 5-chloro-N-(3-cyano-5-methylphenyl)-3-cyclopropylpyrazolo[1,5-a]pyrimidin-7-amine as a brown liquid. TLC system: EtoAc:Hexane (1:4), R_{f} value: ˜0.3.

### Tert-Butyl (5-chloro-3-cyclopropylpyrazolo[1,5-a]pyrimidin-7-yl)(3-cyano-5-methylphenyl)carbamate

To a stirred solution of 5-chloro-N-(3-cyano-5-methylphenyl)-3-cyclopropylpyrazolo[1,5-a]pyrimidin-7-amine (324 mg, 1.00 mmol, 1.0 eq) in DCM (5 mL) at room temperature was added DIPEA (259 mg, 2.00 mmol, 2 eq) and DMAP (2 mg 0.016 mmol), followed by addition of (Boc)₂O (284 mg, 1.30 mmol, 1.3 eq). The reaction was continued at room temperature for 24 h. Then, (Boc)₂O (142 mg, 0.65 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by using combiflash chromatography (4 g column) to afford tert-butyl (5-chloro-3-cyclopropylpyrazolo[1,5-a]pyrimidin-7-yl)(3-cyano-5-methylphenyl)carbamate as yellow oil (410 mg, 97% for two steps). TLC system: EtOAc/hexane (1:8), R_{f} value: ˜0.3; ¹HNMR (500 MHz, CDCl₃) δ 7.85 (s, 1H), 7.41 (s, 1H), 7.35 (s, 1H), 7.33 (s, 1H), 6.62 (s, 1H), 2.36 (s, 3H), 2.09-2.05 (m, 1H), 1.36 (s, 9H), 1.03-0.99 (m, 2H), 0.86-0.83 (m, 2H).

### Tert-Butyl (3R,4R)-4-((7-((tert-butoxycarbonyl)(3-cyano-5-methylphenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclopropylpyrazolo[1,5-a]pyrimidin-7-yl)(3-cyano-5-methylphenyl)carbamate (181 mg, 0.43 mmol, 1.0 eq), tert-butyl (3R,4R)-4-(aminomethyl)-3-hydroxypiperidine-1-carboxylate (108 mg, 0.47 mmol, 1.1 eq) and Cs₂CO₃ (280 mg, 0.86 mmol, 2 eq) were added at room temperature. The reaction mixture was under nitrogen flush for 5 minutes and then the microwave vial was sealed and heated at 90° C. for 48 h. After completion of reaction by TLC, the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude (tert-butyl (3R,4R)-4-((7-((tert-butoxycarbonyl)(3-cyano-5-methylphenyl)amino)-3-cyclobutylpyrazolo[1,5-a]pyrimidin-5-yl)aminomethyl)-3-hydroxypiperidine-1-carboxylate as a brown oil (95 mg, 36%). TLC system: EtOAc/hexane (1:1), R_{f} value: ˜0.30.

### (3R,4R)-3-((3-Cyclopropyl-5-((3-hydroxypiperidin-4-yl)methylamino)pyrazolo[1,5-a]pyrimidin-7-yl)amino)-5-methylbenzonitrile (I-54)

To a solution of crude tert-butyl (3R,4R)-4-((7-((tert-butoxycarbonyl)(3-cyano-5-methylphenyl)amino)-3-cyclobutylpyrazolo[1,5-a]pyrimidin-5-yl)aminomethyl)-3-hydroxypiperidine-1-carboxylate (70 mg, 0.11 mmol, 1.0 eq) in DCM (2 mL) TFA (1 mL) was added. The reaction mixture was stirred at room temperature for 3 days. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-3-((3-cyclopropyl-5-((3-hydroxypiperidin-4-yl)methylamino)pyrazolo[1,5-a]pyrimidin-7-yl)amino)-5-methylbenzonitrile (I-54) as an off white solid (40 mg, yield: 87%). TLC system: DCM/Methanol (1:1), R_{f} value: ˜0.1; ¹HNMR (500 Hz, MeOD-d4) δ 7.56 (s, 1H), 7.51 (s, 1H), 7.48 (s, 1H), 7.37 (s, 1H), 5.77 (s, 1H), 4.02-3.99 (m, 1H), 3.61-3.56 (m, 1H), 3.43-3.36 (m, 2H), 3.28-3.24 (m, 1H), 3.00-2.96 (m, 1H), 2.84-2.79 (m, 1H), 1.98-1.93 (m, 1H), 1.80-1.68 (m, 3H), 0.87-0.84 (m, 2H), 0.65-0.60 (m, 2H). HRMS (ESI) m/z [M+H]⁺ calcd for C₂₃H₂₈N₇O 418.2350, found 418.2361. HPLC: 95%.

### Example 46

### Synthesis of (3R,4R)-3-((3-cyclobutyl-5-((3-hydroxypiperidin-4-yl)methylamino)pyrazolo[1,5-a]pyrimidin-7-yl)amino)-5-fluorobenzonitrile (Compound I-55)

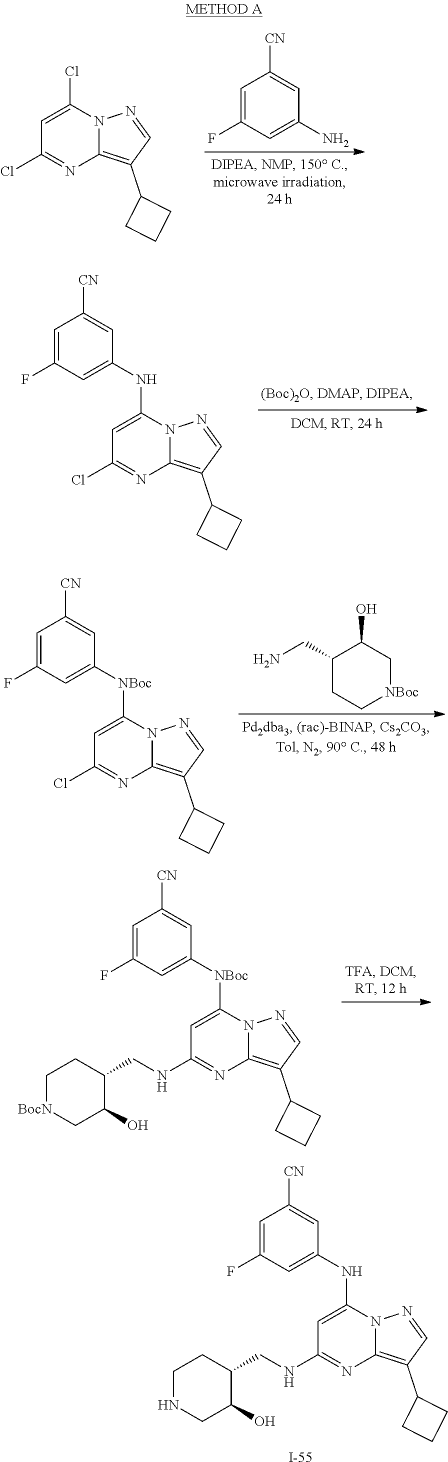

### 5-Chloro-N-(3-cyano-5-fluorophenyl)-3-cyclobutylpyrazolo[1,5-a]pyrimidin-7-amine

To a stirred solution of 5-amino-3-fluorobenzonitrile (64 mg, 0.48 mmol, 0.95 eq) in NMP (4 mL) was added DIPEA (84 mg, 0.65 mmol, 1.3 eq) and stirred for 5 min. To this mixture 5,7-dichloro-3-cyclobutylpyrazolo[1,5-a]pyrimidine (141 mg, 0.50 mmol, 1.0 eq) was added at room temperature and continued stirring at 150° C. for 24 h under microwave irradiation. Then, (Boc)₂O (135 mg, 0.62 mmol, 1.3 eq) was added again to the reaction. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-N-(3-cyano-5-fluorophenyl)-3-cyclobutylpyrazolo[1,5-a]pyrimidin-7-amine as a brown liquid. TLC system: EA:Hexane (1:8), R_{f} value: ˜0.3.

### Tert-Butyl (5-chloro-3-cyclobutylpyrazolo[1,5-a]pyrimidin-7-yl)(3-cyano-5-fluorophenyl)carbamate

To a stirred solution of 5-chloro-N-(3-cyano-5-fluorophenyl)-3-cyclobutylpyrazolo[1,5-a]pyrimidin-7-amine in DCM (5 mL) at room temperature was added DIPEA (124 mg, 0.96 mmol, 2 eq) and DMAP (3 mg 0.025 mmol), followed by addition of (Boc)₂O (135 mg, 0.62 mmol, 1.3 eq). The reaction was continued at room temperature for 24 h. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclobutylpyrazolo[1,5-a]pyrimidin-7-yl)(3-cyano-5-fluorophenyl)carbamate as a yellow oil (170 mg, 80% for two steps). TLC system: EOAc/hexane (1:8), R_{f} value: ˜0.5; ¹HNMR (500 MHz, CDCl₃) δ 8.09 (s, 1H), 7.37-7.34 (m, 2H), 7.25-7.23 (m, 1H), 6.29 (s, 1H), 3.87-3.79 (m, 1H), 2.47-2.41 (m, 2H), 2.34-2.27 (m, 2H), 2.11-2.04 (m, 1H), 1.99-1.93 (m, 1H), 1.38 (s, 9H).

### Tert-Butyl (3R,4R)-4-((7-((tert-butoxycarbonyl)(3-cyano-5-fluoro phenyl)amino)-3-cyclobutylpyrazolo[1,5-a]pyrimidin-5-yl)aminomethyl)-3-hydroxypiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclobutylpyrazolo[1,5-a]pyrimidin-7-yl)(3-cyano-5-fluorophenyl)carbamate (70 mg, 0.16 mmol, 1.0 eq), tert-butyl (3R,4R)-4-(aminomethyl)-3-hydroxypiperidine-1-carboxylate (42 mg, 0.18 mmol, 1.1 eq) and Cs₂CO₃ (339 mg, 1.04 mmol, 2 eq) were added at room temperature. The reaction mixture was under nitrogen flush for 5 minutes and then the microwave vial was sealed and heated at 90° C. for 48 h under microwave irradiation. After completion of reaction by TLC, the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3R,4R)-4-((7-((tert-butoxycarbonyl)(3-cyano-5-fluoro phenyl)amino)-3-cyclobutylpyrazolo[1,5-a]pyrimidin-5-yl)aminomethyl)-3-hydroxypiperidine-1-carboxylate as a brown oil (48 mg, 47%). TLC system: EOAc/hexane (1:1), R_{f} value: ˜0.40.

### (3R,4R)-3-((3-Cyclobutyl-5-((3-hydroxypiperidin-4-yl)methylamino)pyrazolo[1,5-a]pyrimidin-7-yl)amino)-5-fluorobenzonitrile (I-55)

To a solution of crude tert-butyl (3R,4R)-4-((7-((tert-butoxycarbonyl)(3-cyano-5-fluoro phenyl)amino)-3-cyclobutylpyrazolo[1,5-a]pyrimidin-5-yl)aminomethyl)-3-hydroxypiperidine-1-carboxylate (70 mg, 0.11 mmol, 1.0 eq) in DCM (2 mL) TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3R,4R)-3-((3-cyclobutyl-5-((3-hydroxypiperidin-4-yl)methylamino)pyrazolo[1,5-a]pyrimidin-7-yl)amino)-5-fluorobenzonitrile (I-55) as a brown solid (30 mg, yield: 63%). TLC system: DCM/Methanol (1:1), R_{f} value: ˜0.15; ¹HNMR (500 Hz, MeOD-d4) δ 7.80 (s, 1H), 7.55 (s, 1H), 7.46 (dt, J=10.3, 2.2 Hz, 1H), 7.32-7.29 (m, 1H), C6-1H is missing, 3.98-3.94 (m, 1H), 3.63-3.56 (m, 1H), 3.48-3.43 (m, 1H), 3.30-3.29 (m, 1H), 3.27-3.25 (m, 1H), 3.23-3.20 (m, 1H), 2.83-2.78 (m, 1H), 2.68-2.63 (m, 1H), 2.39-2.33 (m, 2H), 2.24-2.17 (m, 2H), 2.06-1.98 (m, 1H), 1.93-1.85 (m, 2H), 1.67-1.56 (m, 2H). HRMS (ESI) m/z [M+H]⁺ calcd for C₂₃H₂₇FN₇O 436.2256, found 436.2264. HPLC: 95%.

### Example 47

### Synthesis of (3S,5S)-5-((3-cyclobutyl-7-((3-fluoro-5-methylphenyl)amino)pyrazolo[1,5-a]pyrimidin-5-yl)amino)piperidin-3-ol (Compound I-56)

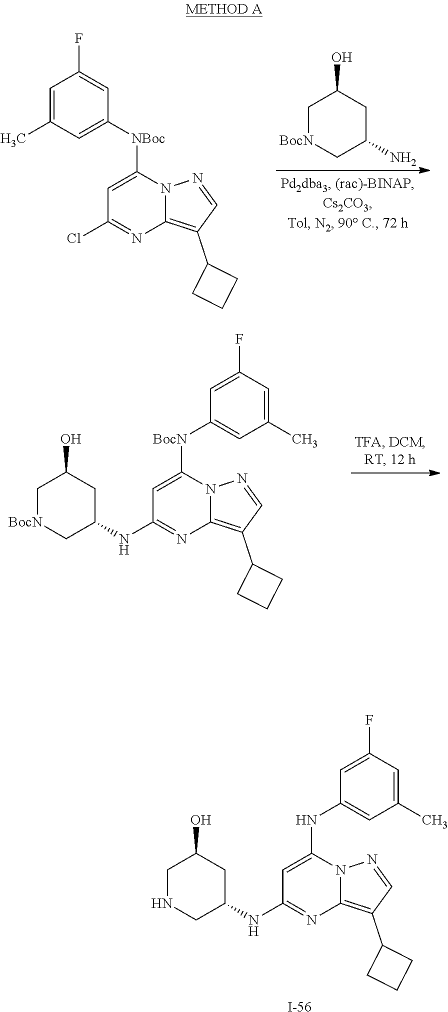

### Tert-Butyl (3S,5S)-3-((7-((tert-butoxycarbonyl)(3-fluoro-5-methylphenyl)amino)-3-cyclobutylpyrazolo[1,5-a]pyrimidin-5-yl)amino)-5-hydroxylpiperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclobutylpyrazolo[1,5-a]pyrimidin-7-yl)(3-fluoro-5-methylphenyl)carbamate, prepared according to Example 43 above (100 mg, 0.23 mmol, 1 eq), and (3S,5S)-3-amino-5-hydroxy-piperidine-1-carboxylic acid tert-butyl ester (54 mg, 0.25 mmol, 1.1 eq) and Cs₂CO₃ (150 mg, 0.46 mmol, 2 eq) were added at room temperature. The reaction mixture was under nitrogen flush for 5 minutes and then the microwave vial was sealed and heated at 90° C. for 72 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (3S,5S)-3-((7-((tert-butoxycarbonyl)(3-fluoro-5-methylphenyl)amino)-3-cyclobutylpyrazolo[1,5-a]pyrimidin-5-yl)amino)-5-hydroxylpiperidine-1-carboxylate as a brown oil (30 mg, 21%). TLC system: EtOAc/hexane (1:1), R_{f} value: ˜0.2.

### (3S,5S)-5-((3-Cyclobutyl-7-((3-fluoro-5-methylphenyl)amino)pyrazolo[1,5-a]pyrimidin-5-yl)amino)piperidin-3-ol (I-56)

To a solution of crude tert-butyl (3S,5S)-3-((7-((tert-butoxycarbonyl)(3-fluoro-5-methylphenyl)amino)-3-cyclobutylpyrazolo[1,5-a]pyrimidin-5-yl)amino)-5-hydroxylpiperidine-1-carboxylate (30 mg, 0.05 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (3S,5S)-5-((3-cyclobutyl-7-((3-fluoro-5-methylphenyl)amino)pyrazolo[1,5-a]pyrimidin-5-yl)amino)piperidin-3-ol (I-56) as a brown solid (15 mg, yield: 75%). TLC system: DCM/Methanol (2:1), R_{f} value: ˜0.4; ¹HNMR (500 Hz, MeOD-d4) δ 7.77 (s, 1H), 7.01 (s, 1H), 6.95 (d, J=10.2 Hz, 1H), 6.77 (d, J=9.3 Hz, 1H), 5.76 (s, 1H), 4.27-4.23 (m, 1H), 3.93-3.88 (m, 1H), 3.68-3.61 (m, 1H), 3.41-3.37 (m, 1H), 3.21-3.18 (m, 1H), 2.71-2.61 (m, 2H), 2.37 (s, 3H), 2.34-2.27 (m, 5H), 2.04-1.97 (m, 1H), 1.95-1.91 (m, 1H), 1.59-1.53 (m, 1H). HRMS (ESI) m/z [M+H]⁺ calcd for C₂₂H₂₇FN₆O 411.2303, found 411.2309. HPLC: 97%.

### Example 48

### Synthesis of TRANS-5-((3-cyclobutyl-7-((3-fluoro-5-methylphenyl)amino)pyrazolo[1,5-a]pyrimidin-5-yl)oxy)piperidin-3-ol (Compound I-57)

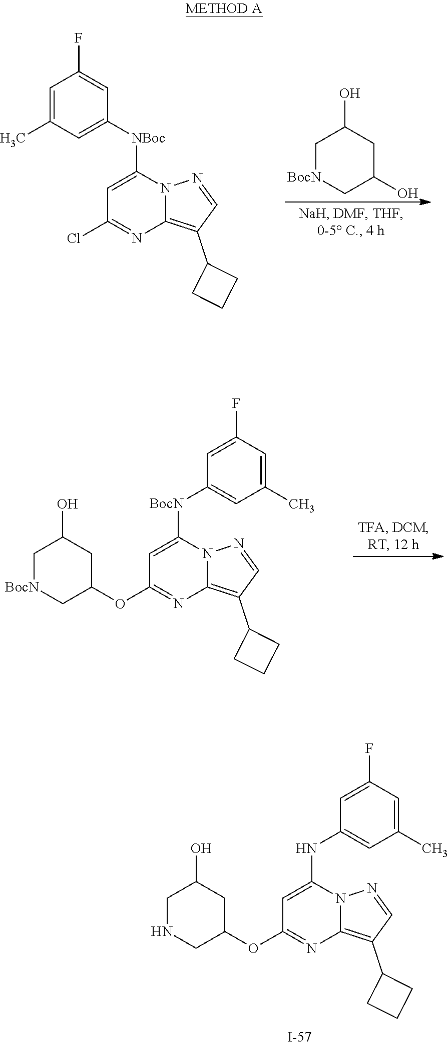

### Tert-Butyl bans-3-((7-((tert-butoxycarbonyl)(3-fluoro-5-methylphenyl)amino)-3-cyclobutylpyrazolo[1,5-a]pyrimidin-5-yl)oxy)-5-hydroxylpiperidine-1-carboxylate

To a stirred solution of NaH (15 mg, 0.37 mmol, 1.6 eq) in DMF (3 mL), the mixture of tert-butyl (5-chloro-3-cyclobutylpyrazolo[1,5-a]pyrimidin-7-yl)(3-fluoro-5-methylphenyl)carbamate, prepared according to Example 43 above (100 mg, 0.23 mmol, 1.0 eq), and trans-3,5-Dihydroxy-piperidine-1-carboxylic acid tert-butyl ester (racemate) (61 mg, 0.28 mmol, 1.2 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl trans-3-((7-((tert-butoxycarbonyl)(3-fluoro-5-methylphenyl)amino)-3-cyclobutylpyrazolo[1,5-a]pyrimidin-5-yl)oxy)-5-hydroxylpiperidine-1-carboxylate as yellow oil (45 mg; 32%). TLC system: EtoAc:Hexane (1:4), R_{f} value: ˜0.3.

### Trans-5-((3-Cyclobutyl-7-((3-fluoro-5-methylphenyl)amino)pyrazolo[1,5-a]pyrimidin-5-yl)oxy)piperidin-3-ol (I-57)

To a solution of crude tert-butyl trans-3-((7-((tert-butoxycarbonyl)(3-fluoro-5-methylphenyl)amino)-3-cyclobutylpyrazolo[1,5-a]pyrimidin-5-yl)oxy)-5-hydroxylpiperidine-1-carboxylate (70 mg, 0.11 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford trans-5-((3-cyclobutyl-7-((3-fluoro-5-methylphenyl)amino)pyrazolo[1,5-a]pyrimidin-5-yl)oxy)piperidin-3-ol (I-57) as a brown solid (18 mg, yield: 40%). TLC system: DCM/Methanol (1:1), R_{f} value: ˜0.3; ¹HNMR (500 Hz, MeOD-d4) δ 7.86 (s, 1H), 7.03 (s, 1H), 6.96 (d, J=10.0 Hz, 1H), 6.80 (d, J=9.3 Hz, 1H), 5.83 (s, 1H), 5.49-5.46 (m, 1H), 4.03-3.98 (m, 1H), 3.70-3.63 (m, 1H), 3.10-2.99 (m, 3H), 2.60-2.56 (m, 1H), 2.38 (s, 3H), 2.36-2.32 (m, 4H), 2.29-2.25 (m, 1H), 2.05-1.99 (m, 1H), 1.98-1.93 (m, 1H), 1.86-1.81 (m, 1H). HRMS (ESI) m/z [M+H]⁺ calcd for C₂₂H₂₆FN₅O₂ 412.2143, found 412.2138. HPLC: 95%.

### Example 49

### Synthesis of (S)-3-cyclobutyl-N-(3-fluoro-5-methylphenyl)-5-((piperidin-3-yl)oxy)pyrazolo[1,5-a]pyrimidin-7-amine (Compound I-58)

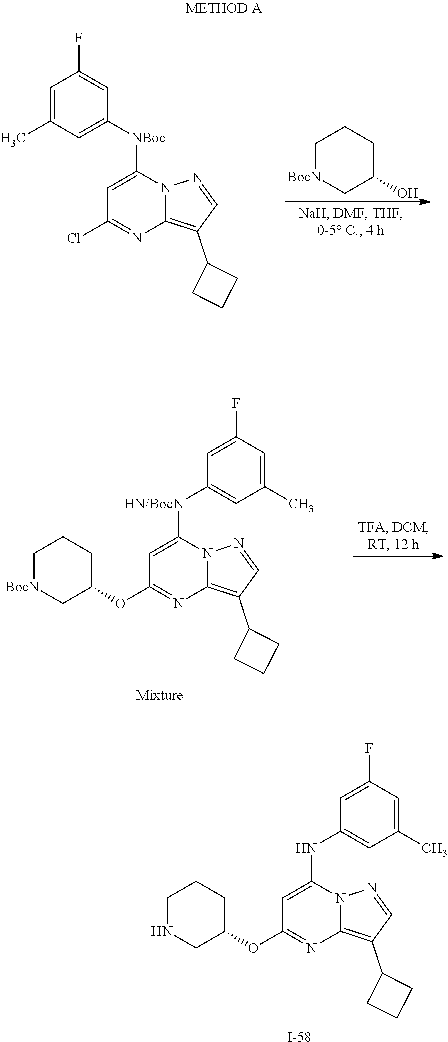

### Tert-Butyl (S)-3-((7-((tert-butoxycarbonyl)(3-fluoro-5-methylphenyl)amino)-3-cyclobutylpyrazolo[1,5-a]pyrimidin-5-yl)oxy)piperidine-1-carboxylate

To a stirred solution of NaH (16 mg, 0.40 mmol, 2.0 eq) in DMF (3 mL), the mixture of tert-butyl (5-chloro-3-cyclobutylpyrazolo[1,5-a]pyrimidin-7-yl)(3-fluoro-5-methylphenyl)carbamate (85 mg, 0.20 mmol, 1.0 eq) and (S)-1-Boc-3-hydroxypiperidine (48 mg, 0.24 mmol, 1.2 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl (S)-3-((7-((tert-butoxycarbonyl)(3-fluoro-5-methylphenyl)amino)-3-cyclobutylpyrazolo[1,5-a]pyrimidin-5-yl)oxy)piperidine-1-carboxylate as yellow oil (75 mg; 63%). TLC system: EtoAc:Hexane (1:8), R_{f} value: ˜0.2.

### (S)-3-Cyclobutyl-N-(3-fluoro-5-methylphenyl)-5-((piperidin-3-yl)oxy)pyrazolo[1,5-a]pyrimidin-7-amine (I-58)

To a solution of crude tert-butyl (S)-3-((7-((tert-butoxycarbonyl)(3-fluoro-5-methylphenyl)amino)-3-cyclobutylpyrazolo[1,5-a]pyrimidin-5-yl)oxy)piperidine-1-carboxylate (67 mg, 0.11 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclobutyl-N-(3-fluoro-5-methylphenyl)-5-((piperidin-3-yl)oxy)pyrazolo[1,5-a]pyrimidin-7-amine (I-58) as a brown solid (28 mg, yield: 64%). TLC system: DCM/Methanol (2:1), R_{f} value: ˜0.2; ¹HNMR (500 Hz, MeOD-d4) δ 7.86 (s, 1H), 7.01 (s, 1H), 6.94 (dt, J=10.0, 1.9 Hz, 1H), 6.80 (d, J=9.3 Hz, 1H), 5.84 (s, 1H), 5.39-5.36 (m, 1H), 3.68-3.61 (m, 1H), 3.33-3.10 (m, 1H), 3.29-3.26 (m, 1H), 3.10-3.06 (m, 1H), 3.03-2.98 (m, 1H), 2.36 (s, 3H), 2.34-2.31 (m, 4H), 2.04-1.91 (m, 5H), 1.74-1.71 (m, 1H). HRMS (ESI) m/z [M+H]⁺ calcd for C₂₂H₂₆FN₅O 396.2194, found 396.2198. HPLC: 96%.

### Example 50

### Synthesis of (S)-3-cyclobutyl-N⁷-(3-fluoro-5-methylphenyl)-N⁵-((piperidin-3-yl)methyl)pyrazolo[1,5-a]pyrimidine-5,7-diamine (Compound I-59)

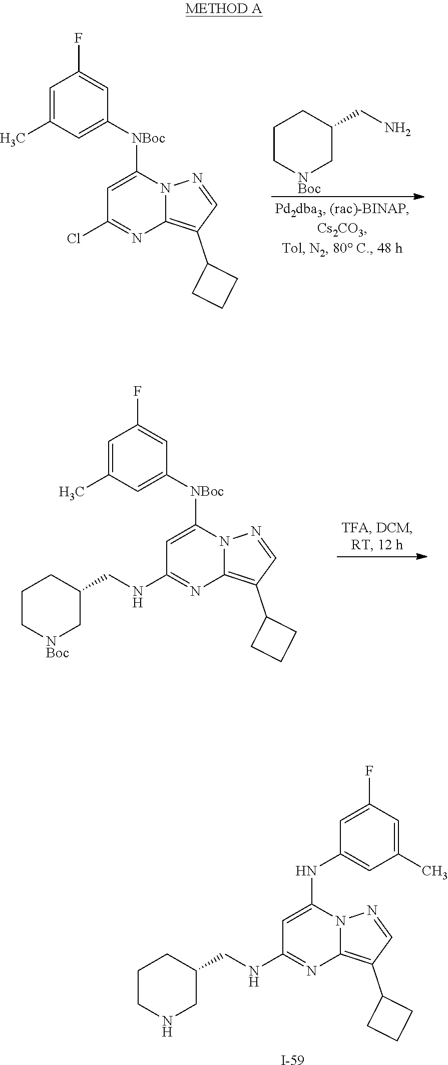

### Tert-Butyl (R)-3-((7-((tert-butoxycarbonyl)(3-fluoro-5-methylphenyl)amino)-3-cyclobutylpyrazolo[1,5-a]pyrimidin-5-yl)aminomethyl)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclobutylpyrazolo[1,5-a]pyrimidin-7-yl)(3-fluoro-5-methylphenyl)carbamate, prepared according to Example 43 above (100 mg, 0.23 mmol, 1 eq), (R)-1-Boc-3-(aminomethyl)piperidine (54 mg, 0.25 mmol, 1.1 eq) and Cs₂CO₃ (150 mg, 0.46 mmol, 2 eq) were added at room temperature. The reaction mixture was under nitrogen flush for 5 minutes and then the microwave vial was sealed and heated at 80° C. for 48 h. After completion of reaction by TLC, the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (R)-3-((7-((tert-butoxycarbonyl)(3-fluoro-5-methylphenyl)amino)-3-cyclobutylpyrazolo[1,5-a]pyrimidin-5-yl)aminomethyl)piperidine-1-carboxylate as a brown oil (28 mg, 20%). TLC system: EtOAc/hexane (1:2), R_{f} value: ˜0.50.

### (S)-3-Cyclobutyl-N⁷-(3-fluoro-5-methylphenyl)-N⁵-((piperidin-3-yl)methyl)pyrazolo[1,5-a]pyrimidine-5,7-diamine (I-59)

To the solution of crude (tert-butyl (R)-3-((7-((tert-butoxycarbonyl)(3-fluoro-5-methylphenyl)amino)-3-cyclobutylpyrazolo[1,5-a]pyrimidin-5-yl)aminomethyl)piperidine-1-carboxylate (60 mg, 0.10 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclobutyl-N⁷-(3-fluoro-5-methylphenyl)-N⁵-((piperidin-3-yl)methyl)pyrazolo[1,5-a]pyrimidine-5,7-diamine (I-59) as a brown solid (25 mg, yield: 63%). TLC system: DCM/Methanol (1:1), R_{f} value: ˜0.3; ¹HNMR (500 Hz, MeOD-d4) δ 7.75 (s, 1H), 7.00 (s, 1H), 6.94 (d, J=10.2 Hz, 1H), 6.76 (d, J=9.4 Hz, 1H), 5.78 (s, 1H), 3.67-3.60 (m, 1H), 3.48-3.36 (m, 3H), 3.34-3.32 (m, 1H), 2.96-2.90 (m, 1H), 2.80-2.75 (m, 1H), 2.37 (s, 3H), 2.35-2.32 (m, 4H), 2.22-2.16 (m, 1H), 2.06-1.99 (m, 1H), 1.98-1.91 (m, 3H), 1.79-1.71 (m, 1H), 1.43-1.34 (m, 1H). HRMS (ESI) m/z [M+H]⁺ calcd for C₂₃H₂₉FN₆ 409.2510, found 409.2509. HPLC: 95%.

### Example 51

### Synthesis of (S)-3-((3-cyclobutyl-5-((piperidin-3-yl)methoxy)pyrazolo[1,5-a]pyrimidin-7-yl)amino)-5-fluorobenzonitrile (Compound I-60)

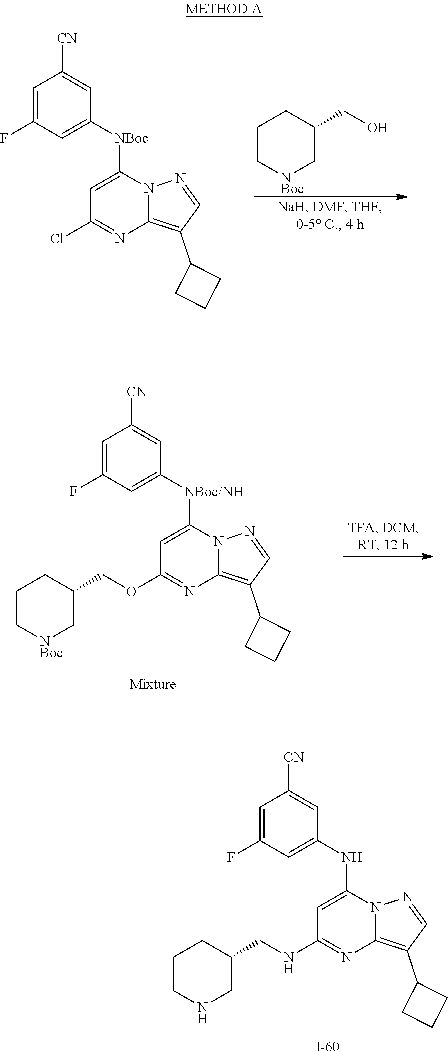

### Tert-Butyl (S)-3-((7-((tert-butoxycarbonyl)(3-cyano-5-fluorophenyl)amino)-3-cyclobutylpyrazolo[1,5-a]pyrimidin-5-yl)oxymethyl)piperidine-1-carboxylate

To a stirred solution of 60% of NaH (17 mg, 0.42 mmol, 2.0 eq) in DMF (3 mL), the mixture of tert-butyl (5-chloro-3-cyclobutylpyrazolo[1,5-a]pyrimidin-7-yl)(3-cyano-5-fluorophenyl)carbamate, prepared according to Example 46 above (92 mg, 0.21 mmol, 1.0 eq), and (S)-1-Boc-3-(hydroxymethyl)piperidine (54 mg, 0.25 mmol, 1.2 eq) in THF (3 mL) was added at 0-5° C. and stirred at room temperature for 12 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford crude product which was purified by using combiflash chromatography (4 g column) to afford crude tert-butyl (S)-3-((7-((tert-butoxycarbonyl)(3-cyano-5-fluorophenyl)amino)-3-cyclobutylpyrazolo[1,5-a]pyrimidin-5-yl)oxymethyl)piperidine-1-carboxylate as yellow oil (30 mg; 23%). TLC system: EtoAc:Hexane (1:4), R_{f} value: ˜0.3.

### (S)-3-((3-Cyclobutyl-5-((piperidin-3-yl)methoxy)pyrazolo[1,5-a]pyrimidin-7-yl)amino)-5-fluorobenzonitrile (I-60)

To a solution of crude tert-butyl (S)-3-((7-((tert-butoxycarbonyl)(3-cyano-5-fluorophenyl)amino)-3-cyclobutylpyrazolo[1,5-a]pyrimidin-5-yl)oxymethyl)piperidine-1-carboxylate (30 mg, 0.05 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-((3-cyclobutyl-5-((piperidin-3-yl)methoxy)pyrazolo[1,5-a]pyrimidin-7-yl)amino)-5-fluorobenzonitrile (I-60) as a brown solid (6 mg, yield: 30%). TLC system: DCM/Methanol (1:1), R_{f} value: ˜0.5; ¹HNMR (500 Hz, MeOD-d4) δ 7.93 (s, 1H), 7.64 (s, 1H), 7.55 (dt, J=10.1, 2.1 Hz, 1H), 7.41 (d, J=9.3 Hz, 1H), 5.94 (s, 1H), 4.46-4.42 (m, 1H), 4.30-4.26 (m, 1H), 3.73-3.66 (m, 1H), 3.53-3.50 (m, 1H), 3.38-3.35 (m, 1H), 2.97-2.87 (m, 2H), 2.39-2.29 (m, 5H), 2.07-1.93 (m, 4H), 1.83-1.73 (m, 1H), 1.56-1.47 (m, 1H). HRMS (ESI) m/z [M+H]⁺ calcd for C₂₃H₂₅FN₆O 421.2147, found 421.2153. HPLC: 95%.

### Example 52

### Synthesis of (S)-3-cyclopropyl-n-(5-methyl-3-trifluoromethylphenyl)-5-((piperidin-3-yl)oxy)pyrazolo[1,5-a]pyrimidin-7-amine (Compound I-61)

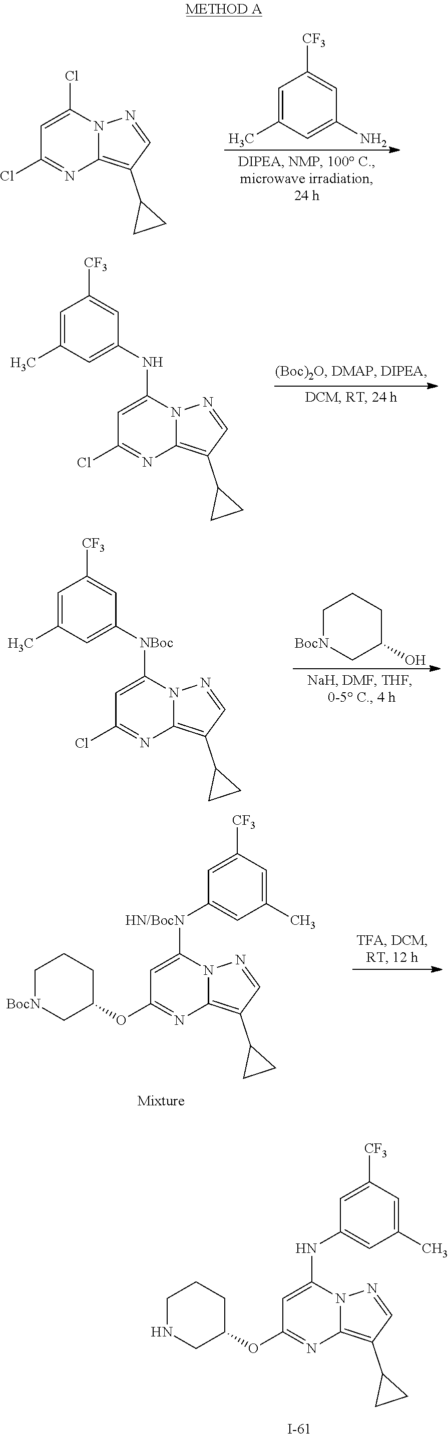

### 5-Chloro-3-cyclopropyl-N-(5-methyl-3-trifluoromethylphenyl) pyrazolo[1,5-a]pyrimidin-7-amine

To a stirred solution of 3-trifluoromethyl-5-methylaniline (210 mg, 1.20 mmol, 1.2 eq) in NMP (4 mL) was added DIPEA (252 mg, 1.95 mmol, 1.3 eq) and stirred for 5 min. To this mixture 5,7-dichloro-3-cyclopropylpyrazolo[1,5-a]pyrimidine (242 mg, 1.00 mmol, 1.0 eq) was added at room temperature and continued stirring at 100° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclopropyl-N-(5-methyl-3-trifluoromethylphenyl)pyrazolo[1,5-a]pyrimidin-7-amine as a brown liquid. TLC system: EA:Hexane (1:8), R_{f} value: ˜0.2.

### Tert-Butyl (5-chloro-3-cyclopropylpyrazolo[1,5-a]pyrimidin-7-yl)(5-methyl-3-trifluoromethylphenyl)carbamate

To a stirred solution of 5-chloro-3-cyclopropyl-N-(5-methyl-3-trifluoromethylphenyl)pyrazolo[1,5-a]pyrimidin-7-amine (367 mg, 1.00 mmol, 1.0 eq) in DCM (5 mL) at room temperature was added DIPEA (259 mg, 2.00 mmol, 2 eq) and DMAP (3 mg 0.025 mmol), followed by addition of (Boc)₂O (284 mg, 1.30 mmol, 1.3 eq). The reaction was continued at room temperature for 24 h. Then, (Boc)₂O (284 mg, 1.30 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclopropylpyrazolo[1,5-a]pyrimidin-7-yl)(5-methyl-3-trifluoromethylphenyl)carbamate as a yellow oil (435 mg, 93% for two steps). TLC system: EtOAc/hexane (1:8), R_{f} value: ˜0.3; ¹HNMR (500 MHz, CDCl₃) δ 7.86 (s, 1H), 7.40 (s, 1H), 7.34 (s, 1H), 7.23 (s, 1H), 6.60 (s, 1H), 2.37 (s, 3H), 2.10-2.05 (m, 1H), 1.37 (s, 9H), 1.03-0.99 (m, 2H), 0.86-0.83 (m, 2H).

### Tert-Butyl (S)-3-((7-((tert-butoxycarbonyl)(5-methyl-3-trifluoromethylphenyl)amino)-3-cyclopropylpyrazolo[1,5-a]pyrimidin-5-yl)oxy)piperidine-1-carboxylate

To a stirred solution of NaH (14 mg, 0.34 mmol, 2.0 eq) in DMF (3 mL), the mixture of tert-butyl (5-chloro-3-cyclopropylpyrazolo[1,5-a]pyrimidin-7-yl)(5-methyl-3-trifluoromethylphenyl)carbamate (80 mg, 0.17 mmol, 1.0 eq) and (S)-1-Boc-3-hydroxypiperidine (40 mg, 0.20 mmol, 1.2 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl (S)-3-((7-((tert-butoxycarbonyl)(5-methyl-3-trifluoromethylphenyl)amino)-3-cyclopropylpyrazolo[1,5-a]pyrimidin-5-yl)oxy)piperidine-1-carboxylate as yellow oil (50 mg; 47%). TLC system: EtoAc:Hexane (1:4), R_{f} value: ˜0.3.

### (S)-3-Cyclopropyl-N-(5-methyl-3-trifluoromethylphenyl)-5-((piperidin-3-yl)oxy)pyrazolo[1,5-a]pyrimidin-7-amine (I-61)

To the solution of crude tert-butyl (S)-3-((7-((tert-butoxycarbonyl)(5-methyl-3-trifluoromethylphenyl)amino)-3-cyclopropylpyrazolo[1,5-a]pyrimidin-5-yl)oxy)piperidine-1-carboxylate (40 mg, 0.06 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclopropyl-N-(5-methyl-3-trifluoromethylphenyl)-5-((piperidin-3-yl)oxy)pyrazolo[1,5-a]pyrimidin-7-amine (I-61) as a brown solid (15 mg, yield: 58%). TLC system: DCM/Methanol (2:1), R_{f} value: ˜0.3; ¹HNMR (500 Hz, MeOD-d4) δ 7.73 (s, 1H), 7.49 (s, 1H), 7.48 (s, 1H), 7.38 (s, 1H), 5.77 (s, 1H), 5.37-5.34 (m, 1H), 3.30-3.28 (m, 1H), 3.23-3.19 (m, 1H), 3.04-2.95 (m, 2H), 2.47 (s, 3H), 2.02-1.96 (m, 3H), 1.91-1.87 (m, 1H), 1.73-1.69 (m, 1H), 0.89-0.81 (m, 4H). HRMS (ESI) m/z [M+H]⁺ calcd for C₂₂H₂₄F₃N₅O 432.2006, found 432.2010. HPLC: 97%.

### Example 53

### Synthesis of (S)-3-cyclopropyl-N⁷-(5-methyl-3-trifluoromethylphenyl)-N⁵-(piperidin-3-yl)pyrazolo[1,5-a]pyrimidine-5,7-diamine (Compound I-62)

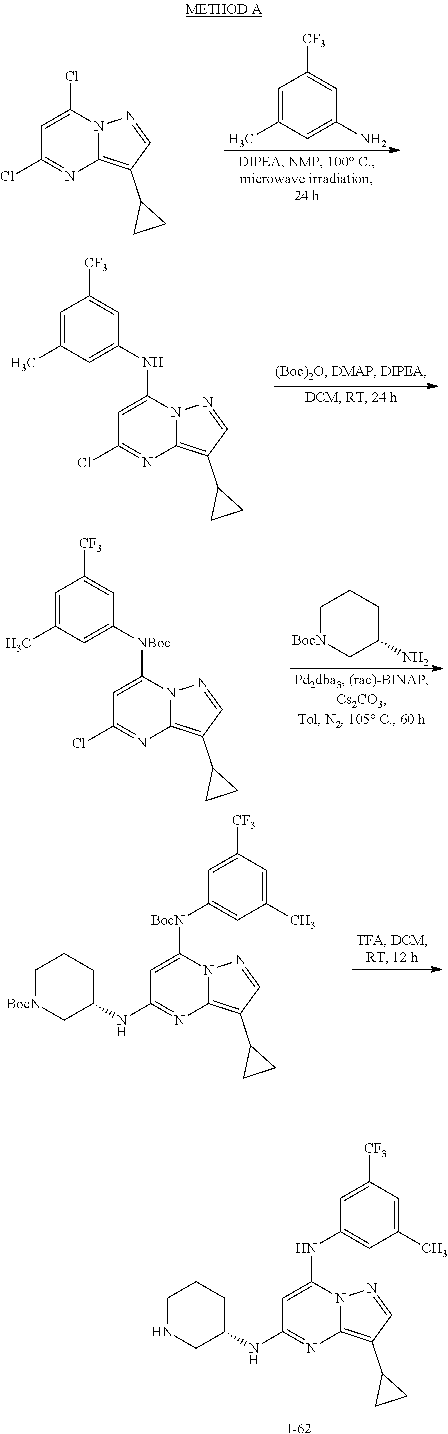

### 5-Chloro-3-cyclopropyl-N-(5-methyl-3-trifluoromethylphenyl)pyrazolo[1,5-a]pyrimidin-7-amine

To a stirred solution of 3-trifluoromethyl-5-methylaniline (210 mg, 1.20 mmol, 1.2 eq) in NMP (4 mL) was added DIPEA (252 mg, 1.95 mmol, 1.3 eq) and stirred for 5 min. To this mixture 5,7-dichloro-3-cyclopropylpyrazolo[1,5-a]pyrimidine (242 mg, 1.00 mmol, 1.0 eq) was added at room temperature and continued stirring at 100° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclopropyl-N-(5-methyl-3-trifluoromethylphenyl)pyrazolo[1,5-a]pyrimidin-7-amine as a brown liquid. TLC system: EA:Hexane (1:8), R_{f} value: ˜0.2.

### Tert-Butyl (5-chloro-3-cyclopropylpyrazolo[1,5-a]pyrimidin-7-yl)(5-methyl-3-trifluoromethylphenyl)carbamate

To a stirred solution of 5-chloro-3-cyclopropyl-N-(5-methyl-3-trifluoromethylphenyl)pyrazolo[1,5-a]pyrimidin-7-amine (367 mg, 1.00 mmol, 1.0 eq) in DCM (5 mL) at room temperature was added DIPEA (259 mg, 2.00 mmol, 2 eq) and DMAP (3 mg 0.025 mmol), followed by addition of (Boc)₂O (284 mg, 1.30 mmol, 1.3 eq). The reaction was continued at room temperature for 24 h. Then, (Boc)₂O (284 mg, 1.30 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclopropylpyrazolo[1,5-a]pyrimidin-7-yl)(5-methyl-3-trifluoromethylphenyl)carbamate as a yellow oil (435 mg, 93% for two steps). TLC system: EtOAc/hexane (1:8), R_{f} value: ˜0.3; ¹HNMR (500 MHz, CDCl₃) δ 7.86 (s, 1H), 7.40 (s, 1H), 7.34 (s, 1H), 7.23 (s, 1H), 6.60 (s, 1H), 2.37 (s, 3H), 2.10-2.05 (m, 1H), 1.37 (s, 9H), 1.03-0.99 (m, 2H), 0.86-0.83 (m, 2H).

### Tert-Butyl (S)-3-((7-((tert-butoxycarbonyl)(5-methyl-3-trifluoromethylphenyl)amino)-3-cyclopropylpyrazolo[1,5-a]pyrimidin-5-yl)amino)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclopropylpyrazolo[1,5-a]pyrimidin-7-yl)(5-methyl-3-trifluoromethylphenyl)carbamate (100 mg, 0.21 mmol, 1 eq), (S)-1-Boc-3-aminopiperidine (46 mg, 0.23 mmol, 1.1 eq) and Cs₂CO₃ (137 mg, 0.42 mmol, 2 eq) were added at room temperature. The reaction mixture was under nitrogen flush for 5 minutes and then the microwave vial was sealed and heated at 105° C. for 60 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (S)-3-((7-((tert-butoxycarbonyl)(5-methyl-3-trifluoromethylphenyl)amino)-3-cyclopropylpyrazolo[1,5-a]pyrimidin-5-yl)amino)piperidine-1-carboxylate as a brown oil (78 mg, 58%). TLC system: EtOAc/hexane (1:2), R_{f} value: ˜0.3.

### (S)-3-Cyclopropyl-N⁷-(5-methyl-3-trifluoromethylphenyl)-N⁵-(piperidin-3-yl)pyrazolo[1,5-a]pyrimidine-5,7-diamine (I-62)

To the solution of crude tert-butyl (S)-3-((7-((tert-butoxycarbonyl)(5-methyl-3-trifluoromethylphenyl)amino)-3-cyclopropylpyrazolo[1,5-a]pyrimidin-5-yl)amino)piperidine-1-carboxylate (45 mg, 0.07 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclopropyl-N⁷-(5-methyl-3-trifluoromethylphenyl)-N⁵-(piperidin-3-yl)pyrazolo[1,5-a]pyrimidine-5,7-diamine (I-62) as a brown solid (10 mg, yield: 29%). TLC system: DCM/Methanol (2:1), R_{f} value: ˜0.3; ¹HNMR (500 Hz, MeOD-d4) δ 7.59 (s, 1H), 7.45 (s, 2H), 7.32 (s, 1H), 5.74 (s, 1H), 4.28-4.23 (m, 1H), 3.61-3.58 (m, 1H), 3.27-3.22 (m, 1H), 3.02-2.97 (m, 2H), 2.46 (s, 3H), 2.12-2.03 (m, 2H), 1.90-1.84 (m, 2H), 1.71-1.64 (m, 1H), 0.87-0.75 (m, 4H). HRMS (ESI) m/z [M+H]⁺ calcd for C₂₂H₂₅F₃N₆ 431.2166, found 431.2175. HPLC: 97%.

### Example 54

### Synthesis of (S)-3-cyclobutyl-N⁷-(5-methyl-3-trifluoromethylphenyl)-N⁵-(piperidin-3-yl)pyrazolo[1,5-a]pyrimidine-5,7-diamine (Compound I-63)

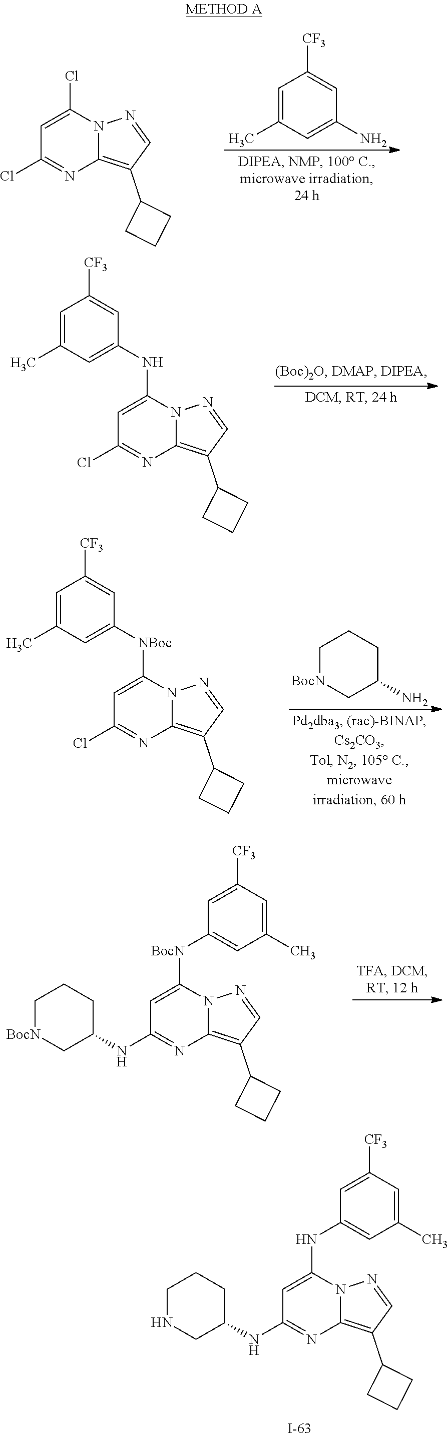

### 5-Chloro-3-cyclobutyl-N-(5-methyl-3-trifluoromethylphenyl)pyrazolo[1,5-a]pyrimidin-7-amine

To a stirred solution of 3-trifluoromethyl-5-methylaniline (210 mg, 1.20 mmol, 1.2 eq) in NMP (4 mL) was added DIPEA (252 mg, 1.95 mmol, 1.3 eq) and stirred for 5 min. To this mixture 5,7-dichloro-3-cyclobutylpyrazolo[1,5-a]pyrimidine (242 mg, 1.00 mmol, 1.0 eq) was added at room temperature and continued stirring at 100° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclobutyl-N-(5-methyl-3-trifluoromethylphenyl)pyrazolo[1,5-a]pyrimidin-7-amine as a brown liquid. TLC system: EA:Hexane (1:4), R_{f} value: ˜0.3.

### Tert-Butyl (5-chloro-3-cyclobutylpyrazolo[1,5-a]pyrimidin-7-yl)(5-methyl-3-trifluoromethylphenyl)carbamate

To a stirred solution of 5-chloro-3-cyclobutyl-N-(5-methyl-3-trifluoromethylphenyl)pyrazolo[1,5-a]pyrimidin-7-amine (381 mg, 1.00 mmol, 1.0 eq) in DCM (5 mL) at room temperature was added DIPEA (259 mg, 2.00 mmol, 2 eq) and DMAP (3 mg 0.025 mmol), followed by addition of (Boc)₂O (284 mg, 1.30 mmol, 1.3 eq). The reaction was continued at room temperature for 24 h. Then, (Boc)₂O (284 mg, 1.30 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclobutylpyrazolo[1,5-a]pyrimidin-7-yl)(5-methyl-3-trifluoromethylphenyl)carbamate as a brown oil (400 mg, 83% for two steps). TLC system: EtOAc/hexane (1:8), R_{f} value: ˜0.4; ¹HNMR (500 MHz, CDCl₃) δ 8.12 (s, 1H), 7.41 (s, 1H), 7.35 (s, 1H), 7.24 (s, 1H), 6.60 (s, 1H), 3.88-3.81 (m, 1H), 2.47-2.41 (m, 2H), 2.38 (s, 3H), 2.33-2.26 (m, 2H), 2.11-2.01 (m, 1H), 1.99-1.93 (m, 1H), 1.38 (s, 9H).

### Tert-Butyl (S)-3-((7-((tert-butoxycarbonyl)(5-methyl-3-trifluoromethylphenyl)amino)-3-cyclobutylpyrazolo[1,5-a]pyrimidin-5-yl)amino)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclobutylpyrazolo[1,5-a]pyrimidin-7-yl)(5-methyl-3-trifluoromethylphenyl)carbamate (100 mg, 0.21 mmol, 1 eq), (S)-1-Boc-3-aminopiperidine (46 mg, 0.23 mmol, 1.1 eq) and Cs₂CO₃ (137 mg, 0.42 mmol, 2 eq) were added at room temperature. The reaction mixture was under nitrogen flush for 5 minutes and then the microwave vial was sealed and heated at 105° C. for 60 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (S)-3-((7-((tert-butoxycarbonyl)(5-methyl-3-trifluoromethylphenyl)amino)-3-cyclobutylpyrazolo[1,5-a]pyrimidin-5-yl)amino)piperidine-1-carboxylate as a brown oil (78 mg, 58%). TLC system: EtOAc/hexane (1:2), R_{f} value: ˜0.3.

### (S)-3-Cyclobutyl-N⁷-(5-methyl-3-trifluoromethylphenyl)-N⁵-(piperidin-3-yl)pyrazolo[1,5-a]pyrimidine-5,7-diamine (I-63)

To the solution of crude tert-butyl (S)-3-((7-((tert-butoxycarbonyl)(5-methyl-3-trifluoromethylphenyl)amino)-3-cyclobutylpyrazolo[1,5-a]pyrimidin-5-yl)amino)piperidine-1-carboxylate (68 mg, 0.11 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclobutyl-N⁷-(5-methyl-3-trifluoromethylphenyl)-N⁵-(piperidin-3-yl)pyrazolo[1,5-a]pyrimidine-5,7-diamine (I-63) as a brown solid (30 mg, yield: 61%). TLC system: DCM/Methanol (2:1), Re value: ˜0.3; ¹HNMR (500 Hz, MeOD-d4) δ 7.76 (s, 1H), 7.41 (s, 2H), 7.29 (s, 1H), 4.15-4.09 (m, 1H), 3.68-3.61 (m, 1H), 3.46-3.43 (m, 1H), 3.10-3.05 (m, 1H), 2.81-2.73 (m, 2H), 2.43 (s, 3H), 2.33-2.28 (m, 4H), 2.09-2.05 (m, 1H), 2.02-1.89 (m, 3H), 1.76-1.68 (i, 1H), 1.60-1.53 (N, 1H). HRMS (ESI)/z [M+H]⁺ calcd for C₂₃H₂₇F₃N₆ 445.2322, found 445.2330. HPLC: 95%.

### Example 55

### Synthesis of (S)-3-cyclopropyl-N-(5-fluoro-3-Trifluoromethylphenyl)-5-((piperidin-3-yl)oxy)pyrazolo[1,5-a]pyrimidin-7-amine (Compound I-64)

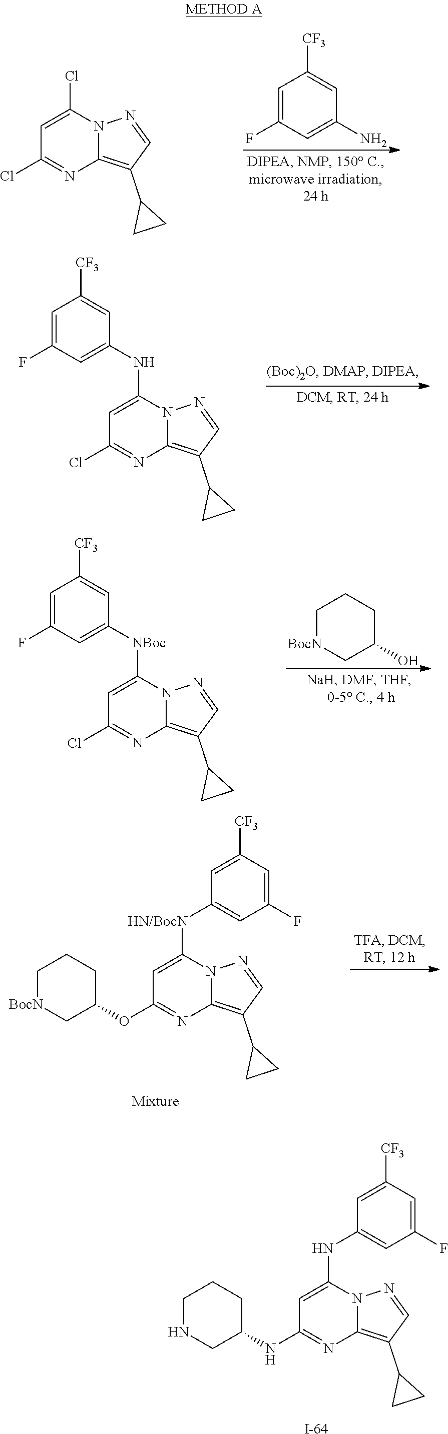

### 5-Chloro-3-cyclopropyl-N-(5-fluoro-3-trifluoromethylphenyl) pyrazolo[1,5-a]pyrimidin-7-amine

To a stirred solution of 3-trifluoromethyl-5-fluoroaniline (90 mg, 0.50 mmol, 1.0 eq) in NMP (4 mL) was added DIPEA (84 mg, 0.65 mmol, 1.3 eq) and stirred for 5 min. To this mixture 5,7-dichloro-3-cyclopropylpyrazolo[1,5-a]pyrimidine (114 mg, 0.50 mmol, 1.0 eq) was added at room temperature and continued stirring at 150° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclopropyl-N-(5-fluoro-3-trifluoromethylphenyl)pyrazolo[1,5-a]pyrimidin-7-amine as a brown liquid. TLC system: EA:Hexane (1:4), R_{f} value: ˜0.5.

### Tert-Butyl (5-chloro-3-cyclopropylpyrazolo[1,5-a]pyrimidin-7-yl)(5-fluoro-3-trifluoromethylphenyl)carbamate

To a stirred solution of 5-chloro-3-cyclopropyl-N-(5-fluoro-3-trifluoromethylphenyl)pyrazolo[1,5-a]pyrimidin-7-amine (185 mg, 0.50 mmol, 1.0 eq) in DCM (5 mL) at room temperature was added DIPEA (129 mg, 1.00 mmol, 2 eq) and DMAP (3 mg 0.025 mmol), followed by addition of (Boc)₂O (142 mg, 0.65 mmol, 1.3 eq). The reaction was continued at room temperature for 24 h. Then, (Boc)₂O (142 mg, 0.65 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclopropylpyrazolo[1,5-a]pyrimidin-7-yl)(5-fluoro-3-trifluoromethylphenyl)carbamate as a yellow oil (235 mg, 83% for two steps). TLC system: EtOAc/hexane (1:16), R_{f} value: ˜0.3; ¹HNMR (500 MHz, CDCl₃) δ 7.85 (s, 1H), 7.38 (s, 1H), 7.24 (s, 1H), 7.22 (s, 1H), 6.67 (s, 1H), 2.10-2.05 (m, 1H), 1.38 (s, 9H), 1.04-0.99 (m, 2H), 0.86-0.83 (m, 2H).

### Tert-Butyl (S)-3-((7-((tert-butoxycarbonyl)(5-fluoro-3-trifluoromethylphenyl)amino)-3-cyclopropylpyrazolo[1,5-a]pyrimidin-5-yl)oxy)piperidine-1-carboxylate

To a stirred solution of NaH (14 mg, 0.34 mmol, 1.6 eq) in DMF (3 mL), the mixture of tert-butyl (5-chloro-3-cyclopropylpyrazolo[1,5-a]pyrimidin-7-yl)(5-fluoro-3-trifluoromethylphenyl)carbamate (100 mg, 0.21 mmol, 1.0 eq) and (S)-1-Boc-3-hydroxypiperidine (85 mg, 0.42 mmol, 2.0 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl (S)-3-((7-((tert-butoxycarbonyl)(5-fluoro-3-trifluoromethylphenyl)amino)-3-cyclopropylpyrazolo[1,5-a]pyrimidin-5-yl)oxy)piperidine-1-carboxylate as yellow oil (70 mg; 53%). TLC system: EtoAc:Hexane (1:4), R_{f} value: ˜0.4.

### (S)-3-Cyclopropyl-N-(5-fluoro-3-trifluoromethylphenyl)-5-((piperidin-3-yl)oxy)pyrazolo[1,5-a]pyrimidin-7-amine (I-64)

To the solution of crude tert-butyl (S)-3-((7-((tert-butoxycarbonyl)(5-fluoro-3-trifluoromethylphenyl)amino)-3-cyclopropylpyrazolo[1,5-a]pyrimidin-5-yl)oxy)piperidine-1-carboxylate (70 mg, 0.11 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclopropyl-N-(5-fluoro-3-trifluoromethylphenyl)-5-((piperidin-3-yl)oxy)pyrazolo[1,5-a]pyrimidin-7-amine (I-64) as a brown solid (8 mg, yield: 17%). TLC system: DCM/Methanol (2:1), R_{f} value: ˜0.6; ¹HNMR (500 Hz, MeOD-d4) δ 7.75 (s, 1H), 7.55 (s, 1H), 7.51 (d, J=10.0 Hz, 1H), 7.31 (d, J=8.3 Hz, 1H), 5.93 (s, 1H), 5.38-5.34 (m, 1H), 3.30-3.27 (m, 1H), 3.23-3.20 (m, 1H), 3.05-2.96 (m, 2H), 2.03-1.96 (m, 3H), 1.92-1.87 (m, 1H), 1.73-1.69 (m, 1H), 0.90-0.82 (m, 4H). HRMS (ESI) m/z [M+H]⁺ calcd for C₂₁H₂₁F₄N₅O 436.1755, found 436.1762. HPLC: 95%.

### Example 56

### Synthesis of (S)-3-cyclobutyl-N-(5-fluoro-3-Trifluoromethylphenyl)-5-((piperidin-3-yl)oxy)pyrazolo[1,5-a]pyrimidin-7-amine (Compound I-65)

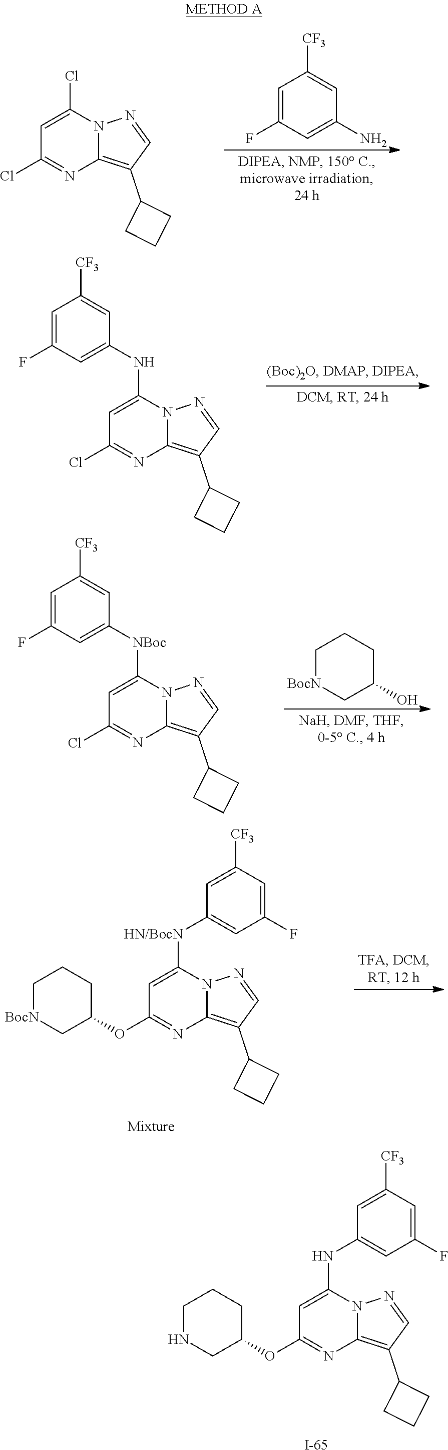

### 5-Chloro-3-cyclobutyl-N-(5-fluoro-3-trifluoromethylphenyl)pyrazolo[1,5-a]pyrimidin-7-amine

To a stirred solution of 3-trifluoromethyl-5-fluoroaniline (90 mg, 0.50 mmol, 1.0 eq) in NMP (4 mL) was added DIPEA (84 mg, 0.65 mmol, 1.3 eq) and stirred for 5 min. To this mixture 5,7-dichloro-3-cyclopropylpyrazolo[1,5-a]pyrimidine (141 mg, 0.50 mmol, 1.0 eq) was added at room temperature and continued stirring at 150° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-3-cyclobutyl-N-(5-fluoro-3-trifluoromethylphenyl)pyrazolo[1,5-a]pyrimidin-7-amine as a brown liquid. TLC system: EA:Hexane (1:4), R_{f} value: ˜0.6.

### Tert-Butyl (5-chloro-3-cyclobutylpyrazolo[1,5-a]pyrimidin-7-yl)(5-fluoro-3-trifluoromethylphenyl)carbamate

To a stirred solution of 5-chloro-3-cyclobutyl-N-(5-fluoro-3-trifluoromethylphenyl)pyrazolo[1,5-a]pyrimidin-7-amine (192 mg, 0.50 mmol, 1.0 eq) in DCM (5 mL) at room temperature was added DIPEA (129 mg, 1.00 mmol, 2 eq) and DMAP (3 mg 0.025 mmol), followed by addition of (Boc)₂O (142 mg, 0.65 mmol, 1.3 eq). The reaction was continued at room temperature for 24 h. Then, (Boc)₂O (142 mg, 0.65 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclobutylpyrazolo[1,5-a]pyrimidin-7-yl)(5-fluoro-3-trifluoromethylphenyl)carbamate as a yellow oil (235 mg, 83% for two steps). TLC system: EtOAc/hexane (1:16), R_{f} value: ˜0.3; ¹HNMR (500 MHz, CDCl₃) δ 8.10 (s, 1H), 7.39 (s, 1H), 7.25 (s, 1H), 7.23 (s, 1H), 6.68 (s, 1H), 3.87-3.80 (m, 1H), 2.48-2.41 (m, 2H), 2.33-2.26 (m, 2H), 2.09-2.03 (m, 1H), 1.99-1.93 (m, 1H), 1.38 (s, 9H).

### Tert-Butyl (S)-3-((7-((tert-butoxycarbonyl)(5-fluoro-3-trifluoromethylphenyl)amino)-3-cyclobutylpyrazolo[1,5-a]pyrimidin-5-yl)oxy)piperidine-1-carboxylate

To a stirred solution of NaH (18 mg, 0.44 mmol, 2.0 eq) in DMF (3 mL), the mixture of tert-butyl (5-chloro-3-cyclobutylpyrazolo[1,5-a]pyrimidin-7-yl)(5-fluoro-3-trifluoromethylphenyl)carbamate (108 mg, 0.22 mmol, 1.0 eq) and (S)-1-Boc-3-hydroxypiperidine (88 mg, 0.44 mmol, 2.0 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl (S)-3-((7-((tert-butoxycarbonyl)(5-fluoro-3-trifluoromethylphenyl)amino)-3-cyclobutylpyrazolo[1,5-a]pyrimidin-5-yl)oxy)piperidine-1-carboxylate as yellow oil (70 mg; 49%). TLC system: EtoAc:Hexane (1:8), R_{f} value: ˜0.2.

### (S)-3-Cyclobutyl-N-(5-fluoro-3-trifluoromethylphenyl)-5-((piperidin-3-yl)oxy)pyrazolo[1,5-a]pyrimidin-7-amine (I-65)

To the solution of crude tert-butyl (S)-3-((7-((tert-butoxycarbonyl)(5-fluoro-3-trifluoromethylphenyl)amino)-3-cyclobutylpyrazolo[1,5-a]pyrimidin-5-yl)oxy)piperidine-1-carboxylate (60 mg, 0.09 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclobutyl-N-(5-fluoro-3-trifluoromethylphenyl)-5-((piperidin-3-yl)oxy)pyrazolo[1,5-a]pyrimidin-7-amine (I-65) as a brown solid (18 mg, yield: 45%). TLC system: DCM/Methanol (2:1), R_{f} value: ˜0.5; ¹HNMR (500 Hz, MeOD-d4) δ 7.90 (s, 1H), 7.55 (s, 1H), 7.50 (d, J=10.0 Hz, 1H), 7.30 (d, J=8.3 Hz, 1H), 5.94 (s, 1H), 5.38-5.34 (m, 1H), 3.71-3.64 (m, 1H), 3.30-3.28 (m, 1H), 3.23-3.20 (m, 1H), 3.04-2.95 (m, 2H), 2.38-2.33 (m, 4H), 2.06-1.91 (m, 5H), 1.73-1.68 (m, 1H). HRMS (ESI) m/z [M+H]⁺ calcd for C₂₂H₂₃F₄N₅O 450.1911, found 450.1916. HPLC: 96%.

### Example 57

### Synthesis of (S)-3-cyclobutyl-N-(5-methyl-3-trifluoromethylphenyl)-5-((piperidin-3-yl)oxy)pyrazolo[1,5-a]pyrimidin-7-amine (Compound I-66)

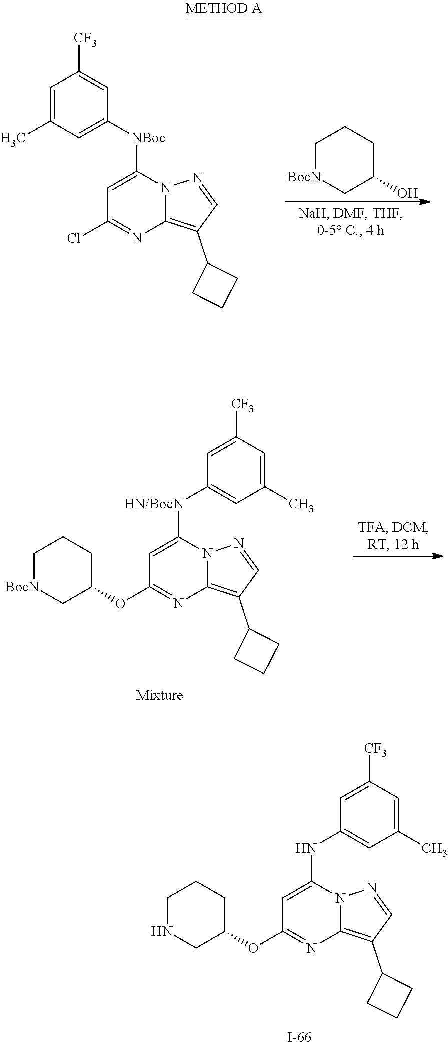

### Tert-Butyl (S)-3-((7-((tert-butoxycarbonyl)(5-methyl-3-trifluoromethylphenyl)amino)-3-cyclobutylpyrazolo[1,5-a]pyrimidin-5-yl)oxy)piperidine-1-carboxylate

To a stirred solution of NaH (10 mg, 0.24 mmol, 2.0 eq) in DMF (3 mL), the mixture of tert-butyl (5-chloro-3-cyclobutylpyrazolo[1,5-a]pyrimidin-7-yl)(5-methyl-3-trifluoromethylphenyl)carbamate, prepared according to Example 54 above (60 mg, 0.12 mmol, 1.0 eq), and (S)-1-Boc-3-hydroxypiperidine (30 mg, 0.15 mmol, 1.2 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl (S)-3-((7-((tert-butoxycarbonyl)(5-methyl-3-trifluoromethylphenyl)amino)-3-cyclobutylpyrazolo[1,5-a]pyrimidin-5-yl)oxy)piperidine-1-carboxylate as yellow oil (60 mg; 78%). TLC system: EtoAc:Hexane (1:4), R_{f} value: ˜0.5.

### (S)-3-Cyclobutyl-N-(5-methyl-3-trifluoromethylphenyl)-5-((piperidin-3-yl)oxy)pyrazolo[1,5-a]pyrimidin-7-amine (I-66)

To the solution of crude tert-butyl (S)-3-((7-((tert-butoxycarbonyl)(5-methyl-3-trifluoromethylphenyl)amino)-3-cyclobutylpyrazolo[1,5-a]pyrimidin-5-yl)oxy)piperidine-1-carboxylate (50 mg, 0.08 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-cyclobutyl-N-(5-methyl-3-trifluoromethylphenyl)-5-((piperidin-3-yl)oxy)pyrazolo[1,5-a]pyrimidin-7-amine (I-66) as a brown solid (14 mg, yield: 39%). TLC system: DCM/Methanol (2:1), R_{f} value: ˜0.6; ¹HNMR (500 Hz, MeOD-d4) δ 7.88 (s, 1H), 7.48 (s, 1H), 7.47 (s, 1H), 7.37 (s, 1H), 5.78 (s, 1H), 5.36-5.32 (m, 1H), 3.70-3.63 (m, 1H), 3.30-3.27 (m, 1H), 3.19-3.16 (m, 1H), 3.00-2.93 (m, 2H), 2.46 (s, 3H), 2.38-2.33 (m, 4H), 2.05-1.92 (m, 5H), 1.71-1.66 (m, 1H). HRMS (ESI) m/z [M+H]⁺ calcd for C₂₃H₂₆F₃N₅O 446.2162, found 446.2170. HPLC: 95%.

### Example 58

### Synthesis of (S)-3-((3-cyclopropyl-5-((piperidin-3-yl)amino)pyrazolo[1,5-a]pyrimidin-7-yl)amino)-5-methylbenzonitrile (Compound I-67)

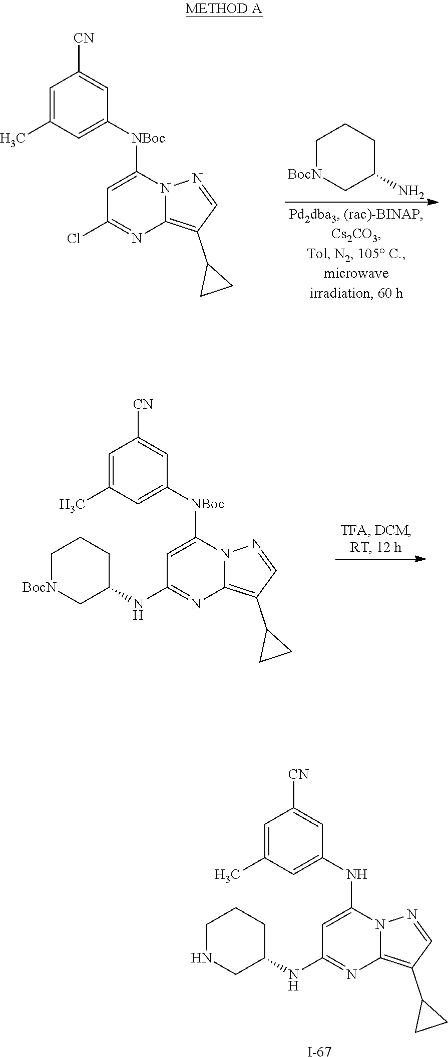

### Tert-Butyl (S)-3-((7-((tert-butoxycarbonyl)(3-cyano-5-methylphenyl)amino)-3-cyclopropylpyrazolo[1,5-a]pyrimidin-5-yl)amino)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclopropylpyrazolo[1,5-a]pyrimidin-7-yl)(3-cyano-5-methylphenyl)carbamate, prepared according to Example 45 above (89 mg, 0.21 mmol, 1 eq), (S)-1-Boc-3-aminopiperidine (46 mg, 0.23 mmol, 1.1 eq), and Cs₂CO₃ (137 mg, 0.42 mmol, 2 eq) were added at room temperature. The reaction mixture was under nitrogen flush for 5 minutes and then the microwave vial was sealed and heated at 105° C. for 60 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (S)-3-((7-((tert-butoxycarbonyl)(3-cyano-5-methylphenyl)amino)-3-cyclopropylpyrazolo[1,5-a]pyrimidin-5-yl)amino)piperidine-1-carboxylate as a brown oil (50 mg, 41%). TLC system: EOAc/hexane (1:2), R_{f} value: ˜0.3.

### (S)-3-((3-Cyclopropyl-5-((piperidin-3-yl)amino)pyrazolo[1,5-a]pyrimidin-7-yl)amino)-5-methylbenzonitrile (I-67)

To the solution of crude tert-butyl (S)-3-((7-((tert-butoxycarbonyl)(3-cyano-5-methylphenyl)amino)-3-cyclopropylpyrazolo[1,5-a]pyrimidin-5-yl)amino)piperidine-1-carboxylate (40 mg, 0.07 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-((3-cyclopropyl-5-((piperidin-3-yl)amino)pyrazolo[1,5-a]pyrimidin-7-yl)amino)-5-methylbenzonitrile (I-67) as a brown solid (13 mg, yield: 48%). TLC system: DCM/Methanol (2:1), R_{f} value: ˜0.3; ¹HNMR (500 Hz, MeOD-d4) δ 7.59 (s, 1H), 7.52 (s, 1H), 7.49 (s, 1H), 7.36 (s, 1H), 5.80 (s, 1H), 4.29-4.24 (m, 1H), 3.63-3.60 (m, 1H), 3.29-3.25 (m, 1H), 3.05-2.99 (m, 2H), 2.42 (s, 3H), 2.14-2.05 (m, 2H), 1.90-1.82 (m, 2H), 1.72-1.66 (m, 1H), 0.86-0.74 (m, 4H). HRMS (ESI) m/z [M+H]⁺ calcd for C₂₂H₂₅N₇ 388.2244, found 388.2254. HPLC: 95%.

### Example 59

### Synthesis of (S)-3-((3-cyclobutyl-5-((piperidin-3-yl)amino)pyrazolo[1,5-a]pyrimidin-7-yl)amino)-5-methylbenzonitrile (Compound I-68)

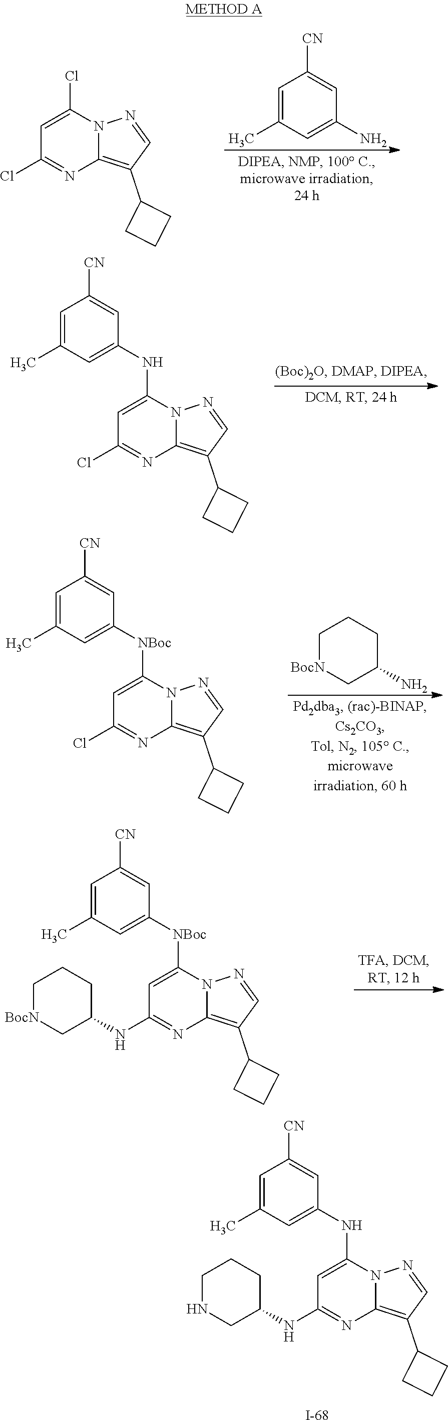

### 5-Chloro-N-(3-cyano-5-methylphenyl)-3-cyclobutylpyrazolo[1,5-a]pyrimidin-7-amine

To a stirred solution of 3-cyano-5-methylaniline (73 mg, 0.55 mmol, 1.1 eq) in NMP (4 mL) was added DIPEA (84 mg, 0.65 mmol, 1.3 eq) and stirred for 5 min. To this mixture 5,7-dichloro-3-cyclobutylpyrazolo[1,5-a]pyrimidine (121 mg, 0.50 mmol, 1.0 eq) was added at room temperature and continued stirring at 100° C. for 24 h under microwave irradiation. After completion of reaction by TLC, the reaction mixture was diluted with ice cold water and extracted with (3×30 mL) ethyl acetate. The combined organic layer was washed with water (50 mL), brine solution (20 mL), dried over sodium sulfate, and concentrated. The crude compound without purification went to the next step, 5-chloro-N-(3-cyano-5-methylphenyl)-3-cyclobutylpyrazolo[1,5-a]pyrimidin-7-amine as a brown liquid. TLC system: EA:Hexane (1:4), R_{f} value: ˜0.2.

### Tert-Butyl (5-chloro-3-cyclobutylpyrazolo[1,5-a]pyrimidin-7-yl)(3-cyano-5-methylphenyl)carbamate

To a stirred solution of 5-chloro-N-(3-cyano-5-methylphenyl)-3-cyclobutylpyrazolo[1,5-a]pyrimidin-7-amine (169 mg, 0.50 mmol, 1.0 eq) in DCM (5 mL) at room temperature was added DIPEA (129 mg, 1.00 mmol, 2 eq) and DMAP (3 mg 0.025 mmol), followed by addition of (Boc)₂O (142 mg, 0.65 mmol, 1.3 eq). The reaction was continued at room temperature for 24 h. Then, (Boc)₂O (142 mg, 0.65 mmol, 1.3 eq) was added again to the reaction. After completion of reaction monitored by TLC, the reaction mixture was quenched with sodium bicarbonate in water and extracted with (2×30 mL) DCM. The combined organic layer was washed with water (20 mL), brine solution (20 mL), dried over sodium sulfate, and concentrated. The crude compound was purified by combiflash chromatography (4 g column) to afford tert-butyl (5-chloro-3-cyclobutylpyrazolo[1,5-a]pyrimidin-7-yl)(3-cyano-5-methylphenyl)carbamate as a brown oil (150 mg, 68% for two steps). TLC system: EtOAc/hexane (1:4), R_{f} value: ˜0.2; 1HNMR (500 Hz, CDCl₃) δ 8.09 (s, 1H), 7.42 (s, 1H), 7.36 (s, 1H), 7.35 (s, 1H), 6.63 (s, 1H), 3.88-3.80 (m, 1H), 2.48-2.42 (m, 2H), 2.37 (s, 3H), 2.33-2.26 (m, 2H), 2.11-2.06 (m, 1H), 2.00-1.94 (m, 1H), 1.37 (s, 9H).

### Tert-Butyl (S)-3-((7-((tert-butoxycarbonyl)(3-cyano-5-methylphenyl)amino)-3-cyclobutylpyrazolo[1,5-a]pyrimidin-5-yl)amino)piperidine-1-carboxylate

To a stirred solution of Tris(dibenzylideneacetone)dipalladium (2 mg, 0.002 mmol) and rac-BINAP (5 mg, 0.008 mmol) in toluene (3 mL) in a microwave reaction vial tert-butyl (5-chloro-3-cyclobutylpyrazolo[1,5-a]pyrimidin-7-yl)(3-cyano-5-methylphenyl)carbamate (92 mg, 0.21 mmol, 1 eq), (S)-1-Boc-3-aminopiperidine (46 mg, 0.23 mmol, 1.1 eq) and Cs₂CO₃ (137 mg, 0.42 mmol, 2 eq) were added at room temperature. The reaction mixture was under nitrogen flush for 5 minutes and then the microwave vial was sealed and heated at 105° C. for 60 h. Then the reaction mixture was diluted with ethyl acetate and water and then filtered with celite. The filtrate was extracted with (2×30 mL) ethyl acetate. The combined organic layer was washed with brine solution (30 mL), dried over sodium sulfate, and concentrated to provide crude product which was purified by using combiflash chromatography (4 g column) to afford crude tert-butyl (S)-3-((7-((tert-butoxycarbonyl)(3-cyano-5-methylphenyl)amino)-3-cyclobutylpyrazolo[1,5-a]pyrimidin-5-yl)amino)piperidine-1-carboxylate as a brown oil (60 mg, 48%). TLC system: EOAc/hexane (1:2), R_{f} value: ˜0.2.

### (S)-3-((3-Cyclobutyl-5-((piperidin-3-yl)amino)pyrazolo[1,5-a]pyrimidin-7-yl)amino)-5-methylbenzonitrile (I-68)

To a solution of crude tert-butyl (S)-3-((7-((tert-butoxycarbonyl)(3-cyano-5-methylphenyl)amino)-3-cyclobutylpyrazolo[1,5-a]pyrimidin-5-yl)amino)piperidine-1-carboxylate (40 mg, 0.07 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-((3-cyclobutyl-5-((piperidin-3-yl)amino)pyrazolo[1,5-a]pyrimidin-7-yl)amino)-5-methylbenzonitrile (I-68) as a brown solid (25 mg, yield: 89%). TLC system: DCM/Methanol (2:1), R_{f} value: ˜0.2; ¹HNMR (500 Hz, MeOD-d4) δ 7.76 (s, 1H), 7.48 (s, 1H), 7.45 (s, 1H), 7.32 (s, 1H), 5.79 (s, 1H, the area is less than 0.5), 4.22-4.17 (m, 1H), 3.67-3.61 (m, 1H), 3.54-3.51 (m, 1H), 3.19-3.14 (m, 1H), 2.93-2.88 (m, 2H), 2.39 (s, 3H), 2.34-2.28 (m, 4H), 2.11-2.07 (m, 1H), 2.02-1.91 (m, 3H), 1.82-1.75 (m, 1H), 1.67-1.59 (m, 1H). HRMS (ESI) m/z [M+H]⁺ calcd for C₂₃H₂₇N₇ 402.2401, found 402.2413. HPLC: 90%.

### Example 60

### Synthesis of (S)-3-((3-cyclopropyl-5-((piperidin-3-yl)oxy)pyrazolo[1,5-a]pyrimidin-7-yl)amino)-5-methylbenzonitrile (Compound I-69)

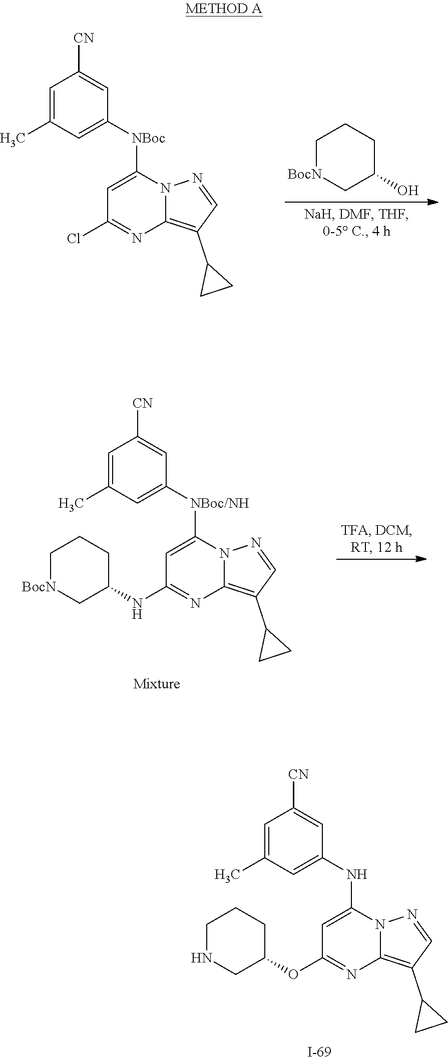

### Tert-Butyl (S)-3-((7-((tert-butoxycarbonyl)(3-cyano-5-methylphenyl)amino)-3-cyclopropylpyrazolo[1,5-a]pyrimidin-5-yl)oxy)piperidine-1-carboxylate

To a stirred solution of NaH (16 mg, 0.40 mmol, 2.0 eq) in DMF (3 mL), the mixture tert-butyl (5-chloro-3-cyclopropylpyrazolo[1,5-a]pyrimidin-7-yl)(3-cyano-5-methylphenyl)carbamate, prepared according to Example 45 above (85 mg, 0.20 mmol, 1.0 eq), and (S)-1-Boc-3-hydroxypiperidine (48 mg, 0.24 mmol, 1.2 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl (S)-3-((7-((tert-butoxycarbonyl)(3-cyano-5-methylphenyl)amino)-3-cyclopropylpyrazolo[1,5-a]pyrimidin-5-yl)oxy)piperidine-1-carboxylate as yellow oil (40 mg; 33%). TLC system: EtoAc:Hexane (1:4), R_{f} value: ˜0.4.

### (S)-3-((3-Cyclopropyl-5-((piperidin-3-yl)oxy)pyrazolo[1,5-a]pyrimidin-7-yl)amino)-5-methylbenzonitrile (I-69)

To a solution of crude tert-butyl (S)-3-((7-((tert-butoxycarbonyl)(3-cyano-5-methylphenyl)amino)-3-cyclopropylpyrazolo[1,5-a]pyrimidin-5-yl)oxy)piperidine-1-carboxylate (60 mg, 0.10 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-((3-cyclopropyl-5-((piperidin-3-yl)oxy)pyrazolo[1,5-a]pyrimidin-7-yl)amino)-5-methylbenzonitrile (I-69) as a brown solid (18 mg, yield: 46%). TLC system: DCM/Methanol (2:1), R_{f} value: ˜0.3; ¹HNMR (500 Hz, MeOD-d4) δ 7.72 (s, 1H), 7.53 (s, 1H), 7.50 (s, 1H), 7.39 (s, 1H), 5.81 (s, 1H, the area is less than 0.5), 5.34-5.31 (m, 1H), 3.29-3.25 (m, 1H), 3.19-3.15 (m, 1H), 3.02-2.94 (m, 2H), 2.42 (s, 3H), 2.03-1.94 (m, 3H), 1.90-1.86 (m, 1H), 1.72-1.66 (m, 1H), 0.89-0.81 (m, 4H). HRMS (ESI) m/z [M+H]⁺ calcd for C₂₂H₂₄N₆O 389.2084, found 389.2091. HPLC: 96%.

### Example 61

### Synthesis of (S)-3-((3-cyclobutyl-5-((piperidin-3-yl)oxy)pyrazolo[1,5-a]pyrimidin-7-yl)amino)-5-methylbenzonitrile (Compound I-70)

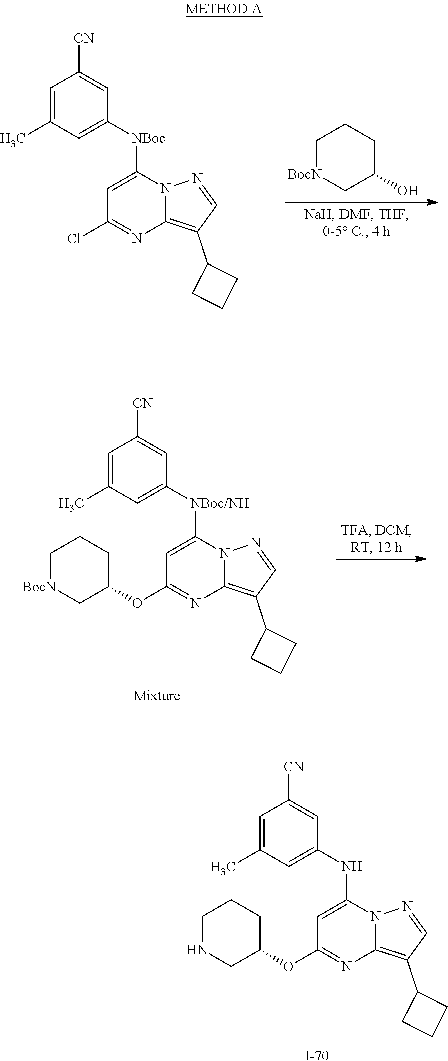

### Tert-Butyl (S)-3-((7-((tert-butoxycarbonyl)(3-cyano-5-methylphenyl)amino)-3-cyclobutylpyrazolo[1,5-a]pyrimidin-5-yl)oxy)piperidine-1-carboxylate

To a stirred solution of NaH (16 mg, 0.40 mmol, 2.0 eq) in DMF (3 mL), the mixture of tert-butyl (5-chloro-3-cyclobutylpyrazolo[1,5-a]pyrimidin-7-yl)(3-cyano-5-methylphenyl)carbamate, prepared according to Example 59 above (88 mg, 0.20 mmol, 1.0 eq), and (S)-1-Boc-3-hydroxypiperidine (48 mg, 0.24 mmol, 1.2 eq) in THF (3 mL) was added at 0-5° C. and stirred at 0-5° C. for 4 h. After completion of reaction by TLC, the reaction mixture was quenched with ice cold water and extracted with (3×50 mL) ethyl acetate. The combined organic layer was washed with water (30 mL), brine solution (30 mL), dried over sodium sulfate, and concentrated to afford product which was purified by using combiflash chromatography (4 g column) to afford crude tert-butyl (S)-3-((7-((tert-butoxycarbonyl)(3-cyano-5-methylphenyl)amino)-3-cyclobutylpyrazolo[1,5-a]pyrimidin-5-yl)oxy)piperidine-1-carboxylate as yellow oil (40 mg; 33%). TLC system: EtoAc:Hexane (1:4), R_{f} value: ˜0.4.

### (S)-3-((3-Cyclobutyl-5-((piperidin-3-yl)oxy)pyrazolo[1,5-a]pyrimidin-7-yl)amino)-5-methylbenzonitrile (I-70)

To the solution of crude tert-butyl (S)-3-((7-((tert-butoxycarbonyl)(3-cyano-5-methylphenyl)amino)-3-cyclobutylpyrazolo[1,5-a]pyrimidin-5-yl)oxy)piperidine-1-carboxylate (30 mg, 0.05 mmol, 1.0 eq) in DCM (2 mL) and TFA (1 mL) was added. The reaction mixture was stirred at room temperature for 12 h. After completion of reaction by TLC, the reaction mixture was concentrated. To the concentrated reaction sodium bicarbonate in water was added and stirred for 30 minutes at 0-5° C. Then the reaction mixture was extracted with (2×30 mL) ethyl acetate. The combined organic layer was washed with water (30 mL), brine solution (30 mL), dried over sodium sulfate, and concentrated to provide crude product which was purified by using combiflash chromatography (4 g column) to afford (S)-3-((3-cyclobutyl-5-((piperidin-3-yl)oxy)pyrazolo[1,5-a]pyrimidin-7-yl)amino)-5-methylbenzonitrile (I-70) as an off white solid (18 mg, yield: 90%). TLC system: DCM/Methanol (2:1), R_{f} value: ˜0.3; ¹HNMR (500 Hz, MeOD-d4) δ 7.87 (s, 1H), 7.53 (s, 1H), 7.50 (s, 1H), 7.38 (s, 1H), 5.80 (s, 1H, the area is less than 0.5), 5.36-5.33 (m, 1H), 3.69-3.62 (m, 1H), 3.30-3.27 (m, 1H), 3.23-3.19 (m, 1H), 3.03-2.97 (m, 2H), 2.42 (s, 3H), 2.37-2.32 (m, 4H), 2.05-1.93 (m, 5H), 1.72-1.68 (m, 1H). HRMS (ESI) m/z [M+H]⁺ calcd for C₂₃H₂₆N₆O 403.22461, found 403.2248. HPLC: 96%.

### Example 62

### Biochemical Assay 1

### Kinase ADP-Glo Assay

### Materials:

Assay Buffer: 50 mM HEPES, 3 mM MgCl₂, 3 mM MnCl₂, 1 mM DTT, 3 μM Na₃VO₄, pH 7.5

Substrate RB: CTF Peptide (ProQinase #: 0040-0000-6)

Enzymes: CDK2/CycE1 (ProQinase #: 0050-0055-1), CDK5/p25NCK (ProQinase #: 0356-0389-1), CDK7/CycH/MAT1 (ProQinase #: 0366-0360-4)

ADP-Glo™ Assay (Promega #: V9101): ATP: 10 mM, ADP-Glo™ Reagent, Kinase Detection Reagent

384-well white assay plates

### Method:

A ten point serial dilution of compound was prepared at 5× concentration in assay buffer with the final assay concentrations starting at 30 μM, 10 μM, 3 μM, 1 μM . . . 0 μM. Enzyme, substrate, and ATP were used at 40 ng, 200 ng (or 400 ng)⁺ and 1 μM, respectively. The assay plate was set up by mixing the components in a total reaction volume of 10 μL per well. The plate was centrifuged gently for 10 seconds and incubated at room temperature (RT) for 60 minutes in the dark. The ADP-Glo Reagent and kinase detection reagent were added and incubated as recommended. The reaction was quantified by measuring luminescence on the Perkin Elmer Envision plate reader. Data was analyzed using Graphpad Prism 7 software. *With CDK2 enzyme, 400 ng of substrate was used. Results for exemplary compounds are provided in Table 2 below.

### Kinase Glo Assay

### Materials:

Assay Buffer: 50 mM HEPES, 3 mM MgCl₂, 3 mM MnCl₂, 1 mM DTT, 3 μM Na₃VO₄, pH 7.5

Enzymes: CDK7/CycH/MAT1 (ProQinase #: 0366-0360-4)

Kinase-Glo™ Assay (Promega #: V6711): ATP: 10 mM, Kinase-Glo™ Reagent 384-well white assay plates

### Method:

A ten point serial dilution of compound was prepared at 5× concentration in assay buffer with the final assay concentrations starting at 30 μM, 10 μM, 3 μM, 1 μM . . . 0 μM. Enzyme and ATP were used at 40 ng and 1 μM, respectively. The assay plate was set up by mixing the components in a total reaction volume of 10 μL per well. The plate was centrifuged gently for 10 seconds and incubated at room temperature (RT) for 60 minutes in the dark. The Kinase-Glo Reagent was added and incubated as recommended. The reaction was quantified by measuring luminescence on the Perkin Elmer Envision plate reader. Data was analyzed using Graphpad Prism 7 software. Results for exemplary compounds of the of structure (I) are provided in Table 2 below.

TABLE 2
List of compounds and IC₅₀ values in nM for CDK7

| Compound | CDK7 |
|---|---|
| I-1* | ++ |
| I-2* | +++ |
| I-3* | + |
| I-4 | +++ |
| I-5* | + |
| I-6 | ++ |
| I-7 | ++ |
| I-8 | +++ |
| I-9 | +++ |
| I-10 | ++ |
| I-11 | +++ |
| I-12 | ++ |
| I-13 | + |
| I-14 | ++ |
| I-15 | ++ |
| I-16 | + |
| I-17 | ++ |
| I-18 | +++ |
| I-19* | ++ |
| I-20 | − |
| I-21 | + |
| I-22 | + |
| I-23* | ++ |
| I-24* | + |
| I-25 | − |
| I-26* | ++ |
| I-27* | ++ |
| I-28* | ++ |
| I-29 | ++ |
| I-30 | ++ |
| I-31* | − |
| I-32* | ++ |
| I-33* | ++ |
| I-45 | +++ |
| I-46 | ++ |
| I-47 | ++ |
| I-48 | +++ |
| I-49 | +++ |
| I-50 | +++ |
| I-51 | +++ |
| I-52 | +++ |
| I-53 | +++ |
| I-54 | +++ |
| I-55 | +++ |
| I-56 | ++ |
| I-57 | +++ |
| I-58 | +++ |
| I-59 | + |
| I-60 | + |
| I-61 | ++ |
| I-62 | ++ |
| I-63 | +++ |
| I-64 | ++ |
| I-65 | +++ |
| I-66 | +++ |
| I-67 | ++ |
| I-68 | +++ |
| I-69 | +++ |
| I-70 | ++++ |

+++ indicates an IC₅₀ value up to 100 nM
++ indicates an IC₅₀ value from 100 to 1,000 nM
+ indicates an IC₅₀ value greater than 1,000 nM
− indicates no data
*Kinase-Glo Assay was used

### Example 63

### Biochemical Assay 2

### Recombinant RNA Polymeriase H Phosphorylation Assay

### Materials:

Assay Buffer: 50 mM HEPES, 3 mM MgCl₂, 3 mM MnCl₂, 1 mM DTT, 3 μM Na₃VO₄, pH 7.5

ATP: 10 mM (Promega #: V915A)

Enzyme: CDK7/CycH/MAT1 (ProQinase #: 0366-0360-4)

Substrate: Recombinant RNA Pol II-CTD protein (Active Motif #: 81036)

Antibodies: Phospho-Rpb1 CTD [Ser2] (Cell Signaling Technology #: 13499S),

Phospho-Rpb1 CTD [Ser5] (Active Motif #: 39233), CDK7 (Santa Cruz #: sc-56284).

### Method:

The compounds were tested at final assay concentrations of 1 μM, 0.1 μM and 0.01 μM with the appropriate negative controls. CDK7 enzyme, RNA Pol II substrate and ATP were used at 80 ng, 10 ng and 1 μM, respectively. The assay was set up by mixing the components in a total reaction volume of 40 μL per tube. The tubes were centrifuged and incubated at 30° C. for 30 minutes. Samples were processed by Western Blotting for the phosphorylated RNA Pol II and imaged on the LiCor Odyssey.

### Protein Quantification by Western Blotting

### Materials:

Cell Lines (Treated according to the ATCC guidelines)

DMSO

6-well Tissue Culture Treated Plates

Antibodies: C-MYC Antibody (Cell Signalling Cat #9402S), MCL-1 Antibody (Cell Signalling Cat #94296S), RNA Pol II Ser 5 Antibody (Active Motif Cat #39233)

### Method:

Cells were seeded in a 6-well tissue culture treated plate at a density of 500,000 cells/well and allowed to settle overnight at 37° C., 5% CO2. Next day, the media was removed and replaced with new media containing drug at the desired concentration, including a DMSO control. The cells were incubated in the presence of drug for 24 hours at 37° C., 5% CO2. After incubation the media was removed, cells were washed and lysed with NP40 lysis buffer at 4° C. for 1 hour. The resulting protein was quantified and 15 μg of protein/well was resolved on a 4-12% Bis-Tris gel. Following Western Blotting, the bands were imaged and quantified using the LI-COR Odyssey CLX Fluorescence Imager.

### Cell Viability Assay

### Materials:

Cell Lines (Treated according to ATCC guidelines)

Cell Titer Glo (Promega Cat #G7572)

DMSO

96-well White Tissue Culture Treated Plates (Perkin Elmer Cat #60005680)

### Method:

Cells were seeded in a white 96-well tissue culture treated plate at a density of 1500 cells in 90 μL of media per well and allowed to settle overnight. A ten point serial dilution of drugs was prepared at 10× concentration in media with the final assay concentrations starting at 30 μM, 10 μM, 3 μM, 1 μM, 0.3 μM . . . 0 μM. A dilution of DMSO was included as a control. The assay was set up by adding 10 μL of the corresponding drug to each well in duplicate, followed by a 72 hour incubation at 37° C., 5% CO2. Cell viability was quantified by adding 90 μL of Cell Titer Glo to each well and incubating at room temperature for 10 minutes. The reaction was quantified by measuring luminescence using the Envision Plate Reader. Data was analyzed using Graphpad Prism 7 software.

All of the U.S. patents, U.S. patent application publications, U.S. patent applications, foreign patents, foreign patent applications and non-patent publications referred to in this specification or the attached Application Data Sheet are incorporated herein by reference, in their entirety to the extent not inconsistent with the present description.

U.S. Provisional Application 62/818,037, filed Mar. 13, 2019 is incorporated herein by reference, in its entirety.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

1. A compound having the following structure (1): 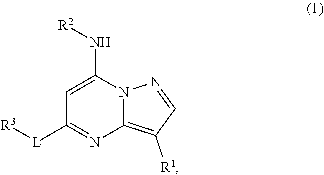
or stereoisomer, tautomer, prodrug, or pharmaceutically acceptable salt thereof, wherein:
R¹ is cycloalkyl, chloro, or cyano;
R² is aryl, or arylalkyl;
R³ is cycloalkyl or heterocyclyl;
L is —O(CH₂)ₙ— or —NH(CR^{a}R^{b})ⁿ— wherein R^{a} and R^{b} are both hydrogen or R^{a} and R^{b} join together with the carbon to which they are attached to form oxo; and
n is 0 or 1,
wherein,
each cycloalkyl is independently unsubstituted or substituted with one or more substituents selected from halo, hydroxyl, hydroxyalkyl, amino, and trialkylsilyl when R¹ is cycloalkyl and each cycloalkyl is independently unsubstituted or substituted with one or more substituents selected from hydroxyl, hydroxyalkyl, and trialkylsilyl when R¹ is chloro or cyano, and
each heterocyclyl, aryl, and arylalkyl is independently unsubstituted or substituted with one or more substituents selected from alkyl, alkenyl, alkynyl, halo, haloalkyl, alkoxy, haloalkoxy, cyano, hydroxyl, hydroxyalkyl, carboxy, heteroaryl, heterocyclyl, amino, —S(O₂)NH₂, —S(O₂)alkyl, —S(O₂)cycloalkyl, and trialkylsilyl.

2. The compound of claim 1, wherein R¹ is chloro, cyano, cyclopropyl, or cyclobutyl.

3. The compound of any one of claim 1 or 2 having one of the following structures (Ia), (Ib), (Ic), or (Id):

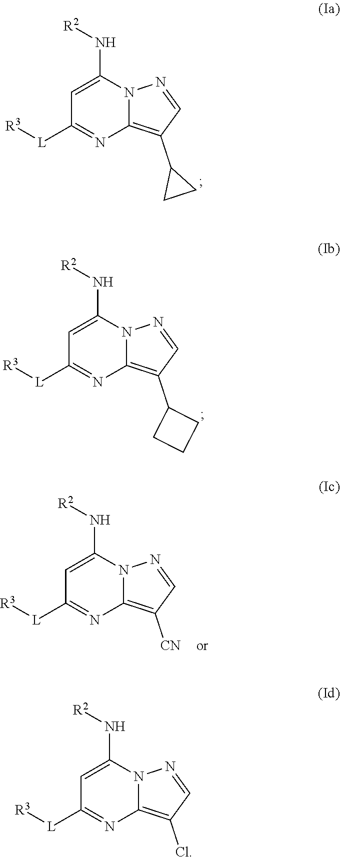

4. The compound of any one of claims 1-3, wherein R² is arylalkyl.

5. The compound of claim 4, wherein R² is benzyl.

6. The compound of any one of claim 4 or 5, wherein R² is substituted.

7. The compound of any one of claims 4-6, wherein R² is substituted with one or more substituents selected from the group consisting of halo and alkyl.

8. The compound of claim 7, wherein R² is substituted with one or more substituents selected from the group consisting of fluoro, chloro, and methyl.

9. The compound of any one of claims 4-8, wherein R² has one of the following structures:

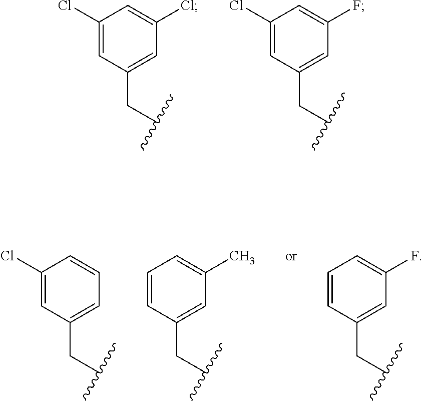

10. The compound of any one of claims 1-3, wherein R² is aryl.

11. The compound of claim 10, wherein R² is phenyl.

12. The compound of any one of claim 10 or 11, wherein R² is substituted.

13. The compound of any one of claims 10-12, wherein R² is substituted with one or more substituents selected from the group consisting of alkyl, halo, haloalkyl, cyano, —S(O₂)NH₂, and —S(O₂)alkyl.

14. The compound of claim 13, wherein R² is substituted with one or more substituents selected from the group consisting of fluoro, chloro, methyl, trifluoromethyl, trifluoroethyl, cyano,

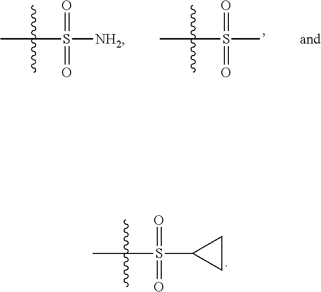

15. The compound of any one of claims 4-8, wherein R² has one of the following structures:

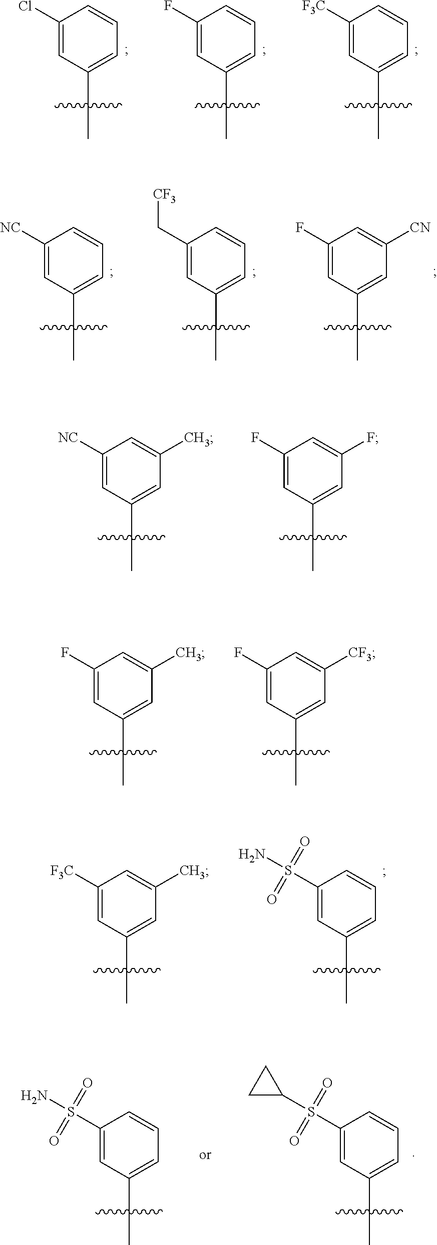

16. The compound of any one of claims 1-15, wherein R³ is heterocyclyl.

17. The compound of claim 16, wherein R³ is piperidinyl, azepinyl, or tetrahydropyranyl.

18. The compound of any one of claim 16 or 17, wherein R³ is unsubstituted.

19. The compound of any one of claims 16-18, wherein R³ has one of the following structures:

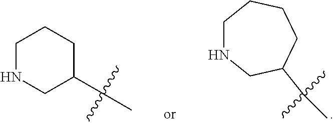 [SMILES: *C1CCCNC1 ; *C1CCCCNC1]

20. The compound of claim 16 or 17, wherein R³ is substituted.

21. The compound of any one of claim 16 or 20, wherein R³ is substituted with one or more substituents selected from the group consisting of hydroxyl and hydroxyalkyl.

22. The compound of any one of claims 16, 20, or 21, wherein R³ has one of the following structures:

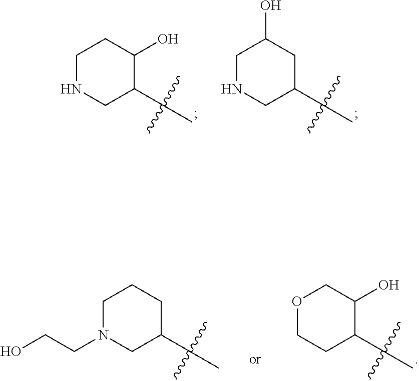 [SMILES: *C1CNCCC1O ; *C1CNCC(O)C1 ; *C1CCCN(CCO)C1 ; *C1CCOCC1O]

23. The compound of any one of claims 1-15, wherein R³ is cycloalkyl.

24. The compound of claim 23, wherein R³ is substituted.

25. The compound of any one of claim 23 or 24, wherein R³ is cyclohexyl or cyclobutyl.

26. The compound of any one of claims 23-25, wherein R³ is substituted with one or more substituents selected from the group consisting of amino and trimethylsilyl.

27. The compound of any one of claims 23-26, wherein R³ has one of the following structures:

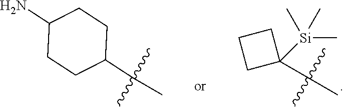

28. The compound of any one of claims 1-27, wherein L is —NH—, —N(H)CH₂— or —N(C═O)—.

29. The compound of any one of claims 1-27, wherein L is —O— or —OCH₂—.

30. The compound of claim 1 having one of the following structures (Ia′) or (Ia″): 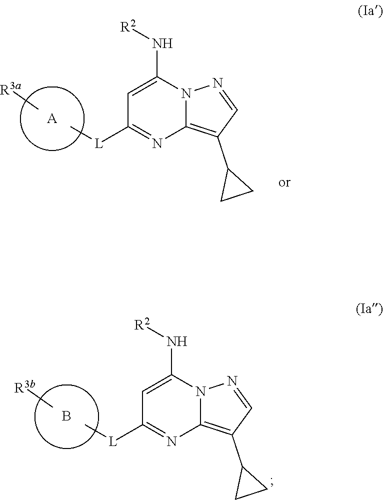
wherein
A is cycloalkyl;
B is heterocyclyl;
R^{3a} is hydrogen, hydroxyl, hydroxyalkyl, or trialkylsilyl; and
R^{3b} is hydrogen, hydroxyl, hydroxyalkyl, amine, and trialkylsilyl.

31. The compound of claim 30 having one of the following structures (Ia1), (Ia2), (Ia3), (Ia4), (Ia5), or (Ia6):

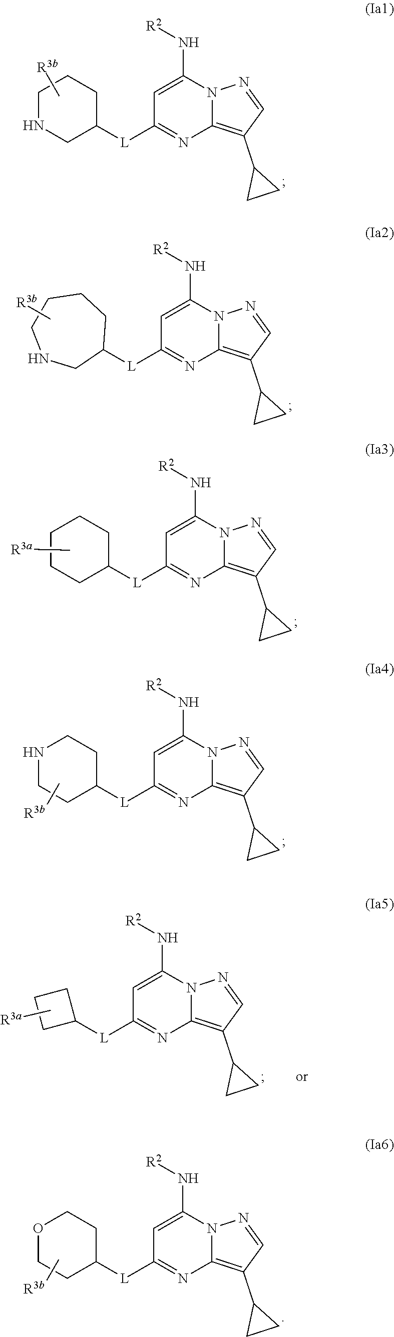

32. The compound of claim 1 having one of the following structures (Ib1), (Ib2), (Ib3), or (Ib4): 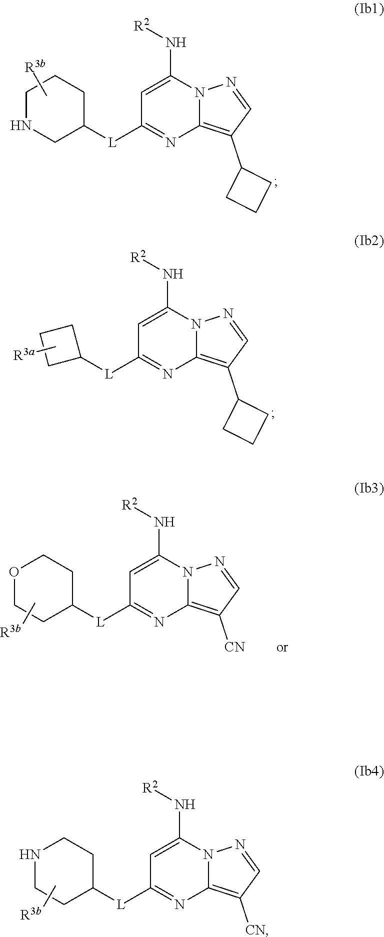
wherein
R^{3a} is hydrogen, hydroxyl, hydroxyalkyl, or trialkylsilyl; and
R^{3b} is hydrogen, hydroxyl, hydroxyalkyl, amine, and trialkylsilyl.

33. The compound of claim 1 having the following structure (Ic1): 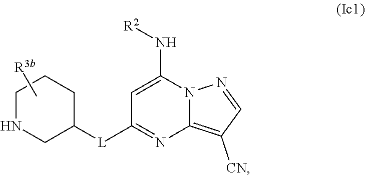
wherein:
R^{3b} is hydrogen, hydroxyl, hydroxyalkyl, amine, and trialkylsilyl.

34. The compound of claim 1 having one of the following structures (Id1) or (Id2):

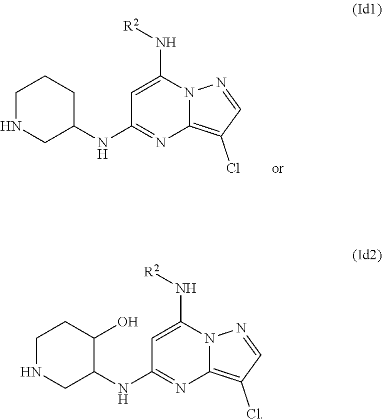

35. A compound selected from Table 1 or stereoisomer, tautomer, prodrug, or pharmaceutically acceptable salt thereof.

36. A pharmaceutically acceptable salt of any one of the compounds according to any one of claims 1-35.

37. The pharmaceutically acceptable salt of claim 36, wherein the pharmaceutically acceptable salt is an acid addition salt.

38. The pharmaceutically acceptable salt of claim 37, wherein the acid addition salt is a trifluoroacetic acid salt or a hydrochloric acid salt.

39. A composition comprising any one of the compounds of claims 1-35 or a pharmaceutically acceptable salt of any one of claims 36-38 and a pharmaceutically acceptable carrier or excipient.

40. A method of treating a CDK7-dependent disease, the method comprising administering a compound of any one of claims 1-35, a pharmaceutically acceptable salt of any one of claims 36-38, or a composition of claim 39 to a mammal in need thereof.

41. The method of claim 40, wherein the CDK7-dependent disease is cancer.

42. The method of claim 41, wherein the cancer is pancreatic cancer.

43. The method of claim 41, wherein the cancer is breast cancer.

44. The method of claim 43, wherein the breast cancer is triple negative breast cancer.

45. The method of claim 41, wherein the cancer is neuroblastoma, medulloblastoma, Ewing sarcoma, chordoma, or combinations thereof.

46. The method of any one of claims 40-45 further comprising administering an additional therapeutic agent selected from the group consisting of gemcitabine, cisplatin, 5-fluorouracil, nutlin, panobinostat, olaparib, and combinations thereof.
